(19) United States
(12) Patent Application Publication
(10) Pub. No.: US 2008/0020984 A1
(43) Pub. Date: Jan. 24, 2008

(54) Crystal Structure of a Receptor-Ligand Complex and Methods of Use

(75) Inventors: Peter Kuhn, Solana Beach, CA (US); Anand Kolatkar, San Diego, CA (US); Alexei Brooun, San Diego, CA (US); Jill Chrencik, San Diego, CA (US); Michelle Kraus, Temecula, CA (US)

Correspondence Address: BIOTACTICA, LLC, 10733 Sunset Office Drive, Suite 261, ST. LOUIS, MO, 63127, US

(73) Assignee: The Scripps Research Institute, La Jolla, CA (US)

(21) Appl. No.: 11/781,738

(22) Filed: Jul. 23, 2007

Related U.S. Application Data
(60) Provisional application No. 60/832,375, filed on Jul. 21, 2006.

(51) Int. Cl. G06G 7/48; A61K 38/00; C07K 1/00; C07K 14/00; C40B 30/04; G01N 33/00

(52) U.S. Cl. 514/13; 436/86; 506/9; 530/325; 530/345; 703/11

(57) ABSTRACT

The invention relates to the three-dimensional structure of a crystal of an EphB4 receptor complexed with a ligand. The three-dimensional structure of a Receptor-Ligand Complex is disclosed. The receptor-ligand crystal structure, wherein the ligand is an inhibitor molecule, is useful for providing structural information that may be integrated into drug screening and drug design processes. Thus, the invention also relates to methods for utilizing the crystal structure of the Receptor-Ligand Complex for identifying, designing, selecting, or testing inhibitors of the EphB4 receptor protein, such inhibitors being useful as therapeutics for the treatment or modulation of i) diseases; ii) disease symptoms; or iii) the effect of other physiological events mediated by the receptor.

### CROSS-REFERENCE TO RELATED APPLICATIONS

This application claims priority from U.S. Provisional Application Ser. No. 60/832,375 filed on Jul. 21, 2006, which is incorporated herein by reference in its entirety.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

The Sequence Listing, which is a part of the present disclosure, includes a computer file “10000-0027_ST25.txt” generated by U.S. Patent & Trademark Office Patent In Version 3.4 software comprising nucleotide and/or amino acid sequences of the present invention. The subject matter of the Sequence Listing is incorporated herein by reference in its entirety.

### FIELD

The present invention relates to a three-dimensional structure of a receptor tyrosine kinase from the erythropoietin-producing hepatocellular carcinoma family of receptor tyrosine kinases (“Eph”), particularly EphB4 or similar polypeptide complexed with an ephrinB2 or ephrinB2 analog (“Receptor-Ligand Complex”), three-dimensional coordinates of a Receptor-Ligand Complex, models thereof, and uses of such structures and models.

### INTRODUCTION

The Eph receptor tyrosine kinases and their ligands, the ephrins, regulate numerous biological processes in developing and adult tissues and have been implicated in cancer progression and in pathological forms of angiogenesis. For example, the Eph receptors and their ligands, the ephrins, play critical roles in angiogenesis during embryonic development as well as in adult tissues (Brantley-Sieders and Chen, 2004; Cheng et al., 2002; Gale and Yancopoulos, 1999; Kullander and Klein, 2002). The Eph family of receptor tyrosine kinases also regulates many other biological processes, including tissue patterning, axonal guidance, and as more recently discovered, tumorigenesis (Carmeliet and Collen, 1999; Ferrara, 1999; Pasquale, 2005; Wilkinson, 2000). Both the Eph receptor and the ephrin ligand are membrane bound, and therefore require cell-cell contact to signal a cellular response. The interaction between Eph receptors and ephrins on adjacent cell surfaces results in multimerization and clustering of the Eph-ephrin complexes, leading to forward signaling in the Eph-expressing cell and reverse signaling in the ephrin-expressing cell. EphB4 belongs to the Eph (erythropoietin-producing hepatocellular carcinoma) family of receptor tyrosine kinases, which is divided into two subclasses, A and B. based on binding preferences and sequence conservation (Gale et al., 1996). In general, EphA receptors (EphA1-EphA10) bind to glycosyl phosphatidyl inositol-(GPI) anchored ephrin-A ligands (ephrin-A1-ephrin-A6), while EphB receptors (EphB1-EphB6) interact with transmembrane ephrin-B ligands (ephrin-B1-ephrin-B3) (Eph Nomenclature Committee, 1997). While interactions between the Eph receptors and ephrin ligands of the same subclass are quite promiscuous, interactions between subclasses are rare. A few cross-subclass exceptions include the EphA4-ephrin-B2/B3 interactions (Takemoto et al., 2002), and the EphB2-ephrinA5 interaction, which has been characterized structurally (Himanen et al., 2004). EphB4 is unique within the Eph family in that it selectively binds ephrin-B2, while demonstrating only weak binding for both ephrin-B1 and ephrin-B3.

Eph receptors have a modular structure, consisting of an N-terminal ephrin binding domain adjacent to a cysteine-rich domain and two fibronectin type III repeats in the extracellular region. The intracellular region consists of a juxtamembrane domain, a conserved tyrosine kinase domain, a C-terminal sterile α-domain (SAM), and a PDZ binding motif. The N-terminal 180 amino acid globular domain is sufficient for high-affinity ligand binding (Himanen et al., 2001).

The EphB4-ephrinB2 interaction is important in angiogenesis and given that EphB4 is overexpressed in several tumor types (Dodelet, V. C., and Pasquale, E. B. (2000) Oncogene 19, 5614-5619; Nakamoto, M., and Bergemann, A. D. (2002) Microsc Res Tech 59, 58-67; Liu, W., Ahmad, S. A., Jung, Y. D., Reinmuth, N., Fan, F., Bucana, C. D., and Ellis, L. M. (2002) Cancer 94, 934-939; Berclaz, G., Karamitopoulou, E., Mazzucchelli, L., Rohrbach, V., Dreher, E., Ziemiecki, A., and Andres, A. C. (2003) Ann Oncol 14, 220-226), modulating this protein-protein interaction is a potential approach to slowing tumor angiogenesis and tumor growth. In mouse models of breast cancer, high EphB4 expression correlates with increased malignancy and tumor aggressiveness (Andres, A. C., Reid, H. H., Zurcher, G., Blaschke, R. J., Albrecht, D., and Ziemiecki, A. (1994) Oncogene 9, 1461-1467; Nikolova, Z., Djonov, V., Zuercher, G., Andres, A. C., and Ziemiecki, A. (1998) J Cell Sci 111 (Pt 18), 2741-2751; Munarini, N., Jager, R., Abderhalden, S., Zuercher, G., Rohrbach, V., Loercher, S., Pfanner-Meyer, B., Andres, A. C., and Ziemiecki, A. (2002) J Cell Sci 115, 25-37). EphB4 expression is also increased in human primary infiltrating ductal breast carcinoma and is correlated to increased malignancy (Berclaz, G., Andres, A. C., Albrecht, D., Dreher, E., Ziemiecki, A., Gusterson, B. A., and Crompton, M. R. (1996) Biochem Biophys Res Commun 226, 869-875). There is evidence that the EphB4 ectodomain stimulates endothelial cell migration and proliferation, suggesting that ephrinB2-expressing endothelial cells interact with the EphB4 ectodomain to promote angiogenesis and tumor progression. Furthermore, a kinase-deficient EphB4 mutant has been shown to increase breast cancer cell growth indicating that downstream forward kinase signaling is not an absolute requirement for tumorigenesis, at least in breast cancer cells (Noren, N. K., Lu, M., Freeman, A. L., Koolpe, M., and Pasquale, E. B. (2004) Proc Natl Acad Sci USA 101, 5583-5588). Several groups have more recently demonstrated that the full extracellular domain of EphB4 is indeed a viable therapeutic target First, the soluble extracellular domain of EphB4 was described to block both forward and reverse signaling, resulting in an inhibition of tumor growth in vivo (Kertesz, N., Krasnoperov, V., Reddy, R., Leshanski, L., Kumar, S. R., Zozulya, S., and Gill, P. S. (2006) Blood 107, 2330-2338; Martiny-Baron, G., Korff, T., Schaffner, F., Esser, N., Eggstein, S., Marme, D., and Augustin, H. G. (2004) Neoplasia 6, 248-257).

Second, phage display studies have identified a peptide (TNYL-RAW) which antagonizes the EphB4-ephrinB2 interaction in the high nanomolar range (Koolpe, M., Burgess, R., Dail, M., and Pasquale, E. B. (2005) J Biol Chem 280, 17301-17311). The crystal structure of the EphB4 receptor in complex with the phage-derived TNYL-RAW peptide (SEQ ID NO: 1) revealed that the peptide binds to the ephrin binding cavity of the receptor, effectively inhibiting interaction with the ligand (Chrencik, J. E., Brooun, A., Recht, M. I., Kraus, M. L., Koolpe, M., Kolatkar, A. R., Bruce, R. H., Martiny-Baron, G., Widmer, H., Pasquale, E. B., and Kuhn, P. (2006) Structure 14, 321-330). However, a more complete understanding of the biological role of EphB4-ephrinB2 signaling in tumorigenesis and in forms of pathological angiogenesis is now required.

Despite attempts to model the structural changes of EphB4 upon ligand binding, a detailed view of conformational arrangements of an EphB4 receptor in complex with ephrinB2 has remained elusive. Thus, the development of useful reagents for treatment or diagnosis of disease was hindered by lack of structural information of such a Receptor-Ligand Complex. Therefore, there is a need in the art to elucidate the three-dimensional structure and models of Receptor-Ligand Complexes, and to use such structures and models in therapeutic strategies, such as drug design.

### DRAWINGS

Those of skill in the art will understand that the drawings, described below, are for illustrative purposes only. The drawings are not intended to limit the scope of the present teachings in any way.

FIG. 1. The ephrin binding domain of the EphB4 receptor in complex with the ephrinB2 extracellular domain. The EphB4 receptor (right) consists of a jellyroll folding topology with 13 anti-parallel B-sheets connected by loops of varying lengths, whereas the ephrin ligand (left) is similar to the Greek key folding topology. The interface is formed by insertion of the ligand G-H loop into the hydrophobic binding cleft of EphB4.

FIG. 2. Stereoview of the superposition of the Eph receptor ligand binding domains from the EphB4.ephrinB2 (thick grey line), EphB2.ephrinB2 (thin grey line), and EphB4.TNYL-RAW complex structures (thick line with spheres). Clear deviation is seen at the J-K loop, whereas more minor changes are seen in the receptor D-E and G-H loops (Protein Data Bank code 1 KGY). The overall root mean square deviation between the EphB4.ephrinB2 and the EphB2.ephrinB2 and EphB4-TNYL-RAW structures is 5.0 and 2.5 Å, respectively. FIG. 3. Stereoview of σA weighted 2 F_{obs}-F_{calc} electron density at 2.0 Å resolution, contoured at 1σ for the EphB4.ephrinB2 interface. The ephrinB2 is the leftmost molecule (labeled) and the EphB4 is at the right (labeled). Clear density of the interface shows Phe-120 in a novel position with respect to previously described structures in order to interact with Leu-95.

FIG. 4. Detailed ligplot diagram of critical EphB4.ephrinB2 interactions. All interactions are less than 4 Å and are indicated by dashed lines. The ligand is depicted with all bonds shown, whereas receptor residues are drawn schematically.

FIG. 5. EphrinB2 specificity region in the EphB2/EphB4.ephrinB2 complexes. Left, the region near the EphB4 Leu-95R of the EphB4.ephrinB2 complex structure is shown in schematic representation. The van der Waals interaction between the ephrinB2 Phe-120L and the EphB4 Leu-95R is depicted as a dotted line. Right, the region near the EphB2 Arg-103R of the EphB2.ephrinB2 complex structure is shown in the same orientation as that on the left. The EphB2 Arg-103R, Ser-156R, and Ser-107R side chains are shown as grey sticks. Hydrogen bonds between Arg-103R and the two serines are shown as dotted lines. The J-K loops of EphB2 and EphB4 are labeled highlighting the change in loop position between the two complexes.

FIG. 6. This figure illustrates binding of fluorescent peptide to wild type EphB4, EphB4 K149Q (A) and EphB4 L95R mutants. Increasing amount of EphB4 protein was added to wells containing 75 nM of fluorescent TNYL-RAW peptide. Fluorescent polarization was measured at room temperature after 30 min of incubation. Based on the structure of EphB4-ephrin-B2 complex, the substitution of L95 was predicted to impair EphB4 binding to ephrin-B2.

FIG. 7. This figure illustrates determination of Kᵢ for TNYL-RAW. Kᵢ were determined for both wild-type EphB4 (filled triangles) and EphB4 (K149Q) mutant (filled squares).

FIG. 8. This figure illustrates binding of fluorescent TNYL-RAW peptide in the presence of increasing concentration of DMSO. Increasing amounts of EphB4 protein were added to wells containing 75 nM of TNYL-RAW-Alexa-532 peptide. Fluorescent polarization was measured at room temperature.

FIG. 9. This figure illustrates Z-factor determination for EphB4-Alexa-532-TNYL-RAW fluorescent polarization assay.

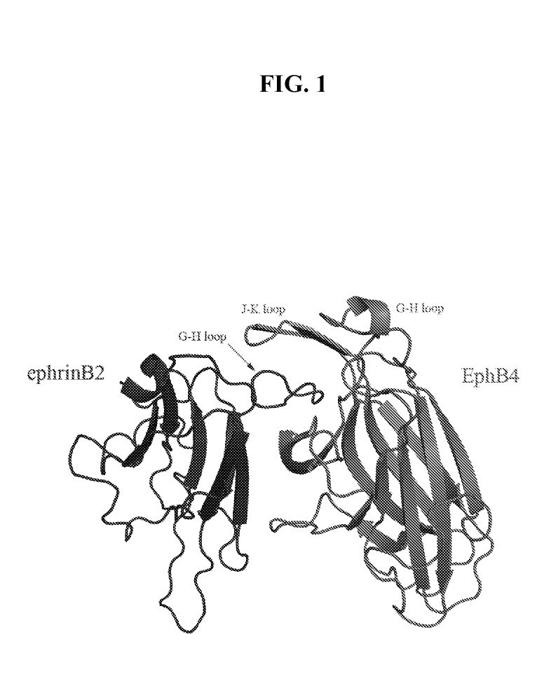

### DETAILED DESCRIPTION

The present invention relates to the discovery of the three-dimensional structure of a Receptor-Ligand Complex, models of such three-dimensional structures, a method of structure-based drug design using such structures, the compounds identified by such methods and the use of such compounds in therapeutic compositions. In particular, the present invention involves the crystal structure of the EphB4 receptor in complex with ephrinB2 at a resolution of 2.0 Å. EphrinB2 is situated in a hydrophobic cleft of EphB4 corresponding to the cleft in EphB2 occupied by the ephrinB2 G-H loop. The crystal reveals critical structural features of EphB4 that, when in complex ephrinB2, provides a basis for antagonist design and modeling.

In particular, the structural and thermodynamic characterization of the EphB4 receptor in complex with ephrinB2 is described. The structure reveals that the flexible J-K loop of EphB4 shifts significantly as compared to previous crystal structures, providing a new network of contacts to secure the interaction. In addition, using biophysical analysis, one amino acid, Leu-95, is identified which lines the ligand binding cavity of the EphB4 receptor and provides the molecular determinants for the unique specificity exhibited by the EphB4 receptor for the ephrinB2 ligand.

A multiple sequence alignment with members of the EphB subclass reveals that the EphB4 receptor lacks a conserved arginine and instead contains a leucine at position 95. A Leu-95-Arg mutation was previously predicted to result in steric interference with the antagonistic TNYL-RAW peptide ligand (Chrencik et al., Structure, 2006, incorporated herein by reference in its entirety; SEQ ID NO: 1). This mutation also results in steric interference with Phe-120 in the G-H loop of ephrinB2 due to the different positioning of the J-K loop of EphB4. A leucine instead of an arginine at position 95 of the EphB4 receptor is sufficient to cause substantial structural rearrangement of the receptor J-K loop. Also provided is a novel position of the conserved Phe-120 in the high affinity FSPN sequence of the ephrinB2 G-H loop, suggesting that although ephrinB2 is conserved in structure in both receptor-bound and apo structures, there is variability within the rigid G-H loop to conform to a specific receptor.

EphB4 binds only weakly to both ephrinB1 and ephrinB3, while exhibiting high affinity for ephrinB2. Considering the B-subclass ephrin G-H loop (ephrinB1-B3), it is interesting to speculate on why EphB4 preferentially binds ephrinB2 over other B-subclass ligands. EphrinB1 shares significant sequence identity with the high affinity ephrinB2 G-H loop, except at position 124, which is a Tyr in ephrinB1 and a Leu in ephrinB2. While Leu-124 forms no integral interactions with EphB4, the small size of the leucine allows tight packing within the receptor binding cavity. A leucine also maintains the hydrophobic nature of the binding cleft. Superposition of a tyrosine on the ephrinB2 structure would require the rearrangement of the EphB4 J-K loop in order to accommodate the bulky tyrosine, and, without being bound by a particular theory, this likely accounts for the reduced affinity of EphB4 for ephrinB1. The ephrinB3 G-H loop is also very similar to the ephrinB2 G-H loop but deviates in the FSPN sequence, which contains a tyrosine instead of the phenylalanine (YSPN). Phe-120 forms critical interactions with residues lining the EphB receptor-ephrinB2 binding cavity in the three complex crystal structures thus far described. In the previous crystal structures, Phe-120 extends to the surface of the binding cavity, adjacent to the receptor G-H loop. Superposition of a tyrosine on the EphB2-ephrinB2 structure would not affect the dynamics of the ligand binding cavity, and this residue is predicted to interact with several water molecules on the surface of the complex. However, in the present crystal structure, the Phe-120 of ephrinB2 is observed in a novel position, buried within the hydrophic binding cleft and forming interactions with Leu-95R and the Cys-61-Cys-184 disulfide bridge. Insertion of a tyrosine at this position would therefore result in both steric interference within the receptor binding cavity and a polar redistribution of the active site.

Thermodynamic discrepancies between Eph receptor and ephrin binding can be considered in the design of therapeutics to treat disease related to the Eph receptor family. Iterative rounds of structure based drug design provide an understanding of the enthalpic and entropic contributions of small molecule compounds. In the case of the ephrin ligand, the G-H loop is predicted to reduce conformational entropy losses due to its rigidity, maximizing the effects of solvation entropy due to the hydrophobic nature of the Eph ligand binding cavity. The ephrin, on the other hand, can experience large losses in conformational entropy upon receptor binding which are compensated by favorable enthalpic gains between receptor and ephrin residues. The ephrin ligand, with entropically-driven binding, can interact with multiple members of the EphB family. In contrast, the TNYL-RAW peptide, with enthalpically-driven binding, is a specific inhibitor of the EphB4-ephrinB2 interaction.

Accordingly, one aspect of the present invention includes a model of a Receptor-Ligand Complex in which the model represents a three-dimensional structure of a Receptor-Ligand Complex. Another aspect of the present invention includes the three-dimensional structure of a Receptor-Ligand Complex. A three-dimensional structure of a Receptor-Ligand Complex substantially conforms with the atomic coordinates represented in Table 1. According to the present invention, the use of the term “substantially conforms” refers to at least a portion of a three-dimensional structure of a Receptor-Ligand Complex which is sufficiently spatially similar to at least a portion of a specified three-dimensional configuration of a particular set of atomic coordinates (e.g., those represented by Table 1) to allow the three-dimensional structure of a Receptor-Ligand Complex to be modeled or calculated using the particular set of atomic coordinates as a basis for determining the atomic coordinates defining the three-dimensional configuration of a Receptor-Ligand Complex.

More particularly, a structure that substantially conforms to a given set of atomic coordinates is a structure wherein at least about 50% of such structure has an average root-mean-square deviation (RMSD) of less than about 2.0 Å for the backbone atoms in secondary structure elements in each domain, and in various aspects, less than about 1.25 Å for the backbone atoms in secondary structure elements in each domain, and, in various aspects less than about 1.0 Å, in other aspects less than about 0.75 Å, less than about 0.5 Å, and, less than about 0.25 Å for the backbone atoms in secondary structure elements in each domain. In one aspect of the present invention, a structure that substantially conforms to a given set of atomic coordinates is a structure wherein at least about 75% of such structure has the recited average RMSD value, and in some aspects, at least about 90% of such structure has the recited average RMSD value, and in some aspects, about 100% of such structure has the recited average RMSD value. In particular, the above definition of “substantially conforms” can be extended to include atoms of amino acid side chains. As used herein, the phrase “common amino acid side chains” refers to amino acid side chains that are common to both the structure which substantially conforms to a given set of atomic coordinates and the structure that is actually represented by such atomic coordinates.

In another aspect of the present invention, a three-dimensional structure that substantially conforms to a given set of atomic coordinates is a structure wherein at least about 50% of the common amino acid side chains have an average RMSD of less than about 2.0 Å, and in various aspects, less than about 1.25 Å, and, in other aspects, less than about 1.0 Å, less than about 0.75 Å, less than about 0.5 Å, and less than about 0.25 Å. In one aspect of the present invention, a structure that substantially conforms to a given set of atomic coordinates is a structure wherein at least about 75% of the common amino acid side chains have the recited average RMSD value, and in some aspects, at least about 90% of the common amino acid side chains have the recited average RMSD value, and in some aspects, about 100% of the common amino acid side chains have the recited average RMSD value.

A three-dimensional structure of a Receptor-Ligand Complex which substantially conforms to a specified set of atomic coordinates can be modeled by a suitable modeling computer program such as MODELER (A. Sali and T. L. Blundell, J. Mol. Biol., vol. 234:779-815, 1993 as implemented in the Insight II software package Insight II, available from Accelerys (San Diego, Calif.)) and those software packages listed in the Examples, using information, for example, derived from the following data: (1) the amino acid sequence of the Receptor-Ligand Complex; (2) the amino acid sequence of the related portion(s) of the protein represented by the specified set of atomic coordinates having a three-dimensional configuration; and, (3) the atomic coordinates of the specified three-dimensional configuration. A three-dimensional structure of a Receptor-Ligand Complex which substantially conforms to a specified set of atomic coordinates can also be calculated by a method such as molecular replacement, which is described in detail below.

A suitable three-dimensional structure of the Receptor-Ligand Complex for use in modeling or calculating the three-dimensional structure of another Receptor-Ligand Complex comprises the set of atomic coordinates represented in Table 1. The set of three-dimensional coordinates set forth in Table 1 is represented in standard Protein Data Bank format. The atomic coordinates have been deposited in the Protein Data Bank, having Accession No. 2HLE. According to the present invention, a Receptor-Ligand Complex has a three-dimensional structure which substantially conforms to the set of atomic coordinates represented by Table 1. As used herein, a three-dimensional structure can also be a most probable, or significant, fit with a set of atomic coordinates. According to the present invention, a most probable or significant fit refers to the fit that a particular Receptor-Ligand Complex has with a set of atomic coordinates derived from that particular Receptor-Ligand Complex. Such atomic coordinates can be derived, for example, from the crystal structure of the protein such as the coordinates determined for the Receptor-Ligand Complex structure provided herein, or from a model of the structure of the protein. For example, the three-dimensional structure of a dimeric protein, including a naturally occurring or recombinantly produced EphB4 receptor protein in complex with ephrinB2, substantially conforms to and is a most probable fit, or significant fit, with the atomic coordinates of Table 1. The three-dimensional crystal structure of the Receptor-Ligand Complex may comprise the atomic coordinates of Table 1. Also as an example, the three-dimensional structure of another Receptor-Ligand Complex would be understood by one of skill in the art to substantially conform to the atomic coordinates of Table 1. This definition can be applied to the other EphB4 receptor proteins in a similar manner.

For example, the structure of the EphB4 receptor establishes the general architecture of the EphB receptor family. Accordingly, in some configurations, EphB4 receptor protein sequence homology across eukaryotes can be used as a basis to predict the structure of such receptors, in particular the structure for such receptor-ligand binding sites and other conserved regions.

In various aspects of the present invention, a structure of a Receptor-Ligand Complex substantially conforms to the atomic coordinates represented in Table 1. Such values as listed in Table 1 can be interpreted by one of skill in the art. In other aspects, a three-dimensional structure of a Receptor-Ligand Complex substantially conforms to the three-dimensional coordinates represented in Table 1. In other aspects, a three-dimensional structure of a Receptor-Ligand Complex is a most probable fit with the three-dimensional coordinates represented in Table 1. Methods to determine a substantially conforming and probable fit are within the expertise of skill in the art and are described herein in the Examples section.

A Receptor-Ligand Complex that has a three-dimensional structure which substantially conforms to the atomic coordinates represented by Table 1 includes an EphB4 receptor protein having an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence of a human EphB4 receptor protein, in particular an amino acid sequence having SEQ ID NO: 4, across the full-length of the EphB4 receptor sequence. A sequence alignment program such as BLAST (available from the National Institutes of Health Internet web site http://www.ncbi.nlm.nih.gov/BLAST) may be used by one of skill in the art to compare sequences of an EphB receptor to the EphB4 receptor.

A three-dimensional structure of any Receptor-Ligand Complex can be modeled using methods generally known in the art based on information obtained from analysis of a Receptor-Ligand Complex crystal, and from other Receptor-Ligand Complex structures which are derived from a Receptor-Ligand Complex crystal. The Examples section below discloses the production of a Receptor-Ligand Complex crystal, in particular a truncated EphB4 receptor having SEQ ID NO: 2 or 3 complexed with ephrinB2 (SEQ ID NO: 6), and a model of a Receptor-Ligand Complex, in particular a truncated EphB4 receptor having SEQ ID NO: 2 or 3 complexed with ephrinB2, using methods generally known in the art based on the information obtained from analysis of a Receptor-Ligand Complex crystal.

An aspect of the present invention comprises using the three-dimensional structure of a crystalline Receptor-Ligand Complex to derive the three-dimensional structure of another Receptor-Ligand Complex. Therefore, the crystalline EphB4 receptor complexed with ephrinB2 (SEQ ID NO: 6), and the three-dimensional structure of EphB4 complexed with ephrinB2 permits one of ordinary skill in the art to now derive the three-dimensional structure, and models thereof, of another Receptor-Ligand Complex having highly specific EphB4 binding characteristics. The derivation of the structure of such Receptor-Ligand Complexes can now be achieved even in the absence of having crystal structure data for such other Receptor-Ligand Complexes, and when the crystal structure of another Receptor-Ligand Complex is available, the modeling of the three-dimensional structure of the new Receptor-Ligand Complex can be refined using the knowledge already gained from the Receptor-Ligand Complex structure.

In some configurations of the present teachings, the absence of crystal structure data for other Receptor-Ligand Complexes, the three-dimensional structures of other Receptor-Ligand Complexes can be modeled, taking into account differences in the amino acid sequence of the other Receptor-Ligand Complex. Moreover, the present invention allows for structure-based drug design of compounds which affect the activity of virtually any EphB receptor, and particularly, of EphB4.

One aspect of the present invention includes a three-dimensional structure of a Receptor-Ligand Complex, in which the atomic coordinates of the Receptor-Ligand Complex are generated by the method comprising: (a) providing an EphB4 receptor complexed with ephrinB2 in crystalline form; (b) generating an electron-density map of the crystalline EphB4 receptor complexed with ephrinB2; and (c) analyzing the electron-density map to produce the atomic coordinates. For example, the structure of human EphB4 receptor in complex with ephrinB2 (SEQ ID NO: 6) is provided herein.

The present invention also provides a three-dimensional structure of the EphB4 receptor protein complexed with ephrinB2 (SEQ ID NO: 6), can be used to derive a model of the three-dimensional structure of another Receptor-Ligand Complex (i.e., a structure to be modeled). As used herein, a “structure” of a protein refers to the components and the manner of arrangement of the components to constitute the protein. As used herein, the term “model” refers to a representation in a tangible medium of the three-dimensional structure of a protein, polypeptide or peptide. For example, a model can be a representation of the three-dimensional structure in an electronic file, on a computer screen, on a piece of paper (i.e., on a two dimensional medium), and/or as a ball-and-stick figure. Physical three-dimensional models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Accelrys, Inc. (San Diego, Calif.). The phrase “providing a picture of the model” refers to the ability to generate a “hard copy” of the model. Hard copies include both motion and still pictures. Computer screen images and pictures of the model can be visualized in a number of formats including space-filling representations, α-carbon traces, ribbon diagrams and electron density maps.

Suitable target Receptor-Ligand Complex structures to model using a method of the present invention include any EphB receptor protein, polypeptide or peptide that is substantially structurally related to an EphB4 receptor protein complexed with ephrinB2. In various embodiments, a target Receptor-Ligand Complex structure that is substantially structurally related to an EphB4 receptor protein includes a target Receptor-Ligand Complex structure having an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence of a human EphB4 receptor protein, in particular an amino acid sequence having SEQ ID NO: 4, across the full-length of the EphB4 receptor sequence when using, for example, a sequence alignment program such as BLAST (supra). In various aspects of the present invention, target Receptor-Ligand Complex structures to model include proteins comprising amino acid sequences that are at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to amino acid sequence of a truncated EphB4 receptor, EphB4(17-196), having SEQ ID NO: 2 or EphB4 17-198, having SEQ ID NO: 3, when comparing suitable regions of the sequence, such as the amino acid sequence for an ephrin binding site of any one of the amino acid sequences, when using an alignment program such as BLAST (supra) to align the amino acid sequences.

According to the present invention, a structure can be modeled using techniques generally described by, for example, Sali, Current Opinions in Biotechnology, vol. 6, pp. 437-451, 1995, and algorithms can be implemented in program packages such as Insight II, available from Accelerys (San Diego, Calif.). Use of Insight II HOMOLOGY requires an alignment of an amino acid sequence of a known structure having a known three-dimensional structure with an amino acid sequence of a target structure to be modeled. The alignment can be a pairwise alignment or a multiple sequence alignment including other related sequences (for example, using the method generally described by Rost, Meth. Enzymol., vol. 266, pp. 525-539, 1996) to improve accuracy. Structurally conserved regions can be identified by comparing related structural features, or by examining the degree of sequence homology between the known structure and the target structure. Certain coordinates for the target structure are assigned using known structures from the known structure. Coordinates for other regions of the target structure can be generated from fragments obtained from known structures such as those found in the Protein Data Bank. Conformation of side chains of the target structure can be assigned with reference to what is sterically allowable and using a library of rotamers and their frequency of occurrence (as generally described in Ponder and Richards, J. Mol. Biol., vol. 193, pp. 775-791, 1987). The resulting model of the target structure, can be refined by molecular mechanics to ensure that the model is chemically and conformationally reasonable.

Accordingly, one embodiment of the present invention is a method to derive a model of the three-dimensional structure of a target Receptor-Ligand Complex structure, the method comprising the steps of: (a) providing an amino acid sequence of a Receptor-Ligand Complex and an amino acid sequence of a target ligand-complexed EphB receptor; (b) identifying structurally conserved regions shared between the Receptor-Ligand Complex amino acid sequence and the target ligand-complexed EphB4 receptor amino acid sequence; (c) determining atomic coordinates for the target ligand-complexed EphB4 receptor by assigning said structurally conserved regions of the target ligand-complexed EphB4 receptor to a three-dimensional structure using a three-dimensional structure of a Receptor-Ligand Complex based on atomic coordinates that substantially conform to the atomic coordinates represented in Table 1, to derive a model of the three-dimensional structure of the target ligand-complexed EphB4 receptor amino acid sequence. A model according to the present invention has been previously described herein. In one aspect, the model comprises a computer model. The method can further comprise the step of electronically simulating the structural assignments to derive a computer model of the three-dimensional structure of the target ligand-complexed EphB4 receptor amino acid sequence.

Another embodiment of the present invention is a method to derive a computer model of the three-dimensional structure of a target ephrinB2-complexed EphB4 receptor structure for which a crystal has been produced (referred to herein as a “crystallized target structure”). A suitable method to produce such a model includes the method comprising molecular replacement. Methods of molecular replacement are generally known by those of skill in the art and are performed in a software program including, for example, X-PLOR available from Accelerys (San Diego, Calif.). In various aspects, a crystallized target ligand-complexed EphB receptor structure useful in a method of molecular replacement according to the present invention has an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the search structure (e.g., human EphB4), when the two amino acid sequences are compared using an alignment program such as BLAST (supra). A suitable search structure of the present invention includes a Receptor-Ligand Complex having a three-dimensional structure that substantially conforms with the atomic coordinates listed in Table 1.

Another aspect of the present invention is a method to determine a three-dimensional structure of a target Receptor-Ligand Complex structure, in which the three-dimensional structure of the target Receptor-Ligand Complex structure is not known. Such a method is useful for identifying structures that are related to the three-dimensional structure of a Receptor-Ligand Complex based only on the three-dimensional structure of the target structure. For example, the present method enables identification of structures that do not have high amino acid identity with an EphB4 receptor protein but which share three-dimensional structure similarities of a ligand-complexed EphB4 receptor. In various aspects of the present invention, a method to determine a three-dimensional structure of a target Receptor-Ligand Complex structure comprises: (a) providing an amino acid sequence of a target structure, wherein the three-dimensional structure of the target structure is not known; (b) analyzing the pattern of folding of the amino acid sequence in a three-dimensional conformation by fold recognition; and (c) comparing the pattern of folding of the target structure amino acid sequence with the three-dimensional structure of a Receptor-Ligand Complex to determine the three-dimensional structure of the target structure, wherein the three-dimensional structure of the Receptor-Ligand Complex substantially conforms to the atomic coordinates represented in Table 1. For example, methods of fold recognition can include the methods generally described in Jones, Curr. Opinion Struc. Biol., vol. 7, pp. 377-387, 1997. Such folding can be analyzed based on hydrophobic and/or hydrophilic properties of a target structure.

One aspect of the present invention includes a three-dimensional computer image of the three-dimensional structure of a Receptor-Ligand Complex. In one aspect, a computer image is created to a structure which substantially conforms with the three-dimensional coordinates listed in Table 1. A computer image of the present invention can be produced using any suitable software program, including, but not limited to, Pymol available from DeLano Scientific, LLC (South San Francisco, Calif.). Suitable computer hardware useful for producing an image of the present invention are known to those of skill in the art.

Another aspect of the present invention relates to a computer-readable medium encoded with a set of three-dimensional coordinates represented in Table 1, wherein, using a graphical display software program, the three-dimensional coordinates create an electronic file that can be visualized on a computer capable of representing said electronic file as a three-dimensional image. Yet another aspect of the present invention relates to a computer-readable medium encoded with a set of three-dimensional coordinates of a three-dimensional structure which substantially conforms to the three-dimensional coordinates represented in Table 1, wherein, using a graphical display software program, the set of three-dimensional coordinates create an electronic file that can be visualized on a computer capable of representing said electronic file as a three-dimensional image.

The present invention also includes a three-dimensional model of the three-dimensional structure of a target structure, such a three-dimensional model being produced by the method comprising: (a) providing an amino acid sequences of an EphB4 receptor comprised by a Receptor-Ligand Complex and an amino acid sequence of a target Receptor-Ligand Complex structure; (b) identifying structurally conserved regions shared between the EphB4 receptor amino acid sequence and the amino acid sequence comprised by the target Receptor-Ligand Complex structure; (c) determining atomic coordinates for the target Receptor-Ligand Complex by assigning the structurally conserved regions of the target Receptor-Ligand Complex to a three-dimensional structure using a three-dimensional structure of the EphB4 receptor comprised by a Receptor-Ligand Complex based on atomic coordinates that substantially conform to the atomic coordinates represented in Table 1 to derive a model of the three-dimensional structure of the target Receptor-Ligand Complex. In one aspect, the model comprises a computer model.

Another isolated EphB receptor protein can be used with the methods of the present invention. An isolated EphB receptor protein can be isolated from its natural milieu or produced using recombinant DNA technology (e.g., polymerase chain reaction (PCR) amplification, cloning) or chemical synthesis. To produce recombinant EphB receptor protein, a nucleic acid molecule encoding EphB receptor protein (e.g., SEQ ID NO: 5) can be inserted into any vector capable of delivering the nucleic acid molecule into a host cell. A nucleic acid molecule of the present invention can encode any portion of an EphB receptor protein, in various aspects a full-length EphB receptor protein, and in various aspects a soluble or truncated form of EphB4 receptor protein (i.e., a form of EphB4 receptor protein capable of being secreted by a cell that produces such protein). A suitable nucleic acid molecule to include in a recombinant vector, and particularly in a recombinant molecule, includes a nucleic acid molecule encoding a protein having the amino acid sequence represented by SEQ ID NOs: 2 or 3 and SEQ ID NO: 4.

A recombinant vector can be either RNA or DNA, either prokaryotic or eukaryotic, and typically is a virus or a plasmid. In various aspects, a nucleic acid molecule encoding an EphB4 receptor protein is inserted into a vector comprising an expression vector to form a recombinant molecule. As used herein, an expression vector is a DNA or RNA vector that is capable of transforming a host cell and of affecting expression of a specified nucleic acid molecule. Expression vectors of the present invention include any vectors that function (i.e., direct gene expression) in recombinant cells of the present invention, including in bacterial, fungal, endoparasite, insect, other animal, and plant cells.

An expression vector can be transformed into any suitable host cell to form a recombinant cell. A suitable host cell includes any cell capable of expressing a nucleic acid molecule inserted into the expression vector. For example, a prokaryotic expression vector can be transformed into a bacterial host cell. One method to isolate EphB4 receptor protein useful for producing ligand-complexed EphB4 receptor crystals includes recovery of recombinant proteins from cell cultures of recombinant cells expressing such EphB4 receptor protein.

EphB4 receptor proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, chromatofocusing and differential solubilization. In various aspects of the present invention, an EphB4 receptor protein is purified in such a manner that the protein is purified sufficiently for formation of crystals useful for obtaining information related to the three-dimensional structure of a Receptor-Ligand Complex. In some aspects, a composition of EphB4 receptor protein is about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% pure.

Another embodiment of the present invention includes a composition comprising a Receptor-Ligand Complex in a crystalline form (i.e., Receptor-Ligand Complex crystals). As used herein, the terms “crystalline Receptor-Ligand Complex” and “Receptor-Ligand Complex crystal” both refer to crystallized a Receptor-Ligand Complex and are intended to be used interchangeably. In various aspects of the present invention, a crystalline Receptor-Ligand Complex is produced using the crystal formation method described in the Examples.

In particular, the present invention includes a composition comprising EphB4 receptor complexed with ephrinB2 in a crystalline form (i.e., ephrinB2-complexed EphB4 crystals). As used herein, the terms “crystalline ephrinB2-complexed EphB4” and “ephrinB2-complexed EphB4 crystal” both refer to crystallized EphB4 receptor complexed with ephrinB2 and are intended to be used interchangeably. In various aspects of the present invention, a crystal ephrinB2-complexed EphB4 is produced using the crystal formation method described in the Examples. In some aspects, a composition of the present invention includes ephrinB2-complexed EphB4 molecules arranged in a crystalline manner in a space group P4₁ so as to form a unit cell of dimensions a=81.09 Å, b=81.09 Å, and c=50.95 Å. A suitable crystal of the present invention provides X-ray diffraction data for determination of atomic coordinates of the ephrinB2-complexed EphB4 to a resolution of about 2.0 Å, and in some aspects about 1.8 Å, and in other aspects at about 1.6 Å.

According to an aspect of the present invention, crystalline Receptor-Ligand Complex can be used to determine the ability of a compound of the present invention to bind to an EphB4 receptor in a manner predicted by a structure based drug design method of the present invention. In various aspects of the present invention, a Receptor-Ligand Complex crystal is soaked in a solution containing a chemical compound of the present invention. Binding of the chemical compound to the crystal is then determined by methods standard in the art.

One aspect of the present invention is a therapeutic composition. A therapeutic composition of the present invention comprises one or more therapeutic compounds. In one aspect, a therapeutic composition is provided that is capable of antagonizing the EphB4 receptor. For example, a therapeutic composition of the present invention can inhibit (i.e., prevent, block) binding of an EphB4 receptor on a cell having an EphB4 receptor (e.g., human cells) to a, e.g., ephrinB2 or ephrinB2 analog by interfering with the ligand binding domain of an EphB4 receptor. As used herein, the term “ligand binding domain” refers to the region of a molecule to which another molecule specifically binds.

Suitable inhibitory compounds of the present invention are compounds that interact directly with an EphB4 receptor protein or truncated EphB4 receptor protein (e.g., SEQ ID NOs: 2 or 3), thereby inhibiting the binding of ephrin-B2 to an EphB4 receptor by blocking the ligand binding domain of an EphB4 receptor (referred to herein as substrate analogs). An EphB4 receptor substrate analog refers to a compound that interacts with (e.g., binds to, associates with, modifies) the ligand binding domain of an EphB4 receptor. An EphB4 receptor substrate analog can, for example, comprise a chemical compound that mimics a polypeptide having SEQ ID NO: 6, truncated polypeptides comprised by SEQ ID NO: 6, or that binds specifically to the ephrin binding globular domain of an EphB4 receptor. Particularly, a substrate analog can comprise the G-H loop of ephrinB2 (SEQ ID NO: 7). Additionally, amino acids 120 through 127 of SEQ ID NO: 6 are useful in various aspects. In various aspects, an EphB4 receptor substrate analog useful in the present invention has an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 7 and amino acids 120 through 127 of SEQ ID NO: 6.

According to the present invention, suitable therapeutic compounds of the present invention include peptides or other organic molecules, and inorganic molecules. Suitable organic molecules include small organic molecules. In various aspects, a therapeutic compound of the present invention is not harmful (e.g., toxic) to an animal when such compound is administered to an animal. Peptides refer to a class of compounds that is small in molecular weight and yields two or more amino acids upon hydrolysis. A polypeptide is comprised of two or more peptides. As used herein, a protein is comprised of one or more polypeptides. Suitable therapeutic compounds to design include peptides composed of “L” and/or “D” amino acids that are configured as normal or retroinverso peptides, peptidomimetic compounds, small organic molecules, or homo- or hetero-polymers thereof, in linear or branched configurations.

Therapeutic compounds of the present invention can be designed using structure based drug design. Structure based drug design refers to the use of computer simulation to predict a conformation of a peptide, polypeptide, protein, or conformational interaction between a peptide or polypeptide, and a therapeutic compound. In the present teachings, knowledge of the three-dimensional structure of the EphB4 ligand binding domain of an EphB4 receptor when bound with ephrinB2 provide one of skill in the art the ability to design a therapeutic compound that binds to EphB4 receptors, is stable and results in inhibition of a biological response, such as tumorigenesis. For example, knowledge of the three-dimensional structure of the EphB4 ligand binding domain of an EphB4 receptor in complex with ephrinB2 provides to a skilled artisan the ability to design a ligand or an analog of a ligand which can function as a substrate or ligand of an EphB4 receptor.

Suitable structures and models useful for structure-based drug design are disclosed herein. Models of target structures to use in a method of structure-based drug design include models produced by any modeling method disclosed herein, such as, for example, molecular replacement and fold recognition related methods. In some aspects of the present invention, structure based drug design can be applied to a structure of EphB4 in complex with ephrinB2 (SEQ ID NO: 6), and to a model of a target EphB receptor structure.

One embodiment of the present invention is a method for designing a drug which interferes with an activity of an EphB4 receptor. In various configurations, the method comprises providing a three-dimensional structure of a Receptor-Ligand Complex comprising the EphB4 receptor and at least one ligand of the receptor; and designing a chemical compound which is predicted to bind to the EphB4 receptor. The designing can comprise using physical models, such as, for example, ball-and-stick representations of atoms and bonds, or on a digital computer equipped with molecular modeling software. In some configurations, these methods can further include synthesizing the chemical compound, and evaluating the chemical compound for ability to interfere with an activity of the EphB4 receptor.

Suitable three-dimensional structures of a Receptor-Ligand Complex and models to use with the present method are disclosed herein. According to the present invention, designing a compound can include creating a new chemical compound or searching databases of libraries of known compounds (e.g., a compound listed in a computational screening database containing three-dimensional structures of known compounds). Designing can also include simulating chemical compounds having substitute moieties at certain structural features. In some configurations, designing can include selecting a chemical compound based on a known function of the compound. In some configurations designing can comprise computational screening of one or more databases of compounds in which three-dimensional structures of the compounds are known. In these configurations, a candidate compound can be interacted virtually (e.g., docked, aligned, matched, interfaced) with the three-dimensional structure of a Receptor-Ligand Complex by computer equipped with software such as, for example, the AutoDock software package, (The Scripps Research Institute, La Jolla, Calif.) or described by Humblet and Dunbar, Animal Reports in Medicinal Chemistry, vol. 28, pp. 275-283, 1993, M Venuti, ed., Academic Press. Methods for synthesizing candidate chemical compounds are known to those of skill in the art.

Various other methods of structure-based drug design are disclosed in references such as Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three-dimensional structures and small fragment probes, followed by linking together of favorable probe sites.

In one aspect, a chemical compound of the present invention that binds to the ligand binding domain of a Receptor-Ligand Complex can be a chemical compound having chemical and/or stereochemical complementarity with an EphB4 receptor, e.g., an EphB4 receptor or ligand such as, for example, ephrinB2. In particular, the amino acid sequence of SEQ ID NO: 7, amino acids 120 through 127 of SEQ ID NO: 6, and analogs thereof can be complimentary. In some configurations, a chemical compound that binds to the ligand binding domain of an EphB4 receptor can associate with an affinity of at least about 10⁻⁶ M, at least about 10⁻⁷ M, or at least about 10⁻⁸ M.

Several sites of an EphB4 receptor can be targeted for structure based drug design. These sites include, in non-limiting example residues which contact ephrin-B2 or a polypeptide having SEQ ID NO: 1, e.g., EphB4 D-E and J-K loops; Leu-48, Cys-61, Leu-95, Ser-99 Leu-100, Pro-101, Thr-147, Lys-149, Ala-155, and Cys-184 of SEQ ID NO: 6. Conversely, the structure based drug design can be based upon the sites of the ligand which bind to the EphB4 receptor, e.g., Phe-120, Pro-122, Leu-124, Trp-125, and Leu-127 of ephrinB2.

Drug design strategies as specifically described above with regard to residues and regions of the ligand-complexed EphB4 receptor crystal can be similarly applied to the other EphB structures, including other EphB receptors disclosed herein. One of ordinary skill in the art, using the art recognized modeling programs and drug design methods, many of which are described herein, can modify the EphB4 design strategy according to differences in amino acid sequence. For example, this strategy can be used to design compounds which regulate a function of the EphB4 receptor in EphB receptors. In addition, one of skill in the art can use lead compound structures derived from one EphB receptor, such as the EphB4 receptor, and take into account differences in amino acid residues in other EphB4 receptors.

In the present method of structure-based drug design, it is not necessary to align a candidate chemical compound (i.e., a chemical compound being analyzed in, for example, a computational screening method of the present invention) to each residue in a target site. Suitable candidate chemical compounds can align to a subset of residues described for a target site. In some configurations of the present invention, a candidate chemical compound can comprise a conformation that promotes the formation of covalent or noncovalent crosslinking between the target site and the candidate chemical compound. In certain aspects, a candidate chemical compound can bind to a surface adjacent to a target site to provide an additional site of interaction in a complex. For example, when designing an antagonist (i.e., a chemical compound that inhibits the binding of ephrinB2 to an EphB4 receptor by blocking a ligand binding domain or interface), the antagonist can be designed to bind with sufficient affinity to the binding site or to substantially prohibit a ligand from binding to a target area. It will be appreciated by one of skill in the art that it is not necessary that the complementarity between a candidate chemical compound and a target site extend over all residues specified here.

In various aspects, the design of a chemical compound possessing stereochemical complementarity can be accomplished by means of techniques that optimize, chemically or geometrically, the “fit” between a chemical compound and a target site. Such techniques are disclosed by, for example, Sheridan and Venkataraghavan, Acc. Chem. Res., vol. 20, p. 322, 1987: Goodford, J. Med. Chem., vol. 27, p. 557, 1984; Beddell, Chem. Soc. Reviews, vol. 279, 1985; Hol, Angew. Chem., vol. 25, p. 767, 1986; and Verlinde and Hol, Structure, vol. 2, p. 577, 1994, each of which are incorporated by this reference herein in their entirety.

Some embodiments of the present invention for structure-based drug design comprise methods of identifying a chemical compound that complements the shape of an EphB4 receptor, particularly one that substantially conforms to the atomic coordinates of Table 1, or a structure that is related to an EphB4 receptor. Such method is referred to herein as a “geometric approach”. In a geometric approach of the present invention, the number of internal degrees of freedom (and the corresponding local minima in the molecular conformation space) can be reduced by considering only the geometric (hard-sphere) interactions of two rigid bodies, where one body (the active site) contains “pockets” or “grooves” that form binding sites for the second body (the complementing molecule, such as a ligand).

The geometric approach is described by Kuntz et al., J. Mol. Biol., vol. 161, p. 269, 1982, which is incorporated by this reference herein in its entirety. The algorithm for chemical compound design can be implemented using a software program such as AutoDock, available from the The Scripps Research Institute (La Jolla, Calif.). One or more extant databases of crystallographic data (e.g., the Cambridge Structural Database System maintained by University Chemical Laboratory, Cambridge University, Lensfield Road, Cambridge CB2 IEW, U.K. or the Protein Data Bank maintained by Rutgers University) can then be searched for chemical compounds that approximate the shape thus defined. Chemical compounds identified by the geometric approach can be modified to satisfy criteria associated with chemical complementarity, such as hydrogen bonding, ionic interactions or Van der Waals interactions.

In some embodiments, a therapeutic composition of the present invention can comprise one or more therapeutic compounds. A therapeutic composition can further comprise other compounds capable of inhibiting an EphB4 receptor. A therapeutic composition of the present invention can be used to treat disease in an animal such as, for example, a human in need of treatment by administering such composition to the human. Non-limiting examples of animals to treat include mammals, reptiles and birds, companion animals, food animals, zoo animals and other economically relevant animals (e.g., race horses and animals valued for their coats, such as minks). Additional animals to treat include dogs, cats, horses, cattle, sheep, swine, chickens, turkeys. Accordingly, in some aspects, animals to treat include humans.

A therapeutic composition of the present invention can also include an excipient, an adjuvant and/or carrier. Suitable excipients include compounds that the animal to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, a therapeutic composition can include a carrier. Carriers include compounds that increase the half-life of a therapeutic composition in the treated animal. Suitable carriers include, but are not limited to, polymeric controlled release vehicles, biodegradable implants, liposomes, bacteria, viruses, other cells, oils, esters, and glycols.

Acceptable protocols to administer therapeutic compositions of the present invention in an effective manner include individual dose size, number of doses, frequency of dose administration, and mode of administration. Determination of such protocols can be accomplished by those skilled in the art. Modes of administration can include, but are not limited to, subcutaneous, intradermal, intravenous, intranasal, oral, transdermal, intraocular and intramuscular routes.

In yet another embodiment, a method is provided for crystallizing an EphB4 receptor which includes providing an EphB4 receptor in contact with a polypeptide having SEQ ID NO: 1, followed by contacting the EphB4 receptor in contact with the polypeptide with a therapeutic compound as provided above, wherein the EphB4 receptor in contact with the polypeptide and the compound forms an EphB4 receptor crystal.

In another embodiment, a composition is provided comprising EphB4 receptor, a ligand, and a therapeutic compound as provided above. The EphB4 receptor can be a polypeptide having SEQ ID NO: 2 or 3. The EphB4 receptor can also consist essentially of EphB4 D-E and J-K loops or Leu-48, Cys-61, Leu-95, Ser-99 Leu-100, Pro-101, Thr-147, Lys-149, Ala-155, and Cys-184 of SEQ ID NO: 6. In certain embodiments, the EphB4 receptor can be a human EphB4 receptor.

In certain embodiments, the ligand can be a polypeptide having SEQ ID NO: 7 and amino acids 120 through 127 of SEQ ID NO: 6. In other embodiments, the ligand can be a polypeptide having at least 50%, 75% or 90% sequence identity to a polypeptide selected from the group consisting of polypeptides having SEQ ID NO: 7 and amino acids 120 through 127 of SEQ ID NO: 6.

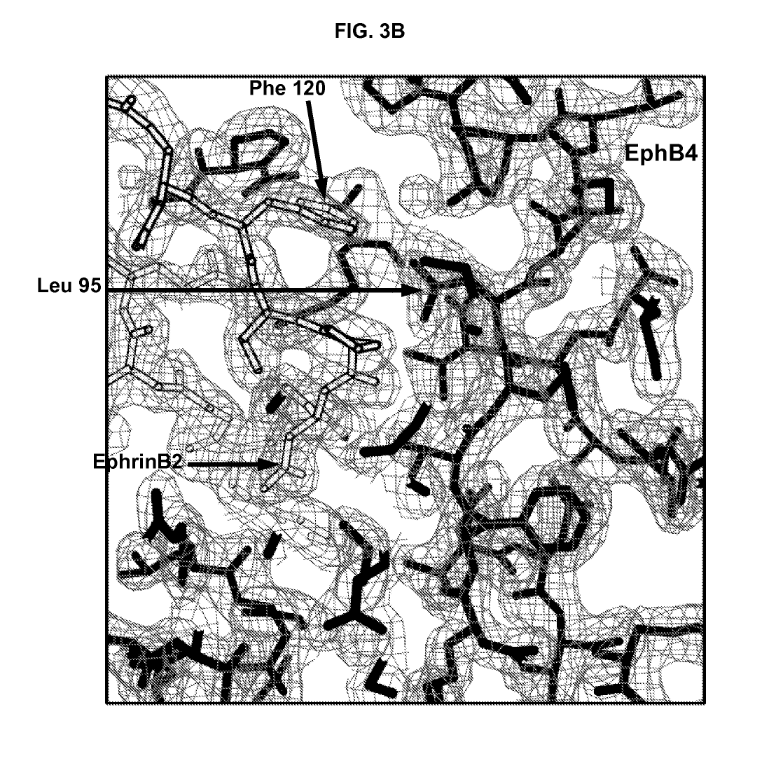

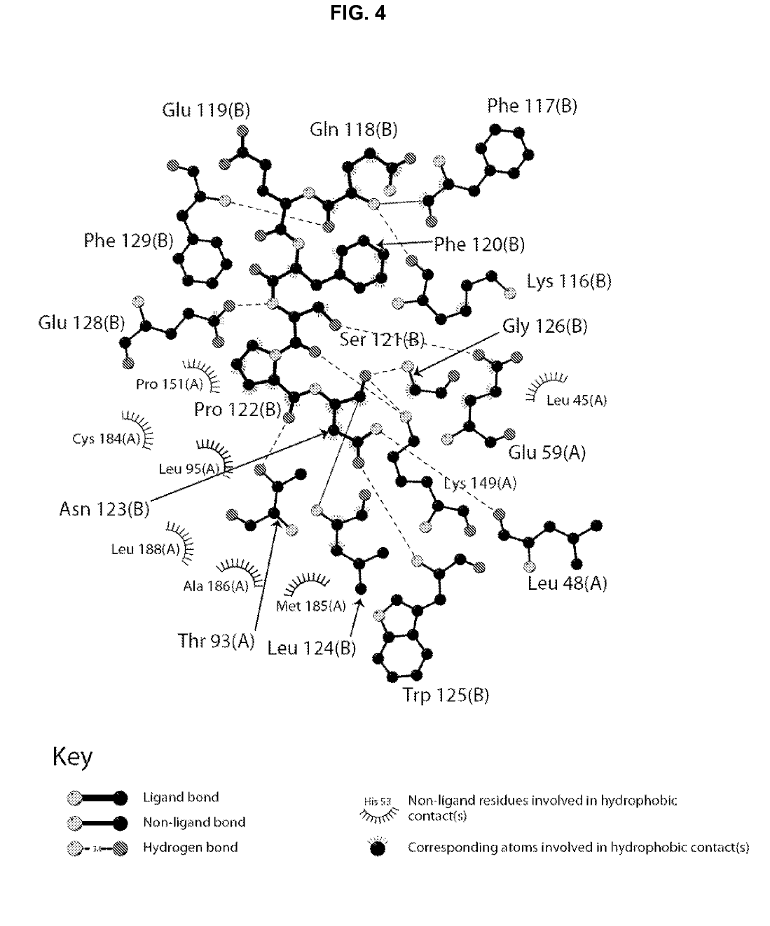

In some aspects, the present teachings include mutants of EphB4. In various configurations, these mutants can include at least one amino acid substitution, at least one amino acid addition, and/or at least one amino acid deletion. Such mutant EphB4 polypeptides and proteins can be constructed by methods well known to skilled artisans, such as site-directed mutagenesis. In some configurations, an EphB4 mutant can exhibit lower binding affinity (compared to wild type) for an EphB4 ligand such as EphrinB2, a TNYL-RAW peptide, or a labeled, e.g., fluorescently tagged, TNYL-RAW peptide. In some aspects, the binding affinity to an EphB4 ligand can be lower than that of wild type EphB4 (wtEphB4), without altering the binding specificity of the EphB4. Some non-limiting examples of EphB4 mutants of these aspects include T147F (i.e., threonine-147 to phenylalanine), K149Q (i.e., lysine-149 to glutamine), and A186S (i.e., alanine-186 to serine) as well as those found in FIG. 4. Accordingly, the dynamic range of binding of an EphB4 ligand to a mutant EphB4 can be greater than that of binding of an EphB4 ligand to wtEphB4. In some configurations, the dynamic range can be greater than about 2-fold (i.e., the dynamic range for a wtEphB4-ligand binding assay), such as, without limitation, a 2, 3, 4, 5, 6, 7, 8, 9, 10, 11, and 12-fold dynamic range.

In some aspects, a mutant EphB4 can be used in a screening assay for an EphB4 ligand, such as an EphB4 agonist or an EphB4 inhibitor. In a non-limiting example, an assay can comprise a fluorescence polarization (FP) assay using a fluorescent ligand such as a TNYL-RAW peptide labeled with a fluorophore such as Alexa-532 (Invitrogen). In various configurations, an assay can comprise contacting a complex comprising a mutant EphB4 and a fluorescent ligand with a candidate EphB4 ligand, and measuring a shift in the FP of the fluorescent ligand (Park, S. H., and Raines, R. T., Methods Mol. Biol. 261: 161-166, 2004). In some configurations, a mutant EphB4 can show a lower specificity to a ligand such as EphrinB2 or a fluorescent TNYL-RAW peptide. A shift in FP in such assays can indicate that a candidate EphB4 ligand binds to the EphB4. A compound identified by such a screening assay can be further tested, e.g., for pharmacological effectiveness and toxicity, using standard cell biological, biochemical and pharmacological tests well known to skilled artisans. Such assays can be used individually with candidate molecules, or at any scale of screening, such as, without limitation, high-throughput screening in which several thousand compounds can be rapidly tested for activity as ligands for EphB4.

TABLE 1
Protein Databank Coordinates
of EphB4 Receptor Complexed ephrinB2

REMARK 3 PROGRAM: REFMAC 5.2.0019
REMARK 3 AUTHORS: MURSHUDOV, VAGIN, DODSON
REMARK 3
REMARK 3 REFINEMENT TARGET: MAXIMUM LIKELIHOOD
REMARK 3
REMARK 3 DATA USED IN REFINEMENT.
REMARK 3 RESOLUTION RANGE HIGH (ANGSTROMS): 1.91
REMARK 3 RESOLUTION RANGE LOW (ANGSTROMS): 36.27
REMARK 3 DATA CUTOFF (SIGMA(F)): NONE
REMARK 3 COMPLETENESS FOR RANGE (%): 99.07
REMARK 3 NUMBER OF REFLECTIONS: 24232
REMARK 3
REMARK 3 FIT TO DATA USED IN REFINEMENT.
REMARK 3 CROSS-VALIDATION METHOD: THROUGHOUT
REMARK 3 FREE R VALUE TEST SET SELECTION: RANDOM
REMARK 3 R VALUE (WORKING + TEST SET): 0.26396
REMARK 3 R VALUE (WORKING SET): 0.26082
REMARK 3 FREE R VALUE: 0.32142
REMARK 3 FREE R VALUE TEST SET SIZE (%): 5.1
REMARK 3 FREE R VALUE TEST SET COUNT: 1304
REMARK 3
REMARK 3 FIT IN THE HIGHEST RESOLUTION BIN.
REMARK 3 TOTAL NUMBER OF BINS USED: 20
REMARK 3 BIN RESOLUTION RANGE HIGH: 1.912
REMARK 3 BIN RESOLUTION RANGE LOW: 1.962
REMARK 3 REFLECTION IN BIN (WORKING SET): 1659
REMARK 3 BIN COMPLETENESS (WORKING + TEST) (%): 92.46
REMARK 3 BIN R VALUE (WORKING SET): 0.331
REMARK 3 BIN FREE R VALUE SET COUNT: 94
REMARK 3 BIN FREE R VALUE: 0.413
REMARK 3
REMARK 3 NUMBER OF NON-HYDROGEN ATOMS USED IN
REFINEMENT.
REMARK 3 ALL ATOMS: 2510
REMARK 3
REMARK 3 B VALUES.
REMARK 3 FROM WILSON PLOT (A**2): NULL
REMARK 3 MEAN B VALUE (OVERALL, A**2): 46.363
REMARK 3 OVERALL ANISOTROPIC B VALUE.
REMARK 3 B11 (A**2): −0.18
REMARK 3 B22 (A**2): −0.18
REMARK 3 B33 (A**2): 0.36
REMARK 3 B12 (A**2): 0.00
REMARK 3 B13 (A**2): 0.00
REMARK 3 B23 (A**2): 0.00
REMARK 3
REMARK 3 ESTIMATED OVERALL COORDINATE ERROR.
REMARK 3 ESU BASED ON R VALUE (A): 0.214
REMARK 3 ESU BASED ON FREE R VALUE (A): 0.202
REMARK 3 ESU BASED ON MAXIMUM LIKELIHOOD (A): 0.220
REMARK 3 ESU FOR B VALUES BASED ON MAXIMUM
LIKELIHOOD (A**2): 16.948
REMARK 3
REMARK 3 CORRELATION COEFFICIENTS.
REMARK 3 CORRELATION COEFFICIENT FO-FC: 0.934
REMARK 3 CORRELATION COEFFICIENT FO-FC FREE: 0.893
REMARK 3
REMARK 3 RMS DEVIATIONS FROM IDEAL VALUES COUNT
RMS WEIGHT
REMARK 3 BOND LENGTHS REFINED ATOMS (A): 2577; 0.013;
0.022
REMARK 3 BOND ANGLES REFINED ATOMS (DEGREES): 3512;
1.579; 1.950
REMARK 3 TORSION ANGLES, PERIOD 1 (DEGREES): 323;
7.895; 5.000
REMARK 3 TORSION ANGLES, PERIOD 2 (DEGREES): 109;
37.322; 24.404
REMARK 3 TORSION ANGLES, PERIOD 3 (DEGREES): 406;
20.218; 15.000
REMARK 3 TORSION ANGLES, PERIOD 4 (DEGREES): 9;
14.327; 15.000
REMARK 3 CHIRAL-CENTER RESTRAINTS (A**3): 392; 0.103;
0.200
REMARK 3 GENERAL PLANES REFINED ATOMS (A): 1955;
0.006; 0.020
REMARK 3 NON-BONDED CONTACTS REFINED ATOMS (A):
976; 0.233; 0.200
REMARK 3 NON-BONDED TORSION REFINED ATOMS (A):
1665; 0.309; 0.200
REMARK 3 H-BOND (X . . . Y) REFINED ATOMS (A): 89; 0.168;
0.200
REMARK 3 SYMMETRY VDW REFINED ATOMS (A): 52; 0.228;
0.200
REMARK 3 SYMMETRY H-BOND REFINED ATOMS (A): 9; 0.218;
0.200
REMARK 3
REMARK 3 ISOTROPIC THERMAL FACTOR RESTRAINTS. COUNT RMS WEIGHT
REMARK 3 MAIN-CHAIN BOND REFINED ATOMS (A**2): 1660;
2.151; 3.000
REMARK 3 MAIN-CHAIN ANGLE REFINED ATOMS (A**2): 2588;
3.180; 5.000
REMARK 3 SIDE-CHAIN BOND REFINED ATOMS (A**2): 1065;
4.640; 7.000
REMARK 3 SIDE-CHAIN ANGLE REFINED ATOMS (A**2): 924;
5.442; 9.000
REMARK 3
REMARK 3 NCS RESTRAINTS STATISTICS
REMARK 3 NUMBER OF NCS GROUPS: NULL
REMARK 3
REMARK 3
REMARK 3 TLS DETAILS
REMARK 3 NUMBER OF TLS GROUPS: 2
REMARK 3 ATOM RECORD CONTAINS RESIDUAL B FACTORS
ONLY
REMARK 3
REMARK 3 TLS GROUP: 1
REMARK 3 NUMBER OF COMPONENTS GROUP: 1
REMARK 3 COMPONENTS C SSSEQI TO C SSSEQI
REMARK 3 RESIDUE RANGE: A 9 A 196
REMARK 3 ORIGIN FOR THE GROUP (A): 6.5459 29.3924 −8.9085
REMARK 3 T TENSOR
REMARK 3 T11: −0.0510 T22: −0.0163
REMARK 3 T33: 0.0412 T12: 0.0109
REMARK 3 T13: −0.0201 T23: 0.0639
REMARK 3 L TENSOR
REMARK 3 L11: 1.0238 L22: 1.0652
REMARK 3 L33: 0.1402 L12: −0.0578
REMARK 3 L13: 0.0466 L23: 0.0816
REMARK 3 S TENSOR
REMARK 3 S11: −0.0398 S12: 0.2633 S13: 0.0085
REMARK 3 S21: 0.0822 S22: 0.0447 S23: 0.2172
REMARK 3 S31: −0.0545 S32: 0.0708 S33: −0.0048
REMARK 3
REMARK 3 TLS GROUP: 2
REMARK 3 NUMBER OF COMPONENTS GROUP: 1
REMARK 3 COMPONENTS C SSSEQI TO C SSSEQI
REMARK 3 RESIDUE RANGE: B 31 B 167
REMARK 3 ORIGIN FOR THE GROUP (A): 24.0838 25.2120
12.7894
REMARK 3 T TENSOR
REMARK 3 T11: −0.0009 T22: −0.0420
REMARK 3 T33: −0.0654 T12: 0.0636
REMARK 3 T13: −0.0312 T23: 0.0420
REMARK 3 L TENSOR
REMARK 3 L11: 1.3724 L22: 1.0673
REMARK 3 L33: 2.6076 L12: 0.8437
REMARK 3 L13: 1.4287 L23: 0.6393
REMARK 3 S TENSOR
REMARK 3 S11: 0.1162 S12: 0.1266 S13: −0.1332
REMARK 3 S21: 0.2137 S22: 0.0292 S23: −0.0248
REMARK 3 S31: 0.1770 S32: 0.2825 S33: −0.1455
REMARK 3
REMARK 3
REMARK 3 BULK SOLVENT MODELLING.
REMARK 3 METHOD USED: MASK
REMARK 3 PARAMETERS FOR MASK CALCULATION
REMARK 3 VDW PROBE RADIUS: 1.20
REMARK 3 ION PROBE RADIUS: 0.80
REMARK 3 SHRINKAGE RADIUS: 0.80
REMARK 3
REMARK 3 OTHER REFINEMENT REMARKS:
REMARK 3 HYDROGENS HAVE BEEN ADDED IN THE RIDING
POSITIONS
REMARK 3
SSBOND 1 CYS A 61 CYS A 184
SSBOND 2 CYS A 97 CYS A 107
SSBOND 3 CYS B 65 CYS B 104
SSBOND 4 CYS B 92 CYS B 156
CISPEP 1 PHE A 35 PRO A 36 0.00
CISPEP 2 THR A 127 PRO A 128 0.00
CISPEP 3 ASN A 133 PRO A 134 0.00
CISPEP 4 GLY A 167 PRO A 168 0.00
CISPEP 5 ASP B 90 ARG B 91 0.00
CRYST1 81.085 81.085 50.945 90.00 90.00 90.00 P 41
SCALE1 0.012333 0.000000 0.000000 0.00000
SCALE2 0.000000 0.012333 0.000000 0.00000
SCALE3 0.000000 0.000000 0.019629 0.00000

| | | | | | | | | | | | |
|---|---|---|---|---|---|---|---|---|---|---|---|
| ATOM | 10 | N | HIS | A | 11 | 24.448 | 54.004 | −27.877 | 1.00 | 49.00 | N |
| ATOM | 11 | CA | HIS | A | 11 | 23.039 | 54.322 | −27.723 | 1.00 | 47.86 | C |
| ATOM | 12 | CB | HIS | A | 11 | 22.253 | 53.916 | −28.974 | 1.00 | 48.48 | C |
| ATOM | 13 | CG | HIS | A | 11 | 22.560 | 54.756 | −30.174 | 1.00 | 46.99 | C |
| ATOM | 14 | ND1 | HIS | A | 11 | 21.891 | 55.929 | −30.454 | 1.00 | 47.84 | N |
| ATOM | 15 | CE1 | HIS | A | 11 | 22.369 | 56.455 | −31.566 | 1.00 | 44.85 | C |
| ATOM | 16 | NE2 | HIS | A | 11 | 23.333 | 55.672 | −32.016 | 1.00 | 48.34 | N |
| ATOM | 17 | CD2 | HIS | A | 11 | 23.473 | 54.602 | −31.163 | 1.00 | 49.20 | C |
| ATOM | 18 | C | HIS | A | 11 | 22.494 | 53.661 | −26.468 | 1.00 | 46.86 | C |
| ATOM | 19 | O | HIS | A | 11 | 22.657 | 52.455 | −26.254 | 1.00 | 46.50 | O |
| ATOM | 20 | N | HIS | A | 12 | 21.858 | 54.459 | −25.620 | 1.00 | 44.51 | N |
| ATOM | 21 | CA | HIS | A | 12 | 21.462 | 53.973 | −24.313 | 1.00 | 43.01 | C |
| ATOM | 22 | CB | HIS | A | 12 | 22.056 | 54.854 | −23.211 | 1.00 | 43.91 | C |
| ATOM | 23 | CG | HIS | A | 12 | 23.320 | 54.305 | −22.635 | 1.00 | 46.33 | C |
| ATOM | 24 | ND1 | HIS | A | 12 | 23.372 | 53.706 | −21.396 | 1.00 | 47.69 | N |
| ATOM | 25 | CE1 | HIS | A | 12 | 24.605 | 53.299 | −21.160 | 1.00 | 52.21 | C |
| ATOM | 26 | NE2 | HIS | A | 12 | 25.351 | 53.602 | −22.207 | 1.00 | 51.06 | N |
| ATOM | 27 | CD2 | HIS | A | 12 | 24.569 | 54.227 | −23.145 | 1.00 | 49.90 | C |
| ATOM | 28 | C | HIS | A | 12 | 19.962 | 53.828 | −24.139 | 1.00 | 40.46 | C |
| ATOM | 29 | O | HIS | A | 12 | 19.191 | 54.727 | −24.500 | 1.00 | 41.03 | O |
| ATOM | 30 | N | HIS | A | 13 | 19.556 | 52.677 | −23.624 | 1.00 | 39.88 | N |
| ATOM | 31 | CA | HIS | A | 13 | 18.184 | 52.506 | −23.152 | 1.00 | 39.98 | C |
| ATOM | 32 | CB | HIS | A | 13 | 17.414 | 51.459 | −23.973 | 1.00 | 39.69 | C |
| ATOM | 33 | CG | HIS | A | 13 | 15.982 | 51.315 | −23.562 | 1.00 | 40.91 | C |
| ATOM | 34 | ND1 | HIS | A | 13 | 15.410 | 50.100 | −23.255 | 1.00 | 43.46 | N |
| ATOM | 35 | CE1 | HIS | A | 13 | 14.154 | 50.287 | −22.890 | 1.00 | 36.43 | C |
| ATOM | 36 | NE2 | HIS | A | 13 | 13.895 | 51.579 | −22.942 | 1.00 | 39.44 | N |
| ATOM | 37 | CD2 | HIS | A | 13 | 15.021 | 52.242 | −23.359 | 1.00 | 38.36 | C |
| ATOM | 38 | C | HIS | A | 13 | 18.174 | 52.172 | −21.661 | 1.00 | 39.78 | C |
| ATOM | 39 | O | HIS | A | 13 | 18.660 | 51.131 | −21.228 | 1.00 | 38.65 | O |
| ATOM | 40 | N | HIS | A | 14 | 17.617 | 53.068 | −20.866 | 1.00 | 38.90 | N |
| ATOM | 41 | CA | HIS | A | 14 | 17.507 | 52.802 | −19.451 | 1.00 | 39.71 | C |
| ATOM | 42 | CB | HIS | A | 14 | 17.974 | 54.017 | −18.639 | 1.00 | 41.36 | C |
| ATOM | 43 | C | HIS | A | 14 | 16.049 | 52.499 | −19.153 | 1.00 | 40.66 | C |
| ATOM | 44 | O | HIS | A | 14 | 15.159 | 53.220 | −19.604 | 1.00 | 38.87 | O |
| ATOM | 45 | N | HIS | A | 15 | 15.797 | 51.424 | −18.419 | 1.00 | 37.90 | N |
| ATOM | 46 | CA | HIS | A | 15 | 14.455 | 51.188 | −17.913 | 1.00 | 39.77 | C |
| ATOM | 47 | CB | HIS | A | 15 | 13.713 | 50.239 | −18.844 | 1.00 | 38.53 | C |
| ATOM | 48 | CG | HIS | A | 15 | 12.236 | 50.372 | −18.753 | 1.00 | 36.65 | C |
| ATOM | 49 | ND1 | HIS | A | 15 | 11.388 | 49.846 | −19.698 | 1.00 | 36.72 | N |
| ATOM | 50 | CE1 | HIS | A | 15 | 10.141 | 50.136 | −19.372 | 1.00 | 37.69 | C |
| ATOM | 51 | NE2 | HIS | A | 15 | 10.155 | 50.844 | −18.256 | 1.00 | 36.35 | N |
| ATOM | 52 | CD2 | HIS | A | 15 | 11.454 | 51.002 | −17.845 | 1.00 | 37.33 | C |
| ATOM | 53 | C | HIS | A | 15 | 14.438 | 50.663 | −16.465 | 1.00 | 42.34 | C |
| ATOM | 54 | O | HIS | A | 15 | 14.945 | 49.583 | −16.197 | 1.00 | 45.21 | O |
| ATOM | 55 | N | HIS | A | 16 | 13.877 | 51.449 | −15.542 | 1.00 | 42.50 | N |
| ATOM | 56 | CA | HIS | A | 16 | 13.849 | 51.102 | −14.115 | 1.00 | 44.89 | C |
| ATOM | 57 | CB | HIS | A | 16 | 13.544 | 52.349 | −13.269 | 1.00 | 46.52 | C |
| ATOM | 58 | CG | HIS | A | 16 | 14.588 | 53.417 | −13.375 | 1.00 | 54.62 | C |
| ATOM | 59 | ND1 | HIS | A | 16 | 14.398 | 54.577 | −14.100 | 1.00 | 57.87 | N |
| ATOM | 60 | CE1 | HIS | A | 16 | 15.486 | 55.323 | −14.023 | 1.00 | 52.34 | C |
| ATOM | 61 | NE2 | HIS | A | 16 | 16.378 | 54.684 | −13.286 | 1.00 | 53.86 | N |
| ATOM | 62 | CD2 | HIS | A | 16 | 15.843 | 53.488 | −12.870 | 1.00 | 51.85 | C |
| ATOM | 63 | C | HIS | A | 16 | 12.856 | 49.985 | −13.768 | 1.00 | 44.45 | C |
| ATOM | 64 | O | HIS | A | 16 | 11.720 | 50.017 | −14.218 | 1.00 | 38.53 | O |
| ATOM | 65 | N | GLU | A | 17 | 13.296 | 49.033 | −12.940 | 1.00 | 43.15 | N |
| ATOM | 66 | CA | GLU | A | 17 | 12.510 | 47.851 | −12.579 | 1.00 | 45.72 | C |
| ATOM | 67 | CB | GLU | A | 17 | 13.325 | 46.576 | −12.794 | 1.00 | 46.52 | C |
| ATOM | 68 | CG | GLU | A | 17 | 13.479 | 46.141 | −14.242 | 1.00 | 45.21 | C |
| ATOM | 69 | CD | GLU | A | 17 | 14.404 | 44.935 | −14.428 | 1.00 | 48.39 | C |
| ATOM | 70 | OE1 | GLU | A | 17 | 14.909 | 44.363 | −13.436 | 1.00 | 53.51 | O |
| ATOM | 71 | OE2 | GLU | A | 17 | 14.645 | 44.557 | −15.587 | 1.00 | 49.07 | O |
| ATOM | 72 | C | GLU | A | 17 | 12.077 | 47.943 | −11.131 | 1.00 | 45.46 | C |
| ATOM | 73 | O | GLU | A | 17 | 12.914 | 48.171 | −10.252 | 1.00 | 47.09 | O |
| ATOM | 74 | N | GLU | A | 18 | 10.775 | 47.812 | −10.879 | 1.00 | 46.25 | N |
| ATOM | 75 | CA | GLU | A | 18 | 10.287 | 47.795 | −9.500 | 1.00 | 47.01 | C |
| ATOM | 76 | CB | GLU | A | 18 | 9.270 | 48.907 | −9.194 | 1.00 | 50.02 | C |
| ATOM | 77 | CG | GLU | A | 18 | 9.553 | 49.578 | −7.827 | 1.00 | 53.61 | C |
| ATOM | 78 | CD | GLU | A | 18 | 8.322 | 50.164 | −7.143 | 1.00 | 58.47 | C |
| ATOM | 79 | OE1 | GLU | A | 18 | 7.339 | 49.415 | −6.922 | 1.00 | 58.46 | O |
| ATOM | 80 | OE2 | GLU | A | 18 | 8.356 | 51.370 | −6.792 | 1.00 | 52.49 | O |
| ATOM | 81 | C | GLU | A | 18 | 9.737 | 46.439 | −9.101 | 1.00 | 45.25 | C |
| ATOM | 82 | O | GLU | A | 18 | 8.859 | 45.885 | −9.760 | 1.00 | 46.79 | O |
| ATOM | 83 | N | THR | A | 19 | 10.265 | 45.937 | −7.993 | 1.00 | 43.60 | N |
| ATOM | 84 | CA | THR | A | 19 | 10.022 | 44.578 | −7.547 | 1.00 | 40.92 | C |
| ATOM | 85 | CB | THR | A | 19 | 11.154 | 44.128 | −6.611 | 1.00 | 40.82 | C |
| ATOM | 86 | OG1 | THR | A | 19 | 12.355 | 43.937 | −7.372 | 1.00 | 46.16 | O |
| ATOM | 87 | CG2 | THR | A | 19 | 10.790 | 42.835 | −5.855 | 1.00 | 41.51 | C |
| ATOM | 88 | C | THR | A | 19 | 8.719 | 44.532 | −6.793 | 1.00 | 38.18 | C |
| ATOM | 89 | O | THR | A | 19 | 8.506 | 45.332 | −5.877 | 1.00 | 38.71 | O |
| ATOM | 90 | N | LEU | A | 20 | 7.869 | 43.573 | −7.155 | 1.00 | 35.11 | N |
| ATOM | 91 | CA | LEU | A | 20 | 6.598 | 43.341 | −6.454 | 1.00 | 35.09 | C |
| ATOM | 92 | CB | LEU | A | 20 | 5.582 | 42.814 | −7.455 | 1.00 | 32.21 | C |
| ATOM | 93 | CG | LEU | A | 20 | 5.228 | 43.763 | −8.593 | 1.00 | 42.07 | C |
| ATOM | 94 | CD1 | LEU | A | 20 | 4.775 | 43.011 | −9.829 | 1.00 | 41.64 | C |
| ATOM | 95 | CD2 | LEU | A | 20 | 4.192 | 44.745 | −8.158 | 1.00 | 31.95 | C |
| ATOM | 96 | C | LEU | A | 20 | 6.783 | 42.315 | −5.308 | 1.00 | 35.93 | C |
| ATOM | 97 | O | LEU | A | 20 | 6.192 | 42.424 | −4.227 | 1.00 | 39.65 | O |
| ATOM | 98 | N | LEU | A | 21 | 7.647 | 41.347 | −5.560 | 1.00 | 37.15 | N |
| ATOM | 99 | CA | LEU | A | 21 | 7.866 | 40.212 | −4.662 | 1.00 | 36.59 | C |
| ATOM | 100 | CB | LEU | A | 21 | 6.704 | 39.210 | −4.790 | 1.00 | 35.23 | C |
| ATOM | 101 | CG | LEU | A | 21 | 6.824 | 37.819 | −4.076 | 1.00 | 30.98 | C |
| ATOM | 102 | CD1 | LEU | A | 21 | 7.141 | 37.996 | −2.622 | 1.00 | 42.83 | C |
| ATOM | 103 | CD2 | LEU | A | 21 | 5.549 | 37.070 | −4.163 | 1.00 | 31.48 | C |
| ATOM | 104 | C | LEU | A | 21 | 9.193 | 39.556 | −5.034 | 1.00 | 37.39 | C |
| ATOM | 105 | O | LEU | A | 21 | 9.445 | 39.256 | −6.193 | 1.00 | 39.08 | O |
| ATOM | 106 | N | ASN | A | 22 | 10.053 | 39.357 | −4.050 | 1.00 | 39.54 | N |
| ATOM | 107 | CA | ASN | A | 22 | 11.326 | 38.676 | −4.273 | 1.00 | 40.39 | C |
| ATOM | 108 | CB | ASN | A | 22 | 12.473 | 39.693 | −4.295 | 1.00 | 36.59 | C |
| ATOM | 109 | CG | ASN | A | 22 | 13.795 | 39.073 | −4.663 | 1.00 | 42.16 | C |
| ATOM | 110 | OD1 | ASN | A | 22 | 13.963 | 37.854 | −4.578 | 1.00 | 37.60 | O |
| ATOM | 111 | ND2 | ASN | A | 22 | 14.759 | 39.910 | −5.068 | 1.00 | 32.02 | N |
| ATOM | 112 | C | ASN | A | 22 | 11.525 | 37.720 | −3.110 | 1.00 | 39.65 | C |
| ATOM | 113 | O | ASN | A | 22 | 11.721 | 38.195 | −1.992 | 1.00 | 40.60 | O |
| ATOM | 114 | N | THR | A | 23 | 11.447 | 36.403 | −3.362 | 1.00 | 43.19 | N |
| ATOM | 115 | CA | THR | A | 23 | 11.631 | 35.394 | −2.304 | 1.00 | 39.47 | C |
| ATOM | 116 | CB | THR | A | 23 | 11.247 | 33.931 | −2.746 | 1.00 | 39.83 | C |
| ATOM | 117 | OG1 | THR | A | 23 | 12.103 | 33.496 | −3.803 | 1.00 | 29.00 | O |
| ATOM | 118 | CG2 | THR | A | 23 | 9.793 | 33.845 | −3.201 | 1.00 | 37.70 | C |
| ATOM | 119 | C | THR | A | 23 | 13.062 | 35.354 | −1.755 | 1.00 | 41.75 | C |
| ATOM | 120 | O | THR | A | 23 | 13.241 | 35.056 | −0.571 | 1.00 | 39.34 | O |
| ATOM | 121 | N | LYS | A | 24 | 14.058 | 35.605 | −2.613 | 1.00 | 42.15 | N |
| ATOM | 122 | CA | LYS | A | 24 | 15.497 | 35.577 | −2.231 | 1.00 | 47.84 | C |
| ATOM | 123 | CB | LYS | A | 24 | 16.430 | 35.906 | −3.417 | 1.00 | 44.41 | C |
| ATOM | 124 | CG | LYS | A | 24 | 16.713 | 34.791 | −4.408 | 1.00 | 36.81 | C |
| ATOM | 125 | CD | LYS | A | 24 | 17.426 | 35.348 | −5.632 | 1.00 | 51.46 | C |
| ATOM | 126 | CE | LYS | A | 24 | 16.468 | 36.179 | −6.495 | 1.00 | 59.42 | C |
| ATOM | 127 | NZ | LYS | A | 24 | 17.187 | 37.004 | −7.498 | 1.00 | 60.14 | N |
| ATOM | 128 | C | LYS | A | 24 | 15.843 | 36.550 | −1.107 | 1.00 | 53.13 | C |
| ATOM | 129 | O | LYS | A | 24 | 16.809 | 36.334 | −0.372 | 1.00 | 57.22 | O |
| ATOM | 130 | N | LEU | A | 25 | 15.069 | 37.619 | −0.974 | 1.00 | 54.53 | N |
| ATOM | 131 | CA | LEU | A | 25 | 15.320 | 38.588 | 0.085 | 1.00 | 59.86 | C |
| ATOM | 132 | CB | LEU | A | 25 | 15.531 | 40.002 | −0.491 | 1.00 | 60.08 | C |
| ATOM | 133 | CG | LEU | A | 25 | 16.786 | 40.121 | −1.390 | 1.00 | 60.60 | C |
| ATOM | 134 | CD1 | LEU | A | 25 | 16.740 | 41.310 | −2.344 | 1.00 | 63.05 | C |
| ATOM | 135 | CD2 | LEU | A | 25 | 18.090 | 40.118 | −0.596 | 1.00 | 66.00 | C |
| ATOM | 136 | C | LEU | A | 25 | 14.269 | 38.521 | 1.205 | 1.00 | 62.83 | C |
| ATOM | 137 | O | LEU | A | 25 | 13.371 | 39.363 | 1.295 | 1.00 | 65.96 | O |
| ATOM | 138 | N | GLU | A | 26 | 14.417 | 37.481 | 2.034 | 1.00 | 63.60 | N |
| ATOM | 139 | CA | GLU | A | 26 | 13.557 | 37.151 | 3.180 | 1.00 | 64.36 | C |
| ATOM | 140 | CB | GLU | A | 26 | 12.164 | 36.740 | 2.703 | 1.00 | 65.26 | C |
| ATOM | 141 | CG | GLU | A | 26 | 11.181 | 36.462 | 3.840 | 1.00 | 68.35 | C |
| ATOM | 142 | CD | GLU | A | 26 | 10.195 | 35.365 | 3.503 | 1.00 | 69.03 | C |
| ATOM | 143 | OE1 | GLU | A | 26 | 10.611 | 34.373 | 2.866 | 1.00 | 72.16 | O |
| ATOM | 144 | OE2 | GLU | A | 26 | 9.006 | 35.483 | 3.885 | 1.00 | 70.04 | O |
| ATOM | 145 | C | GLU | A | 26 | 14.182 | 35.967 | 3.926 | 1.00 | 62.58 | C |
| ATOM | 146 | O | GLU | A | 26 | 14.503 | 34.958 | 3.303 | 1.00 | 61.58 | O |
| ATOM | 147 | N | THR | A | 27 | 14.358 | 36.078 | 5.244 | 1.00 | 62.75 | N |
| ATOM | 148 | CA | THR | A | 27 | 14.928 | 34.965 | 6.031 | 1.00 | 62.27 | C |
| ATOM | 149 | CB | THR | A | 27 | 16.365 | 35.278 | 6.593 | 1.00 | 62.72 | C |
| ATOM | 150 | OG1 | THR | A | 27 | 17.247 | 35.654 | 5.526 | 1.00 | 60.03 | O |
| ATOM | 151 | CG2 | THR | A | 27 | 16.959 | 34.054 | 7.245 | 1.00 | 56.16 | C |
| ATOM | 152 | C | THR | A | 27 | 13.979 | 34.389 | 7.117 | 1.00 | 62.81 | C |
| ATOM | 153 | O | THR | A | 27 | 14.427 | 33.864 | 8.148 | 1.00 | 63.93 | O |
| ATOM | 154 | N | ALA | A | 28 | 12.673 | 34.504 | 6.868 | 1.00 | 62.26 | N |
| ATOM | 155 | CA | ALA | A | 28 | 11.630 | 33.803 | 7.634 | 1.00 | 60.61 | C |
| ATOM | 156 | CB | ALA | A | 28 | 10.916 | 34.758 | 8.558 | 1.00 | 62.26 | C |
| ATOM | 157 | C | ALA | A | 28 | 10.646 | 33.186 | 6.647 | 1.00 | 58.58 | C |
| ATOM | 158 | O | ALA | A | 28 | 10.582 | 33.623 | 5.516 | 1.00 | 61.77 | O |
| ATOM | 159 | N | ASP | A | 29 | 9.867 | 32.197 | 7.080 | 1.00 | 57.43 | N |
| ATOM | 160 | CA | ASP | A | 29 | 9.059 | 31.336 | 6.188 | 1.00 | 51.76 | C |
| ATOM | 161 | CB | ASP | A | 29 | 8.248 | 30.372 | 7.029 | 1.00 | 52.60 | C |
| ATOM | 162 | CG | ASP | A | 29 | 9.024 | 29.852 | 8.206 | 1.00 | 54.73 | C |
| ATOM | 163 | OD1 | ASP | A | 29 | 9.472 | 28.696 | 8.133 | 1.00 | 65.52 | O |
| ATOM | 164 | OD2 | ASP | A | 29 | 9.221 | 30.597 | 9.195 | 1.00 | 57.50 | O |
| ATOM | 165 | C | ASP | A | 29 | 8.133 | 32.114 | 5.268 | 1.00 | 48.96 | C |
| ATOM | 166 | O | ASP | A | 29 | 7.583 | 33.121 | 5.686 | 1.00 | 50.09 | O |
| ATOM | 167 | N | LEU | A | 30 | 7.954 | 31.637 | 4.027 | 1.00 | 44.57 | N |
| ATOM | 168 | CA | LEU | A | 30 | 7.173 | 32.357 | 2.999 | 1.00 | 38.90 | C |
| ATOM | 169 | CB | LEU | A | 30 | 7.469 | 31.824 | 1.593 | 1.00 | 33.40 | C |
| ATOM | 170 | CG | LEU | A | 30 | 8.847 | 32.055 | 0.993 | 1.00 | 31.55 | C |
| ATOM | 171 | CD1 | LEU | A | 30 | 9.081 | 30.975 | −0.047 | 1.00 | 34.59 | C |
| ATOM | 172 | CD2 | LEU | A | 30 | 8.873 | 33.455 | 0.382 | 1.00 | 35.00 | C |
| ATOM | 173 | C | LEU | A | 30 | 5.692 | 32.186 | 3.288 | 1.00 | 37.96 | C |
| ATOM | 174 | O | LEU | A | 30 | 4.886 | 33.060 | 2.968 | 1.00 | 38.81 | O |
| ATOM | 175 | N | LYS | A | 31 | 5.352 | 31.042 | 3.872 | 1.00 | 36.60 | N |
| ATOM | 176 | CA | LYS | A | 31 | 3.986 | 30.689 | 4.309 | 1.00 | 36.65 | C |
| ATOM | 177 | CB | LYS | A | 31 | 3.524 | 31.555 | 5.506 | 1.00 | 38.57 | C |
| ATOM | 178 | CG | LYS | A | 31 | 4.378 | 31.527 | 6.791 | 1.00 | 46.53 | C |
| ATOM | 179 | CD | LYS | A | 31 | 4.249 | 30.238 | 7.593 | 1.00 | 57.30 | C |
| ATOM | 180 | CE | LYS | A | 31 | 4.170 | 30.493 | 9.118 | 1.00 | 60.83 | C |
| ATOM | 181 | NZ | LYS | A | 31 | 5.181 | 31.459 | 9.679 | 1.00 | 65.93 | N |
| ATOM | 182 | C | LYS | A | 31 | 2.977 | 30.782 | 3.181 | 1.00 | 39.42 | C |
| ATOM | 183 | O | LYS | A | 31 | 1.858 | 31.305 | 3.381 | 1.00 | 41.43 | O |
| ATOM | 184 | N | TRP | A | 32 | 3.371 | 30.358 | 1.973 | 1.00 | 35.13 | N |
| ATOM | 185 | CA | TRP | A | 32 | 2.453 | 30.416 | 0.834 | 1.00 | 33.15 | C |
| ATOM | 186 | CB | TRP | A | 32 | 3.161 | 30.207 | −0.500 | 1.00 | 28.95 | C |
| ATOM | 187 | CG | TRP | A | 32 | 4.039 | 31.317 | −0.919 | 1.00 | 34.75 | C |
| ATOM | 188 | CD1 | TRP | A | 32 | 4.205 | 32.537 | −0.312 | 1.00 | 33.11 | C |
| ATOM | 189 | NE1 | TRP | A | 32 | 5.093 | 33.291 | −1.021 | 1.00 | 29.19 | N |
| ATOM | 190 | CE2 | TRP | A | 32 | 5.511 | 32.583 | −2.117 | 1.00 | 31.35 | C |
| ATOM | 191 | CD2 | TRP | A | 32 | 4.872 | 31.332 | −2.081 | 1.00 | 32.07 | C |
| ATOM | 192 | CE3 | TRP | A | 32 | 5.140 | 30.397 | −3.090 | 1.00 | 28.33 | C |
| ATOM | 193 | CZ3 | TRP | A | 32 | 6.044 | 30.747 | −4.096 | 1.00 | 29.85 | C |
| ATOM | 194 | CH2 | TRP | A | 32 | 6.681 | 32.008 | −4.088 | 1.00 | 33.09 | C |
| ATOM | 195 | CZ2 | TRP | A | 32 | 6.443 | 32.928 | −3.108 | 1.00 | 29.70 | C |
| ATOM | 196 | C | TRP | A | 32 | 1.381 | 29.360 | 1.045 | 1.00 | 34.03 | C |
| ATOM | 197 | O | TRP | A | 32 | 1.475 | 28.597 | 1.979 | 1.00 | 31.83 | O |
| ATOM | 198 | N | VAL | A | 33 | 0.387 | 29.316 | 0.166 | 1.00 | 36.83 | N |
| ATOM | 199 | CA | VAL | A | 33 | −0.813 | 28.472 | 0.377 | 1.00 | 34.51 | C |
| ATOM | 200 | CB | VAL | A | 33 | −2.075 | 29.329 | 0.158 | 1.00 | 39.47 | C |
| ATOM | 201 | CG1 | VAL | A | 33 | −3.367 | 28.480 | 0.048 | 1.00 | 38.42 | C |
| ATOM | 202 | CG2 | VAL | A | 33 | −2.180 | 30.345 | 1.283 | 1.00 | 29.62 | C |
| ATOM | 203 | C | VAL | A | 33 | −0.811 | 27.254 | −0.516 | 1.00 | 35.90 | C |
| ATOM | 204 | O | VAL | A | 33 | −0.618 | 27.372 | −1.721 | 1.00 | 35.14 | O |
| ATOM | 205 | N | THR | A | 34 | −1.031 | 26.075 | 0.077 | 1.00 | 36.36 | N |
| ATOM | 206 | CA | THR | A | 34 | −1.112 | 24.856 | −0.726 | 1.00 | 36.51 | C |
| ATOM | 207 | CB | THR | A | 34 | −0.110 | 23.848 | −0.259 | 1.00 | 29.05 | C |
| ATOM | 208 | OG1 | THR | A | 34 | −0.276 | 23.679 | 1.146 | 1.00 | 30.25 | O |
| ATOM | 209 | CG2 | THR | A | 34 | 1.324 | 24.336 | −0.559 | 1.00 | 36.15 | C |
| ATOM | 210 | C | THR | A | 34 | −2.504 | 24.202 | −0.691 | 1.00 | 36.01 | C |
| ATOM | 211 | O | THR | A | 34 | −3.244 | 24.330 | 0.293 | 1.00 | 39.89 | O |
| ATOM | 212 | N | PHE | A | 35 | −2.836 | 23.508 | −1.777 | 1.00 | 40.71 | N |
| ATOM | 213 | CA | PHE | A | 35 | −4.038 | 22.661 | −1.865 | 1.00 | 37.31 | C |
| ATOM | 214 | CB | PHE | A | 35 | −5.242 | 23.488 | −2.352 | 1.00 | 39.56 | C |
| ATOM | 215 | CG | PHE | A | 35 | −6.458 | 22.656 | −2.745 | 1.00 | 31.37 | C |
| ATOM | 216 | CD1 | PHE | A | 35 | −7.425 | 22.323 | −1.800 | 1.00 | 38.00 | C |
| ATOM | 217 | CE1 | PHE | A | 35 | −8.536 | 21.564 | −2.155 | 1.00 | 34.26 | C |
| ATOM | 218 | CZ | PHE | A | 35 | −8.710 | 21.162 | −3.467 | 1.00 | 41.72 | C |
| ATOM | 219 | CE2 | PHE | A | 35 | −7.770 | 21.507 | −4.424 | 1.00 | 38.74 | C |
| ATOM | 220 | CD2 | PHE | A | 35 | −6.652 | 22.255 | −4.061 | 1.00 | 37.81 | C |
| ATOM | 221 | C | PHE | A | 35 | −3.799 | 21.477 | −2.807 | 1.00 | 38.54 | C |
| ATOM | 222 | O | PHE | A | 35 | −3.222 | 21.649 | −3.876 | 1.00 | 41.98 | O |
| ATOM | 223 | N | PRO | A | 36 | −4.272 | 20.275 | −2.435 | 1.00 | 36.71 | N |
| ATOM | 224 | CA | PRO | A | 36 | −4.947 | 19.954 | −1.169 | 1.00 | 37.64 | C |
| ATOM | 225 | CB | PRO | A | 36 | −5.768 | 18.692 | −1.504 | 1.00 | 39.48 | C |
| ATOM | 226 | CG | PRO | A | 36 | −5.523 | 18.410 | −2.972 | 1.00 | 38.36 | C |
| ATOM | 227 | CD | PRO | A | 36 | −4.236 | 19.098 | −3.318 | 1.00 | 40.07 | C |
| ATOM | 228 | C | PRO | A | 36 | −3.904 | 19.700 | −0.087 | 1.00 | 40.22 | C |
| ATOM | 229 | O | PRO | A | 36 | −2.750 | 19.379 | −0.420 | 1.00 | 41.14 | O |
| ATOM | 230 | N | GLN | A | 37 | −4.267 | 19.883 | 1.186 | 1.00 | 36.93 | N |
| ATOM | 231 | CA | GLN | A | 37 | −3.271 | 19.770 | 2.261 | 1.00 | 39.87 | C |
| ATOM | 232 | CB | GLN | A | 37 | −3.542 | 20.763 | 3.406 | 1.00 | 40.23 | C |
| ATOM | 233 | CG | GLN | A | 37 | −2.965 | 22.128 | 3.109 | 1.00 | 42.03 | C |
| ATOM | 234 | CD | GLN | A | 37 | −3.673 | 23.293 | 3.765 | 1.00 | 44.82 | C |
| ATOM | 235 | OE1 | GLN | A | 37 | −4.184 | 23.192 | 4.876 | 1.00 | 50.61 | O |
| ATOM | 236 | NE2 | GLN | A | 37 | −3.697 | 24.422 | 3.068 | 1.00 | 47.44 | N |
| ATOM | 237 | C | GLN | A | 37 | −3.103 | 18.329 | 2.732 | 1.00 | 41.85 | C |
| ATOM | 238 | O | GLN | A | 37 | −3.605 | 17.935 | 3.802 | 1.00 | 43.80 | O |
| ATOM | 239 | N | VAL | A | 38 | −2.413 | 17.550 | 1.889 | 1.00 | 40.81 | N |
| ATOM | 240 | CA | VAL | A | 38 | −2.108 | 16.133 | 2.121 | 1.00 | 40.56 | C |
| ATOM | 241 | CB | VAL | A | 38 | −2.824 | 15.199 | 1.098 | 1.00 | 44.60 | C |
| ATOM | 242 | CG1 | VAL | A | 38 | −4.312 | 15.348 | 1.189 | 1.00 | 41.50 | C |
| ATOM | 243 | CG2 | VAL | A | 38 | −2.366 | 15.498 | −0.335 | 1.00 | 46.63 | C |
| ATOM | 244 | C | VAL | A | 38 | −0.623 | 15.866 | 1.983 | 1.00 | 40.11 | C |
| ATOM | 245 | O | VAL | A | 38 | 0.122 | 16.692 | 1.460 | 1.00 | 33.72 | O |
| ATOM | 246 | N | ASP | A | 39 | −0.191 | 14.686 | 2.413 | 1.00 | 39.32 | N |
| ATOM | 247 | CA | ASP | A | 39 | 1.187 | 14.302 | 2.188 | 1.00 | 39.86 | C |
| ATOM | 248 | CB | ASP | A | 39 | 1.489 | 12.950 | 2.845 | 1.00 | 42.53 | C |
| ATOM | 249 | CG | ASP | A | 39 | 2.889 | 12.892 | 3.446 | 1.00 | 54.40 | C |
| ATOM | 250 | OD1 | ASP | A | 39 | 3.849 | 13.397 | 2.806 | 1.00 | 56.20 | O |
| ATOM | 251 | OD2 | ASP | A | 39 | 3.032 | 12.338 | 4.564 | 1.00 | 62.34 | O |
| ATOM | 252 | C | ASP | A | 39 | 1.494 | 14.312 | 0.682 | 1.00 | 38.22 | C |
| ATOM | 253 | O | ASP | A | 39 | 0.734 | 13.790 | −0.121 | 1.00 | 39.89 | O |
| ATOM | 254 | N | GLY | A | 40 | 2.598 | 14.938 | 0.294 | 1.00 | 36.82 | N |
| ATOM | 255 | CA | GLY | A | 40 | 2.906 | 15.094 | −1.115 | 1.00 | 36.32 | C |
| ATOM | 256 | C | GLY | A | 40 | 2.726 | 16.504 | −1.637 | 1.00 | 36.89 | C |
| ATOM | 257 | O | GLY | A | 40 | 3.230 | 16.826 | −2.713 | 1.00 | 40.19 | O |
| ATOM | 258 | N | GLN | A | 41 | 1.959 | 17.320 | −0.910 | 1.00 | 34.90 | N |
| ATOM | 259 | CA | GLN | A | 41 | 1.794 | 18.743 | −1.202 | 1.00 | 31.38 | C |
| ATOM | 260 | CB | GLN | A | 41 | 0.938 | 19.401 | −0.098 | 1.00 | 34.79 | C |
| ATOM | 261 | CG | GLN | A | 41 | 1.706 | 19.597 | 1.237 | 1.00 | 31.27 | C |
| ATOM | 262 | CD | GLN | A | 41 | 0.855 | 19.958 | 2.420 | 1.00 | 32.64 | C |
| ATOM | 263 | OE1 | GLN | A | 41 | 0.175 | 20.974 | 2.428 | 1.00 | 37.65 | O |
| ATOM | 264 | NE2 | GLN | A | 41 | 0.921 | 19.135 | 3.456 | 1.00 | 36.27 | N |
| ATOM | 265 | C | GLN | A | 41 | 3.165 | 19.451 | −1.292 | 1.00 | 32.83 | C |
| ATOM | 266 | O | GLN | A | 41 | 4.113 | 19.037 | −0.633 | 1.00 | 30.94 | O |
| ATOM | 267 | N | TRP | A | 42 | 3.246 | 20.579 | −2.015 | 1.00 | 27.39 | N |
| ATOM | 268 | CA | TRP | A | 42 | 4.499 | 21.331 | −2.053 | 1.00 | 30.30 | C |
| ATOM | 269 | CB | TRP | A | 42 | 4.362 | 22.691 | −2.791 | 1.00 | 30.12 | C |
| ATOM | 270 | CG | TRP | A | 42 | 4.056 | 22.655 | −4.294 | 1.00 | 24.67 | C |
| ATOM | 271 | CD1 | TRP | A | 42 | 2.866 | 22.342 | −4.887 | 1.00 | 29.46 | C |
| ATOM | 272 | NE1 | TRP | A | 42 | 2.993 | 22.421 | −6.268 | 1.00 | 26.03 | N |
| ATOM | 273 | CE2 | TRP | A | 42 | 4.270 | 22.836 | −6.574 | 1.00 | 26.75 | C |
| ATOM | 274 | CD2 | TRP | A | 42 | 4.969 | 22.988 | −5.367 | 1.00 | 22.65 | C |
| ATOM | 275 | CE3 | TRP | A | 42 | 6.310 | 23.374 | −5.402 | 1.00 | 35.38 | C |
| ATOM | 276 | CZ3 | TRP | A | 42 | 6.902 | 23.641 | −6.633 | 1.00 | 25.44 | C |
| ATOM | 277 | CH2 | TRP | A | 42 | 6.173 | 23.493 | −7.821 | 1.00 | 33.53 | C |
| ATOM | 278 | CZ2 | TRP | A | 42 | 4.838 | 23.106 | −7.808 | 1.00 | 26.55 | C |
| ATOM | 279 | C | TRP | A | 42 | 4.829 | 21.626 | −0.614 | 1.00 | 25.05 | C |
| ATOM | 280 | O | TRP | A | 42 | 3.940 | 21.655 | 0.198 | 1.00 | 26.88 | O |
| ATOM | 281 | N | GLU | A | 43 | 6.102 | 21.934 | −0.351 | 1.00 | 28.52 | N |
| ATOM | 282 | CA | GLU | A | 43 | 6.648 | 22.065 | 0.960 | 1.00 | 28.30 | C |
| ATOM | 283 | CB | GLU | A | 43 | 7.221 | 20.708 | 1.368 | 0.50 | 29.79 | C |
| ATOM | 284 | CG | GLU | A | 43 | 7.855 | 20.711 | 2.685 | 0.50 | 27.80 | C |
| ATOM | 285 | CD | GLU | A | 43 | 7.621 | 19.437 | 3.393 | 0.50 | 29.66 | C |
| ATOM | 286 | OE1 | GLU | A | 43 | 7.052 | 19.510 | 4.485 | 0.50 | 28.25 | O |
| ATOM | 287 | OE2 | GLU | A | 43 | 7.976 | 18.362 | 2.850 | 0.50 | 33.51 | O |
| ATOM | 288 | C | GLU | A | 43 | 7.764 | 23.091 | 0.953 | 1.00 | 29.96 | C |
| ATOM | 289 | O | GLU | A | 43 | 8.672 | 23.045 | 0.108 | 1.00 | 30.23 | O |
| ATOM | 290 | N | GLU | A | 44 | 7.694 | 23.981 | 1.942 | 1.00 | 28.74 | N |
| ATOM | 291 | CA | GLU | A | 44 | 8.696 | 24.984 | 2.273 | 1.00 | 32.50 | C |
| ATOM | 292 | CB | GLU | A | 44 | 7.990 | 26.178 | 2.925 | 1.00 | 29.06 | C |
| ATOM | 293 | CG | GLU | A | 44 | 8.921 | 27.058 | 3.729 | 1.00 | 42.19 | C |
| ATOM | 294 | CD | GLU | A | 44 | 8.514 | 28.502 | 3.654 | 1.00 | 35.09 | C |
| ATOM | 295 | OE1 | GLU | A | 44 | 7.329 | 28.813 | 3.928 | 1.00 | 45.10 | O |
| ATOM | 296 | OE2 | GLU | A | 44 | 9.387 | 29.325 | 3.332 | 1.00 | 44.69 | O |
| ATOM | 297 | C | GLU | A | 44 | 9.898 | 24.539 | 3.154 | 1.00 | 27.75 | C |
| ATOM | 298 | O | GLU | A | 44 | 9.733 | 23.944 | 4.232 | 1.00 | 33.26 | O |
| ATOM | 299 | N | LEU | A | 45 | 11.102 | 24.921 | 2.734 | 1.00 | 28.38 | N |
| ATOM | 300 | CA | LEU | A | 45 | 12.330 | 24.400 | 3.255 | 1.00 | 33.31 | C |
| ATOM | 301 | CB | LEU | A | 45 | 12.556 | 23.055 | 2.501 | 1.00 | 37.82 | C |
| ATOM | 302 | CG | LEU | A | 45 | 13.623 | 21.985 | 2.685 | 1.00 | 45.75 | C |
| ATOM | 303 | CD1 | LEU | A | 45 | 13.124 | 20.793 | 1.949 | 1.00 | 39.39 | C |
| ATOM | 304 | CD2 | LEU | A | 45 | 14.894 | 22.391 | 2.058 | 1.00 | 47.39 | C |
| ATOM | 305 | C | LEU | A | 45 | 13.422 | 25.417 | 2.902 | 1.00 | 32.67 | C |
| ATOM | 306 | O | LEU | A | 45 | 13.383 | 26.031 | 1.850 | 1.00 | 31.80 | O |
| ATOM | 307 | N | SER | A | 46 | 14.375 | 25.625 | 3.804 | 0.50 | 25.99 | N |
| ATOM | 308 | CA | SER | A | 46 | 15.545 | 26.385 | 3.455 | 0.50 | 25.69 | C |
| ATOM | 309 | CB | SER | A | 46 | 16.319 | 26.790 | 4.722 | 0.50 | 21.08 | C |
| ATOM | 310 | OG | SER | A | 46 | 15.673 | 27.845 | 5.444 | 0.50 | 17.40 | O |
| ATOM | 311 | C | SER | A | 46 | 16.411 | 25.501 | 2.538 | 0.50 | 25.37 | C |
| ATOM | 312 | O | SER | A | 46 | 16.470 | 24.281 | 2.688 | 0.50 | 27.19 | O |
| ATOM | 313 | N | GLY | A | 47 | 17.042 | 26.131 | 1.567 | 1.00 | 35.25 | N |
| ATOM | 314 | CA | GLY | A | 47 | 18.069 | 25.515 | 0.770 | 1.00 | 37.20 | C |
| ATOM | 315 | C | GLY | A | 47 | 19.112 | 26.509 | 0.272 | 1.00 | 40.71 | C |
| ATOM | 316 | O | GLY | A | 47 | 19.031 | 27.723 | 0.479 | 1.00 | 37.07 | O |
| ATOM | 317 | N | LEU | A | 48 | 20.083 | 25.974 | −0.444 | 1.00 | 42.16 | N |
| ATOM | 318 | CA | LEU | A | 48 | 21.090 | 26.788 | −1.071 | 1.00 | 46.36 | C |
| ATOM | 319 | CB | LEU | A | 48 | 22.472 | 26.147 | −0.882 | 1.00 | 47.59 | C |
| ATOM | 320 | CG | LEU | A | 48 | 23.420 | 26.858 | 0.095 | 1.00 | 46.13 | C |
| ATOM | 321 | CD1 | LEU | A | 48 | 22.890 | 26.860 | 1.523 | 1.00 | 40.81 | C |
| ATOM | 322 | CD2 | LEU | A | 48 | 24.840 | 26.268 | 0.015 | 1.00 | 45.26 | C |
| ATOM | 323 | C | LEU | A | 48 | 20.769 | 26.947 | −2.526 | 1.00 | 47.20 | C |
| ATOM | 324 | O | LEU | A | 48 | 20.149 | 26.061 | −3.134 | 1.00 | 47.47 | O |
| ATOM | 325 | N | ASP | A | 49 | 21.135 | 28.103 | −3.069 | 1.00 | 48.27 | N |
| ATOM | 326 | CA | ASP | A | 49 | 21.056 | 28.332 | −4.499 | 1.00 | 51.38 | C |
| ATOM | 327 | CB | ASP | A | 49 | 20.188 | 29.567 | −4.845 | 1.00 | 50.70 | C |
| ATOM | 328 | CG | ASP | A | 49 | 20.680 | 30.869 | −4.195 | 1.00 | 53.56 | C |
| ATOM | 329 | OD1 | ASP | A | 49 | 21.425 | 30.828 | −3.196 | 1.00 | 53.57 | O |
| ATOM | 330 | OD2 | ASP | A | 49 | 20.299 | 31.962 | −4.694 | 1.00 | 58.12 | O |
| ATOM | 331 | C | ASP | A | 49 | 22.474 | 28.435 | −5.069 | 1.00 | 52.11 | C |
| ATOM | 332 | O | ASP | A | 49 | 23.446 | 28.314 | −4.329 | 1.00 | 53.92 | O |
| ATOM | 333 | N | GLU | A | 50 | 22.576 | 28.657 | −6.375 | 1.00 | 54.51 | N |
| ATOM | 334 | CA | GLU | A | 50 | 23.860 | 28.606 | −7.063 | 1.00 | 56.83 | C |
| ATOM | 335 | CB | GLU | A | 50 | 23.663 | 28.272 | −8.543 | 1.00 | 57.48 | C |
| ATOM | 336 | CG | GLU | A | 50 | 24.955 | 28.198 | −9.341 | 1.00 | 58.63 | C |
| ATOM | 337 | CD | GLU | A | 50 | 24.747 | 28.485 | −10.815 | 1.00 | 63.28 | C |
| ATOM | 338 | OE1 | GLU | A | 50 | 23.826 | 29.261 | −11.147 | 1.00 | 62.81 | O |
| ATOM | 339 | OE2 | GLU | A | 50 | 25.505 | 27.935 | −11.641 | 1.00 | 64.02 | O |
| ATOM | 340 | C | GLU | A | 50 | 24.615 | 29.924 | −6.920 | 1.00 | 58.52 | C |
| ATOM | 341 | O | GLU | A | 50 | 25.752 | 30.052 | −7.375 | 1.00 | 58.64 | O |
| ATOM | 342 | N | GLU | A | 51 | 24.016 | 30.884 | −6.229 | 1.00 | 60.08 | N |
| ATOM | 343 | CA | GLU | A | 51 | 24.755 | 32.014 | −5.680 | 1.00 | 61.33 | C |
| ATOM | 344 | CB | GLU | A | 51 | 23.862 | 33.246 | −5.596 | 1.00 | 60.98 | C |
| ATOM | 345 | CG | GLU | A | 51 | 24.093 | 34.229 | −6.720 | 1.00 | 65.33 | C |
| ATOM | 346 | CD | GLU | A | 51 | 25.552 | 34.338 | −7.099 | 1.00 | 66.61 | C |
| ATOM | 347 | OE1 | GLU | A | 51 | 26.153 | 33.321 | −7.491 | 1.00 | 63.39 | O |
| ATOM | 348 | OE2 | GLU | A | 51 | 26.100 | 35.448 | −7.000 | 1.00 | 70.97 | O |
| ATOM | 349 | C | GLU | A | 51 | 25.355 | 31.726 | −4.313 | 1.00 | 60.91 | C |
| ATOM | 350 | O | GLU | A | 51 | 26.225 | 32.444 | −3.835 | 1.00 | 59.55 | O |
| ATOM | 351 | N | GLN | A | 52 | 24.893 | 30.666 | −3.681 | 1.00 | 60.48 | N |
| ATOM | 352 | CA | GLN | A | 52 | 25.602 | 30.174 | −2.532 | 1.00 | 60.49 | C |
| ATOM | 353 | CB | GLN | A | 52 | 25.917 | 28.705 | −2.692 | 1.00 | 61.75 | C |
| ATOM | 354 | C | GLN | A | 52 | 24.854 | 30.424 | −1.242 | 1.00 | 59.29 | C |
| ATOM | 355 | O | GLN | A | 52 | 25.237 | 29.908 | −0.197 | 1.00 | 60.10 | O |
| ATOM | 356 | N | HIS | A | 53 | 23.804 | 31.235 | −1.297 | 1.00 | 56.75 | N |
| ATOM | 357 | CA | HIS | A | 53 | 23.253 | 31.737 | −0.044 | 1.00 | 54.55 | C |
| ATOM | 358 | CB | HIS | A | 53 | 23.006 | 33.245 | −0.134 | 1.00 | 52.15 | C |
| ATOM | 359 | CG | HIS | A | 53 | 24.232 | 34.039 | −0.458 | 1.00 | 59.55 | C |
| ATOM | 360 | ND1 | HIS | A | 53 | 24.213 | 35.409 | −0.608 | 1.00 | 58.63 | N |
| ATOM | 361 | CE1 | HIS | A | 53 | 25.431 | 35.837 | −0.890 | 1.00 | 60.64 | C |
| ATOM | 362 | NE2 | HIS | A | 53 | 26.239 | 34.793 | −0.929 | 1.00 | 65.96 | N |
| ATOM | 363 | CD2 | HIS | A | 53 | 25.515 | 33.657 | −0.662 | 1.00 | 62.27 | C |
| ATOM | 364 | C | HIS | A | 53 | 21.959 | 31.015 | 0.317 | 1.00 | 52.31 | C |
| ATOM | 365 | O | HIS | A | 53 | 21.175 | 30.649 | −0.558 | 1.00 | 50.31 | O |
| ATOM | 366 | N | SER | A | 54 | 21.743 | 30.812 | 1.613 | 1.00 | 50.47 | N |
| ATOM | 367 | CA | SER | A | 54 | 20.530 | 30.169 | 2.094 | 1.00 | 47.56 | C |
| ATOM | 368 | CB | SER | A | 54 | 20.596 | 29.927 | 3.613 | 1.00 | 48.43 | C |
| ATOM | 369 | OG | SER | A | 54 | 19.286 | 29.843 | 4.172 | 1.00 | 45.67 | O |
| ATOM | 370 | C | SER | A | 54 | 19.287 | 30.986 | 1.758 | 1.00 | 45.87 | C |
| ATOM | 371 | O | SER | A | 54 | 19.123 | 32.128 | 2.237 | 1.00 | 48.05 | O |
| ATOM | 372 | N | VAL | A | 55 | 18.402 | 30.407 | 0.953 | 1.00 | 40.45 | N |
| ATOM | 373 | CA | VAL | A | 55 | 17.105 | 31.085 | 0.673 | 1.00 | 39.01 | C |
| ATOM | 374 | CB | VAL | A | 55 | 17.015 | 31.617 | −0.788 | 1.00 | 36.82 | C |
| ATOM | 375 | CG1 | VAL | A | 55 | 17.864 | 32.882 | −0.951 | 1.00 | 44.80 | C |
| ATOM | 376 | CG2 | VAL | A | 55 | 17.440 | 30.537 | −1.781 | 1.00 | 37.51 | C |
| ATOM | 377 | C | VAL | A | 55 | 15.919 | 30.179 | 1.016 | 1.00 | 34.20 | C |
| ATOM | 378 | O | VAL | A | 55 | 16.091 | 28.989 | 1.260 | 1.00 | 37.43 | O |
| ATOM | 379 | N | ARG | A | 56 | 14.731 | 30.761 | 1.043 | 1.00 | 31.69 | N |
| ATOM | 380 | CA | ARG | A | 56 | 13.461 | 30.033 | 1.228 | 1.00 | 34.24 | C |
| ATOM | 381 | CB | ARG | A | 56 | 12.380 | 31.019 | 1.670 | 1.00 | 34.51 | C |
| ATOM | 382 | CG | ARG | A | 56 | 12.499 | 31.406 | 3.120 | 1.00 | 40.35 | C |
| ATOM | 383 | CD | ARG | A | 56 | 11.870 | 30.286 | 3.920 | 1.00 | 42.68 | C |
| ATOM | 384 | NE | ARG | A | 56 | 12.569 | 30.118 | 5.162 | 1.00 | 54.07 | N |
| ATOM | 385 | CZ | ARG | A | 56 | 12.264 | 29.246 | 6.110 | 1.00 | 45.17 | C |
| ATOM | 386 | NH1 | ARG | A | 56 | 11.208 | 28.431 | 6.026 | 1.00 | 32.64 | N |
| ATOM | 387 | NH2 | ARG | A | 56 | 13.020 | 29.249 | 7.184 | 1.00 | 43.41 | N |
| ATOM | 388 | C | ARG | A | 56 | 13.050 | 29.358 | −0.068 | 1.00 | 36.72 | C |
| ATOM | 389 | O | ARG | A | 56 | 12.933 | 30.049 | −1.074 | 1.00 | 37.99 | O |
| ATOM | 390 | N | THR | A | 57 | 12.898 | 28.028 | −0.054 | 1.00 | 37.40 | N |
| ATOM | 391 | CA | THR | A | 57 | 12.555 | 27.264 | −1.269 | 1.00 | 36.12 | C |
| ATOM | 392 | CB | THR | A | 57 | 13.657 | 26.239 | −1.669 | 1.00 | 31.88 | C |
| ATOM | 393 | OG1 | THR | A | 57 | 13.665 | 25.129 | −0.746 | 1.00 | 32.23 | O |
| ATOM | 394 | CG2 | THR | A | 57 | 15.055 | 26.937 | −1.774 | 1.00 | 30.55 | C |
| ATOM | 395 | C | THR | A | 57 | 11.198 | 26.536 | −1.150 | 1.00 | 30.38 | C |
| ATOM | 396 | O | THR | A | 57 | 10.690 | 26.366 | −0.049 | 1.00 | 32.87 | O |
| ATOM | 397 | N | TYR | A | 58 | 10.671 | 26.073 | −2.281 | 1.00 | 29.60 | N |
| ATOM | 398 | CA | TYR | A | 58 | 9.463 | 25.211 | −2.336 | 1.00 | 25.84 | C |
| ATOM | 399 | CB | TYR | A | 58 | 8.204 | 25.927 | −2.875 | 1.00 | 33.81 | C |
| ATOM | 400 | CG | TYR | A | 58 | 7.373 | 26.568 | −1.769 | 1.00 | 32.75 | C |
| ATOM | 401 | CD1 | TYR | A | 58 | 6.581 | 25.762 | −0.919 | 1.00 | 36.07 | C |
| ATOM | 402 | CE1 | TYR | A | 58 | 5.857 | 26.309 | 0.107 | 1.00 | 33.85 | C |
| ATOM | 403 | CZ | TYR | A | 58 | 5.909 | 27.681 | 0.318 | 1.00 | 28.13 | C |
| ATOM | 404 | OH | TYR | A | 58 | 5.143 | 28.196 | 1.340 | 1.00 | 39.16 | O |
| ATOM | 405 | CE2 | TYR | A | 58 | 6.675 | 28.495 | −0.494 | 1.00 | 32.86 | C |
| ATOM | 406 | CD2 | TYR | A | 58 | 7.385 | 27.940 | −1.549 | 1.00 | 30.61 | C |
| ATOM | 407 | C | TYR | A | 58 | 9.759 | 23.931 | −3.119 | 1.00 | 27.75 | C |
| ATOM | 408 | O | TYR | A | 58 | 10.237 | 23.944 | −4.251 | 1.00 | 30.30 | O |
| ATOM | 409 | N | GLU | A | 59 | 9.493 | 22.808 | −2.518 | 1.00 | 26.84 | N |
| ATOM | 410 | CA | GLU | A | 59 | 9.743 | 21.517 | −3.207 | 1.00 | 28.50 | C |
| ATOM | 411 | CB | GLU | A | 59 | 10.734 | 20.698 | −2.395 | 1.00 | 25.37 | C |
| ATOM | 412 | CG | GLU | A | 59 | 12.194 | 21.194 | −2.554 | 1.00 | 32.71 | C |
| ATOM | 413 | CD | GLU | A | 59 | 13.169 | 20.434 | −1.708 | 1.00 | 35.65 | C |
| ATOM | 414 | OE1 | GLU | A | 59 | 12.752 | 19.458 | −1.043 | 1.00 | 42.68 | O |
| ATOM | 415 | OE2 | GLU | A | 59 | 14.373 | 20.788 | −1.734 | 1.00 | 38.99 | O |
| ATOM | 416 | C | GLU | A | 59 | 8.445 | 20.719 | −3.429 | 1.00 | 29.63 | C |
| ATOM | 417 | O | GLU | A | 59 | 7.573 | 20.699 | −2.581 | 1.00 | 29.86 | O |
| ATOM | 418 | N | VAL | A | 60 | 8.304 | 20.114 | −4.597 | 1.00 | 32.15 | N |
| ATOM | 419 | CA | VAL | A | 60 | 7.342 | 19.043 | −4.786 | 1.00 | 28.58 | C |
| ATOM | 420 | CB | VAL | A | 60 | 6.074 | 19.527 | −5.482 | 1.00 | 30.98 | C |
| ATOM | 421 | CG1 | VAL | A | 60 | 6.353 | 19.831 | −6.910 | 1.00 | 23.51 | C |
| ATOM | 422 | CG2 | VAL | A | 60 | 4.903 | 18.501 | −5.270 | 1.00 | 28.38 | C |
| ATOM | 423 | C | VAL | A | 60 | 8.023 | 17.825 | −5.495 | 1.00 | 32.59 | C |
| ATOM | 424 | O | VAL | A | 60 | 8.836 | 17.970 | −6.418 | 1.00 | 31.84 | O |
| ATOM | 425 | N | CYS | A | 61 | 7.706 | 16.628 | −5.036 | 1.00 | 32.96 | N |
| ATOM | 426 | CA | CYS | A | 61 | 8.172 | 15.412 | −5.731 | 1.00 | 35.72 | C |
| ATOM | 427 | CB | CYS | A | 61 | 9.667 | 15.181 | −5.499 | 1.00 | 36.99 | C |
| ATOM | 428 | SG | CYS | A | 61 | 10.357 | 13.789 | −6.460 | 1.00 | 35.75 | S |
| ATOM | 429 | C | CYS | A | 61 | 7.320 | 14.168 | −5.440 | 1.00 | 39.90 | C |
| ATOM | 430 | O | CYS | A | 61 | 7.784 | 13.212 | −4.847 | 1.00 | 37.78 | O |
| ATOM | 431 | N | ASP | A | 62 | 6.070 | 14.192 | −5.902 | 1.00 | 42.20 | N |
| ATOM | 432 | CA | ASP | A | 62 | 5.098 | 13.145 | −5.583 | 1.00 | 46.74 | C |
| ATOM | 433 | CB | ASP | A | 62 | 3.882 | 13.839 | −4.937 | 1.00 | 44.78 | C |
| ATOM | 434 | CG | ASP | A | 62 | 2.847 | 12.878 | −4.360 | 1.00 | 47.88 | C |
| ATOM | 435 | OD1 | ASP | A | 62 | 3.210 | 11.852 | −3.750 | 1.00 | 41.91 | O |
| ATOM | 436 | OD2 | ASP | A | 62 | 1.648 | 13.198 | −4.502 | 1.00 | 40.24 | O |
| ATOM | 437 | C | ASP | A | 62 | 4.748 | 12.325 | −6.856 | 1.00 | 50.85 | C |
| ATOM | 438 | O | ASP | A | 62 | 3.567 | 12.170 | −7.223 | 1.00 | 49.30 | O |
| ATOM | 439 | N | VAL | A | 63 | 5.792 | 11.771 | −7.494 | 1.00 | 51.88 | N |
| ATOM | 440 | CA | VAL | A | 63 | 5.747 | 11.308 | −8.913 | 1.00 | 54.74 | C |
| ATOM | 441 | CB | VAL | A | 63 | 7.166 | 11.296 | −9.566 | 1.00 | 54.31 | C |
| ATOM | 442 | CG1 | VAL | A | 63 | 7.692 | 12.700 | −9.757 | 1.00 | 51.43 | C |
| ATOM | 443 | CG2 | VAL | A | 63 | 8.139 | 10.455 | −8.736 | 1.00 | 54.86 | C |
| ATOM | 444 | C | VAL | A | 63 | 5.123 | 9.933 | −9.169 | 1.00 | 59.46 | C |
| ATOM | 445 | O | VAL | A | 63 | 4.803 | 9.596 | −10.319 | 1.00 | 59.09 | O |
| ATOM | 446 | N | GLN | A | 64 | 4.992 | 9.134 | −8.108 | 1.00 | 62.97 | N |
| ATOM | 447 | CA | GLN | A | 64 | 4.441 | 7.787 | −8.205 | 1.00 | 67.89 | C |
| ATOM | 448 | CB | GLN | A | 64 | 5.523 | 6.735 | −7.964 | 1.00 | 69.08 | C |
| ATOM | 449 | C | GLN | A | 64 | 3.331 | 7.600 | −7.193 | 1.00 | 70.54 | C |
| ATOM | 450 | O | GLN | A | 64 | 2.381 | 6.841 | −7.433 | 1.00 | 71.49 | O |
| ATOM | 451 | N | ARG | A | 65 | 3.469 | 8.300 | −6.065 | 1.00 | 72.75 | N |
| ATOM | 452 | CA | ARG | A | 65 | 2.538 | 8.217 | −4.937 | 1.00 | 73.43 | C |
| ATOM | 453 | CB | ARG | A | 65 | 3.107 | 8.979 | −3.739 | 1.00 | 73.53 | C |
| ATOM | 454 | CG | ARG | A | 65 | 4.643 | 8.994 | −3.671 | 1.00 | 74.34 | C |
| ATOM | 455 | CD | ARG | A | 65 | 5.151 | 9.895 | −2.553 | 1.00 | 74.16 | C |
| ATOM | 456 | NE | ARG | A | 65 | 6.539 | 9.612 | −2.185 | 1.00 | 78.12 | N |
| ATOM | 457 | CZ | ARG | A | 65 | 6.912 | 8.709 | −1.276 | 1.00 | 81.38 | C |
| ATOM | 458 | NH1 | ARG | A | 65 | 8.203 | 8.535 | −1.008 | 1.00 | 81.84 | N |
| ATOM | 459 | NH2 | ARG | A | 65 | 6.001 | 7.979 | −0.630 | 1.00 | 80.28 | N |
| ATOM | 460 | C | ARG | A | 65 | 1.175 | 8.781 | −5.319 | 1.00 | 74.40 | C |
| ATOM | 461 | O | ARG | A | 65 | 0.137 | 8.308 | −4.848 | 1.00 | 75.23 | O |
| ATOM | 462 | N | ALA | A | 66 | 1.187 | 9.752 | −6.221 | 1.00 | 75.02 | N |
| ATOM | 463 | CA | ALA | A | 66 | −0.022 | 10.410 | −6.687 | 1.00 | 75.51 | C |
| ATOM | 464 | CB | ALA | A | 66 | 0.324 | 11.758 | −7.268 | 1.00 | 74.75 | C |
| ATOM | 465 | C | ALA | A | 66 | −0.772 | 9.571 | −7.719 | 1.00 | 76.66 | C |
| ATOM | 466 | O | ALA | A | 66 | −0.182 | 8.738 | −8.406 | 1.00 | 75.96 | O |
| ATOM | 467 | N | PRO | A | 67 | −2.081 | 9.805 | −7.831 | 1.00 | 76.02 | N |
| ATOM | 468 | CA | PRO | A | 67 | −2.860 | 9.213 | −8.920 | 1.00 | 76.11 | C |
| ATOM | 469 | CB | PRO | A | 67 | −3.444 | 7.964 | −8.264 | 1.00 | 76.99 | C |
| ATOM | 470 | CG | PRO | A | 67 | −3.465 | 8.286 | −6.762 | 1.00 | 76.64 | C |
| ATOM | 471 | CD | PRO | A | 67 | −2.737 | 9.576 | −6.533 | 1.00 | 75.96 | C |
| ATOM | 472 | C | PRO | A | 67 | −4.001 | 10.095 | −9.427 | 1.00 | 73.84 | C |
| ATOM | 473 | O | PRO | A | 67 | −5.117 | 9.990 | −8.927 | 1.00 | 74.12 | O |
| ATOM | 474 | N | GLY | A | 68 | −3.731 | 10.931 | −10.422 | 1.00 | 71.47 | N |
| ATOM | 475 | CA | GLY | A | 68 | −4.781 | 11.685 | −11.088 | 1.00 | 68.41 | C |
| ATOM | 476 | C | GLY | A | 68 | −4.893 | 13.125 | −10.618 | 1.00 | 65.77 | C |
| ATOM | 477 | O | GLY | A | 68 | −5.894 | 13.791 | −10.859 | 1.00 | 65.68 | O |
| ATOM | 478 | N | GLN | A | 69 | −3.854 | 13.607 | −9.946 | 1.00 | 62.00 | N |
| ATOM | 479 | CA | GLN | A | 69 | −4.019 | 14.608 | −8.911 | 1.00 | 58.03 | C |
| ATOM | 480 | CB | GLN | A | 69 | −3.807 | 13.999 | −7.541 | 1.00 | 57.70 | C |
| ATOM | 481 | CG | GLN | A | 69 | −4.617 | 14.674 | −6.473 | 1.00 | 60.32 | C |
| ATOM | 482 | CD | GLN | A | 69 | −3.768 | 15.263 | −5.391 | 1.00 | 63.50 | C |
| ATOM | 483 | OE1 | GLN | A | 69 | −2.571 | 15.035 | −5.343 | 1.00 | 69.20 | O |
| ATOM | 484 | NE2 | GLN | A | 69 | −4.383 | 16.027 | −4.509 | 1.00 | 64.13 | N |
| ATOM | 485 | C | GLN | A | 69 | −3.103 | 15.810 | −9.075 | 1.00 | 54.71 | C |
| ATOM | 486 | O | GLN | A | 69 | −2.022 | 15.710 | −9.640 | 1.00 | 52.38 | O |
| ATOM | 487 | N | ALA | A | 70 | −3.554 | 16.948 | −8.567 | 1.00 | 50.26 | N |
| ATOM | 488 | CA | ALA | A | 70 | −2.866 | 18.217 | −8.769 | 1.00 | 45.28 | C |
| ATOM | 489 | CB | ALA | A | 70 | −3.709 | 19.146 | −9.653 | 1.00 | 46.12 | C |
| ATOM | 490 | C | ALA | A | 70 | −2.513 | 18.911 | −7.461 | 1.00 | 40.85 | C |
| ATOM | 491 | O | ALA | A | 70 | −3.392 | 19.227 | −6.667 | 1.00 | 40.28 | O |
| ATOM | 492 | N | HIS | A | 71 | −1.226 | 19.190 | −7.258 | 1.00 | 38.57 | N |
| ATOM | 493 | CA | HIS | A | 71 | −0.776 | 19.932 | −6.072 | 1.00 | 34.83 | C |
| ATOM | 494 | CB | HIS | A | 71 | 0.567 | 19.415 | −5.531 | 1.00 | 33.42 | C |
| ATOM | 495 | CG | HIS | A | 71 | 0.567 | 17.965 | −5.101 | 1.00 | 36.67 | C |
| ATOM | 496 | ND1 | HIS | A | 71 | −0.248 | 17.483 | −4.094 | 1.00 | 42.49 | N |
| ATOM | 497 | CE1 | HIS | A | 71 | −0.032 | 16.189 | −3.928 | 1.00 | 37.72 | C |
| ATOM | 498 | NE2 | HIS | A | 71 | 0.924 | 15.816 | −4.764 | 1.00 | 35.72 | N |
| ATOM | 499 | CD2 | HIS | A | 71 | 1.314 | 16.912 | −5.507 | 1.00 | 40.69 | C |
| ATOM | 500 | C | HIS | A | 71 | −0.633 | 21.421 | −6.445 | 1.00 | 36.72 | C |
| ATOM | 501 | O | HIS | A | 71 | 0.255 | 21.811 | −7.240 | 1.00 | 30.07 | O |
| ATOM | 502 | N | TRP | A | 72 | −1.458 | 22.244 | −5.819 | 1.00 | 35.14 | N |
| ATOM | 503 | CA | TRP | A | 72 | −1.437 | 23.690 | −6.072 | 1.00 | 34.04 | C |
| ATOM | 504 | CB | TRP | A | 72 | −2.853 | 24.250 | −6.076 | 1.00 | 35.63 | C |
| ATOM | 505 | CG | TRP | A | 72 | −3.670 | 23.874 | −7.269 | 1.00 | 30.19 | C |
| ATOM | 506 | CD1 | TRP | A | 72 | −4.373 | 22.721 | −7.443 | 1.00 | 37.72 | C |
| ATOM | 507 | NE1 | TRP | A | 72 | −4.994 | 22.715 | −8.654 | 1.00 | 37.02 | N |
| ATOM | 508 | CE2 | TRP | A | 72 | −4.722 | 23.892 | −9.300 | 1.00 | 41.37 | C |
| ATOM | 509 | CD2 | TRP | A | 72 | −3.877 | 24.648 | −8.455 | 1.00 | 43.82 | C |
| ATOM | 510 | CE3 | TRP | A | 72 | −3.443 | 25.912 | −8.885 | 1.00 | 41.53 | C |
| ATOM | 511 | CZ3 | TRP | A | 72 | −3.857 | 26.370 | −10.124 | 1.00 | 37.00 | C |
| ATOM | 512 | CH2 | TRP | A | 72 | −4.701 | 25.594 | −10.945 | 1.00 | 37.71 | C |
| ATOM | 513 | CZ2 | TRP | A | 72 | −5.139 | 24.352 | −10.549 | 1.00 | 45.05 | C |
| ATOM | 514 | C | TRP | A | 72 | −0.591 | 24.390 | −5.020 | 1.00 | 32.97 | C |
| ATOM | 515 | O | TRP | A | 72 | −0.574 | 23.956 | −3.862 | 1.00 | 30.07 | C |
| ATOM | 516 | N | LEU | A | 73 | 0.198 | 25.379 | −5.463 | 1.00 | 29.15 | N |
| ATOM | 517 | CA | LEU | A | 73 | 0.924 | 26.326 | −4.587 | 1.00 | 28.01 | C |
| ATOM | 518 | CB | LEU | A | 73 | 2.450 | 26.140 | −4.720 | 1.00 | 24.79 | C |
| ATOM | 519 | CG | LEU | A | 73 | 3.354 | 27.007 | −3.870 | 1.00 | 28.60 | C |
| ATOM | 520 | CD1 | LEU | A | 73 | 3.116 | 26.818 | −2.389 | 1.00 | 28.15 | C |
| ATOM | 521 | CD2 | LEU | A | 73 | 4.848 | 26.781 | −4.220 | 1.00 | 30.48 | C |
| ATOM | 522 | C | LEU | A | 73 | 0.587 | 27.771 | −4.989 | 1.00 | 28.82 | C |
| ATOM | 523 | O | LEU | A | 73 | 0.741 | 28.143 | −6.166 | 1.00 | 32.47 | O |
| ATOM | 524 | N | ARG | A | 74 | 0.229 | 28.601 | −4.016 | 1.00 | 26.25 | N |
| ATOM | 525 | CA | ARG | A | 74 | −0.171 | 29.997 | −4.349 | 1.00 | 31.34 | C |
| ATOM | 526 | CB | ARG | A | 74 | −1.692 | 30.180 | −4.242 | 1.00 | 32.48 | C |
| ATOM | 527 | CG | ARG | A | 74 | −2.258 | 31.453 | −4.942 | 1.00 | 30.02 | C |
| ATOM | 528 | CD | ARG | A | 74 | −3.720 | 31.633 | −4.574 | 1.00 | 36.24 | C |
| ATOM | 529 | NE | ARG | A | 74 | −3.803 | 31.821 | −3.132 | 1.00 | 39.56 | N |
| ATOM | 530 | CZ | ARG | A | 74 | −4.863 | 31.571 | −2.383 | 1.00 | 40.38 | C |
| ATOM | 531 | NH1 | ARG | A | 74 | −6.001 | 31.116 | −2.916 | 1.00 | 38.53 | N |
| ATOM | 532 | NH2 | ARG | A | 74 | −4.767 | 31.798 | −1.085 | 1.00 | 37.96 | N |
| ATOM | 533 | C | ARG | A | 74 | 0.553 | 31.020 | −3.470 | 1.00 | 31.04 | C |
| ATOM | 534 | O | ARG | A | 74 | 0.638 | 30.847 | −2.258 | 1.00 | 32.22 | O |
| ATOM | 535 | N | THR | A | 75 | 1.075 | 32.077 | −4.084 | 1.00 | 33.16 | N |
| ATOM | 536 | CA | THR | A | 75 | 1.679 | 33.193 | −3.289 | 1.00 | 34.65 | C |
| ATOM | 537 | CB | THR | A | 75 | 2.319 | 34.284 | −4.147 | 1.00 | 34.94 | C |
| ATOM | 538 | OG1 | THR | A | 75 | 1.299 | 34.906 | −4.943 | 1.00 | 31.24 | O |
| ATOM | 539 | CG2 | THR | A | 75 | 3.385 | 33.710 | −5.035 | 1.00 | 38.97 | C |
| ATOM | 540 | C | THR | A | 75 | 0.649 | 33.872 | −2.415 | 1.00 | 32.75 | C |
| ATOM | 541 | O | THR | A | 75 | −0.566 | 33.639 | −2.562 | 1.00 | 30.37 | O |
| ATOM | 542 | N | GLY | A | 76 | 1.134 | 34.708 | −1.493 | 1.00 | 36.03 | N |
| ATOM | 543 | CA | GLY | A | 76 | 0.280 | 35.638 | −0.751 | 1.00 | 34.99 | C |
| ATOM | 544 | C | GLY | A | 76 | −0.168 | 36.709 | −1.711 | 1.00 | 34.57 | C |
| ATOM | 545 | O | GLY | A | 76 | 0.262 | 36.700 | −2.865 | 1.00 | 35.49 | O |
| ATOM | 546 | N | TRP | A | 77 | −1.032 | 37.612 | −1.238 | 1.00 | 32.47 | N |
| ATOM | 547 | CA | TRP | A | 77 | −1.626 | 38.650 | −2.085 | 1.00 | 34.91 | C |
| ATOM | 548 | CB | TRP | A | 77 | −2.565 | 39.510 | −1.255 | 1.00 | 33.83 | C |
| ATOM | 549 | CG | TRP | A | 77 | −3.507 | 40.357 | −2.092 | 1.00 | 38.76 | C |
| ATOM | 550 | CD1 | TRP | A | 77 | −4.293 | 39.951 | −3.139 | 1.00 | 36.42 | C |
| ATOM | 551 | NE1 | TRP | A | 77 | −4.986 | 41.040 | −3.659 | 1.00 | 35.07 | N |
| ATOM | 552 | CE2 | TRP | A | 77 | −4.662 | 42.153 | −2.933 | 1.00 | 38.58 | C |
| ATOM | 553 | CD2 | TRP | A | 77 | −3.735 | 41.760 | −1.937 | 1.00 | 42.72 | C |
| ATOM | 554 | CE3 | TRP | A | 77 | −3.244 | 42.726 | −1.047 | 1.00 | 45.06 | C |
| ATOM | 555 | CZ3 | TRP | A | 77 | −3.675 | 44.034 | −1.182 | 1.00 | 37.42 | C |
| ATOM | 556 | CH2 | TRP | A | 77 | −4.611 | 44.396 | −2.177 | 1.00 | 39.82 | C |
| ATOM | 557 | CZ2 | TRP | A | 77 | −5.117 | 43.471 | −3.055 | 1.00 | 41.18 | C |
| ATOM | 558 | C | TRP | A | 77 | −0.528 | 39.545 | −2.587 | 1.00 | 34.98 | C |
| ATOM | 559 | O | TRP | A | 77 | 0.240 | 40.023 | −1.792 | 1.00 | 35.74 | O |
| ATOM | 560 | N | VAL | A | 78 | −0.450 | 39.804 | −3.888 | 1.00 | 38.30 | N |
| ATOM | 561 | CA | VAL | A | 78 | 0.580 | 40.746 | −4.342 | 1.00 | 39.04 | C |
| ATOM | 562 | CB | VAL | A | 78 | 1.558 | 40.141 | −5.392 | 1.00 | 39.37 | C |
| ATOM | 563 | CG1 | VAL | A | 78 | 2.797 | 41.050 | −5.563 | 1.00 | 39.09 | C |
| ATOM | 564 | CG2 | VAL | A | 78 | 1.994 | 38.700 | −4.983 | 1.00 | 33.31 | C |
| ATOM | 565 | C | VAL | A | 78 | −0.020 | 42.079 | −4.854 | 1.00 | 42.68 | C |
| ATOM | 566 | O | VAL | A | 78 | −0.694 | 42.105 | −5.910 | 1.00 | 41.94 | O |
| ATOM | 567 | N | PRO | A | 79 | 0.235 | 43.176 | −4.110 | 1.00 | 42.96 | N |
| ATOM | 568 | CA | PRO | A | 79 | −0.097 | 44.504 | −4.588 | 1.00 | 46.35 | C |
| ATOM | 569 | CB | PRO | A | 79 | 0.378 | 45.420 | −3.449 | 1.00 | 44.48 | C |
| ATOM | 570 | CG | PRO | A | 79 | 0.432 | 44.527 | −2.236 | 1.00 | 38.83 | C |
| ATOM | 571 | CD | PRO | A | 79 | 0.876 | 43.222 | −2.777 | 1.00 | 41.53 | C |
| ATOM | 572 | C | PRO | A | 79 | 0.611 | 44.806 | −5.934 | 1.00 | 49.47 | C |
| ATOM | 573 | O | PRO | A | 79 | 1.832 | 44.949 | −6.009 | 1.00 | 51.20 | O |
| ATOM | 574 | N | ARG | A | 80 | −0.183 | 44.829 | −6.997 | 1.00 | 53.14 | N |
| ATOM | 575 | CA | ARG | A | 80 | 0.249 | 45.329 | −8.281 | 1.00 | 54.18 | C |
| ATOM | 576 | CB | ARG | A | 80 | −0.769 | 44.923 | −9.340 | 1.00 | 55.34 | C |
| ATOM | 577 | CG | ARG | A | 80 | −0.888 | 45.833 | −10.533 | 1.00 | 51.62 | C |
| ATOM | 578 | CD | ARG | A | 80 | −2.346 | 45.943 | −10.982 | 1.00 | 47.92 | C |
| ATOM | 579 | NE | ARG | A | 80 | −3.010 | 47.189 | −10.538 | 1.00 | 43.68 | N |
| ATOM | 580 | CZ | ARG | A | 80 | −2.977 | 48.363 | −11.174 | 1.00 | 53.21 | C |
| ATOM | 581 | NH1 | ARG | A | 80 | −2.282 | 48.541 | −12.304 | 1.00 | 48.15 | N |
| ATOM | 582 | NH2 | ARG | A | 80 | −3.642 | 49.389 | −10.660 | 1.00 | 50.73 | N |
| ATOM | 583 | C | ARG | A | 80 | 0.281 | 46.825 | −8.058 | 1.00 | 56.38 | C |
| ATOM | 584 | O | ARG | A | 80 | −0.765 | 47.478 | −8.004 | 1.00 | 57.66 | O |
| ATOM | 585 | N | ARG | A | 81 | 1.476 | 47.362 | −7.852 | 1.00 | 57.34 | N |
| ATOM | 586 | CA | ARG | A | 81 | 1.607 | 48.768 | −7.490 | 1.00 | 58.23 | C |
| ATOM | 587 | CB | ARG | A | 81 | 3.054 | 49.083 | −7.124 | 1.00 | 58.75 | C |
| ATOM | 588 | CG | ARG | A | 81 | 3.430 | 48.515 | −5.765 | 1.00 | 57.81 | C |
| ATOM | 589 | CD | ARG | A | 81 | 4.811 | 47.914 | −5.769 | 1.00 | 65.79 | C |
| ATOM | 590 | NE | ARG | A | 81 | 4.846 | 46.682 | −4.989 | 1.00 | 69.25 | N |
| ATOM | 591 | CZ | ARG | A | 81 | 5.910 | 46.228 | −4.334 | 1.00 | 71.96 | C |
| ATOM | 592 | NH1 | ARG | A | 81 | 7.049 | 46.912 | −4.349 | 1.00 | 73.08 | N |
| ATOM | 593 | NH2 | ARG | A | 81 | 5.830 | 45.086 | −3.655 | 1.00 | 75.90 | N |
| ATOM | 594 | C | ARG | A | 81 | 1.057 | 49.687 | −8.584 | 1.00 | 59.36 | C |
| ATOM | 595 | O | ARG | A | 81 | −0.012 | 50.285 | −8.418 | 1.00 | 60.97 | O |
| ATOM | 596 | N | GLY | A | 82 | 1.775 | 49.786 | −9.698 | 1.00 | 57.47 | N |
| ATOM | 597 | CA | GLY | A | 82 | 1.291 | 50.512 | −10.849 | 1.00 | 56.99 | C |
| ATOM | 598 | C | GLY | A | 82 | 1.528 | 49.695 | −12.092 | 1.00 | 56.65 | C |
| ATOM | 599 | O | GLY | A | 82 | 1.475 | 50.219 | −13.206 | 1.00 | 56.18 | O |
| ATOM | 600 | N | ALA | A | 83 | 1.801 | 48.407 | −11.892 | 1.00 | 55.51 | N |
| ATOM | 601 | CA | ALA | A | 83 | 2.078 | 47.474 | −12.984 | 1.00 | 55.06 | C |
| ATOM | 602 | CB | ALA | A | 83 | 2.622 | 46.159 | −12.439 | 1.00 | 53.08 | C |
| ATOM | 603 | C | ALA | A | 83 | 0.877 | 47.208 | −13.868 | 1.00 | 54.91 | C |
| ATOM | 604 | O | ALA | A | 83 | −0.272 | 47.183 | −13.416 | 1.00 | 56.83 | O |
| ATOM | 605 | N | VAL | A | 84 | 1.156 | 46.989 | −15.140 | 1.00 | 55.31 | N |
| ATOM | 606 | CA | VAL | A | 84 | 0.123 | 46.541 | −16.053 | 1.00 | 56.06 | C |
| ATOM | 607 | CB | VAL | A | 84 | −0.135 | 47.546 | −17.203 | 1.00 | 56.51 | C |
| ATOM | 608 | CG1 | VAL | A | 84 | −0.933 | 46.892 | −18.338 | 1.00 | 54.38 | C |
| ATOM | 609 | CG2 | VAL | A | 84 | −0.863 | 48.773 | −16.661 | 1.00 | 51.88 | C |
| ATOM | 610 | C | VAL | A | 84 | 0.555 | 45.187 | −16.569 | 1.00 | 56.01 | C |
| ATOM | 611 | O | VAL | A | 84 | −0.244 | 44.242 | −16.603 | 1.00 | 57.12 | O |
| ATOM | 612 | N | HIS | A | 85 | 1.818 | 45.103 | −16.965 | 1.00 | 52.37 | N |
| ATOM | 613 | CA | HIS | A | 85 | 2.404 | 43.814 | −17.268 | 1.00 | 52.72 | C |
| ATOM | 614 | CB | HIS | A | 85 | 2.900 | 43.727 | −18.711 | 1.00 | 52.43 | C |
| ATOM | 615 | CG | HIS | A | 85 | 1.810 | 43.911 | −19.713 | 1.00 | 54.23 | C |
| ATOM | 616 | ND1 | HIS | A | 85 | 1.059 | 42.864 | −20.195 | 1.00 | 50.36 | N |
| ATOM | 617 | CE1 | HIS | A | 85 | 0.154 | 43.327 | −21.038 | 1.00 | 53.21 | C |
| ATOM | 618 | NE2 | HIS | A | 85 | 0.284 | 44.639 | −21.109 | 1.00 | 54.30 | N |
| ATOM | 619 | CD2 | HIS | A | 85 | 1.308 | 45.031 | −20.283 | 1.00 | 52.00 | C |
| ATOM | 620 | C | HIS | A | 85 | 3.485 | 43.516 | −16.267 | 1.00 | 49.96 | C |
| ATOM | 621 | O | HIS | A | 85 | 4.397 | 44.310 | −16.049 | 1.00 | 51.17 | O |
| ATOM | 622 | N | VAL | A | 86 | 3.332 | 42.366 | −15.630 | 1.00 | 47.91 | N |
| ATOM | 623 | CA | VAL | A | 86 | 4.242 | 41.911 | −14.610 | 1.00 | 47.10 | C |
| ATOM | 624 | CB | VAL | A | 86 | 3.431 | 41.339 | −13.414 | 1.00 | 49.81 | C |
| ATOM | 625 | CG1 | VAL | A | 86 | 4.221 | 40.335 | −12.580 | 1.00 | 46.34 | C |
| ATOM | 626 | CG2 | VAL | A | 86 | 2.883 | 42.469 | −12.557 | 1.00 | 49.43 | C |
| ATOM | 627 | C | VAL | A | 86 | 5.190 | 40.892 | −15.246 | 1.00 | 43.83 | C |
| ATOM | 628 | O | VAL | A | 86 | 4.804 | 40.171 | −16.172 | 1.00 | 42.11 | O |
| ATOM | 629 | N | TYR | A | 87 | 6.438 | 40.879 | −14.781 | 1.00 | 39.95 | N |
| ATOM | 630 | CA | TYR | A | 87 | 7.384 | 39.865 | −15.203 | 1.00 | 37.91 | C |
| ATOM | 631 | CB | TYR | A | 87 | 8.724 | 40.475 | −15.541 | 1.00 | 35.88 | C |
| ATOM | 632 | CG | TYR | A | 87 | 8.811 | 41.281 | −16.830 | 1.00 | 42.37 | C |
| ATOM | 633 | CD1 | TYR | A | 87 | 9.646 | 40.873 | −17.871 | 1.00 | 44.12 | C |
| ATOM | 634 | CE1 | TYR | A | 87 | 9.779 | 41.618 | −19.021 | 1.00 | 47.12 | C |
| ATOM | 635 | CZ | TYR | A | 87 | 9.074 | 42.791 | −19.142 | 1.00 | 39.76 | C |
| ATOM | 636 | OH | TYR | A | 87 | 9.179 | 43.539 | −20.295 | 1.00 | 50.68 | O |
| ATOM | 637 | CE2 | TYR | A | 87 | 8.238 | 43.218 | −18.129 | 1.00 | 35.52 | C |
| ATOM | 638 | CD2 | TYR | A | 87 | 8.134 | 42.478 | −16.972 | 1.00 | 42.43 | C |
| ATOM | 639 | C | TYR | A | 87 | 7.576 | 38.915 | −14.037 | 1.00 | 31.73 | C |
| ATOM | 640 | O | TYR | A | 87 | 7.516 | 39.330 | −12.899 | 1.00 | 32.92 | O |
| ATOM | 641 | N | ALA | A | 88 | 7.833 | 37.653 | −14.327 | 1.00 | 35.28 | N |
| ATOM | 642 | CA | ALA | A | 88 | 7.884 | 36.654 | −13.274 | 1.00 | 35.57 | C |
| ATOM | 643 | CB | ALA | A | 88 | 6.521 | 35.941 | −13.123 | 1.00 | 35.79 | C |
| ATOM | 644 | C | ALA | A | 88 | 8.986 | 35.707 | −13.607 | 1.00 | 35.70 | C |
| ATOM | 645 | O | ALA | A | 88 | 8.839 | 34.833 | −14.436 | 1.00 | 37.89 | O |
| ATOM | 646 | N | THR | A | 89 | 10.126 | 35.931 | −12.958 | 1.00 | 39.19 | N |
| ATOM | 647 | CA | THR | A | 89 | 11.313 | 35.161 | −13.225 | 1.00 | 38.72 | C |
| ATOM | 648 | CB | THR | A | 89 | 12.560 | 36.058 | −13.114 | 1.00 | 41.64 | C |
| ATOM | 649 | OG1 | THR | A | 89 | 12.479 | 37.065 | −14.125 | 1.00 | 39.96 | O |
| ATOM | 650 | CG2 | THR | A | 89 | 13.850 | 35.262 | −13.291 | 1.00 | 40.09 | C |
| ATOM | 651 | C | THR | A | 89 | 11.350 | 34.037 | −12.208 | 1.00 | 38.27 | C |
| ATOM | 652 | O | THR | A | 89 | 11.327 | 34.284 | −11.016 | 1.00 | 36.78 | O |
| ATOM | 653 | N | LEU | A | 90 | 11.391 | 32.810 | −12.712 | 1.00 | 41.20 | N |
| ATOM | 654 | CA | LEU | A | 90 | 11.473 | 31.597 | −11.899 | 1.00 | 37.67 | C |
| ATOM | 655 | CB | LEU | A | 90 | 10.400 | 30.599 | −12.345 | 1.00 | 35.91 | C |
| ATOM | 656 | CG | LEU | A | 90 | 8.978 | 31.122 | −12.557 | 1.00 | 42.98 | C |
| ATOM | 657 | CD1 | LEU | A | 90 | 8.040 | 30.101 | −13.249 | 1.00 | 37.76 | C |
| ATOM | 658 | CD2 | LEU | A | 90 | 8.416 | 31.577 | −11.249 | 1.00 | 37.89 | C |
| ATOM | 659 | C | LEU | A | 90 | 12.845 | 30.939 | −12.031 | 1.00 | 35.29 | C |
| ATOM | 660 | O | LEU | A | 90 | 13.357 | 30.750 | −13.129 | 1.00 | 34.84 | O |
| ATOM | 661 | N | ARG | A | 91 | 13.414 | 30.524 | −10.908 | 1.00 | 32.23 | N |
| ATOM | 662 | CA | ARG | A | 91 | 14.578 | 29.704 | −10.970 | 1.00 | 31.68 | C |
| ATOM | 663 | CB | ARG | A | 91 | 15.729 | 30.367 | −10.213 | 1.00 | 29.26 | C |
| ATOM | 664 | CG | ARG | A | 91 | 16.186 | 31.702 | −10.714 | 1.00 | 39.83 | C |
| ATOM | 665 | CD | ARG | A | 91 | 17.422 | 32.123 | −9.918 | 1.00 | 48.82 | C |
| ATOM | 666 | NE | ARG | A | 91 | 18.182 | 33.162 | −10.597 | 1.00 | 48.28 | N |
| ATOM | 667 | CZ | ARG | A | 91 | 19.207 | 32.929 | −11.415 | 1.00 | 54.96 | C |
| ATOM | 668 | NH1 | ARG | A | 91 | 19.832 | 33.957 | −11.994 | 1.00 | 48.94 | N |
| ATOM | 669 | NH2 | ARG | A | 91 | 19.607 | 31.674 | −11.657 | 1.00 | 41.81 | N |
| ATOM | 670 | C | ARG | A | 91 | 14.230 | 28.388 | −10.333 | 1.00 | 29.39 | C |
| ATOM | 671 | O | ARG | A | 91 | 13.650 | 28.379 | −9.267 | 1.00 | 30.64 | O |
| ATOM | 672 | N | PHE | A | 92 | 14.603 | 27.283 | −10.965 | 1.00 | 29.19 | N |
| ATOM | 673 | CA | PHE | A | 92 | 14.179 | 25.976 | −10.483 | 1.00 | 28.13 | C |
| ATOM | 674 | CB | PHE | A | 92 | 12.672 | 25.695 | −10.853 | 1.00 | 25.35 | C |
| ATOM | 675 | CG | PHE | A | 92 | 12.392 | 25.582 | −12.330 | 1.00 | 31.38 | C |
| ATOM | 676 | CD1 | PHE | A | 92 | 12.298 | 24.318 | −12.957 | 1.00 | 30.18 | C |
| ATOM | 677 | CE1 | PHE | A | 92 | 12.057 | 24.206 | −14.311 | 1.00 | 29.16 | C |
| ATOM | 678 | CZ | PHE | A | 92 | 11.846 | 25.370 | −15.072 | 1.00 | 39.78 | C |
| ATOM | 679 | CE2 | PHE | A | 92 | 11.894 | 26.646 | −14.463 | 1.00 | 28.29 | C |
| ATOM | 680 | CD2 | PHE | A | 92 | 12.161 | 26.743 | −13.098 | 1.00 | 36.30 | C |
| ATOM | 681 | C | PHE | A | 92 | 15.075 | 24.896 | −10.979 | 1.00 | 28.40 | C |
| ATOM | 682 | O | PHE | A | 92 | 15.770 | 25.050 | −11.991 | 1.00 | 33.15 | O |
| ATOM | 683 | N | THR | A | 93 | 15.024 | 23.775 | −10.304 | 1.00 | 28.13 | N |
| ATOM | 684 | CA | THR | A | 93 | 15.649 | 22.555 | −10.797 | 1.00 | 32.26 | C |
| ATOM | 685 | CB | THR | A | 93 | 16.770 | 22.043 | −9.843 | 0.50 | 30.79 | C |
| ATOM | 686 | OG1 | THR | A | 93 | 16.238 | 21.847 | −8.539 | 0.50 | 24.15 | O |
| ATOM | 687 | CG2 | THR | A | 93 | 17.892 | 23.084 | −9.725 | 0.50 | 28.99 | C |
| ATOM | 688 | C | THR | A | 93 | 14.528 | 21.525 | −11.044 | 1.00 | 35.09 | C |
| ATOM | 689 | O | THR | A | 93 | 13.461 | 21.541 | −10.390 | 1.00 | 37.83 | O |
| ATOM | 690 | N | MET | A | 94 | 14.748 | 20.661 | −12.022 | 1.00 | 37.88 | N |
| ATOM | 691 | CA | MET | A | 94 | 13.738 | 19.691 | −12.440 | 1.00 | 34.08 | C |
| ATOM | 692 | CB | MET | A | 94 | 13.082 | 20.163 | −13.741 | 1.00 | 37.68 | C |
| ATOM | 693 | CG | MET | A | 94 | 12.132 | 19.187 | −14.384 | 1.00 | 31.33 | C |
| ATOM | 694 | SD | MET | A | 94 | 10.563 | 19.099 | −13.517 | 1.00 | 37.28 | S |
| ATOM | 695 | CE | MET | A | 94 | 10.084 | 20.829 | −13.643 | 1.00 | 24.37 | C |
| ATOM | 696 | C | MET | A | 94 | 14.466 | 18.376 | −12.660 | 1.00 | 37.15 | C |
| ATOM | 697 | O | MET | A | 94 | 15.519 | 18.350 | −13.326 | 1.00 | 37.61 | O |
| ATOM | 698 | N | LEU | A | 95 | 13.968 | 17.313 | −12.049 | 1.00 | 32.63 | N |
| ATOM | 699 | CA | LEU | A | 95 | 14.611 | 16.001 | −12.199 | 1.00 | 36.65 | C |
| ATOM | 700 | CB | LEU | A | 95 | 14.500 | 15.178 | −10.912 | 1.00 | 35.07 | C |
| ATOM | 701 | CG | LEU | A | 95 | 15.630 | 15.458 | −9.894 | 1.00 | 40.28 | C |
| ATOM | 702 | CD1 | LEU | A | 95 | 15.447 | 16.766 | −9.120 | 1.00 | 39.97 | C |
| ATOM | 703 | CD2 | LEU | A | 95 | 15.799 | 14.331 | −8.946 | 1.00 | 41.34 | C |
| ATOM | 704 | C | LEU | A | 95 | 14.126 | 15.196 | −13.401 | 1.00 | 37.05 | C |
| ATOM | 705 | O | LEU | A | 95 | 12.951 | 15.165 | −13.680 | 1.00 | 38.21 | O |
| ATOM | 706 | N | GLU | A | 96 | 15.064 | 14.546 | −14.093 | 1.00 | 40.05 | N |
| ATOM | 707 | CA | GLU | A | 96 | 14.788 | 13.649 | −15.213 | 1.00 | 39.08 | C |
| ATOM | 708 | CB | GLU | A | 96 | 16.103 | 13.106 | −15.779 | 1.00 | 40.73 | C |
| ATOM | 709 | CG | GLU | A | 96 | 15.936 | 12.029 | −16.850 | 1.00 | 41.96 | C |
| ATOM | 710 | CD | GLU | A | 96 | 17.259 | 11.583 | −17.456 | 1.00 | 43.00 | C |
| ATOM | 711 | OE1 | GLU | A | 96 | 18.244 | 11.364 | −16.718 | 1.00 | 46.73 | O |
| ATOM | 712 | OE2 | GLU | A | 96 | 17.299 | 11.443 | −18.693 | 1.00 | 54.87 | O |
| ATOM | 713 | C | GLU | A | 96 | 13.931 | 12.497 | −14.738 | 1.00 | 38.39 | C |
| ATOM | 714 | O | GLU | A | 96 | 14.320 | 11.774 | −13.818 | 1.00 | 40.49 | O |
| ATOM | 715 | N | CYS | A | 97 | 12.764 | 12.313 | −15.372 | 1.00 | 40.12 | N |
| ATOM | 716 | CA | CYS | A | 97 | 11.810 | 11.294 | −14.934 | 1.00 | 39.96 | C |
| ATOM | 717 | CB | CYS | A | 97 | 10.527 | 11.350 | −15.772 | 1.00 | 43.34 | C |
| ATOM | 718 | SG | CYS | A | 97 | 9.358 | 12.560 | −15.204 | 1.00 | 46.32 | S |
| ATOM | 719 | C | CYS | A | 97 | 12.397 | 9.893 | −14.973 | 1.00 | 41.63 | C |
| ATOM | 720 | O | CYS | A | 97 | 12.295 | 9.135 | −13.992 | 1.00 | 41.45 | O |
| ATOM | 721 | N | LEU | A | 98 | 13.045 | 9.563 | −16.089 | 1.00 | 45.11 | N |
| ATOM | 722 | CA | LEU | A | 98 | 13.628 | 8.226 | −16.258 | 1.00 | 49.04 | C |
| ATOM | 723 | CB | LEU | A | 98 | 13.860 | 7.892 | −17.743 | 1.00 | 51.09 | C |
| ATOM | 724 | CG | LEU | A | 98 | 12.548 | 7.587 | −18.510 | 1.00 | 51.68 | C |
| ATOM | 725 | CD1 | LEU | A | 98 | 12.708 | 7.541 | −20.017 | 1.00 | 49.01 | C |
| ATOM | 726 | CD2 | LEU | A | 98 | 11.843 | 6.315 | −18.019 | 1.00 | 57.57 | C |
| ATOM | 727 | C | LEU | A | 98 | 14.859 | 7.951 | −15.405 | 1.00 | 49.53 | C |
| ATOM | 728 | O | LEU | A | 98 | 15.338 | 6.828 | −15.373 | 1.00 | 47.03 | O |
| ATOM | 729 | N | SER | A | 99 | 15.338 | 8.969 | −14.688 | 1.00 | 52.06 | N |
| ATOM | 730 | CA | SER | A | 99 | 16.509 | 8.844 | −13.803 | 1.00 | 53.05 | C |
| ATOM | 731 | CB | SER | A | 99 | 17.356 | 10.125 | −13.826 | 1.00 | 53.42 | C |
| ATOM | 732 | OG | SER | A | 99 | 16.784 | 11.140 | −12.992 | 1.00 | 59.05 | O |
| ATOM | 733 | C | SER | A | 99 | 16.081 | 8.591 | −12.366 | 1.00 | 55.03 | C |
| ATOM | 734 | O | SER | A | 99 | 16.879 | 8.135 | −11.542 | 1.00 | 53.98 | O |
| ATOM | 735 | N | LEU | A | 100 | 14.828 | 8.926 | −12.060 | 1.00 | 57.16 | N |
| ATOM | 736 | CA | LEU | A | 100 | 14.355 | 8.903 | −10.689 | 1.00 | 58.91 | C |
| ATOM | 737 | CB | LEU | A | 100 | 13.036 | 9.658 | −10.554 | 1.00 | 57.21 | C |
| ATOM | 738 | CG | LEU | A | 100 | 12.907 | 11.112 | −10.971 | 1.00 | 51.47 | C |
| ATOM | 739 | CD1 | LEU | A | 100 | 11.448 | 11.472 | −10.926 | 1.00 | 45.58 | C |
| ATOM | 740 | CD2 | LEU | A | 100 | 13.689 | 12.003 | −10.050 | 1.00 | 48.85 | C |
| ATOM | 741 | C | LEU | A | 100 | 14.147 | 7.471 | −10.250 | 1.00 | 63.37 | C |
| ATOM | 742 | O | LEU | A | 100 | 13.775 | 6.629 | −11.062 | 1.00 | 63.99 | O |
| ATOM | 743 | N | PRO | A | 101 | 14.391 | 7.187 | −8.960 | 1.00 | 66.45 | N |
| ATOM | 744 | CA | PRO | A | 101 | 13.936 | 5.891 | −8.477 | 1.00 | 67.84 | C |
| ATOM | 745 | CB | PRO | A | 101 | 14.775 | 5.656 | −7.218 | 1.00 | 68.56 | C |
| ATOM | 746 | CG | PRO | A | 101 | 15.326 | 7.009 | −6.814 | 1.00 | 68.23 | C |
| ATOM | 747 | CD | PRO | A | 101 | 15.054 | 7.995 | −7.916 | 1.00 | 66.48 | C |
| ATOM | 748 | C | PRO | A | 101 | 12.440 | 5.954 | −8.161 | 1.00 | 69.98 | C |
| ATOM | 749 | O | PRO | A | 101 | 11.956 | 6.967 | −7.632 | 1.00 | 72.74 | O |
| ATOM | 750 | N | ARG | A | 102 | 11.721 | 4.899 | −8.540 | 1.00 | 69.84 | N |
| ATOM | 751 | CA | ARG | A | 102 | 10.292 | 4.709 | −8.226 | 1.00 | 69.18 | C |
| ATOM | 752 | CB | ARG | A | 102 | 10.066 | 4.608 | −6.705 | 1.00 | 68.31 | C |
| ATOM | 753 | C | ARG | A | 102 | 9.309 | 5.706 | −8.876 | 1.00 | 68.61 | C |
| ATOM | 754 | O | ARG | A | 102 | 8.196 | 5.884 | −8.384 | 1.00 | 66.90 | O |
| ATOM | 755 | N | ALA | A | 103 | 9.712 | 6.321 | −9.991 | 1.00 | 69.15 | N |
| ATOM | 756 | CA | ALA | A | 103 | 8.840 | 7.246 | −10.735 | 1.00 | 68.27 | C |
| ATOM | 757 | CB | ALA | A | 103 | 9.659 | 8.135 | −11.660 | 1.00 | 68.20 | C |
| ATOM | 758 | C | ALA | A | 103 | 7.751 | 6.512 | −11.519 | 1.00 | 67.94 | C |
| ATOM | 759 | O | ALA | A | 103 | 8.043 | 5.742 | −12.430 | 1.00 | 69.95 | O |
| ATOM | 760 | N | GLY | A | 104 | 6.496 | 6.761 | −11.159 | 1.00 | 67.03 | N |
| ATOM | 761 | CA | GLY | A | 104 | 5.353 | 6.130 | −11.813 | 1.00 | 65.29 | C |
| ATOM | 762 | C | GLY | A | 104 | 5.013 | 6.728 | −13.168 | 1.00 | 64.79 | C |
| ATOM | 763 | O | GLY | A | 104 | 5.795 | 7.490 | −13.734 | 1.00 | 64.68 | O |
| ATOM | 764 | N | ARG | A | 105 | 3.835 | 6.371 | −13.681 | 1.00 | 64.75 | N |
| ATOM | 765 | CA | ARG | A | 105 | 3.313 | 6.884 | −14.958 | 1.00 | 62.98 | C |
| ATOM | 766 | CB | ARG | A | 105 | 2.082 | 6.076 | −15.373 | 1.00 | 63.28 | C |
| ATOM | 767 | C | ARG | A | 105 | 2.960 | 8.380 | −14.900 | 1.00 | 60.94 | C |
| ATOM | 768 | O | ARG | A | 105 | 2.906 | 9.062 | −15.932 | 1.00 | 61.36 | O |
| ATOM | 769 | N | SER | A | 106 | 2.738 | 8.884 | −13.690 | 1.00 | 58.17 | N |
| ATOM | 770 | CA | SER | A | 106 | 2.346 | 10.276 | −13.477 | 1.00 | 56.11 | C |
| ATOM | 771 | CB | SER | A | 106 | 1.752 | 10.438 | −12.066 | 1.00 | 56.25 | C |
| ATOM | 772 | OG | SER | A | 106 | 1.128 | 11.702 | −11.881 | 1.00 | 62.27 | O |
| ATOM | 773 | C | SER | A | 106 | 3.492 | 11.282 | −13.733 | 1.00 | 52.57 | C |
| ATOM | 774 | O | SER | A | 106 | 3.240 | 12.458 | −13.955 | 1.00 | 51.79 | O |
| ATOM | 775 | N | CYS | A | 107 | 4.736 | 10.802 | −13.767 | 1.00 | 48.78 | N |
| ATOM | 776 | CA | CYS | A | 107 | 5.914 | 11.679 | −13.824 | 1.00 | 45.90 | C |
| ATOM | 777 | CB | CYS | A | 107 | 7.197 | 10.838 | −13.856 | 1.00 | 41.55 | C |
| ATOM | 778 | SG | CYS | A | 107 | 8.708 | 11.772 | −13.436 | 1.00 | 45.62 | S |
| ATOM | 779 | C | CYS | A | 107 | 5.902 | 12.687 | −14.992 | 1.00 | 44.80 | C |
| ATOM | 780 | O | CYS | A | 107 | 5.721 | 12.296 | −16.133 | 1.00 | 45.74 | O |
| ATOM | 781 | N | LYS | A | 108 | 6.108 | 13.973 | −14.680 | 1.00 | 42.56 | N |
| ATOM | 782 | CA | LYS | A | 108 | 6.171 | 15.074 | −15.656 | 1.00 | 38.35 | C |
| ATOM | 783 | CB | LYS | A | 108 | 5.080 | 16.126 | −15.390 | 1.00 | 38.46 | C |
| ATOM | 784 | CG | LYS | A | 108 | 3.617 | 15.631 | −15.344 | 1.00 | 41.65 | C |
| ATOM | 785 | CD | LYS | A | 108 | 3.130 | 15.220 | −16.728 | 1.00 | 48.57 | C |
| ATOM | 786 | CE | LYS | A | 108 | 1.623 | 15.055 | −16.780 | 1.00 | 49.47 | C |
| ATOM | 787 | NZ | LYS | A | 108 | 1.183 | 13.739 | −16.226 | 1.00 | 51.52 | N |
| ATOM | 788 | C | LYS | A | 108 | 7.512 | 15.802 | −15.536 | 1.00 | 39.54 | C |
| ATOM | 789 | O | LYS | A | 108 | 8.128 | 15.818 | −14.465 | 1.00 | 30.07 | O |
| ATOM | 790 | N | GLU | A | 109 | 7.944 | 16.427 | −16.625 | 1.00 | 38.56 | N |
| ATOM | 791 | CA | GLU | A | 109 | 9.128 | 17.239 | −16.574 | 1.00 | 38.64 | C |
| ATOM | 792 | CB | GLU | A | 109 | 10.181 | 16.730 | −17.560 | 1.00 | 39.02 | C |
| ATOM | 793 | CG | GLU | A | 109 | 11.197 | 15.800 | −16.920 | 1.00 | 38.15 | C |
| ATOM | 794 | CD | GLU | A | 109 | 11.851 | 14.822 | −17.899 | 1.00 | 44.21 | C |
| ATOM | 795 | OE1 | GLU | A | 109 | 11.980 | 15.168 | −19.088 | 1.00 | 43.35 | O |
| ATOM | 796 | OE2 | GLU | A | 109 | 12.272 | 13.717 | −17.467 | 1.00 | 42.06 | O |
| ATOM | 797 | C | GLU | A | 109 | 8.712 | 18.710 | −16.798 | 1.00 | 36.56 | C |
| ATOM | 798 | O | GLU | A | 109 | 9.471 | 19.530 | −17.304 | 1.00 | 34.15 | O |
| ATOM | 799 | N | THR | A | 110 | 7.487 | 19.022 | −16.383 | 1.00 | 38.64 | N |
| ATOM | 800 | CA | THR | A | 110 | 6.890 | 20.354 | −16.584 | 1.00 | 36.73 | C |
| ATOM | 801 | CB | THR | A | 110 | 6.025 | 20.451 | −17.885 | 1.00 | 39.68 | C |
| ATOM | 802 | OG1 | THR | A | 110 | 4.742 | 19.859 | −17.655 | 1.00 | 36.91 | O |
| ATOM | 803 | CG2 | THR | A | 110 | 6.686 | 19.780 | −19.096 | 1.00 | 41.93 | C |
| ATOM | 804 | C | THR | A | 110 | 5.997 | 20.727 | −15.410 | 1.00 | 37.56 | C |
| ATOM | 805 | O | THR | A | 110 | 5.683 | 19.887 | −14.551 | 1.00 | 38.82 | O |
| ATOM | 806 | N | PHE | A | 111 | 5.558 | 21.984 | −15.397 | 1.00 | 34.68 | N |
| ATOM | 807 | CA | PHE | A | 111 | 4.613 | 22.485 | −14.422 | 1.00 | 36.33 | C |
| ATOM | 808 | CB | PHE | A | 111 | 5.316 | 22.834 | −13.062 | 1.00 | 30.29 | C |
| ATOM | 809 | CG | PHE | A | 111 | 6.288 | 24.008 | −13.127 | 1.00 | 37.23 | C |
| ATOM | 810 | CD1 | PHE | A | 111 | 5.861 | 25.295 | −12.849 | 1.00 | 29.32 | C |
| ATOM | 811 | CE1 | PHE | A | 111 | 6.719 | 26.368 | −12.916 | 1.00 | 32.48 | C |
| ATOM | 812 | CZ | PHE | A | 111 | 8.045 | 26.184 | −13.242 | 1.00 | 30.71 | C |
| ATOM | 813 | CE2 | PHE | A | 111 | 8.505 | 24.895 | −13.522 | 1.00 | 28.43 | C |
| ATOM | 814 | CD2 | PHE | A | 111 | 7.604 | 23.816 | −13.469 | 1.00 | 30.30 | C |
| ATOM | 815 | C | PHE | A | 111 | 3.880 | 23.675 | −15.070 | 1.00 | 35.66 | C |
| ATOM | 816 | O | PHE | A | 111 | 4.361 | 24.226 | −16.043 | 1.00 | 37.01 | O |
| ATOM | 817 | N | THR | A | 112 | 2.709 | 24.032 | −14.559 | 1.00 | 34.15 | N |
| ATOM | 818 | CA | THR | A | 112 | 1.902 | 25.099 | −15.158 | 1.00 | 38.08 | C |
| ATOM | 819 | CB | THR | A | 112 | 0.476 | 24.609 | −15.513 | 1.00 | 33.33 | C |
| ATOM | 820 | OG1 | THR | A | 112 | 0.534 | 23.264 | −15.995 | 1.00 | 47.68 | O |
| ATOM | 821 | CG2 | THR | A | 112 | −0.151 | 25.509 | −16.580 | 1.00 | 43.08 | C |
| ATOM | 822 | C | THR | A | 112 | 1.757 | 26.252 | −14.190 | 1.00 | 33.68 | C |
| ATOM | 823 | O | THR | A | 112 | 1.636 | 26.024 | −12.992 | 1.00 | 37.19 | O |
| ATOM | 824 | N | VAL | A | 113 | 1.739 | 27.477 | −14.725 | 1.00 | 34.66 | N |
| ATOM | 825 | CA | VAL | A | 113 | 1.595 | 28.699 | −13.935 | 1.00 | 34.20 | C |
| ATOM | 826 | CB | VAL | A | 113 | 2.762 | 29.695 | −14.172 | 1.00 | 34.07 | C |
| ATOM | 827 | CG1 | VAL | A | 113 | 2.620 | 30.929 | −13.250 | 1.00 | 34.89 | C |
| ATOM | 828 | CG2 | VAL | A | 113 | 4.084 | 29.039 | −13.945 | 1.00 | 33.82 | C |
| ATOM | 829 | C | VAL | A | 113 | 0.278 | 29.398 | −14.298 | 1.00 | 39.57 | C |
| ATOM | 830 | O | VAL | A | 113 | −0.137 | 29.379 | −15.457 | 1.00 | 42.20 | O |
| ATOM | 831 | N | PHE | A | 114 | −0.343 | 30.032 | −13.301 | 1.00 | 38.25 | N |
| ATOM | 832 | CA | PHE | A | 114 | −1.679 | 30.608 | −13.385 | 1.00 | 40.31 | C |
| ATOM | 833 | CB | PHE | A | 114 | −2.721 | 29.729 | −12.651 | 1.00 | 37.24 | C |
| ATOM | 834 | CG | PHE | A | 114 | −3.128 | 28.487 | −13.382 | 1.00 | 41.83 | C |
| ATOM | 835 | CD1 | PHE | A | 114 | −4.214 | 28.508 | −14.269 | 1.00 | 36.65 | C |
| ATOM | 836 | CE1 | PHE | A | 114 | −4.609 | 27.380 | −14.934 | 1.00 | 39.00 | C |
| ATOM | 837 | CZ | PHE | A | 114 | −3.969 | 26.144 | −14.676 | 1.00 | 45.82 | C |
| ATOM | 838 | CE2 | PHE | A | 114 | −2.897 | 26.096 | −13.756 | 1.00 | 47.68 | C |
| ATOM | 839 | CD2 | PHE | A | 114 | −2.500 | 27.271 | −13.113 | 1.00 | 46.54 | C |
| ATOM | 840 | C | PHE | A | 114 | −1.569 | 31.905 | −12.622 | 1.00 | 41.06 | C |
| ATOM | 841 | O | PHE | A | 114 | −0.599 | 32.136 | −11.923 | 1.00 | 38.95 | O |
| ATOM | 842 | N | TYR | A | 115 | −2.559 | 32.774 | −12.778 | 1.00 | 45.00 | N |
| ATOM | 843 | CA | TYR | A | 115 | −2.713 | 33.895 | −11.855 | 1.00 | 42.70 | C |
| ATOM | 844 | CB | TYR | A | 115 | −1.955 | 35.146 | −12.293 | 1.00 | 42.83 | C |
| ATOM | 845 | CG | TYR | A | 115 | −2.521 | 35.945 | −13.454 | 1.00 | 40.85 | C |
| ATOM | 846 | CD1 | TYR | A | 115 | −3.338 | 37.058 | −13.234 | 1.00 | 41.69 | C |
| ATOM | 847 | CE1 | TYR | A | 115 | −3.841 | 37.820 | −14.326 | 1.00 | 48.11 | C |
| ATOM | 848 | CZ | TYR | A | 115 | −3.491 | 37.468 | −15.640 | 1.00 | 49.33 | C |
| ATOM | 849 | OH | TYR | A | 115 | −3.957 | 38.193 | −16.727 | 1.00 | 51.63 | O |
| ATOM | 850 | CE2 | TYR | A | 115 | −2.657 | 36.384 | −15.876 | 1.00 | 42.04 | C |
| ATOM | 851 | CD2 | TYR | A | 115 | −2.175 | 35.626 | −14.771 | 1.00 | 46.72 | C |
| ATOM | 852 | C | TYR | A | 115 | −4.167 | 34.175 | −11.557 | 1.00 | 44.47 | C |
| ATOM | 853 | O | TYR | A | 115 | −5.062 | 33.587 | −12.167 | 1.00 | 44.61 | O |
| ATOM | 854 | N | TYR | A | 116 | −4.389 | 35.025 | −10.565 | 1.00 | 42.10 | N |
| ATOM | 855 | CA | TYR | A | 116 | −5.712 | 35.412 | −10.169 | 1.00 | 41.46 | C |
| ATOM | 856 | CB | TYR | A | 116 | −6.248 | 34.439 | −9.125 | 1.00 | 45.18 | C |
| ATOM | 857 | CG | TYR | A | 116 | −7.594 | 34.821 | −8.566 | 1.00 | 44.71 | C |
| ATOM | 858 | CD1 | TYR | A | 116 | −8.756 | 34.651 | −9.322 | 1.00 | 48.20 | C |
| ATOM | 859 | CE1 | TYR | A | 116 | −9.997 | 34.998 | −8.815 | 1.00 | 51.58 | C |
| ATOM | 860 | CZ | TYR | A | 116 | −10.091 | 35.515 | −7.532 | 1.00 | 48.34 | C |
| ATOM | 861 | OH | TYR | A | 116 | −11.322 | 35.864 | −7.042 | 1.00 | 50.58 | O |
| ATOM | 862 | CE2 | TYR | A | 116 | −8.953 | 35.692 | −6.758 | 1.00 | 48.96 | C |
| ATOM | 863 | CD2 | TYR | A | 116 | −7.709 | 35.339 | −7.279 | 1.00 | 43.17 | C |
| ATOM | 864 | C | TYR | A | 116 | −5.627 | 36.809 | −9.594 | 1.00 | 39.41 | C |
| ATOM | 865 | O | TYR | A | 116 | −4.892 | 37.039 | −8.643 | 1.00 | 39.20 | O |
| ATOM | 866 | N | GLU | A | 117 | −6.354 | 37.755 | −10.187 | 1.00 | 41.63 | N |
| ATOM | 867 | CA | GLU | A | 117 | −6.451 | 39.109 | −9.619 | 1.00 | 39.85 | C |
| ATOM | 868 | CB | GLU | A | 117 | −6.656 | 40.172 | −10.689 | 1.00 | 44.57 | C |
| ATOM | 869 | CG | GLU | A | 117 | −5.696 | 40.238 | −11.861 | 1.00 | 41.68 | C |
| ATOM | 870 | CD | GLU | A | 117 | −5.987 | 41.478 | −12.708 | 1.00 | 43.45 | C |
| ATOM | 871 | OE1 | GLU | A | 117 | −6.151 | 42.558 | −12.099 | 1.00 | 39.22 | O |
| ATOM | 872 | OE2 | GLU | A | 117 | −6.076 | 41.379 | −13.958 | 1.00 | 47.74 | O |
| ATOM | 873 | C | GLU | A | 117 | −7.645 | 39.214 | −8.703 | 1.00 | 40.05 | C |
| ATOM | 874 | O | GLU | A | 117 | −8.720 | 38.631 | −8.972 | 1.00 | 40.21 | O |
| ATOM | 875 | N | SER | A | 118 | −7.496 | 40.010 | −7.649 | 1.00 | 38.81 | N |
| ATOM | 876 | CA | SER | A | 118 | −8.635 | 40.341 | −6.784 | 1.00 | 40.06 | C |
| ATOM | 877 | CB | SER | A | 118 | −8.714 | 39.353 | −5.647 | 1.00 | 36.38 | C |
| ATOM | 878 | OG | SER | A | 118 | −7.464 | 39.353 | −4.975 | 1.00 | 41.44 | O |
| ATOM | 879 | C | SER | A | 118 | −8.408 | 41.723 | −6.204 | 1.00 | 41.25 | C |
| ATOM | 880 | O | SER | A | 118 | −7.277 | 42.107 | −5.969 | 1.00 | 41.14 | O |
| ATOM | 881 | N | ASP | A | 119 | −9.476 | 42.466 | −5.945 | 1.00 | 43.52 | N |
| ATOM | 882 | CA | ASP | A | 119 | −9.331 | 43.849 | −5.464 | 1.00 | 44.39 | C |
| ATOM | 883 | CB | ASP | A | 119 | −10.632 | 44.642 | −5.659 | 1.00 | 42.41 | C |
| ATOM | 884 | CG | ASP | A | 119 | −10.730 | 45.267 | −7.050 | 1.00 | 46.99 | C |
| ATOM | 885 | OD1 | ASP | A | 119 | −9.777 | 45.987 | −7.479 | 1.00 | 44.22 | O |
| ATOM | 886 | OD2 | ASP | A | 119 | −11.768 | 45.046 | −7.708 | 1.00 | 53.14 | O |
| ATOM | 887 | C | ASP | A | 119 | −8.795 | 43.944 | −4.011 | 1.00 | 45.57 | C |
| ATOM | 888 | O | ASP | A | 119 | −8.150 | 44.928 | −3.645 | 1.00 | 46.79 | O |
| ATOM | 889 | N | ALA | A | 120 | −9.059 | 42.912 | −3.208 | 1.00 | 42.24 | N |
| ATOM | 890 | CA | ALA | A | 120 | −8.488 | 42.778 | −1.873 | 1.00 | 41.91 | C |
| ATOM | 891 | CB | ALA | A | 120 | −9.523 | 43.085 | −0.814 | 1.00 | 41.29 | C |
| ATOM | 892 | C | ALA | A | 120 | −7.958 | 41.342 | −1.718 | 1.00 | 41.62 | C |
| ATOM | 893 | O | ALA | A | 120 | −8.149 | 40.506 | −2.605 | 1.00 | 41.95 | O |
| ATOM | 894 | N | ASP | A | 121 | −7.279 | 41.080 | −0.607 | 1.00 | 40.39 | N |
| ATOM | 895 | CA | ASP | A | 121 | −6.760 | 39.750 | −0.304 | 1.00 | 38.92 | C |
| ATOM | 896 | CB | ASP | A | 121 | −5.603 | 39.842 | 0.695 | 1.00 | 39.88 | C |
| ATOM | 897 | CG | ASP | A | 121 | −4.986 | 38.485 | 0.986 | 1.00 | 38.88 | C |
| ATOM | 898 | OD1 | ASP | A | 121 | −5.368 | 37.528 | 0.286 | 1.00 | 43.21 | O |
| ATOM | 899 | OD2 | ASP | A | 121 | −4.135 | 38.370 | 1.901 | 1.00 | 35.63 | O |
| ATOM | 900 | C | ASP | A | 121 | −7.877 | 38.869 | 0.267 | 1.00 | 39.74 | C |
| ATOM | 901 | O | ASP | A | 121 | −7.956 | 38.631 | 1.469 | 1.00 | 40.54 | O |
| ATOM | 902 | N | THR | A | 122 | −8.724 | 38.356 | −0.608 | 1.00 | 38.18 | N |
| ATOM | 903 | CA | THR | A | 122 | −9.920 | 37.670 | −0.167 | 1.00 | 42.04 | C |
| ATOM | 904 | CB | THR | A | 122 | −11.102 | 38.158 | −0.958 | 1.00 | 39.56 | C |
| ATOM | 905 | OG1 | THR | A | 122 | −10.799 | 38.009 | −2.350 | 1.00 | 39.83 | O |
| ATOM | 906 | CG2 | THR | A | 122 | −11.348 | 39.643 | −0.668 | 1.00 | 47.23 | C |
| ATOM | 907 | C | THR | A | 122 | −9.819 | 36.149 | −0.322 | 1.00 | 43.00 | C |
| ATOM | 908 | O | THR | A | 122 | −10.737 | 35.417 | 0.059 | 1.00 | 45.82 | O |
| ATOM | 909 | N | ALA | A | 123 | −8.712 | 35.668 | −0.880 | 1.00 | 43.76 | N |
| ATOM | 910 | CA | ALA | A | 123 | −8.527 | 34.207 | −1.066 | 1.00 | 39.96 | C |
| ATOM | 911 | CB | ALA | A | 123 | −7.500 | 33.946 | −2.149 | 1.00 | 38.69 | C |
| ATOM | 912 | C | ALA | A | 123 | −8.162 | 33.452 | 0.231 | 1.00 | 39.42 | C |
| ATOM | 913 | O | ALA | A | 123 | −7.456 | 33.976 | 1.079 | 1.00 | 38.76 | O |
| ATOM | 914 | N | THR | A | 124 | −8.630 | 32.207 | 0.354 | 1.00 | 37.16 | N |
| ATOM | 915 | CA | THR | A | 124 | −8.381 | 31.380 | 1.544 | 1.00 | 30.14 | C |
| ATOM | 916 | CB | THR | A | 124 | −9.759 | 31.027 | 2.267 | 1.00 | 27.28 | C |
| ATOM | 917 | OG1 | THR | A | 124 | −10.609 | 30.385 | 1.321 | 1.00 | 37.96 | O |
| ATOM | 918 | CG2 | THR | A | 124 | −10.449 | 32.264 | 2.782 | 1.00 | 34.09 | C |
| ATOM | 919 | C | THR | A | 124 | −7.628 | 30.114 | 1.121 | 1.00 | 31.09 | C |
| ATOM | 920 | O | THR | A | 124 | −7.161 | 29.992 | −0.002 | 1.00 | 34.92 | O |
| ATOM | 921 | N | ALA | A | 125 | −7.516 | 29.143 | 2.006 | 1.00 | 34.69 | N |
| ATOM | 922 | CA | ALA | A | 125 | −6.966 | 27.857 | 1.594 | 1.00 | 35.09 | C |
| ATOM | 923 | CB | ALA | A | 125 | −6.557 | 27.037 | 2.822 | 1.00 | 35.57 | C |
| ATOM | 924 | C | ALA | A | 125 | −7.905 | 27.045 | 0.668 | 1.00 | 35.61 | C |
| ATOM | 925 | O | ALA | A | 125 | −7.450 | 26.100 | 0.025 | 1.00 | 35.88 | O |
| ATOM | 926 | N | LEU | A | 126 | −9.183 | 27.433 | 0.580 | 1.00 | 35.52 | N |
| ATOM | 927 | CA | LEU | A | 126 | −10.170 | 26.725 | −0.244 | 1.00 | 38.01 | C |
| ATOM | 928 | CB | LEU | A | 126 | −11.158 | 25.926 | 0.629 | 1.00 | 40.30 | C |
| ATOM | 929 | CG | LEU | A | 126 | −10.687 | 24.875 | 1.642 | 1.00 | 35.50 | C |
| ATOM | 930 | CD1 | LEU | A | 126 | −11.925 | 24.464 | 2.443 | 1.00 | 35.12 | C |
| ATOM | 931 | CD2 | LEU | A | 126 | −10.053 | 23.662 | 0.975 | 1.00 | 43.01 | C |
| ATOM | 932 | C | LEU | A | 126 | −10.956 | 27.560 | −1.259 | 1.00 | 39.41 | C |
| ATOM | 933 | O | LEU | A | 126 | −11.697 | 26.987 | −2.064 | 1.00 | 38.78 | O |
| ATOM | 934 | N | THR | A | 127 | −10.819 | 28.892 | −1.208 | 1.00 | 39.87 | N |
| ATOM | 935 | CA | THR | A | 127 | −11.417 | 29.795 | −2.201 | 1.00 | 40.08 | C |
| ATOM | 936 | CB | THR | A | 127 | −12.495 | 30.751 | −1.616 | 1.00 | 37.17 | C |
| ATOM | 937 | OG1 | THR | A | 127 | −11.920 | 31.565 | −0.589 | 1.00 | 37.12 | O |
| ATOM | 938 | CG2 | THR | A | 127 | −13.710 | 29.964 | −1.068 | 1.00 | 36.23 | C |
| ATOM | 939 | C | THR | A | 127 | −10.309 | 30.639 | −2.849 | 1.00 | 38.11 | C |
| ATOM | 940 | O | THR | A | 127 | −9.397 | 31.072 | −2.167 | 1.00 | 39.91 | O |
| ATOM | 941 | N | PRO | A | 128 | −10.389 | 30.879 | −4.160 | 1.00 | 38.97 | N |
| ATOM | 942 | CA | PRO | A | 128 | −11.419 | 30.490 | −5.120 | 1.00 | 38.25 | C |
| ATOM | 943 | CB | PRO | A | 128 | −11.309 | 31.579 | −6.193 | 1.00 | 37.19 | C |
| ATOM | 944 | CG | PRO | A | 128 | −9.917 | 32.012 | −6.167 | 1.00 | 36.47 | C |
| ATOM | 945 | CD | PRO | A | 128 | −9.318 | 31.665 | −4.813 | 1.00 | 41.36 | C |
| ATOM | 946 | C | PRO | A | 128 | −11.145 | 29.112 | −5.707 | 1.00 | 37.82 | C |
| ATOM | 947 | O | PRO | A | 128 | −10.157 | 28.501 | −5.356 | 1.00 | 39.53 | O |
| ATOM | 948 | N | ALA | A | 129 | −12.025 | 28.630 | −6.581 | 1.00 | 39.78 | N |
| ATOM | 949 | CA | ALA | A | 129 | −11.854 | 27.318 | −7.208 | 1.00 | 41.22 | C |
| ATOM | 950 | CB | ALA | A | 129 | −12.845 | 27.149 | −8.367 | 1.00 | 44.36 | C |
| ATOM | 951 | C | ALA | A | 129 | −10.430 | 27.106 | −7.684 | 1.00 | 41.46 | C |
| ATOM | 952 | O | ALA | A | 129 | −9.931 | 27.854 | −8.514 | 1.00 | 42.01 | O |
| ATOM | 953 | N | TRP | A | 130 | −9.781 | 26.070 | −7.148 | 1.00 | 41.79 | N |
| ATOM | 954 | CA | TRP | A | 130 | −8.420 | 25.722 | −7.528 | 1.00 | 40.28 | C |
| ATOM | 955 | CB | TRP | A | 130 | −7.839 | 24.792 | −6.481 | 1.00 | 40.07 | C |
| ATOM | 956 | CG | TRP | A | 130 | −7.519 | 25.415 | −5.177 | 1.00 | 37.59 | C |
| ATOM | 957 | CD1 | TRP | A | 130 | −8.231 | 25.310 | −4.029 | 1.00 | 36.75 | C |
| ATOM | 958 | NE1 | TRP | A | 130 | −7.599 | 25.972 | −3.007 | 1.00 | 41.38 | N |
| ATOM | 959 | CE2 | TRP | A | 130 | −6.447 | 26.532 | −3.490 | 1.00 | 37.52 | C |
| ATOM | 960 | CD2 | TRP | A | 130 | −6.355 | 26.184 | −4.859 | 1.00 | 39.29 | C |
| ATOM | 961 | CE3 | TRP | A | 130 | −5.265 | 26.625 | −5.594 | 1.00 | 36.95 | C |
| ATOM | 962 | CZ3 | TRP | A | 130 | −4.283 | 27.382 | −4.939 | 1.00 | 37.64 | C |
| ATOM | 963 | CH2 | TRP | A | 130 | −4.390 | 27.671 | −3.578 | 1.00 | 27.48 | C |
| ATOM | 964 | CZ2 | TRP | A | 130 | −5.468 | 27.269 | −2.837 | 1.00 | 38.20 | C |
| ATOM | 965 | C | TRP | A | 130 | −8.357 | 25.051 | −8.897 | 1.00 | 42.27 | C |
| ATOM | 966 | O | TRP | A | 130 | −8.089 | 23.840 | −9.012 | 1.00 | 43.07 | O |
| ATOM | 967 | N | MET | A | 131 | −8.590 | 25.839 | −9.940 | 1.00 | 42.81 | N |
| ATOM | 968 | CA | MET | A | 131 | −8.682 | 25.324 | −11.305 | 1.00 | 46.53 | C |
| ATOM | 969 | CB | MET | A | 131 | −9.995 | 24.548 | −11.502 | 1.00 | 44.82 | C |
| ATOM | 970 | CG | MET | A | 131 | −11.234 | 25.302 | −11.084 | 1.00 | 50.50 | C |
| ATOM | 971 | SD | MET | A | 131 | −12.714 | 24.275 | −10.951 | 1.00 | 56.25 | S |
| ATOM | 972 | CE | MET | A | 131 | −12.308 | 23.173 | −9.583 | 1.00 | 56.94 | C |
| ATOM | 973 | C | MET | A | 131 | −8.664 | 26.499 | −12.258 | 1.00 | 44.91 | C |
| ATOM | 974 | O | MET | A | 131 | −9.043 | 27.602 | −11.872 | 1.00 | 42.46 | O |
| ATOM | 975 | N | GLU | A | 132 | −8.228 | 26.265 | −13.491 | 1.00 | 46.13 | N |
| ATOM | 976 | CA | GLU | A | 132 | −8.370 | 27.253 | −14.554 | 1.00 | 47.13 | C |
| ATOM | 977 | CB | GLU | A | 132 | −7.908 | 26.671 | −15.892 | 1.00 | 47.45 | C |
| ATOM | 978 | CG | GLU | A | 132 | −7.460 | 27.716 | −16.901 | 1.00 | 47.99 | C |
| ATOM | 979 | CD | GLU | A | 132 | −6.957 | 27.101 | −18.192 | 1.00 | 49.03 | C |
| ATOM | 980 | OE1 | GLU | A | 132 | −6.709 | 25.877 | −18.212 | 1.00 | 56.50 | O |
| ATOM | 981 | OE2 | GLU | A | 132 | −6.810 | 27.841 | −19.187 | 1.00 | 59.16 | O |
| ATOM | 982 | C | GLU | A | 132 | −9.811 | 27.741 | −14.665 | 1.00 | 50.42 | C |
| ATOM | 983 | O | GLU | A | 132 | −10.747 | 26.942 | −14.675 | 1.00 | 49.36 | O |
| ATOM | 984 | N | ASN | A | 133 | −9.980 | 29.056 | −14.747 | 1.00 | 51.67 | N |
| ATOM | 985 | CA | ASN | A | 133 | −11.273 | 29.680 | −14.489 | 1.00 | 52.45 | C |
| ATOM | 986 | CB | ASN | A | 133 | −12.287 | 29.272 | −15.559 | 1.00 | 53.87 | C |
| ATOM | 987 | CG | ASN | A | 133 | −12.849 | 30.462 | −16.312 | 1.00 | 58.00 | C |
| ATOM | 988 | OD1 | ASN | A | 133 | −12.264 | 30.925 | −17.291 | 1.00 | 55.56 | O |
| ATOM | 989 | ND2 | ASN | A | 133 | −13.991 | 30.965 | −15.857 | 1.00 | 60.24 | N |
| ATOM | 990 | C | ASN | A | 133 | −11.810 | 29.341 | −13.102 | 1.00 | 51.46 | C |
| ATOM | 991 | O | ASN | A | 133 | −12.643 | 28.449 | −12.949 | 1.00 | 53.60 | O |
| ATOM | 992 | N | PRO | A | 134 | −11.326 | 30.060 | −12.094 | 1.00 | 48.44 | N |
| ATOM | 993 | CA | PRO | A | 134 | −11.125 | 31.508 | −12.207 | 1.00 | 46.49 | C |
| ATOM | 994 | CB | PRO | A | 134 | −11.608 | 32.030 | −10.853 | 1.00 | 48.19 | C |
| ATOM | 995 | CG | PRO | A | 134 | −11.380 | 30.898 | −9.917 | 1.00 | 44.82 | C |
| ATOM | 996 | CD | PRO | A | 134 | −11.634 | 29.649 | −10.713 | 1.00 | 45.15 | C |
| ATOM | 997 | C | PRO | A | 134 | −9.656 | 31.862 | −12.412 | 1.00 | 46.83 | C |
| ATOM | 998 | O | PRO | A | 134 | −9.334 | 33.013 | −12.705 | 1.00 | 42.89 | O |
| ATOM | 999 | N | TYR | A | 135 | −8.778 | 30.876 | −12.256 | 1.00 | 43.64 | N |
| ATOM | 1000 | CA | TYR | A | 135 | −7.359 | 31.064 | −12.529 | 1.00 | 41.33 | C |
| ATOM | 1001 | CB | TYR | A | 135 | −6.551 | 29.927 | −11.896 | 1.00 | 42.40 | C |
| ATOM | 1002 | CG | TYR | A | 135 | −6.391 | 30.116 | −10.420 | 1.00 | 38.37 | C |
| ATOM | 1003 | CD1 | TYR | A | 135 | −5.328 | 30.856 | −9.917 | 1.00 | 43.89 | C |
| ATOM | 1004 | CE1 | TYR | A | 135 | −5.165 | 31.047 | −8.565 | 1.00 | 35.67 | C |
| ATOM | 1005 | CZ | TYR | A | 135 | −6.073 | 30.497 | −7.686 | 1.00 | 40.40 | C |
| ATOM | 1006 | OH | TYR | A | 135 | −5.892 | 30.710 | −6.340 | 1.00 | 41.61 | O |
| ATOM | 1007 | CE2 | TYR | A | 135 | −7.143 | 29.734 | −8.147 | 1.00 | 40.34 | C |
| ATOM | 1008 | CD2 | TYR | A | 135 | −7.289 | 29.543 | −9.518 | 1.00 | 46.85 | C |
| ATOM | 1009 | C | TYR | A | 135 | −7.035 | 31.247 | −14.014 | 1.00 | 43.30 | C |
| ATOM | 1010 | O | TYR | A | 135 | −7.694 | 30.675 | −14.873 | 1.00 | 42.09 | O |
| ATOM | 1011 | N | ILE | A | 136 | −6.031 | 32.078 | −14.308 | 1.00 | 45.98 | N |
| ATOM | 1012 | CA | ILE | A | 136 | −5.689 | 32.424 | −15.693 | 1.00 | 45.22 | C |
| ATOM | 1013 | CB | ILE | A | 136 | −5.611 | 33.966 | −15.879 | 1.00 | 45.25 | C |
| ATOM | 1014 | CG1 | ILE | A | 136 | −6.674 | 34.667 | −15.018 | 1.00 | 43.90 | C |
| ATOM | 1015 | CD1 | ILE | A | 136 | −6.852 | 36.169 | −15.294 | 1.00 | 44.67 | C |
| ATOM | 1016 | CG2 | ILE | A | 136 | −5.625 | 34.329 | −17.376 | 1.00 | 45.40 | C |
| ATOM | 1017 | C | ILE | A | 136 | −4.360 | 31.802 | −16.125 | 1.00 | 44.75 | C |
| ATOM | 1018 | O | ILE | A | 136 | −3.317 | 32.244 | −15.674 | 1.00 | 49.92 | O |
| ATOM | 1019 | N | LYS | A | 137 | −4.405 | 30.788 | −16.996 | 1.00 | 45.67 | N |
| ATOM | 1020 | CA | LYS | A | 137 | −3.193 | 30.056 | −17.437 | 1.00 | 45.23 | C |
| ATOM | 1021 | CB | LYS | A | 137 | −3.550 | 29.029 | −18.517 | 1.00 | 46.80 | C |
| ATOM | 1022 | CG | LYS | A | 137 | −2.568 | 27.881 | −18.636 | 1.00 | 51.30 | C |
| ATOM | 1023 | CD | LYS | A | 137 | −2.822 | 27.038 | −19.864 | 1.00 | 43.64 | C |
| ATOM | 1024 | CE | LYS | A | 137 | −1.485 | 26.684 | −20.499 | 1.00 | 52.43 | C |
| ATOM | 1025 | NZ | LYS | A | 137 | −1.427 | 25.316 | −21.121 | 1.00 | 52.54 | N |
| ATOM | 1026 | C | LYS | A | 137 | −2.165 | 31.036 | −17.974 | 1.00 | 44.80 | C |
| ATOM | 1027 | O | LYS | A | 137 | −2.483 | 31.823 | −18.865 | 1.00 | 44.54 | O |
| ATOM | 1028 | N | VAL | A | 138 | −0.968 | 31.044 | −17.371 | 1.00 | 44.85 | N |
| ATOM | 1029 | CA | VAL | A | 138 | 0.159 | 31.840 | −17.855 | 1.00 | 42.62 | C |
| ATOM | 1030 | CB | VAL | A | 138 | 1.112 | 32.308 | −16.722 | 1.00 | 44.21 | C |
| ATOM | 1031 | CG1 | VAL | A | 138 | 2.292 | 33.090 | −17.295 | 1.00 | 42.19 | C |
| ATOM | 1032 | CG2 | VAL | A | 138 | 0.391 | 33.175 | −15.727 | 1.00 | 39.45 | C |
| ATOM | 1033 | C | VAL | A | 138 | 0.926 | 31.034 | −18.905 | 1.00 | 47.65 | C |
| ATOM | 1034 | O | VAL | A | 138 | 1.017 | 31.471 | −20.063 | 1.00 | 50.24 | O |
| ATOM | 1035 | N | ASP | A | 139 | 1.449 | 29.855 | −18.516 | 1.00 | 48.26 | N |
| ATOM | 1036 | CA | ASP | A | 139 | 2.147 | 28.942 | −19.445 | 1.00 | 47.50 | C |
| ATOM | 1037 | CB | ASP | A | 139 | 3.486 | 29.565 | −19.865 | 1.00 | 49.54 | C |
| ATOM | 1038 | CG | ASP | A | 139 | 3.863 | 29.271 | −21.313 | 1.00 | 55.87 | C |
| ATOM | 1039 | OD1 | ASP | A | 139 | 2.962 | 29.260 | −22.189 | 1.00 | 55.59 | O |
| ATOM | 1040 | OD2 | ASP | A | 139 | 5.078 | 29.094 | −21.572 | 1.00 | 56.27 | O |
| ATOM | 1041 | C | ASP | A | 139 | 2.384 | 27.557 | −18.816 | 1.00 | 46.73 | C |
| ATOM | 1042 | O | ASP | A | 139 | 2.370 | 27.425 | −17.603 | 1.00 | 46.01 | O |
| ATOM | 1043 | N | THR | A | 140 | 2.571 | 26.533 | −19.648 | 1.00 | 46.52 | N |
| ATOM | 1044 | CA | THR | A | 140 | 3.020 | 25.213 | −19.200 | 1.00 | 46.71 | C |
| ATOM | 1045 | CB | THR | A | 140 | 2.359 | 24.089 | −20.025 | 1.00 | 45.93 | C |
| ATOM | 1046 | OG1 | THR | A | 140 | 0.941 | 24.093 | −19.804 | 1.00 | 54.04 | O |
| ATOM | 1047 | CG2 | THR | A | 140 | 2.891 | 22.720 | −19.639 | 1.00 | 47.66 | C |
| ATOM | 1048 | C | THR | A | 140 | 4.552 | 25.235 | −19.364 | 1.00 | 47.71 | C |
| ATOM | 1049 | O | THR | A | 140 | 5.044 | 25.308 | −20.489 | 1.00 | 51.63 | O |
| ATOM | 1050 | N | VAL | A | 141 | 5.286 | 25.212 | −18.245 | 1.00 | 45.47 | N |
| ATOM | 1051 | CA | VAL | A | 141 | 6.731 | 25.536 | −18.205 | 1.00 | 43.47 | C |
| ATOM | 1052 | CB | VAL | A | 141 | 7.122 | 26.334 | −16.913 | 1.00 | 43.73 | C |
| ATOM | 1053 | CG1 | VAL | A | 141 | 8.620 | 26.701 | −16.885 | 1.00 | 39.47 | C |
| ATOM | 1054 | CG2 | VAL | A | 141 | 6.309 | 27.599 | −16.782 | 1.00 | 41.94 | C |
| ATOM | 1055 | C | VAL | A | 141 | 7.551 | 24.264 | −18.277 | 1.00 | 45.44 | C |
| ATOM | 1056 | O | VAL | A | 141 | 7.346 | 23.347 | −17.484 | 1.00 | 42.78 | O |
| ATOM | 1057 | N | ALA | A | 142 | 8.479 | 24.214 | −19.232 | 1.00 | 46.34 | N |
| ATOM | 1058 | CA | ALA | A | 142 | 9.357 | 23.061 | −19.394 | 1.00 | 44.60 | C |
| ATOM | 1059 | CB | ALA | A | 142 | 9.303 | 22.534 | −20.807 | 1.00 | 43.31 | C |
| ATOM | 1060 | C | ALA | A | 142 | 10.784 | 23.412 | −19.003 | 1.00 | 44.91 | C |
| ATOM | 1061 | O | ALA | A | 142 | 11.166 | 24.586 | −18.984 | 1.00 | 44.97 | O |
| ATOM | 1062 | N | ALA | A | 143 | 11.548 | 22.371 | −18.673 | 1.00 | 45.80 | N |
| ATOM | 1063 | CA | ALA | A | 143 | 12.940 | 22.477 | −18.238 | 1.00 | 43.20 | C |
| ATOM | 1064 | CB | ALA | A | 143 | 13.218 | 21.450 | −17.176 | 1.00 | 42.14 | C |
| ATOM | 1065 | C | ALA | A | 143 | 13.871 | 22.258 | −19.422 | 1.00 | 44.18 | C |
| ATOM | 1066 | O | ALA | A | 143 | 13.738 | 21.272 | −20.138 | 1.00 | 44.34 | O |
| ATOM | 1067 | N | GLU | A | 144 | 14.822 | 23.174 | −19.606 | 1.00 | 45.90 | N |
| ATOM | 1068 | CA | GLU | A | 144 | 15.860 | 23.046 | −20.625 | 1.00 | 45.98 | C |
| ATOM | 1069 | CB | GLU | A | 144 | 16.443 | 24.409 | −21.000 | 1.00 | 46.26 | C |
| ATOM | 1070 | C | GLU | A | 144 | 16.952 | 22.147 | −20.105 | 1.00 | 46.83 | C |
| ATOM | 1071 | O | GLU | A | 144 | 17.680 | 21.548 | −20.886 | 1.00 | 47.83 | O |
| ATOM | 1072 | N | HIS | A | 145 | 17.062 | 22.066 | −18.771 | 1.00 | 47.47 | N |
| ATOM | 1073 | CA | HIS | A | 145 | 18.045 | 21.228 | −18.103 | 1.00 | 43.60 | C |
| ATOM | 1074 | CB | HIS | A | 145 | 19.171 | 22.083 | −17.531 | 1.00 | 41.41 | C |
| ATOM | 1075 | CG | HIS | A | 145 | 19.817 | 22.965 | −18.550 | 1.00 | 49.97 | C |
| ATOM | 1076 | ND1 | HIS | A | 145 | 20.679 | 22.482 | −19.514 | 1.00 | 50.33 | N |
| ATOM | 1077 | CE1 | HIS | A | 145 | 21.067 | 23.480 | −20.288 | 1.00 | 55.83 | C |
| ATOM | 1078 | NE2 | HIS | A | 145 | 20.470 | 24.585 | −19.876 | 1.00 | 50.08 | N |
| ATOM | 1079 | CD2 | HIS | A | 145 | 19.686 | 24.289 | −18.790 | 1.00 | 50.75 | C |
| ATOM | 1080 | C | HIS | A | 145 | 17.436 | 20.361 | −17.002 | 1.00 | 42.09 | C |
| ATOM | 1081 | O | HIS | A | 145 | 16.716 | 20.847 | −16.135 | 1.00 | 39.62 | O |
| ATOM | 1082 | N | LEU | A | 146 | 17.765 | 19.075 | −17.051 | 1.00 | 40.69 | N |
| ATOM | 1083 | CA | LEU | A | 146 | 17.326 | 18.092 | −16.075 | 1.00 | 41.21 | C |
| ATOM | 1084 | CB | LEU | A | 146 | 16.898 | 16.793 | −16.786 | 1.00 | 43.31 | C |
| ATOM | 1085 | CG | LEU | A | 146 | 15.739 | 16.923 | −17.800 | 1.00 | 45.78 | C |
| ATOM | 1086 | CD1 | LEU | A | 146 | 15.507 | 15.612 | −18.551 | 1.00 | 52.48 | C |
| ATOM | 1087 | CD2 | LEU | A | 146 | 14.444 | 17.386 | −17.131 | 1.00 | 42.70 | C |
| ATOM | 1088 | C | LEU | A | 146 | 18.414 | 17.824 | −15.032 | 1.00 | 39.66 | C |
| ATOM | 1089 | O | LEU | A | 146 | 19.626 | 17.831 | −15.328 | 1.00 | 40.07 | O |
| ATOM | 1090 | N | THR | A | 147 | 17.986 | 17.616 | −13.794 | 1.00 | 39.07 | N |
| ATOM | 1091 | CA | THR | A | 147 | 18.914 | 17.270 | −12.723 | 1.00 | 36.70 | C |
| ATOM | 1092 | CB | THR | A | 147 | 18.558 | 18.011 | −11.401 | 1.00 | 37.46 | C |
| ATOM | 1093 | OG1 | THR | A | 147 | 18.771 | 19.419 | −11.542 | 1.00 | 32.66 | O |
| ATOM | 1094 | CG2 | THR | A | 147 | 19.418 | 17.501 | −10.239 | 1.00 | 36.18 | C |
| ATOM | 1095 | C | THR | A | 147 | 18.847 | 15.737 | −12.552 | 1.00 | 39.09 | C |
| ATOM | 1096 | O | THR | A | 147 | 17.742 | 15.142 | −12.547 | 1.00 | 35.10 | O |
| ATOM | 1097 | N | ARG | A | 148 | 20.014 | 15.098 | −12.483 | 1.00 | 37.07 | N |
| ATOM | 1098 | CA | ARG | A | 148 | 20.090 | 13.707 | −12.031 | 1.00 | 37.30 | C |
| ATOM | 1099 | CB | ARG | A | 148 | 20.887 | 12.827 | −13.001 | 1.00 | 36.01 | C |
| ATOM | 1100 | CG | ARG | A | 148 | 20.231 | 12.730 | −14.351 | 1.00 | 42.87 | C |
| ATOM | 1101 | CD | ARG | A | 148 | 21.219 | 12.497 | −15.435 | 1.00 | 46.15 | C |
| ATOM | 1102 | NE | ARG | A | 148 | 20.558 | 12.599 | −16.732 | 1.00 | 49.73 | N |
| ATOM | 1103 | CZ | ARG | A | 148 | 20.535 | 13.696 | −17.480 | 1.00 | 54.09 | C |
| ATOM | 1104 | NH1 | ARG | A | 148 | 21.159 | 14.796 | −17.073 | 1.00 | 55.32 | N |
| ATOM | 1105 | NH2 | ARG | A | 148 | 19.886 | 13.687 | −18.643 | 1.00 | 48.49 | N |
| ATOM | 1106 | C | ARG | A | 148 | 20.781 | 13.718 | −10.689 | 1.00 | 37.62 | C |
| ATOM | 1107 | O | ARG | A | 148 | 21.837 | 14.357 | −10.536 | 1.00 | 40.62 | O |
| ATOM | 1108 | N | LYS | A | 149 | 20.197 | 13.006 | −9.729 | 1.00 | 33.82 | N |
| ATOM | 1109 | CA | LYS | A | 149 | 20.717 | 12.989 | −8.374 | 1.00 | 38.43 | C |
| ATOM | 1110 | CB | LYS | A | 149 | 19.724 | 13.631 | −7.391 | 1.00 | 36.45 | C |
| ATOM | 1111 | CG | LYS | A | 149 | 19.349 | 15.063 | −7.677 | 1.00 | 39.04 | C |
| ATOM | 1112 | CD | LYS | A | 149 | 18.851 | 15.705 | −6.408 | 1.00 | 31.27 | C |
| ATOM | 1113 | CE | LYS | A | 149 | 20.019 | 16.113 | −5.543 | 1.00 | 33.52 | C |
| ATOM | 1114 | NZ | LYS | A | 149 | 19.519 | 16.702 | −4.262 | 1.00 | 31.74 | N |
| ATOM | 1115 | C | LYS | A | 149 | 20.888 | 11.548 | −7.968 | 1.00 | 39.40 | C |
| ATOM | 1116 | O | LYS | A | 149 | 20.194 | 10.675 | −8.488 | 1.00 | 43.92 | O |
| ATOM | 1117 | N | ARG | A | 150 | 21.794 | 11.293 | −7.032 | 1.00 | 38.52 | N |
| ATOM | 1118 | CA | ARG | A | 150 | 21.841 | 9.999 | −6.426 | 1.00 | 37.72 | C |
| ATOM | 1119 | CB | ARG | A | 150 | 23.048 | 9.212 | −6.913 | 1.00 | 39.83 | C |
| ATOM | 1120 | CG | ARG | A | 150 | 22.677 | 7.815 | −7.416 | 1.00 | 40.68 | C |
| ATOM | 1121 | CD | ARG | A | 150 | 23.919 | 7.066 | −7.884 | 1.00 | 50.69 | C |
| ATOM | 1122 | NE | ARG | A | 150 | 25.109 | 7.751 | −7.419 | 1.00 | 48.61 | N |
| ATOM | 1123 | CZ | ARG | A | 150 | 25.889 | 7.366 | −6.419 | 1.00 | 50.11 | C |
| ATOM | 1124 | NH1 | ARG | A | 150 | 25.659 | 6.244 | −5.744 | 1.00 | 40.80 | N |
| ATOM | 1125 | NH2 | ARG | A | 150 | 26.919 | 8.124 | −6.113 | 1.00 | 45.14 | N |
| ATOM | 1126 | C | ARG | A | 150 | 21.770 | 10.108 | −4.899 | 1.00 | 41.36 | C |
| ATOM | 1127 | O | ARG | A | 150 | 22.707 | 10.607 | −4.252 | 1.00 | 42.41 | O |
| ATOM | 1128 | N | PRO | A | 151 | 20.643 | 9.645 | −4.314 | 1.00 | 40.04 | N |
| ATOM | 1129 | CA | PRO | A | 151 | 20.352 | 9.965 | −2.926 | 1.00 | 43.16 | C |
| ATOM | 1130 | CB | PRO | A | 151 | 19.275 | 8.954 | −2.566 | 1.00 | 44.97 | C |
| ATOM | 1131 | CG | PRO | A | 151 | 18.535 | 8.737 | −3.854 | 1.00 | 42.32 | C |
| ATOM | 1132 | CD | PRO | A | 151 | 19.592 | 8.813 | −4.923 | 1.00 | 40.05 | C |
| ATOM | 1133 | C | PRO | A | 151 | 21.600 | 9.831 | −2.018 | 1.00 | 47.19 | C |
| ATOM | 1134 | O | PRO | A | 151 | 22.261 | 8.794 | −2.040 | 1.00 | 45.98 | O |
| ATOM | 1135 | N | GLY | A | 152 | 21.922 | 10.913 | −1.299 | 1.00 | 47.18 | N |
| ATOM | 1136 | CA | GLY | A | 152 | 22.973 | 10.945 | −0.277 | 1.00 | 44.93 | C |
| ATOM | 1137 | C | GLY | A | 152 | 24.383 | 11.075 | −0.813 | 1.00 | 43.32 | C |
| ATOM | 1138 | O | GLY | A | 152 | 25.353 | 11.273 | −0.062 | 1.00 | 45.52 | O |
| ATOM | 1139 | N | ALA | A | 153 | 24.503 | 10.996 | −2.122 | 1.00 | 42.03 | N |
| ATOM | 1140 | CA | ALA | A | 153 | 25.791 | 10.730 | −2.711 | 1.00 | 42.81 | C |
| ATOM | 1141 | CB | ALA | A | 153 | 25.779 | 9.348 | −3.358 | 1.00 | 41.08 | C |
| ATOM | 1142 | C | ALA | A | 153 | 26.205 | 11.781 | −3.717 | 1.00 | 43.37 | C |
| ATOM | 1143 | O | ALA | A | 153 | 27.284 | 12.352 | −3.606 | 1.00 | 45.89 | O |
| ATOM | 1144 | N | GLU | A | 154 | 25.372 | 12.042 | −4.716 | 1.00 | 38.19 | N |
| ATOM | 1145 | CA | GLU | A | 154 | 25.889 | 12.780 | −5.851 | 1.00 | 36.09 | C |
| ATOM | 1146 | CB | GLU | A | 154 | 26.600 | 11.797 | −6.763 | 1.00 | 34.29 | C |
| ATOM | 1147 | CG | GLU | A | 154 | 27.493 | 12.370 | −7.838 | 1.00 | 38.73 | C |
| ATOM | 1148 | CD | GLU | A | 154 | 28.005 | 11.307 | −8.808 | 1.00 | 37.40 | C |
| ATOM | 1149 | OE1 | GLU | A | 154 | 27.908 | 10.093 | −8.512 | 1.00 | 44.61 | O |
| ATOM | 1150 | OE2 | GLU | A | 154 | 28.460 | 11.691 | −9.898 | 1.00 | 40.39 | O |
| ATOM | 1151 | C | GLU | A | 154 | 24.793 | 13.458 | −6.631 | 1.00 | 40.06 | C |
| ATOM | 1152 | O | GLU | A | 154 | 23.649 | 12.986 | −6.641 | 1.00 | 36.30 | O |
| ATOM | 1153 | N | ALA | A | 155 | 25.169 | 14.505 | −7.359 | 1.00 | 38.96 | N |
| ATOM | 1154 | CA | ALA | A | 155 | 24.204 | 15.221 | −8.171 | 1.00 | 37.58 | C |
| ATOM | 1155 | CB | ALA | A | 155 | 23.518 | 16.297 | −7.339 | 1.00 | 41.18 | C |
| ATOM | 1156 | C | ALA | A | 155 | 24.837 | 15.832 | −9.388 | 1.00 | 35.95 | C |
| ATOM | 1157 | O | ALA | A | 155 | 25.998 | 16.229 | −9.358 | 1.00 | 38.79 | O |
| ATOM | 1158 | N | THR | A | 156 | 24.083 | 15.898 | −10.476 | 1.00 | 36.21 | N |
| ATOM | 1159 | CA | THR | A | 156 | 24.470 | 16.730 | −11.607 | 1.00 | 39.46 | C |
| ATOM | 1160 | CB | THR | A | 156 | 24.920 | 15.915 | −12.849 | 1.00 | 41.34 | C |
| ATOM | 1161 | OG1 | THR | A | 156 | 23.904 | 14.953 | −13.171 | 1.00 | 43.78 | O |
| ATOM | 1162 | CG2 | THR | A | 156 | 26.249 | 15.214 | −12.622 | 1.00 | 38.27 | C |
| ATOM | 1163 | C | THR | A | 156 | 23.276 | 17.509 | −12.046 | 1.00 | 39.87 | C |
| ATOM | 1164 | O | THR | A | 156 | 22.122 | 17.050 | −11.933 | 1.00 | 37.90 | O |
| ATOM | 1165 | N | GLY | A | 157 | 23.529 | 18.690 | −12.578 | 1.00 | 39.18 | N |
| ATOM | 1166 | CA | GLY | A | 157 | 22.428 | 19.490 | −13.084 | 1.00 | 37.13 | C |
| ATOM | 1167 | C | GLY | A | 157 | 22.780 | 20.950 | −13.226 | 1.00 | 35.38 | C |
| ATOM | 1168 | O | GLY | A | 157 | 23.866 | 21.396 | −12.823 | 1.00 | 33.71 | O |
| ATOM | 1169 | N | LYS | A | 158 | 21.850 | 21.680 | −13.825 | 1.00 | 37.34 | N |
| ATOM | 1170 | CA | LYS | A | 158 | 21.886 | 23.131 | −13.921 | 1.00 | 37.93 | C |
| ATOM | 1171 | CB | LYS | A | 158 | 22.192 | 23.560 | −15.358 | 1.00 | 40.53 | C |
| ATOM | 1172 | C | LYS | A | 158 | 20.519 | 23.694 | −13.496 | 1.00 | 39.02 | C |
| ATOM | 1173 | O | LYS | A | 158 | 19.456 | 23.080 | −13.744 | 1.00 | 37.62 | O |
| ATOM | 1174 | N | VAL | A | 159 | 20.553 | 24.859 | −12.861 | 1.00 | 38.06 | N |
| ATOM | 1175 | CA | VAL | A | 159 | 19.336 | 25.622 | −12.551 | 1.00 | 39.03 | C |
| ATOM | 1176 | CB | VAL | A | 159 | 19.653 | 26.888 | −11.699 | 1.00 | 38.12 | C |
| ATOM | 1177 | CG1 | VAL | A | 159 | 18.378 | 27.596 | −11.228 | 1.00 | 31.20 | C |
| ATOM | 1178 | CG2 | VAL | A | 159 | 20.479 | 26.510 | −10.502 | 1.00 | 42.87 | C |
| ATOM | 1179 | C | VAL | A | 159 | 18.653 | 26.083 | −13.829 | 1.00 | 38.78 | C |
| ATOM | 1180 | O | VAL | A | 159 | 19.295 | 26.642 | −14.719 | 1.00 | 37.57 | O |
| ATOM | 1181 | N | ASN | A | 160 | 17.347 | 25.864 | −13.911 | 1.00 | 37.96 | N |
| ATOM | 1182 | CA | ASN | A | 160 | 16.555 | 26.480 | −14.968 | 1.00 | 40.09 | C |
| ATOM | 1183 | CB | ASN | A | 160 | 15.339 | 25.630 | −15.291 | 1.00 | 37.65 | C |
| ATOM | 1184 | CG | ASN | A | 160 | 15.735 | 24.343 | −15.924 | 1.00 | 41.03 | C |
| ATOM | 1185 | OD1 | ASN | A | 160 | 16.012 | 24.295 | −17.115 | 1.00 | 40.31 | O |
| ATOM | 1186 | ND2 | ASN | A | 160 | 15.850 | 23.304 | −15.127 | 1.00 | 39.22 | N |
| ATOM | 1187 | C | ASN | A | 160 | 16.195 | 27.909 | −14.653 | 1.00 | 39.02 | C |
| ATOM | 1188 | O | ASN | A | 160 | 16.020 | 28.284 | −13.504 | 1.00 | 40.50 | O |
| ATOM | 1189 | N | VAL | A | 161 | 16.152 | 28.736 | −15.676 | 1.00 | 41.64 | N |
| ATOM | 1190 | CA | VAL | A | 161 | 15.680 | 30.090 | −15.492 | 1.00 | 41.07 | C |
| ATOM | 1191 | CB | VAL | A | 161 | 16.807 | 31.143 | −15.573 | 1.00 | 44.12 | C |
| ATOM | 1192 | CG1 | VAL | A | 161 | 16.272 | 32.503 | −15.082 | 1.00 | 42.61 | C |
| ATOM | 1193 | CG2 | VAL | A | 161 | 18.065 | 30.722 | −14.755 | 1.00 | 39.53 | C |
| ATOM | 1194 | C | VAL | A | 161 | 14.604 | 30.348 | −16.546 | 1.00 | 42.83 | C |
| ATOM | 1195 | O | VAL | A | 161 | 14.835 | 30.185 | −17.737 | 1.00 | 43.10 | O |
| ATOM | 1196 | N | LYS | A | 162 | 13.401 | 30.676 | −16.090 | 1.00 | 43.32 | N |
| ATOM | 1197 | CA | LYS | A | 162 | 12.305 | 31.009 | −17.004 | 1.00 | 44.78 | C |
| ATOM | 1198 | CB | LYS | A | 162 | 11.214 | 29.918 | −17.004 | 1.00 | 42.48 | C |
| ATOM | 1199 | CG | LYS | A | 162 | 10.145 | 30.062 | −18.115 | 1.00 | 45.76 | C |
| ATOM | 1200 | CD | LYS | A | 162 | 10.761 | 29.861 | −19.514 | 1.00 | 47.76 | C |
| ATOM | 1201 | CE | LYS | A | 162 | 10.063 | 30.707 | −20.577 | 1.00 | 45.12 | C |
| ATOM | 1202 | NZ | LYS | A | 162 | 10.982 | 30.966 | −21.720 | 1.00 | 45.05 | N |
| ATOM | 1203 | C | LYS | A | 162 | 11.740 | 32.350 | −16.563 | 1.00 | 43.17 | C |
| ATOM | 1204 | O | LYS | A | 162 | 11.532 | 32.581 | −15.374 | 1.00 | 42.04 | O |
| ATOM | 1205 | N | THR | A | 163 | 11.538 | 33.254 | −17.512 | 1.00 | 43.54 | N |
| ATOM | 1206 | CA | THR | A | 163 | 10.833 | 34.481 | −17.193 | 1.00 | 39.40 | C |
| ATOM | 1207 | CB | THR | A | 163 | 11.658 | 35.711 | −17.533 | 1.00 | 42.58 | C |
| ATOM | 1208 | OG1 | THR | A | 163 | 12.816 | 35.742 | −16.676 | 1.00 | 44.30 | O |
| ATOM | 1209 | CG2 | THR | A | 163 | 10.846 | 37.006 | −17.339 | 1.00 | 29.43 | C |
| ATOM | 1210 | C | THR | A | 163 | 9.516 | 34.409 | −17.951 | 1.00 | 40.40 | C |
| ATOM | 1211 | O | THR | A | 163 | 9.499 | 34.187 | −19.154 | 1.00 | 42.46 | O |
| ATOM | 1212 | N | LEU | A | 164 | 8.427 | 34.532 | −17.211 | 1.00 | 39.29 | N |
| ATOM | 1213 | CA | LEU | A | 164 | 7.082 | 34.513 | −17.756 | 1.00 | 41.09 | C |
| ATOM | 1214 | CB | LEU | A | 164 | 6.223 | 33.580 | −16.903 | 1.00 | 37.65 | C |
| ATOM | 1215 | CG | LEU | A | 164 | 6.527 | 32.079 | −17.062 | 1.00 | 34.39 | C |
| ATOM | 1216 | CD1 | LEU | A | 164 | 5.534 | 31.295 | −16.280 | 1.00 | 33.44 | C |
| ATOM | 1217 | CD2 | LEU | A | 164 | 6.491 | 31.624 | −18.505 | 1.00 | 30.97 | C |
| ATOM | 1218 | C | LEU | A | 164 | 6.520 | 35.944 | −17.753 | 1.00 | 41.72 | C |
| ATOM | 1219 | O | LEU | A | 164 | 6.925 | 36.765 | −16.935 | 1.00 | 41.65 | O |
| ATOM | 1220 | N | ARG | A | 165 | 5.618 | 36.240 | −18.685 | 1.00 | 46.79 | N |
| ATOM | 1221 | CA | ARG | A | 165 | 4.950 | 37.552 | −18.728 | 1.00 | 48.66 | C |
| ATOM | 1222 | CB | ARG | A | 165 | 5.210 | 38.285 | −20.048 | 1.00 | 49.34 | C |
| ATOM | 1223 | CG | ARG | A | 165 | 4.630 | 39.706 | −20.067 | 1.00 | 54.33 | C |
| ATOM | 1224 | CD | ARG | A | 165 | 5.512 | 40.673 | −19.295 | 1.00 | 55.25 | C |
| ATOM | 1225 | NE | ARG | A | 165 | 6.735 | 40.850 | −20.050 | 1.00 | 63.03 | N |
| ATOM | 1226 | CZ | ARG | A | 165 | 6.834 | 41.649 | −21.098 | 1.00 | 59.03 | C |
| ATOM | 1227 | NH1 | ARG | A | 165 | 5.794 | 42.374 | −21.471 | 1.00 | 63.76 | N |
| ATOM | 1228 | NH2 | ARG | A | 165 | 7.978 | 41.732 | −21.761 | 1.00 | 63.22 | N |
| ATOM | 1229 | C | ARG | A | 165 | 3.452 | 37.432 | −18.484 | 1.00 | 48.66 | C |
| ATOM | 1230 | O | ARG | A | 165 | 2.830 | 36.449 | −18.860 | 1.00 | 52.45 | O |
| ATOM | 1231 | N | LEU | A | 166 | 2.871 | 38.466 | −17.891 | 1.00 | 46.26 | N |
| ATOM | 1232 | CA | LEU | A | 166 | 1.573 | 38.344 | −17.273 | 1.00 | 45.06 | C |
| ATOM | 1233 | CB | LEU | A | 166 | 1.798 | 37.906 | −15.831 | 1.00 | 46.66 | C |
| ATOM | 1234 | CG | LEU | A | 166 | 0.794 | 37.731 | −14.709 | 1.00 | 47.10 | C |
| ATOM | 1235 | CD1 | LEU | A | 166 | 1.252 | 36.507 | −13.939 | 1.00 | 47.69 | C |
| ATOM | 1236 | CD2 | LEU | A | 166 | 0.767 | 38.977 | −13.813 | 1.00 | 42.28 | C |
| ATOM | 1237 | C | LEU | A | 166 | 0.835 | 39.685 | −17.337 | 1.00 | 44.50 | C |
| ATOM | 1238 | O | LEU | A | 166 | 1.422 | 40.738 | −17.068 | 1.00 | 43.18 | O |
| ATOM | 1239 | N | GLY | A | 167 | −0.444 | 39.627 | −17.692 | 1.00 | 44.34 | N |
| ATOM | 1240 | CA | GLY | A | 167 | −1.286 | 40.830 | −17.818 | 1.00 | 46.04 | C |
| ATOM | 1241 | C | GLY | A | 167 | −2.089 | 40.951 | −19.117 | 1.00 | 46.16 | C |
| ATOM | 1242 | O | GLY | A | 167 | −2.277 | 39.969 | −19.851 | 1.00 | 48.83 | O |
| ATOM | 1243 | N | PRO | A | 168 | −2.604 | 42.155 | −19.404 | 1.00 | 44.04 | N |
| ATOM | 1244 | CA | PRO | A | 168 | −2.568 | 43.359 | −18.579 | 1.00 | 42.19 | C |
| ATOM | 1245 | CB | PRO | A | 168 | −3.125 | 44.437 | −19.517 | 1.00 | 42.07 | C |
| ATOM | 1246 | CG | PRO | A | 168 | −3.995 | 43.694 | −20.446 | 1.00 | 47.01 | C |
| ATOM | 1247 | CD | PRO | A | 168 | −3.278 | 42.402 | −20.690 | 1.00 | 45.76 | C |
| ATOM | 1248 | C | PRO | A | 168 | −3.411 | 43.262 | −17.308 | 1.00 | 40.54 | C |
| ATOM | 1249 | O | PRO | A | 168 | −4.488 | 42.655 | −17.313 | 1.00 | 38.18 | O |
| ATOM | 1250 | N | LEU | A | 169 | −2.921 | 43.906 | −16.250 | 1.00 | 43.31 | N |
| ATOM | 1251 | CA | LEU | A | 169 | −3.546 | 43.889 | −14.918 | 1.00 | 45.25 | C |
| ATOM | 1252 | CB | LEU | A | 169 | −2.505 | 43.511 | −13.880 | 1.00 | 45.68 | C |
| ATOM | 1253 | CG | LEU | A | 169 | −1.920 | 42.103 | −14.059 | 1.00 | 45.04 | C |
| ATOM | 1254 | CD1 | LEU | A | 169 | −0.397 | 42.117 | −13.926 | 1.00 | 50.43 | C |
| ATOM | 1255 | CD2 | LEU | A | 169 | −2.553 | 41.196 | −13.072 | 1.00 | 44.32 | C |
| ATOM | 1256 | C | LEU | A | 169 | −4.225 | 45.212 | −14.519 | 1.00 | 45.55 | C |
| ATOM | 1257 | O | LEU | A | 169 | −3.719 | 46.295 | −14.828 | 1.00 | 47.38 | O |
| ATOM | 1258 | N | SER | A | 170 | −5.337 | 45.087 | −13.794 | 1.00 | 43.84 | N |
| ATOM | 1259 | CA | SER | A | 170 | −6.237 | 46.193 | −13.435 | 1.00 | 48.14 | C |
| ATOM | 1260 | CB | SER | A | 170 | −7.617 | 45.992 | −14.085 | 1.00 | 44.83 | C |
| ATOM | 1261 | OG | SER | A | 170 | −7.590 | 46.294 | −15.470 | 1.00 | 50.91 | O |
| ATOM | 1262 | C | SER | A | 170 | −6.484 | 46.371 | −11.941 | 1.00 | 47.63 | C |
| ATOM | 1263 | O | SER | A | 170 | −6.701 | 47.502 | −11.490 | 1.00 | 47.42 | O |
| ATOM | 1264 | N | LYS | A | 171 | −6.486 | 45.257 | −11.195 | 1.00 | 44.02 | N |
| ATOM | 1265 | CA | LYS | A | 171 | −6.991 | 45.256 | −9.817 | 1.00 | 43.48 | C |
| ATOM | 1266 | CB | LYS | A | 171 | −7.645 | 43.905 | −9.455 | 1.00 | 43.41 | C |
| ATOM | 1267 | CG | LYS | A | 171 | −8.737 | 43.410 | −10.428 | 1.00 | 42.89 | C |
| ATOM | 1268 | CD | LYS | A | 171 | −9.921 | 42.790 | −9.677 | 1.00 | 45.77 | C |
| ATOM | 1269 | CE | LYS | A | 171 | −10.862 | 42.045 | −10.613 | 1.00 | 48.97 | C |
| ATOM | 1270 | NZ | LYS | A | 171 | −10.337 | 40.673 | −10.938 | 1.00 | 43.91 | N |
| ATOM | 1271 | C | LYS | A | 171 | −5.904 | 45.615 | −8.823 | 1.00 | 42.35 | C |
| ATOM | 1272 | O | LYS | A | 171 | −4.744 | 45.750 | −9.203 | 1.00 | 43.62 | O |
| ATOM | 1273 | N | ALA | A | 172 | −6.262 | 45.775 | −7.554 | 1.00 | 40.15 | N |
| ATOM | 1274 | CA | ALA | A | 172 | −5.296 | 46.286 | −6.592 | 1.00 | 40.64 | C |
| ATOM | 1275 | CB | ALA | A | 172 | −5.963 | 46.540 | −5.291 | 1.00 | 40.23 | C |
| ATOM | 1276 | C | ALA | A | 172 | −4.120 | 45.317 | −6.425 | 1.00 | 39.80 | C |
| ATOM | 1277 | O | ALA | A | 172 | −2.984 | 45.726 | −6.184 | 1.00 | 43.11 | O |
| ATOM | 1278 | N | GLY | A | 173 | −4.399 | 44.031 | −6.543 | 1.00 | 40.73 | N |
| ATOM | 1279 | CA | GLY | A | 173 | −3.365 | 43.015 | −6.434 | 1.00 | 37.01 | C |
| ATOM | 1280 | C | GLY | A | 173 | −3.745 | 41.677 | −7.017 | 1.00 | 38.09 | C |
| ATOM | 1281 | O | GLY | A | 173 | −4.871 | 41.495 | −7.536 | 1.00 | 35.36 | O |
| ATOM | 1282 | N | PHE | A | 174 | −2.814 | 40.720 | −6.921 | 1.00 | 30.39 | N |
| ATOM | 1283 | CA | PHE | A | 174 | −3.020 | 39.450 | −7.557 | 1.00 | 32.18 | C |
| ATOM | 1284 | CB | PHE | A | 174 | −2.507 | 39.466 | −9.014 | 1.00 | 31.00 | C |
| ATOM | 1285 | CG | PHE | A | 174 | −1.004 | 39.557 | −9.138 | 1.00 | 33.53 | C |
| ATOM | 1286 | CD1 | PHE | A | 174 | −0.220 | 38.387 | −9.184 | 1.00 | 39.87 | C |
| ATOM | 1287 | CE1 | PHE | A | 174 | 1.160 | 38.447 | −9.279 | 1.00 | 40.76 | C |
| ATOM | 1288 | CZ | PHE | A | 174 | 1.804 | 39.678 | −9.356 | 1.00 | 36.30 | C |
| ATOM | 1289 | CE2 | PHE | A | 174 | 1.038 | 40.872 | −9.305 | 1.00 | 40.79 | C |
| ATOM | 1290 | CD2 | PHE | A | 174 | −0.372 | 40.794 | −9.206 | 1.00 | 34.74 | C |
| ATOM | 1291 | C | PHE | A | 174 | −2.364 | 38.312 | −6.746 | 1.00 | 30.68 | C |
| ATOM | 1292 | O | PHE | A | 174 | −1.698 | 38.568 | −5.748 | 1.00 | 34.70 | O |
| ATOM | 1293 | N | TYR | A | 175 | −2.504 | 37.100 | −7.254 | 1.00 | 34.65 | N |
| ATOM | 1294 | CA | TYR | A | 175 | −1.825 | 35.917 | −6.697 | 1.00 | 31.11 | C |
| ATOM | 1295 | CB | TYR | A | 175 | −2.859 | 34.963 | −6.139 | 1.00 | 36.87 | C |
| ATOM | 1296 | CG | TYR | A | 175 | −3.695 | 35.498 | −5.041 | 1.00 | 36.55 | C |
| ATOM | 1297 | CD1 | TYR | A | 175 | −3.246 | 35.442 | −3.722 | 1.00 | 34.78 | C |
| ATOM | 1298 | CE1 | TYR | A | 175 | −4.004 | 35.942 | −2.709 | 1.00 | 37.57 | C |
| ATOM | 1299 | CZ | TYR | A | 175 | −5.241 | 36.488 | −2.985 | 1.00 | 35.98 | C |
| ATOM | 1300 | OH | TYR | A | 175 | −6.003 | 36.966 | −1.953 | 1.00 | 34.98 | O |
| ATOM | 1301 | CE2 | TYR | A | 175 | −5.719 | 36.555 | −4.281 | 1.00 | 39.64 | C |
| ATOM | 1302 | CD2 | TYR | A | 175 | −4.939 | 36.044 | −5.306 | 1.00 | 34.73 | C |
| ATOM | 1303 | C | TYR | A | 175 | −1.215 | 35.129 | −7.800 | 1.00 | 34.83 | C |
| ATOM | 1304 | O | TYR | A | 175 | −1.844 | 34.963 | −8.839 | 1.00 | 36.19 | O |
| ATOM | 1305 | N | LEU | A | 176 | −0.063 | 34.506 | −7.533 | 1.00 | 34.99 | N |
| ATOM | 1306 | CA | LEU | A | 176 | 0.505 | 33.635 | −8.516 | 1.00 | 36.02 | C |
| ATOM | 1307 | CB | LEU | A | 176 | 1.978 | 33.991 | −8.765 | 1.00 | 32.58 | C |
| ATOM | 1308 | CG | LEU | A | 176 | 2.568 | 33.556 | −10.093 | 1.00 | 33.93 | C |
| ATOM | 1309 | CD1 | LEU | A | 176 | 1.971 | 34.305 | −11.256 | 1.00 | 35.00 | C |
| ATOM | 1310 | CD2 | LEU | A | 176 | 4.068 | 33.794 | −10.077 | 1.00 | 34.45 | C |
| ATOM | 1311 | C | LEU | A | 176 | 0.340 | 32.206 | −8.048 | 1.00 | 33.83 | C |
| ATOM | 1312 | O | LEU | A | 176 | 0.550 | 31.923 | −6.871 | 1.00 | 34.45 | O |
| ATOM | 1313 | N | ALA | A | 177 | −0.007 | 31.307 | −8.961 | 1.00 | 31.16 | N |
| ATOM | 1314 | CA | ALA | A | 177 | −0.165 | 29.924 | −8.570 | 1.00 | 31.94 | C |
| ATOM | 1315 | CB | ALA | A | 177 | −1.672 | 29.506 | −8.480 | 1.00 | 33.96 | C |
| ATOM | 1316 | C | ALA | A | 177 | 0.632 | 28.995 | −9.461 | 1.00 | 35.75 | C |
| ATOM | 1317 | O | ALA | A | 177 | 0.909 | 29.258 | −10.658 | 1.00 | 35.44 | O |
| ATOM | 1318 | N | PHE | A | 178 | 1.033 | 27.900 | −8.836 | 1.00 | 34.91 | N |
| ATOM | 1319 | CA | PHE | A | 178 | 1.835 | 26.896 | −9.493 | 1.00 | 36.95 | C |
| ATOM | 1320 | CB | PHE | A | 178 | 3.173 | 26.699 | −8.768 | 1.00 | 37.01 | C |
| ATOM | 1321 | CG | PHE | A | 178 | 4.025 | 27.931 | −8.725 | 1.00 | 40.04 | C |
| ATOM | 1322 | CD1 | PHE | A | 178 | 5.022 | 28.135 | −9.690 | 1.00 | 34.45 | C |
| ATOM | 1323 | CE1 | PHE | A | 178 | 5.826 | 29.267 | −9.655 | 1.00 | 41.39 | C |
| ATOM | 1324 | CZ | PHE | A | 178 | 5.630 | 30.221 | −8.645 | 1.00 | 38.98 | C |
| ATOM | 1325 | CE2 | PHE | A | 178 | 4.617 | 30.043 | −7.695 | 1.00 | 38.28 | C |
| ATOM | 1326 | CD2 | PHE | A | 178 | 3.830 | 28.896 | −7.727 | 1.00 | 31.99 | C |
| ATOM | 1327 | C | PHE | A | 178 | 1.019 | 25.652 | −9.361 | 1.00 | 36.21 | C |
| ATOM | 1328 | O | PHE | A | 178 | 0.613 | 25.292 | −8.255 | 1.00 | 37.32 | O |
| ATOM | 1329 | N | GLN | A | 179 | 0.771 | 25.001 | −10.490 | 1.00 | 35.99 | N |
| ATOM | 1330 | CA | GLN | A | 179 | 0.151 | 23.696 | −10.481 | 1.00 | 37.46 | C |
| ATOM | 1331 | CB | GLN | A | 179 | −1.193 | 23.726 | −11.248 | 1.00 | 37.59 | C |
| ATOM | 1332 | CG | GLN | A | 179 | −1.779 | 22.345 | −11.528 | 1.00 | 34.22 | C |
| ATOM | 1333 | CD | GLN | A | 179 | −2.739 | 22.326 | −12.728 | 1.00 | 39.93 | C |
| ATOM | 1334 | OE1 | GLN | A | 179 | −2.403 | 22.777 | −13.817 | 1.00 | 45.97 | O |
| ATOM | 1335 | NE2 | GLN | A | 179 | −3.922 | 21.754 | −12.529 | 1.00 | 45.45 | N |
| ATOM | 1336 | C | GLN | A | 179 | 1.068 | 22.583 | −10.991 | 1.00 | 35.94 | C |
| ATOM | 1337 | O | GLN | A | 179 | 1.540 | 22.601 | −12.105 | 1.00 | 33.60 | O |
| ATOM | 1338 | N | ASP | A | 180 | 1.271 | 21.592 | −10.138 | 1.00 | 39.76 | N |
| ATOM | 1339 | CA | ASP | A | 180 | 2.028 | 20.410 | −10.456 | 1.00 | 40.59 | C |
| ATOM | 1340 | CB | ASP | A | 180 | 2.898 | 20.092 | −9.250 | 1.00 | 37.86 | C |
| ATOM | 1341 | CG | ASP | A | 180 | 3.454 | 18.706 | −9.294 | 1.00 | 39.57 | C |
| ATOM | 1342 | OD1 | ASP | A | 180 | 4.226 | 18.446 | −10.237 | 1.00 | 39.93 | O |
| ATOM | 1343 | OD2 | ASP | A | 180 | 3.141 | 17.893 | −8.384 | 1.00 | 35.30 | O |
| ATOM | 1344 | C | ASP | A | 180 | 1.106 | 19.214 | −10.744 | 1.00 | 40.58 | C |
| ATOM | 1345 | O | ASP | A | 180 | 0.119 | 19.009 | −10.048 | 1.00 | 41.81 | O |
| ATOM | 1346 | N | GLN | A | 181 | 1.417 | 18.427 | −11.769 | 1.00 | 41.23 | N |
| ATOM | 1347 | CA | GLN | A | 181 | 0.612 | 17.229 | −12.057 | 1.00 | 40.16 | C |
| ATOM | 1348 | CB | GLN | A | 181 | −0.197 | 17.361 | −13.365 | 1.00 | 38.15 | C |
| ATOM | 1349 | CG | GLN | A | 181 | −0.923 | 18.687 | −13.561 | 1.00 | 41.10 | C |
| ATOM | 1350 | CD | GLN | A | 181 | −1.592 | 18.821 | −14.926 | 1.00 | 43.97 | C |
| ATOM | 1351 | OE1 | GLN | A | 181 | −1.923 | 17.826 | −15.577 | 1.00 | 43.86 | O |
| ATOM | 1352 | NE2 | GLN | A | 181 | −1.807 | 20.066 | −15.362 | 1.00 | 46.46 | N |
| ATOM | 1353 | C | GLN | A | 181 | 1.478 | 15.981 | −12.087 | 1.00 | 37.45 | C |
| ATOM | 1354 | O | GLN | A | 181 | 1.018 | 14.916 | −12.448 | 1.00 | 38.23 | O |
| ATOM | 1355 | N | GLY | A | 182 | 2.733 | 16.097 | −11.679 | 1.00 | 35.03 | N |
| ATOM | 1356 | CA | GLY | A | 182 | 3.569 | 14.916 | −11.563 | 1.00 | 31.33 | C |
| ATOM | 1357 | C | GLY | A | 182 | 5.061 | 15.219 | −11.657 | 1.00 | 32.94 | C |
| ATOM | 1358 | O | GLY | A | 182 | 5.853 | 14.337 | −11.965 | 1.00 | 33.01 | O |
| ATOM | 1359 | N | ALA | A | 183 | 5.455 | 16.446 | −11.376 | 1.00 | 27.42 | N |
| ATOM | 1360 | CA | ALA | A | 183 | 6.916 | 16.789 | −11.503 | 1.00 | 34.16 | C |
| ATOM | 1361 | CB | ALA | A | 183 | 7.084 | 18.245 | −11.905 | 1.00 | 31.51 | C |
| ATOM | 1362 | C | ALA | A | 183 | 7.730 | 16.465 | −10.224 | 1.00 | 36.88 | C |
| ATOM | 1363 | O | ALA | A | 183 | 7.165 | 16.305 | −9.146 | 1.00 | 38.15 | O |
| ATOM | 1364 | N | CYS | A | 184 | 9.054 | 16.330 | −10.362 | 1.00 | 38.55 | N |
| ATOM | 1365 | CA | CYS | A | 184 | 9.983 | 16.326 | −9.232 | 1.00 | 37.77 | C |
| ATOM | 1366 | CB | CYS | A | 184 | 10.821 | 15.026 | −9.216 | 1.00 | 40.69 | C |
| ATOM | 1367 | SG | CYS | A | 184 | 11.729 | 14.712 | −7.696 | 1.00 | 37.47 | S |
| ATOM | 1368 | C | CYS | A | 184 | 10.866 | 17.571 | −9.390 | 1.00 | 36.58 | C |
| ATOM | 1369 | O | CYS | A | 184 | 11.808 | 17.554 | −10.162 | 1.00 | 34.19 | O |
| ATOM | 1370 | N | MET | A | 185 | 10.509 | 18.667 | −8.705 | 1.00 | 35.34 | N |
| ATOM | 1371 | CA | MET | A | 185 | 11.161 | 19.976 | −8.863 | 1.00 | 36.03 | C |
| ATOM | 1372 | CB | MET | A | 185 | 10.379 | 20.827 | −9.856 | 1.00 | 34.42 | C |
| ATOM | 1373 | CG | MET | A | 185 | 8.902 | 21.024 | −9.488 | 1.00 | 34.05 | C |
| ATOM | 1374 | SD | MET | A | 185 | 8.089 | 22.180 | −10.598 | 1.00 | 36.69 | S |
| ATOM | 1375 | CE | MET | A | 185 | 8.821 | 23.708 | −10.012 | 1.00 | 44.23 | C |
| ATOM | 1376 | C | MET | A | 185 | 11.317 | 20.774 | −7.567 | 1.00 | 29.84 | C |
| ATOM | 1377 | O | MET | A | 185 | 10.536 | 20.592 | −6.637 | 1.00 | 29.27 | O |
| ATOM | 1378 | N | ALA | A | 186 | 12.397 | 21.553 | −7.464 | 1.00 | 30.72 | N |
| ATOM | 1379 | CA | ALA | A | 186 | 12.518 | 22.596 | −6.450 | 1.00 | 27.77 | C |
| ATOM | 1380 | CB | ALA | A | 186 | 13.910 | 22.553 | −5.745 | 1.00 | 25.18 | C |
| ATOM | 1381 | C | ALA | A | 186 | 12.336 | 23.979 | −7.125 | 1.00 | 24.78 | C |
| ATOM | 1382 | O | ALA | A | 186 | 12.961 | 24.251 | −8.150 | 1.00 | 31.56 | O |
| ATOM | 1383 | N | LEU | A | 187 | 11.565 | 24.864 | −6.507 | 1.00 | 27.48 | N |
| ATOM | 1384 | CA | LEU | A | 187 | 11.519 | 26.299 | −6.879 | 1.00 | 27.38 | C |
| ATOM | 1385 | CB | LEU | A | 187 | 10.101 | 26.887 | −6.703 | 1.00 | 30.71 | C |
| ATOM | 1386 | CG | LEU | A | 187 | 9.980 | 28.385 | −7.052 | 1.00 | 29.76 | C |
| ATOM | 1387 | CD1 | LEU | A | 187 | 9.960 | 28.497 | −8.550 | 1.00 | 25.31 | C |
| ATOM | 1388 | CD2 | LEU | A | 187 | 8.780 | 29.029 | −6.446 | 1.00 | 38.88 | C |
| ATOM | 1389 | C | LEU | A | 187 | 12.439 | 27.016 | −5.931 | 1.00 | 26.96 | C |
| ATOM | 1390 | O | LEU | A | 187 | 12.136 | 27.168 | −4.765 | 1.00 | 31.63 | O |
| ATOM | 1391 | N | LEU | A | 188 | 13.585 | 27.419 | −6.445 | 1.00 | 23.82 | N |
| ATOM | 1392 | CA | LEU | A | 188 | 14.649 | 28.033 | −5.680 | 1.00 | 22.42 | C |
| ATOM | 1393 | CB | LEU | A | 188 | 15.952 | 27.928 | −6.492 | 1.00 | 18.60 | C |
| ATOM | 1394 | CG | LEU | A | 188 | 16.336 | 26.464 | −6.872 | 1.00 | 22.31 | C |
| ATOM | 1395 | CD1 | LEU | A | 188 | 17.713 | 26.386 | −7.539 | 1.00 | 28.31 | C |
| ATOM | 1396 | CD2 | LEU | A | 188 | 16.255 | 25.517 | −5.679 | 1.00 | 28.43 | C |
| ATOM | 1397 | C | LEU | A | 188 | 14.302 | 29.477 | −5.412 | 1.00 | 22.74 | C |
| ATOM | 1398 | O | LEU | A | 188 | 14.529 | 29.936 | −4.329 | 1.00 | 28.62 | O |
| ATOM | 1399 | N | SER | A | 189 | 13.673 | 30.153 | −6.382 | 1.00 | 27.21 | N |
| ATOM | 1400 | CA | SER | A | 189 | 13.270 | 31.519 | −6.173 | 1.00 | 31.26 | C |
| ATOM | 1401 | CB | SER | A | 189 | 14.515 | 32.436 | −6.114 | 1.00 | 36.03 | C |
| ATOM | 1402 | OG | SER | A | 189 | 15.019 | 32.681 | −7.411 | 1.00 | 31.51 | O |
| ATOM | 1403 | C | SER | A | 189 | 12.252 | 32.016 | −7.211 | 1.00 | 32.45 | C |
| ATOM | 1404 | O | SER | A | 189 | 12.127 | 31.446 | −8.302 | 1.00 | 30.70 | O |
| ATOM | 1405 | N | LEU | A | 190 | 11.481 | 33.035 | −6.801 | 1.00 | 33.70 | N |
| ATOM | 1406 | CA | LEU | A | 190 | 10.455 | 33.696 | −7.616 | 1.00 | 35.24 | C |
| ATOM | 1407 | CB | LEU | A | 190 | 8.999 | 33.318 | −7.175 | 1.00 | 33.65 | C |
| ATOM | 1408 | CG | LEU | A | 190 | 7.879 | 34.252 | −7.704 | 1.00 | 37.98 | C |
| ATOM | 1409 | CD1 | LEU | A | 190 | 7.758 | 34.196 | −9.248 | 1.00 | 38.61 | C |
| ATOM | 1410 | CD2 | LEU | A | 190 | 6.494 | 34.037 | −7.064 | 1.00 | 34.04 | C |
| ATOM | 1411 | C | LEU | A | 190 | 10.680 | 35.193 | −7.476 | 1.00 | 35.25 | C |
| ATOM | 1412 | O | LEU | A | 190 | 10.779 | 35.711 | −6.376 | 1.00 | 33.56 | O |
| ATOM | 1413 | N | HIS | A | 191 | 10.780 | 35.893 | −8.594 | 1.00 | 37.40 | N |
| ATOM | 1414 | CA | HIS | A | 191 | 10.920 | 37.330 | −8.519 | 1.00 | 38.52 | C |
| ATOM | 1415 | CB | HIS | A | 191 | 12.402 | 37.758 | −8.720 | 1.00 | 37.86 | C |
| ATOM | 1416 | CG | HIS | A | 191 | 12.617 | 39.246 | −8.734 | 1.00 | 41.14 | C |
| ATOM | 1417 | ND1 | HIS | A | 191 | 13.565 | 39.845 | −9.531 | 1.00 | 38.74 | N |
| ATOM | 1418 | CE1 | HIS | A | 191 | 13.539 | 41.156 | −9.344 | 1.00 | 41.60 | C |
| ATOM | 1419 | NE2 | HIS | A | 191 | 12.627 | 41.424 | −8.430 | 1.00 | 40.15 | N |
| ATOM | 1420 | CD2 | HIS | A | 191 | 12.039 | 40.244 | −8.024 | 1.00 | 30.90 | C |
| ATOM | 1421 | C | HIS | A | 191 | 9.921 | 37.957 | −9.487 | 1.00 | 39.05 | C |
| ATOM | 1422 | O | HIS | A | 191 | 9.977 | 37.744 | −10.702 | 1.00 | 37.50 | O |
| ATOM | 1423 | N | LEU | A | 192 | 8.976 | 38.674 | −8.890 | 1.00 | 38.54 | N |
| ATOM | 1424 | CA | LEU | A | 192 | 7.912 | 39.401 | −9.592 | 1.00 | 40.13 | C |
| ATOM | 1425 | CB | LEU | A | 192 | 6.566 | 39.221 | −8.876 | 1.00 | 39.90 | C |
| ATOM | 1426 | CG | LEU | A | 192 | 5.960 | 37.835 | −8.772 | 1.00 | 36.90 | C |
| ATOM | 1427 | CD1 | LEU | A | 192 | 4.835 | 37.864 | −7.800 | 1.00 | 42.75 | C |
| ATOM | 1428 | CD2 | LEU | A | 192 | 5.498 | 37.403 | −10.150 | 1.00 | 33.58 | C |
| ATOM | 1429 | C | LEU | A | 192 | 8.274 | 40.859 | −9.531 | 1.00 | 37.03 | C |
| ATOM | 1430 | O | LEU | A | 192 | 8.557 | 41.381 | −8.457 | 1.00 | 36.49 | O |
| ATOM | 1431 | N | PHE | A | 193 | 8.241 | 41.502 | −10.687 | 1.00 | 38.07 | N |
| ATOM | 1432 | CA | PHE | A | 193 | 8.650 | 42.894 | −10.841 | 1.00 | 36.70 | C |
| ATOM | 1433 | CB | PHE | A | 193 | 10.216 | 43.035 | −10.959 | 1.00 | 29.97 | C |
| ATOM | 1434 | CG | PHE | A | 193 | 10.802 | 42.363 | −12.180 | 1.00 | 31.12 | C |
| ATOM | 1435 | CD1 | PHE | A | 193 | 11.097 | 43.105 | −13.328 | 1.00 | 37.50 | C |
| ATOM | 1436 | CE1 | PHE | A | 193 | 11.596 | 42.500 | −14.467 | 1.00 | 34.28 | C |
| ATOM | 1437 | CZ | PHE | A | 193 | 11.853 | 41.124 | −14.481 | 1.00 | 45.74 | C |
| ATOM | 1438 | CE2 | PHE | A | 193 | 11.563 | 40.350 | −13.352 | 1.00 | 30.73 | C |
| ATOM | 1439 | CD2 | PHE | A | 193 | 11.067 | 40.985 | −12.190 | 1.00 | 37.59 | C |
| ATOM | 1440 | C | PHE | A | 193 | 7.950 | 43.450 | −12.090 | 1.00 | 40.02 | C |
| ATOM | 1441 | O | PHE | A | 193 | 7.271 | 42.732 | −12.840 | 1.00 | 44.09 | O |
| ATOM | 1442 | N | TYR | A | 194 | 8.097 | 44.747 | −12.297 | 1.00 | 41.11 | N |
| ATOM | 1443 | CA | TYR | A | 194 | 7.673 | 45.348 | −13.580 | 1.00 | 41.03 | C |
| ATOM | 1444 | CB | TYR | A | 194 | 6.236 | 45.880 | −13.495 | 1.00 | 42.85 | C |
| ATOM | 1445 | CG | TYR | A | 194 | 6.023 | 47.133 | −12.652 | 1.00 | 45.26 | C |
| ATOM | 1446 | CD1 | TYR | A | 194 | 5.865 | 47.056 | −11.268 | 1.00 | 45.47 | C |
| ATOM | 1447 | CE1 | TYR | A | 194 | 5.635 | 48.201 | −10.499 | 1.00 | 41.30 | C |
| ATOM | 1448 | CZ | TYR | A | 194 | 5.566 | 49.425 | −11.108 | 1.00 | 41.62 | C |
| ATOM | 1449 | OH | TYR | A | 194 | 5.318 | 50.549 | −10.355 | 1.00 | 41.25 | O |
| ATOM | 1450 | CE2 | TYR | A | 194 | 5.703 | 49.532 | −12.480 | 1.00 | 44.15 | C |
| ATOM | 1451 | CD2 | TYR | A | 194 | 5.928 | 48.391 | −13.248 | 1.00 | 45.95 | C |
| ATOM | 1452 | C | TYR | A | 194 | 8.650 | 46.430 | −13.974 | 1.00 | 41.04 | C |
| ATOM | 1453 | O | TYR | A | 194 | 9.456 | 46.856 | −13.147 | 1.00 | 40.79 | O |
| ATOM | 1454 | N | LYS | A | 195 | 8.592 | 46.864 | −15.236 | 1.00 | 44.73 | N |
| ATOM | 1455 | CA | LYS | A | 195 | 9.456 | 47.945 | −15.729 | 1.00 | 46.28 | C |
| ATOM | 1456 | CB | LYS | A | 195 | 10.152 | 47.561 | −17.037 | 1.00 | 43.48 | C |
| ATOM | 1457 | CG | LYS | A | 195 | 10.979 | 46.305 | −16.912 | 1.00 | 47.45 | C |
| ATOM | 1458 | CD | LYS | A | 195 | 11.676 | 45.924 | −18.202 | 1.00 | 50.29 | C |
| ATOM | 1459 | CE | LYS | A | 195 | 12.404 | 44.596 | −18.004 | 1.00 | 50.32 | C |
| ATOM | 1460 | NZ | LYS | A | 195 | 12.635 | 43.846 | −19.282 | 1.00 | 59.34 | N |
| ATOM | 1461 | C | LYS | A | 195 | 8.624 | 49.217 | −15.860 | 1.00 | 47.80 | C |
| ATOM | 1462 | O | LYS | A | 195 | 7.537 | 49.218 | −16.454 | 1.00 | 45.96 | O |
| ATOM | 1463 | N | LYS | A | 196 | 9.144 | 50.290 | −15.277 | 1.00 | 49.37 | N |
| ATOM | 1464 | CA | LYS | A | 196 | 8.323 | 51.432 | −14.872 | 1.00 | 53.18 | C |
| ATOM | 1465 | CB | LYS | A | 196 | 9.160 | 52.333 | −13.968 | 1.00 | 53.61 | C |
| ATOM | 1466 | CG | LYS | A | 196 | 8.409 | 52.877 | −12.787 | 1.00 | 57.05 | C |
| ATOM | 1467 | CD | LYS | A | 196 | 9.326 | 52.905 | −11.589 | 1.00 | 56.96 | C |
| ATOM | 1468 | CE | LYS | A | 196 | 9.150 | 54.183 | −10.795 | 1.00 | 56.02 | C |
| ATOM | 1469 | NZ | LYS | A | 196 | 10.319 | 54.447 | −9.912 | 1.00 | 49.28 | N |
| ATOM | 1470 | C | LYS | A | 196 | 7.673 | 52.246 | −16.004 | 1.00 | 54.46 | C |
| ATOM | 1471 | O | LYS | A | 196 | 8.045 | 52.167 | −17.184 | 1.00 | 52.26 | O |
| ATOM | 1472 | OXT | LYS | A | 196 | 6.733 | 53.018 | −15.746 | 1.00 | 55.97 | O |
| ATOM | 1473 | N | ILE | B | 31 | 6.202 | 17.357 | 11.661 | 1.00 | 52.51 | N |
| ATOM | 1474 | CA | ILE | B | 31 | 6.652 | 17.466 | 13.083 | 1.00 | 51.68 | C |
| ATOM | 1475 | CB | ILE | B | 31 | 7.195 | 16.099 | 13.642 | 1.00 | 51.99 | C |
| ATOM | 1476 | CG1 | ILE | B | 31 | 8.524 | 15.693 | 12.959 | 1.00 | 56.45 | C |
| ATOM | 1477 | CD1 | ILE | B | 31 | 9.378 | 14.656 | 13.701 | 1.00 | 48.38 | C |
| ATOM | 1478 | CG2 | ILE | B | 31 | 6.139 | 15.008 | 13.502 | 1.00 | 57.59 | C |
| ATOM | 1479 | C | ILE | B | 31 | 7.694 | 18.588 | 13.242 | 1.00 | 51.74 | C |
| ATOM | 1480 | O | ILE | B | 31 | 8.867 | 18.423 | 12.904 | 1.00 | 49.06 | O |
| ATOM | 1481 | N | VAL | B | 32 | 7.254 | 19.737 | 13.751 | 1.00 | 51.69 | N |
| ATOM | 1482 | CA | VAL | B | 32 | 8.155 | 20.860 | 13.927 | 1.00 | 49.76 | C |
| ATOM | 1483 | CB | VAL | B | 32 | 7.409 | 22.216 | 13.764 | 1.00 | 49.19 | C |
| ATOM | 1484 | CG1 | VAL | B | 32 | 8.381 | 23.405 | 13.799 | 1.00 | 48.91 | C |
| ATOM | 1485 | CG2 | VAL | B | 32 | 6.606 | 22.225 | 12.458 | 1.00 | 50.33 | C |
| ATOM | 1486 | C | VAL | B | 32 | 8.812 | 20.697 | 15.292 | 1.00 | 51.68 | C |
| ATOM | 1487 | O | VAL | B | 32 | 8.130 | 20.724 | 16.318 | 1.00 | 55.62 | O |
| ATOM | 1488 | N | LEU | B | 33 | 10.130 | 20.483 | 15.307 | 1.00 | 51.40 | N |
| ATOM | 1489 | CA | LEU | B | 33 | 10.881 | 20.426 | 16.570 | 1.00 | 49.84 | C |
| ATOM | 1490 | CB | LEU | B | 33 | 12.181 | 19.648 | 16.400 | 1.00 | 49.11 | C |
| ATOM | 1491 | CG | LEU | B | 33 | 12.026 | 18.219 | 15.866 | 1.00 | 50.26 | C |
| ATOM | 1492 | CD1 | LEU | B | 33 | 13.092 | 17.915 | 14.844 | 1.00 | 45.27 | C |
| ATOM | 1493 | CD2 | LEU | B | 33 | 12.059 | 17.216 | 17.001 | 1.00 | 49.51 | C |
| ATOM | 1494 | C | LEU | B | 33 | 11.157 | 21.838 | 17.081 | 1.00 | 51.22 | C |
| ATOM | 1495 | O | LEU | B | 33 | 10.961 | 22.817 | 16.350 | 1.00 | 51.79 | O |
| ATOM | 1496 | N | GLU | B | 34 | 11.587 | 21.956 | 18.334 | 1.00 | 48.63 | N |
| ATOM | 1497 | CA | GLU | B | 34 | 11.756 | 23.283 | 18.942 | 1.00 | 50.49 | C |
| ATOM | 1498 | CB | GLU | B | 34 | 11.951 | 23.178 | 20.458 | 1.00 | 52.60 | C |
| ATOM | 1499 | C | GLU | B | 34 | 12.914 | 24.043 | 18.303 | 1.00 | 47.30 | C |
| ATOM | 1500 | O | GLU | B | 34 | 14.019 | 23.514 | 18.216 | 1.00 | 45.62 | O |
| ATOM | 1501 | N | PRO | B | 35 | 12.651 | 25.282 | 17.841 | 1.00 | 46.68 | N |
| ATOM | 1502 | CA | PRO | B | 35 | 13.663 | 26.137 | 17.253 | 1.00 | 44.35 | C |
| ATOM | 1503 | CB | PRO | B | 35 | 12.961 | 27.492 | 17.191 | 1.00 | 46.18 | C |
| ATOM | 1504 | CG | PRO | B | 35 | 11.535 | 27.136 | 16.959 | 1.00 | 44.55 | C |
| ATOM | 1505 | CD | PRO | B | 35 | 11.319 | 25.926 | 17.817 | 1.00 | 46.72 | C |
| ATOM | 1506 | C | PRO | B | 35 | 14.940 | 26.229 | 18.079 | 1.00 | 46.44 | C |
| ATOM | 1507 | O | PRO | B | 35 | 14.912 | 26.212 | 19.326 | 1.00 | 46.98 | O |
| ATOM | 1508 | N | ILE | B | 36 | 16.058 | 26.291 | 17.377 | 1.00 | 47.07 | N |
| ATOM | 1509 | CA | ILE | B | 36 | 17.354 | 26.348 | 18.024 | 1.00 | 47.75 | C |
| ATOM | 1510 | CB | ILE | B | 36 | 18.272 | 25.214 | 17.548 | 1.00 | 45.95 | C |
| ATOM | 1511 | CG1 | ILE | B | 36 | 17.727 | 23.879 | 18.073 | 1.00 | 46.11 | C |
| ATOM | 1512 | CD1 | ILE | B | 36 | 18.656 | 22.714 | 17.974 | 1.00 | 48.49 | C |
| ATOM | 1513 | CG2 | ILE | B | 36 | 19.708 | 25.452 | 18.031 | 1.00 | 52.98 | C |
| ATOM | 1514 | C | ILE | B | 36 | 17.994 | 27.717 | 17.845 | 1.00 | 47.45 | C |
| ATOM | 1515 | O | ILE | B | 36 | 18.367 | 28.116 | 16.739 | 1.00 | 47.44 | O |
| ATOM | 1516 | N | TYR | B | 37 | 18.090 | 28.444 | 18.951 | 1.00 | 48.70 | N |
| ATOM | 1517 | CA | TYR | B | 37 | 18.714 | 29.751 | 18.920 | 1.00 | 49.10 | C |
| ATOM | 1518 | CB | TYR | B | 37 | 18.089 | 30.679 | 19.948 | 1.00 | 48.08 | C |
| ATOM | 1519 | CG | TYR | B | 37 | 16.632 | 30.929 | 19.657 | 1.00 | 53.20 | C |
| ATOM | 1520 | CD1 | TYR | B | 37 | 16.201 | 32.116 | 19.037 | 1.00 | 55.88 | C |
| ATOM | 1521 | CE1 | TYR | B | 37 | 14.841 | 32.323 | 18.784 | 1.00 | 52.31 | C |
| ATOM | 1522 | CZ | TYR | B | 37 | 13.933 | 31.333 | 19.131 | 1.00 | 53.25 | C |
| ATOM | 1523 | OH | TYR | B | 37 | 12.585 | 31.480 | 18.900 | 1.00 | 55.81 | O |
| ATOM | 1524 | CE2 | TYR | B | 37 | 14.343 | 30.168 | 19.727 | 1.00 | 49.90 | C |
| ATOM | 1525 | CD2 | TYR | B | 37 | 15.675 | 29.969 | 19.988 | 1.00 | 55.92 | C |
| ATOM | 1526 | C | TYR | B | 37 | 20.208 | 29.644 | 19.095 | 1.00 | 48.06 | C |
| ATOM | 1527 | O | TYR | B | 37 | 20.705 | 29.188 | 20.135 | 1.00 | 47.00 | O |
| ATOM | 1528 | N | TRP | B | 38 | 20.902 | 30.081 | 18.055 | 1.00 | 44.34 | N |
| ATOM | 1529 | CA | TRP | B | 38 | 22.343 | 30.031 | 17.993 | 1.00 | 44.89 | C |
| ATOM | 1530 | CB | TRP | B | 38 | 22.794 | 29.823 | 16.533 | 1.00 | 45.89 | C |
| ATOM | 1531 | CG | TRP | B | 38 | 24.191 | 29.337 | 16.424 | 1.00 | 43.47 | C |
| ATOM | 1532 | CD1 | TRP | B | 38 | 25.320 | 30.086 | 16.476 | 1.00 | 46.09 | C |
| ATOM | 1533 | NE1 | TRP | B | 38 | 26.432 | 29.279 | 16.364 | 1.00 | 48.98 | N |
| ATOM | 1534 | CE2 | TRP | B | 38 | 26.015 | 27.980 | 16.247 | 1.00 | 42.45 | C |
| ATOM | 1535 | CD2 | TRP | B | 38 | 24.610 | 27.980 | 16.292 | 1.00 | 42.52 | C |
| ATOM | 1536 | CE3 | TRP | B | 38 | 23.930 | 26.763 | 16.179 | 1.00 | 47.79 | C |
| ATOM | 1537 | CZ3 | TRP | B | 38 | 24.653 | 25.621 | 16.043 | 1.00 | 46.68 | C |
| ATOM | 1538 | CH2 | TRP | B | 38 | 26.051 | 25.649 | 16.007 | 1.00 | 47.89 | C |
| ATOM | 1539 | CZ2 | TRP | B | 38 | 26.746 | 26.821 | 16.101 | 1.00 | 45.11 | C |
| ATOM | 1540 | C | TRP | B | 38 | 22.912 | 31.312 | 18.589 | 1.00 | 45.43 | C |
| ATOM | 1541 | O | TRP | B | 38 | 23.237 | 32.274 | 17.882 | 1.00 | 41.46 | O |
| ATOM | 1542 | N | ASN | B | 39 | 22.980 | 31.315 | 19.914 | 1.00 | 43.47 | N |
| ATOM | 1543 | CA | ASN | B | 39 | 23.587 | 32.390 | 20.669 | 1.00 | 47.07 | C |
| ATOM | 1544 | CB | ASN | B | 39 | 22.561 | 33.489 | 21.025 | 1.00 | 48.07 | C |
| ATOM | 1545 | CG | ASN | B | 39 | 21.403 | 32.981 | 21.887 | 1.00 | 48.17 | C |
| ATOM | 1546 | OD1 | ASN | B | 39 | 21.589 | 32.193 | 22.806 | 1.00 | 61.41 | O |
| ATOM | 1547 | ND2 | ASN | B | 39 | 20.207 | 33.467 | 21.607 | 1.00 | 56.57 | N |
| ATOM | 1548 | C | ASN | B | 39 | 24.274 | 31.798 | 21.893 | 1.00 | 47.83 | C |
| ATOM | 1549 | O | ASN | B | 39 | 23.880 | 30.722 | 22.380 | 1.00 | 48.70 | O |
| ATOM | 1550 | N | SER | B | 40 | 25.332 | 32.453 | 22.361 | 1.00 | 50.06 | N |
| ATOM | 1551 | CA | SER | B | 40 | 26.136 | 31.882 | 23.459 | 1.00 | 49.21 | C |
| ATOM | 1552 | CB | SER | B | 40 | 27.442 | 32.647 | 23.649 | 1.00 | 48.57 | C |
| ATOM | 1553 | OG | SER | B | 40 | 27.218 | 33.898 | 24.280 | 1.00 | 47.90 | O |
| ATOM | 1554 | C | SER | B | 40 | 25.336 | 31.785 | 24.776 | 1.00 | 50.37 | C |
| ATOM | 1555 | O | SER | B | 40 | 25.692 | 30.999 | 25.677 | 1.00 | 51.05 | O |
| ATOM | 1556 | N | SER | B | 41 | 24.249 | 32.554 | 24.875 | 1.00 | 48.80 | N |
| ATOM | 1557 | CA | SER | B | 41 | 23.325 | 32.428 | 26.008 | 1.00 | 52.76 | C |
| ATOM | 1558 | CB | SER | B | 41 | 22.504 | 33.714 | 26.211 | 1.00 | 51.67 | C |
| ATOM | 1559 | OG | SER | B | 41 | 21.575 | 33.924 | 25.161 | 1.00 | 57.98 | O |
| ATOM | 1560 | C | SER | B | 41 | 22.424 | 31.174 | 25.920 | 1.00 | 54.80 | C |
| ATOM | 1561 | O | SER | B | 41 | 21.563 | 30.955 | 26.782 | 1.00 | 55.52 | O |
| ATOM | 1562 | N | ASN | B | 42 | 22.665 | 30.329 | 24.914 | 1.00 | 53.48 | N |
| ATOM | 1563 | CA | ASN | B | 42 | 21.954 | 29.058 | 24.777 | 1.00 | 52.76 | C |
| ATOM | 1564 | CB | ASN | B | 42 | 21.929 | 28.601 | 23.303 | 1.00 | 52.32 | C |
| ATOM | 1565 | CG | ASN | B | 42 | 20.840 | 27.578 | 23.016 | 1.00 | 50.95 | C |
| ATOM | 1566 | OD1 | ASN | B | 42 | 20.252 | 26.984 | 23.931 | 1.00 | 61.15 | O |
| ATOM | 1567 | ND2 | ASN | B | 42 | 20.567 | 27.360 | 21.736 | 1.00 | 51.55 | N |
| ATOM | 1568 | C | ASN | B | 42 | 22.582 | 27.975 | 25.657 | 1.00 | 52.92 | C |
| ATOM | 1569 | O | ASN | B | 42 | 23.527 | 27.296 | 25.236 | 1.00 | 52.05 | O |
| ATOM | 1570 | N | SER | B | 43 | 22.051 | 27.813 | 26.868 | 1.00 | 52.45 | N |
| ATOM | 1571 | CA | SER | B | 43 | 22.571 | 26.817 | 27.819 | 1.00 | 54.85 | C |
| ATOM | 1572 | CB | SER | B | 43 | 22.026 | 27.063 | 29.231 | 1.00 | 53.84 | C |
| ATOM | 1573 | OG | SER | B | 43 | 20.659 | 26.694 | 29.333 | 1.00 | 57.96 | O |
| ATOM | 1574 | C | SER | B | 43 | 22.343 | 25.358 | 27.378 | 1.00 | 54.62 | C |
| ATOM | 1575 | O | SER | B | 43 | 22.959 | 24.447 | 27.926 | 1.00 | 56.78 | O |
| ATOM | 1576 | N | LYS | B | 44 | 21.479 | 25.152 | 26.378 | 1.00 | 53.79 | N |
| ATOM | 1577 | CA | LYS | B | 44 | 21.264 | 23.840 | 25.758 | 1.00 | 51.82 | C |
| ATOM | 1578 | CB | LYS | B | 44 | 20.148 | 23.919 | 24.720 | 1.00 | 52.94 | C |
| ATOM | 1579 | C | LYS | B | 44 | 22.530 | 23.276 | 25.109 | 1.00 | 50.94 | C |
| ATOM | 1580 | O | LYS | B | 44 | 22.605 | 22.071 | 24.860 | 1.00 | 50.22 | O |
| ATOM | 1581 | N | PHE | B | 45 | 23.504 | 24.152 | 24.821 | 1.00 | 49.62 | N |
| ATOM | 1582 | CA | PHE | B | 45 | 24.847 | 23.737 | 24.377 | 1.00 | 48.32 | C |
| ATOM | 1583 | CB | PHE | B | 45 | 25.553 | 24.826 | 23.572 | 1.00 | 47.65 | C |
| ATOM | 1584 | CG | PHE | B | 45 | 24.955 | 25.064 | 22.207 | 1.00 | 45.89 | C |
| ATOM | 1585 | CD1 | PHE | B | 45 | 25.111 | 24.129 | 21.172 | 1.00 | 45.96 | C |
| ATOM | 1586 | CE1 | PHE | B | 45 | 24.572 | 24.365 | 19.904 | 1.00 | 50.10 | C |
| ATOM | 1587 | CZ | PHE | B | 45 | 23.862 | 25.541 | 19.669 | 1.00 | 47.05 | C |
| ATOM | 1588 | CE2 | PHE | B | 45 | 23.710 | 26.476 | 20.686 | 1.00 | 40.41 | C |
| ATOM | 1589 | CD2 | PHE | B | 45 | 24.253 | 26.226 | 21.951 | 1.00 | 47.70 | C |
| ATOM | 1590 | C | PHE | B | 45 | 25.692 | 23.372 | 25.580 | 1.00 | 49.49 | C |
| ATOM | 1591 | O | PHE | B | 45 | 26.274 | 24.243 | 26.245 | 1.00 | 50.60 | O |
| ATOM | 1592 | N | LEU | B | 46 | 25.767 | 22.079 | 25.849 | 1.00 | 50.95 | N |
| ATOM | 1593 | CA | LEU | B | 46 | 26.364 | 21.592 | 27.087 | 1.00 | 51.22 | C |
| ATOM | 1594 | CB | LEU | B | 46 | 25.730 | 20.261 | 27.487 | 1.00 | 51.08 | C |
| ATOM | 1595 | CG | LEU | B | 46 | 24.203 | 20.190 | 27.619 | 1.00 | 46.10 | C |
| ATOM | 1596 | CD1 | LEU | B | 46 | 23.796 | 18.761 | 27.883 | 1.00 | 43.82 | C |
| ATOM | 1597 | CD2 | LEU | B | 46 | 23.662 | 21.111 | 28.694 | 1.00 | 48.08 | C |
| ATOM | 1598 | C | LEU | B | 46 | 27.878 | 21.452 | 26.985 | 1.00 | 54.27 | C |
| ATOM | 1599 | O | LEU | B | 46 | 28.397 | 21.095 | 25.924 | 1.00 | 55.19 | O |
| ATOM | 1600 | N | PRO | B | 47 | 28.592 | 21.756 | 28.088 | 1.00 | 55.99 | N |
| ATOM | 1601 | CA | PRO | B | 47 | 30.002 | 21.439 | 28.289 | 1.00 | 58.57 | C |
| ATOM | 1602 | CB | PRO | B | 47 | 30.085 | 21.210 | 29.807 | 1.00 | 58.59 | C |
| ATOM | 1603 | CG | PRO | B | 47 | 28.908 | 21.978 | 30.394 | 1.00 | 59.61 | C |
| ATOM | 1604 | CD | PRO | B | 47 | 28.053 | 22.487 | 29.245 | 1.00 | 55.29 | C |
| ATOM | 1605 | C | PRO | B | 47 | 30.423 | 20.159 | 27.568 | 1.00 | 60.23 | C |
| ATOM | 1606 | O | PRO | B | 47 | 29.753 | 19.124 | 27.703 | 1.00 | 60.06 | O |
| ATOM | 1607 | N | GLY | B | 48 | 31.512 | 20.242 | 26.801 | 1.00 | 60.39 | N |
| ATOM | 1608 | CA | GLY | B | 48 | 32.012 | 19.118 | 26.020 | 1.00 | 60.52 | C |
| ATOM | 1609 | C | GLY | B | 48 | 31.089 | 18.599 | 24.925 | 1.00 | 62.42 | C |
| ATOM | 1610 | O | GLY | B | 48 | 31.405 | 18.716 | 23.725 | 1.00 | 62.21 | O |
| ATOM | 1611 | N | ALA | B | 49 | 29.954 | 18.032 | 25.348 | 1.00 | 61.80 | N |
| ATOM | 1612 | CA | ALA | B | 49 | 28.988 | 17.361 | 24.474 | 1.00 | 61.62 | C |
| ATOM | 1613 | CB | ALA | B | 49 | 27.858 | 16.757 | 25.302 | 1.00 | 60.60 | C |
| ATOM | 1614 | C | ALA | B | 49 | 28.420 | 18.229 | 23.345 | 1.00 | 62.36 | C |
| ATOM | 1615 | O | ALA | B | 49 | 28.463 | 17.833 | 22.180 | 1.00 | 64.81 | O |
| ATOM | 1616 | N | GLY | B | 50 | 27.894 | 19.404 | 23.690 | 1.00 | 61.93 | N |
| ATOM | 1617 | CA | GLY | B | 50 | 27.206 | 20.263 | 22.722 | 1.00 | 60.75 | C |
| ATOM | 1618 | C | GLY | B | 50 | 25.706 | 20.038 | 22.758 | 1.00 | 59.01 | C |
| ATOM | 1619 | O | GLY | B | 50 | 25.154 | 19.714 | 23.818 | 1.00 | 58.98 | O |
| ATOM | 1620 | N | LEU | B | 51 | 25.051 | 20.190 | 21.607 | 1.00 | 56.06 | N |
| ATOM | 1621 | CA | LEU | B | 51 | 23.600 | 19.988 | 21.513 | 1.00 | 55.72 | C |
| ATOM | 1622 | CB | LEU | B | 51 | 22.923 | 21.197 | 20.861 | 1.00 | 55.78 | C |
| ATOM | 1623 | CG | LEU | B | 51 | 21.510 | 21.607 | 21.317 | 1.00 | 56.57 | C |
| ATOM | 1624 | CD1 | LEU | B | 51 | 21.260 | 23.086 | 21.016 | 1.00 | 54.35 | C |
| ATOM | 1625 | CD2 | LEU | B | 51 | 20.410 | 20.740 | 20.712 | 1.00 | 51.01 | C |
| ATOM | 1626 | C | LEU | B | 51 | 23.231 | 18.687 | 20.783 | 1.00 | 55.37 | C |
| ATOM | 1627 | O | LEU | B | 51 | 23.654 | 18.454 | 19.650 | 1.00 | 56.89 | O |
| ATOM | 1628 | N | VAL | B | 52 | 22.455 | 17.834 | 21.449 | 1.00 | 52.14 | N |
| ATOM | 1629 | CA | VAL | B | 52 | 22.091 | 16.532 | 20.898 | 1.00 | 49.64 | C |
| ATOM | 1630 | CB | VAL | B | 52 | 22.656 | 15.331 | 21.729 | 1.00 | 49.25 | C |
| ATOM | 1631 | CG1 | VAL | B | 52 | 22.280 | 13.976 | 21.106 | 1.00 | 47.08 | C |
| ATOM | 1632 | CG2 | VAL | B | 52 | 24.181 | 15.425 | 21.866 | 1.00 | 51.63 | C |
| ATOM | 1633 | C | VAL | B | 52 | 20.576 | 16.444 | 20.713 | 1.00 | 47.40 | C |
| ATOM | 1634 | O | VAL | B | 52 | 19.796 | 16.668 | 21.658 | 1.00 | 44.14 | O |
| ATOM | 1635 | N | LEU | B | 53 | 20.206 | 16.136 | 19.465 | 1.00 | 46.22 | N |
| ATOM | 1636 | CA | LEU | B | 53 | 18.839 | 16.015 | 18.990 | 1.00 | 46.52 | C |
| ATOM | 1637 | CB | LEU | B | 53 | 18.545 | 17.067 | 17.899 | 1.00 | 45.04 | C |
| ATOM | 1638 | CG | LEU | B | 53 | 18.416 | 18.522 | 18.330 | 1.00 | 49.63 | C |
| ATOM | 1639 | CD1 | LEU | B | 53 | 18.373 | 19.404 | 17.093 | 1.00 | 41.47 | C |
| ATOM | 1640 | CD2 | LEU | B | 53 | 17.178 | 18.711 | 19.223 | 1.00 | 46.52 | C |
| ATOM | 1641 | C | LEU | B | 53 | 18.710 | 14.674 | 18.333 | 1.00 | 46.61 | C |
| ATOM | 1642 | O | LEU | B | 53 | 19.693 | 14.160 | 17.789 | 1.00 | 45.23 | O |
| ATOM | 1643 | N | ALA | B | 54 | 17.488 | 14.136 | 18.377 | 1.00 | 48.27 | N |
| ATOM | 1644 | CA | ALA | B | 54 | 17.104 | 12.922 | 17.662 | 1.00 | 51.02 | C |
| ATOM | 1645 | CB | ALA | B | 54 | 16.871 | 11.748 | 18.636 | 1.00 | 54.27 | C |
| ATOM | 1646 | C | ALA | B | 54 | 15.841 | 13.229 | 16.845 | 1.00 | 52.58 | C |
| ATOM | 1647 | O | ALA | B | 54 | 14.711 | 13.093 | 17.344 | 1.00 | 50.37 | O |
| ATOM | 1648 | N | PRO | B | 55 | 16.035 | 13.722 | 15.606 | 1.00 | 51.74 | N |
| ATOM | 1649 | CA | PRO | B | 55 | 14.941 | 14.004 | 14.667 | 1.00 | 50.37 | C |
| ATOM | 1650 | CB | PRO | B | 55 | 15.546 | 15.065 | 13.753 | 1.00 | 48.25 | C |
| ATOM | 1651 | CG | PRO | B | 55 | 17.011 | 14.775 | 13.743 | 1.00 | 49.23 | C |
| ATOM | 1652 | CD | PRO | B | 55 | 17.352 | 14.098 | 15.044 | 1.00 | 51.76 | C |
| ATOM | 1653 | C | PRO | B | 55 | 14.538 | 12.782 | 13.846 | 1.00 | 49.88 | C |
| ATOM | 1654 | O | PRO | B | 55 | 15.357 | 11.906 | 13.592 | 1.00 | 49.10 | O |
| ATOM | 1655 | N | GLN | B | 56 | 13.281 | 12.748 | 13.422 | 1.00 | 50.05 | N |
| ATOM | 1656 | CA | GLN | B | 56 | 12.789 | 11.680 | 12.590 | 1.00 | 49.58 | C |
| ATOM | 1657 | CB | GLN | B | 56 | 11.439 | 11.207 | 13.119 | 1.00 | 49.76 | C |
| ATOM | 1658 | CG | GLN | B | 56 | 11.343 | 9.697 | 13.288 | 1.00 | 51.96 | C |
| ATOM | 1659 | CD | GLN | B | 56 | 12.022 | 9.182 | 14.553 | 1.00 | 53.57 | C |
| ATOM | 1660 | OE1 | GLN | B | 56 | 12.667 | 9.934 | 15.296 | 1.00 | 48.95 | O |
| ATOM | 1661 | NE2 | GLN | B | 56 | 11.867 | 7.884 | 14.805 | 1.00 | 44.22 | N |
| ATOM | 1662 | C | GLN | B | 56 | 12.647 | 12.193 | 11.165 | 1.00 | 50.12 | C |
| ATOM | 1663 | O | GLN | B | 56 | 12.295 | 13.349 | 10.967 | 1.00 | 49.01 | O |
| ATOM | 1664 | N | ILE | B | 57 | 12.940 | 11.338 | 10.185 | 1.00 | 50.19 | N |
| ATOM | 1665 | CA | ILE | B | 57 | 12.631 | 11.616 | 8.774 | 1.00 | 52.05 | C |
| ATOM | 1666 | CB | ILE | B | 57 | 12.797 | 10.340 | 7.884 | 1.00 | 50.70 | C |
| ATOM | 1667 | CG1 | ILE | B | 57 | 14.270 | 9.879 | 7.814 | 1.00 | 52.97 | C |
| ATOM | 1668 | CD1 | ILE | B | 57 | 15.269 | 10.917 | 7.322 | 1.00 | 50.34 | C |
| ATOM | 1669 | CG2 | ILE | B | 57 | 12.200 | 10.527 | 6.505 | 1.00 | 55.73 | C |
| ATOM | 1670 | C | ILE | B | 57 | 11.211 | 12.192 | 8.661 | 1.00 | 53.21 | C |
| ATOM | 1671 | O | ILE | B | 57 | 10.235 | 11.604 | 9.151 | 1.00 | 52.05 | O |
| ATOM | 1672 | N | GLY | B | 58 | 11.116 | 13.365 | 8.041 | 1.00 | 54.38 | N |
| ATOM | 1673 | CA | GLY | B | 58 | 9.867 | 14.110 | 7.975 | 1.00 | 56.00 | C |
| ATOM | 1674 | C | GLY | B | 58 | 9.901 | 15.371 | 8.822 | 1.00 | 56.89 | C |
| ATOM | 1675 | O | GLY | B | 58 | 9.095 | 16.282 | 8.619 | 1.00 | 57.36 | O |
| ATOM | 1676 | N | ASP | B | 59 | 10.839 | 15.415 | 9.769 | 1.00 | 57.12 | N |
| ATOM | 1677 | CA | ASP | B | 59 | 10.966 | 16.522 | 10.724 | 1.00 | 55.47 | C |
| ATOM | 1678 | CB | ASP | B | 59 | 12.019 | 16.195 | 11.775 | 1.00 | 55.05 | C |
| ATOM | 1679 | C | ASP | B | 59 | 11.341 | 17.828 | 10.060 | 1.00 | 53.03 | C |
| ATOM | 1680 | O | ASP | B | 59 | 11.925 | 17.823 | 8.984 | 1.00 | 53.44 | O |
| ATOM | 1681 | N | LYS | B | 60 | 10.992 | 18.933 | 10.723 | 1.00 | 51.30 | N |
| ATOM | 1682 | CA | LYS | B | 60 | 11.409 | 20.285 | 10.345 | 1.00 | 47.93 | C |
| ATOM | 1683 | CB | LYS | B | 60 | 10.238 | 21.096 | 9.783 | 1.00 | 48.26 | C |
| ATOM | 1684 | CG | LYS | B | 60 | 9.889 | 20.884 | 8.297 | 1.00 | 46.39 | C |
| ATOM | 1685 | CD | LYS | B | 60 | 9.229 | 22.177 | 7.740 | 1.00 | 47.01 | C |
| ATOM | 1686 | CE | LYS | B | 60 | 8.534 | 21.992 | 6.395 | 1.00 | 52.74 | C |
| ATOM | 1687 | NZ | LYS | B | 60 | 7.991 | 23.324 | 5.847 | 1.00 | 44.78 | N |
| ATOM | 1688 | C | LYS | B | 60 | 11.979 | 20.988 | 11.586 | 1.00 | 46.25 | C |
| ATOM | 1689 | O | LYS | B | 60 | 11.384 | 20.949 | 12.655 | 1.00 | 45.18 | O |
| ATOM | 1690 | N | LEU | B | 61 | 13.142 | 21.608 | 11.435 | 1.00 | 45.65 | N |
| ATOM | 1691 | CA | LEU | B | 61 | 13.799 | 22.335 | 12.521 | 1.00 | 44.72 | C |
| ATOM | 1692 | CB | LEU | B | 61 | 14.975 | 21.527 | 13.077 | 1.00 | 45.50 | C |
| ATOM | 1693 | CG | LEU | B | 61 | 15.939 | 22.130 | 14.107 | 1.00 | 47.54 | C |
| ATOM | 1694 | CD1 | LEU | B | 61 | 15.354 | 22.084 | 15.519 | 1.00 | 39.59 | C |
| ATOM | 1695 | CD2 | LEU | B | 61 | 17.286 | 21.432 | 14.072 | 1.00 | 46.47 | C |
| ATOM | 1696 | C | LEU | B | 61 | 14.325 | 23.667 | 12.016 | 1.00 | 42.76 | C |
| ATOM | 1697 | O | LEU | B | 61 | 14.990 | 23.713 | 10.980 | 1.00 | 42.46 | O |
| ATOM | 1698 | N | ASP | B | 62 | 14.022 | 24.732 | 12.754 | 1.00 | 39.20 | N |
| ATOM | 1699 | CA | ASP | B | 62 | 14.588 | 26.045 | 12.507 | 1.00 | 40.51 | C |
| ATOM | 1700 | CB | ASP | B | 62 | 13.553 | 27.146 | 12.773 | 1.00 | 38.33 | C |
| ATOM | 1701 | CG | ASP | B | 62 | 12.304 | 27.020 | 11.914 | 1.00 | 43.05 | C |
| ATOM | 1702 | OD1 | ASP | B | 62 | 11.233 | 27.448 | 12.381 | 1.00 | 53.67 | O |
| ATOM | 1703 | OD2 | ASP | B | 62 | 12.363 | 26.508 | 10.779 | 1.00 | 50.75 | O |
| ATOM | 1704 | C | ASP | B | 62 | 15.791 | 26.280 | 13.419 | 1.00 | 40.29 | C |
| ATOM | 1705 | O | ASP | B | 62 | 15.676 | 26.176 | 14.649 | 1.00 | 44.71 | O |
| ATOM | 1706 | N | ILE | B | 63 | 16.941 | 26.612 | 12.824 | 1.00 | 39.54 | N |
| ATOM | 1707 | CA | ILE | B | 63 | 18.070 | 27.149 | 13.567 | 1.00 | 40.77 | C |
| ATOM | 1708 | CB | ILE | B | 63 | 19.371 | 26.483 | 13.157 | 1.00 | 41.56 | C |
| ATOM | 1709 | CG1 | ILE | B | 63 | 19.243 | 24.966 | 13.300 | 1.00 | 38.50 | C |
| ATOM | 1710 | CD1 | ILE | B | 63 | 20.358 | 24.219 | 12.616 | 1.00 | 50.40 | C |
| ATOM | 1711 | CG2 | ILE | B | 63 | 20.556 | 27.016 | 13.981 | 1.00 | 41.42 | C |
| ATOM | 1712 | C | ILE | B | 63 | 18.112 | 28.688 | 13.321 | 1.00 | 40.07 | C |
| ATOM | 1713 | O | ILE | B | 63 | 18.179 | 29.139 | 12.167 | 1.00 | 42.17 | O |
| ATOM | 1714 | N | ILE | B | 64 | 18.039 | 29.468 | 14.402 | 1.00 | 40.38 | N |
| ATOM | 1715 | CA | ILE | B | 64 | 17.839 | 30.952 | 14.299 | 1.00 | 42.37 | C |
| ATOM | 1716 | CB | ILE | B | 64 | 16.539 | 31.409 | 15.011 | 1.00 | 44.09 | C |
| ATOM | 1717 | CG1 | ILE | B | 64 | 15.325 | 30.676 | 14.458 | 1.00 | 40.39 | C |
| ATOM | 1718 | CD1 | ILE | B | 64 | 15.048 | 29.389 | 15.178 | 1.00 | 47.96 | C |
| ATOM | 1719 | CG2 | ILE | B | 64 | 16.338 | 32.965 | 14.883 | 1.00 | 40.44 | C |
| ATOM | 1720 | C | ILE | B | 64 | 18.945 | 31.742 | 14.953 | 1.00 | 45.33 | C |
| ATOM | 1721 | O | ILE | B | 64 | 19.215 | 31.530 | 16.138 | 1.00 | 44.27 | O |
| ATOM | 1722 | N | CYS | B | 65 | 19.576 | 32.658 | 14.212 | 1.00 | 47.81 | N |
| ATOM | 1723 | CA | CYS | B | 65 | 20.440 | 33.650 | 14.849 | 1.00 | 50.81 | C |
| ATOM | 1724 | CB | CYS | B | 65 | 21.625 | 34.041 | 13.976 | 1.00 | 52.46 | C |
| ATOM | 1725 | SG | CYS | B | 65 | 22.840 | 32.737 | 13.887 | 1.00 | 57.22 | S |
| ATOM | 1726 | C | CYS | B | 65 | 19.604 | 34.870 | 15.232 | 1.00 | 52.50 | C |
| ATOM | 1727 | O | CYS | B | 65 | 19.169 | 35.632 | 14.361 | 1.00 | 51.32 | O |
| ATOM | 1728 | N | PRO | B | 66 | 19.427 | 35.090 | 16.523 | 1.00 | 52.38 | N |
| ATOM | 1729 | CA | PRO | B | 66 | 18.436 | 36.051 | 17.007 | 1.00 | 56.14 | C |
| ATOM | 1730 | CB | PRO | B | 66 | 18.428 | 35.810 | 18.510 | 1.00 | 56.30 | C |
| ATOM | 1731 | CG | PRO | B | 66 | 18.947 | 34.448 | 18.672 | 1.00 | 55.10 | C |
| ATOM | 1732 | CD | PRO | B | 66 | 19.955 | 34.262 | 17.611 | 1.00 | 53.36 | C |
| ATOM | 1733 | C | PRO | B | 66 | 18.780 | 37.503 | 16.701 | 1.00 | 57.91 | C |
| ATOM | 1734 | O | PRO | B | 66 | 19.943 | 37.887 | 16.680 | 1.00 | 55.40 | O |
| ATOM | 1735 | N | LYS | B | 67 | 17.742 | 38.289 | 16.445 | 1.00 | 61.44 | N |
| ATOM | 1736 | CA | LYS | B | 67 | 17.760 | 39.738 | 16.596 | 1.00 | 63.19 | C |
| ATOM | 1737 | CB | LYS | B | 67 | 16.324 | 40.238 | 16.522 | 1.00 | 63.03 | C |
| ATOM | 1738 | CG | LYS | B | 67 | 16.105 | 41.449 | 15.671 | 1.00 | 63.45 | C |
| ATOM | 1739 | CD | LYS | B | 67 | 14.634 | 41.608 | 15.386 | 1.00 | 61.61 | C |
| ATOM | 1740 | CE | LYS | B | 67 | 14.307 | 43.020 | 14.962 | 1.00 | 65.44 | C |
| ATOM | 1741 | NZ | LYS | B | 67 | 13.998 | 43.916 | 16.102 | 1.00 | 65.01 | N |
| ATOM | 1742 | C | LYS | B | 67 | 18.341 | 40.152 | 17.937 | 1.00 | 65.68 | C |
| ATOM | 1743 | O | LYS | B | 67 | 17.926 | 39.645 | 18.972 | 1.00 | 66.30 | O |
| ATOM | 1744 | N | VAL | B | 68 | 19.271 | 41.098 | 17.938 | 1.00 | 66.29 | N |
| ATOM | 1745 | CA | VAL | B | 68 | 19.561 | 41.816 | 19.174 | 1.00 | 67.49 | C |
| ATOM | 1746 | CB | VAL | B | 68 | 20.942 | 41.501 | 19.750 | 1.00 | 66.82 | C |
| ATOM | 1747 | CG1 | VAL | B | 68 | 21.073 | 40.009 | 20.042 | 1.00 | 66.77 | C |
| ATOM | 1748 | CG2 | VAL | B | 68 | 22.021 | 41.977 | 18.826 | 1.00 | 63.95 | C |
| ATOM | 1749 | C | VAL | B | 68 | 19.320 | 43.312 | 19.154 | 1.00 | 68.26 | C |
| ATOM | 1750 | O | VAL | B | 68 | 20.061 | 44.068 | 18.532 | 1.00 | 68.98 | O |
| ATOM | 1751 | N | ASP | B | 69 | 18.285 | 43.722 | 19.874 | 1.00 | 69.20 | N |
| ATOM | 1752 | CA | ASP | B | 69 | 17.631 | 45.029 | 19.687 | 1.00 | 70.92 | C |
| ATOM | 1753 | CB | ASP | B | 69 | 16.119 | 44.881 | 19.934 | 1.00 | 69.99 | C |
| ATOM | 1754 | CG | ASP | B | 69 | 15.638 | 43.433 | 19.795 | 1.00 | 67.08 | C |
| ATOM | 1755 | OD1 | ASP | B | 69 | 15.978 | 42.783 | 18.785 | 1.00 | 72.72 | O |
| ATOM | 1756 | OD2 | ASP | B | 69 | 14.922 | 42.943 | 20.695 | 1.00 | 57.27 | O |
| ATOM | 1757 | C | ASP | B | 69 | 18.207 | 46.160 | 20.561 | 1.00 | 72.50 | C |
| ATOM | 1758 | O | ASP | B | 69 | 19.151 | 45.945 | 21.321 | 1.00 | 72.58 | O |
| ATOM | 1759 | N | SER | B | 70 | 17.640 | 47.363 | 20.434 | 1.00 | 74.73 | N |
| ATOM | 1760 | CA | SER | B | 70 | 18.004 | 48.508 | 21.287 | 1.00 | 77.16 | C |
| ATOM | 1761 | C | SER | B | 70 | 17.679 | 48.211 | 22.755 | 1.00 | 79.24 | C |
| ATOM | 1762 | O | SER | B | 70 | 18.492 | 48.469 | 23.649 | 1.00 | 78.89 | O |
| ATOM | 1763 | N | LYS | B | 71 | 16.472 | 47.687 | 22.980 | 1.00 | 81.34 | N |
| ATOM | 1764 | CA | LYS | B | 71 | 16.098 | 47.035 | 24.233 | 1.00 | 82.67 | C |
| ATOM | 1765 | CB | LYS | B | 71 | 14.669 | 47.409 | 24.636 | 1.00 | 82.35 | C |
| ATOM | 1766 | C | LYS | B | 71 | 16.220 | 45.520 | 24.021 | 1.00 | 84.04 | C |
| ATOM | 1767 | O | LYS | B | 71 | 15.557 | 44.961 | 23.145 | 1.00 | 84.71 | O |
| ATOM | 1768 | N | THR | B | 72 | 17.059 | 44.882 | 24.841 | 1.00 | 84.98 | N |
| ATOM | 1769 | CA | THR | B | 72 | 17.545 | 43.485 | 24.692 | 1.00 | 85.61 | C |
| ATOM | 1770 | CB | THR | B | 72 | 16.758 | 42.603 | 23.646 | 1.00 | 85.57 | C |
| ATOM | 1771 | OG1 | THR | B | 72 | 15.358 | 42.607 | 23.953 | 1.00 | 85.29 | O |
| ATOM | 1772 | CG2 | THR | B | 72 | 17.245 | 41.149 | 23.662 | 1.00 | 85.71 | C |
| ATOM | 1773 | C | THR | B | 72 | 19.066 | 43.520 | 24.425 | 1.00 | 85.75 | C |
| ATOM | 1774 | O | THR | B | 72 | 19.580 | 42.865 | 23.508 | 1.00 | 85.63 | O |
| ATOM | 1775 | N | VAL | B | 73 | 19.754 | 44.316 | 25.251 | 1.00 | 85.63 | N |
| ATOM | 1776 | CA | VAL | B | 73 | 21.222 | 44.480 | 25.269 | 1.00 | 85.19 | C |
| ATOM | 1777 | CB | VAL | B | 73 | 21.959 | 43.217 | 25.841 | 1.00 | 85.28 | C |
| ATOM | 1778 | CG1 | VAL | B | 73 | 23.410 | 43.546 | 26.233 | 1.00 | 83.57 | C |
| ATOM | 1779 | CG2 | VAL | B | 73 | 21.210 | 42.646 | 27.045 | 1.00 | 85.78 | C |
| ATOM | 1780 | C | VAL | B | 73 | 21.832 | 44.916 | 23.925 | 1.00 | 84.59 | C |
| ATOM | 1781 | O | VAL | B | 73 | 21.193 | 44.830 | 22.878 | 1.00 | 83.42 | O |
| ATOM | 1782 | N | GLY | B | 74 | 23.063 | 45.414 | 23.979 | 1.00 | 84.51 | N |
| ATOM | 1783 | CA | GLY | B | 74 | 23.854 | 45.647 | 22.777 | 1.00 | 84.58 | C |
| ATOM | 1784 | C | GLY | B | 74 | 24.647 | 44.398 | 22.419 | 1.00 | 84.25 | C |
| ATOM | 1785 | O | GLY | B | 74 | 24.482 | 43.338 | 23.053 | 1.00 | 85.08 | O |
| ATOM | 1786 | N | GLN | B | 75 | 25.505 | 44.526 | 21.403 | 1.00 | 81.57 | N |
| ATOM | 1787 | CA | GLN | B | 75 | 26.417 | 43.453 | 20.965 | 1.00 | 78.55 | C |
| ATOM | 1788 | CB | GLN | B | 75 | 27.350 | 42.999 | 22.102 | 1.00 | 78.75 | C |
| ATOM | 1789 | CG | GLN | B | 75 | 28.402 | 44.045 | 22.503 | 1.00 | 80.96 | C |
| ATOM | 1790 | CD | GLN | B | 75 | 28.668 | 44.097 | 24.009 | 1.00 | 83.13 | C |
| ATOM | 1791 | OE1 | GLN | B | 75 | 27.757 | 43.914 | 24.820 | 1.00 | 84.28 | O |
| ATOM | 1792 | NE2 | GLN | B | 75 | 29.919 | 44.367 | 24.384 | 1.00 | 81.04 | N |
| ATOM | 1793 | C | GLN | B | 75 | 25.697 | 42.268 | 20.311 | 1.00 | 74.88 | C |
| ATOM | 1794 | O | GLN | B | 75 | 24.789 | 41.663 | 20.884 | 1.00 | 75.60 | O |
| ATOM | 1795 | N | TYR | B | 76 | 26.122 | 41.964 | 19.091 | 1.00 | 70.64 | N |
| ATOM | 1796 | CA | TYR | B | 76 | 25.579 | 40.870 | 18.305 | 1.00 | 65.82 | C |
| ATOM | 1797 | CB | TYR | B | 76 | 25.256 | 41.387 | 16.895 | 1.00 | 62.96 | C |
| ATOM | 1798 | CG | TYR | B | 76 | 24.610 | 40.413 | 15.931 | 1.00 | 60.95 | C |
| ATOM | 1799 | CD1 | TYR | B | 76 | 23.314 | 39.923 | 16.135 | 1.00 | 59.30 | C |
| ATOM | 1800 | CE1 | TYR | B | 76 | 22.729 | 39.027 | 15.221 | 1.00 | 60.32 | C |
| ATOM | 1801 | CZ | TYR | B | 76 | 23.449 | 38.640 | 14.090 | 1.00 | 58.00 | C |
| ATOM | 1802 | OH | TYR | B | 76 | 22.931 | 37.778 | 13.142 | 1.00 | 58.90 | O |
| ATOM | 1803 | CE2 | TYR | B | 76 | 24.721 | 39.138 | 13.879 | 1.00 | 60.57 | C |
| ATOM | 1804 | CD2 | TYR | B | 76 | 25.285 | 40.017 | 14.784 | 1.00 | 54.01 | C |
| ATOM | 1805 | C | TYR | B | 76 | 26.608 | 39.745 | 18.286 | 1.00 | 63.37 | C |
| ATOM | 1806 | O | TYR | B | 76 | 27.806 | 39.995 | 18.413 | 1.00 | 62.55 | O |
| ATOM | 1807 | N | GLU | B | 77 | 26.138 | 38.508 | 18.149 | 1.00 | 59.87 | N |
| ATOM | 1808 | CA | GLU | B | 77 | 27.034 | 37.352 | 18.091 | 1.00 | 57.95 | C |
| ATOM | 1809 | CB | GLU | B | 77 | 26.501 | 36.220 | 18.980 | 1.00 | 58.47 | C |
| ATOM | 1810 | C | GLU | B | 77 | 27.268 | 36.895 | 16.648 | 1.00 | 54.89 | C |
| ATOM | 1811 | O | GLU | B | 77 | 26.396 | 36.283 | 16.024 | 1.00 | 53.41 | O |
| ATOM | 1812 | N | TYR | B | 78 | 28.445 | 37.217 | 16.117 | 1.00 | 52.94 | N |
| ATOM | 1813 | CA | TYR | B | 78 | 28.764 | 36.914 | 14.721 | 1.00 | 52.55 | C |
| ATOM | 1814 | CB | TYR | B | 78 | 29.841 | 37.877 | 14.187 | 1.00 | 53.56 | C |
| ATOM | 1815 | CG | TYR | B | 78 | 29.332 | 39.285 | 13.930 | 1.00 | 49.81 | C |
| ATOM | 1816 | CD1 | TYR | B | 78 | 29.291 | 40.237 | 14.950 | 1.00 | 55.06 | C |
| ATOM | 1817 | CE1 | TYR | B | 78 | 28.813 | 41.535 | 14.712 | 1.00 | 51.22 | C |
| ATOM | 1818 | CZ | TYR | B | 78 | 28.382 | 41.875 | 13.446 | 1.00 | 48.74 | C |
| ATOM | 1819 | OH | TYR | B | 78 | 27.919 | 43.147 | 13.187 | 1.00 | 53.04 | O |
| ATOM | 1820 | CE2 | TYR | B | 78 | 28.427 | 40.953 | 12.419 | 1.00 | 46.58 | C |
| ATOM | 1821 | CD2 | TYR | B | 78 | 28.904 | 39.670 | 12.660 | 1.00 | 51.58 | C |
| ATOM | 1822 | C | TYR | B | 78 | 29.184 | 35.451 | 14.537 | 1.00 | 55.29 | C |
| ATOM | 1823 | O | TYR | B | 78 | 30.229 | 35.031 | 15.035 | 1.00 | 55.26 | O |
| ATOM | 1824 | N | TYR | B | 79 | 28.352 | 34.674 | 13.835 | 1.00 | 54.89 | N |
| ATOM | 1825 | CA | TYR | B | 79 | 28.705 | 33.305 | 13.441 | 1.00 | 52.92 | C |
| ATOM | 1826 | CB | TYR | B | 79 | 27.939 | 32.276 | 14.265 | 1.00 | 53.97 | C |
| ATOM | 1827 | CG | TYR | B | 79 | 27.973 | 32.471 | 15.754 | 1.00 | 55.14 | C |
| ATOM | 1828 | CD1 | TYR | B | 79 | 26.875 | 33.015 | 16.422 | 1.00 | 55.96 | C |
| ATOM | 1829 | CE1 | TYR | B | 79 | 26.891 | 33.184 | 17.792 | 1.00 | 58.12 | C |
| ATOM | 1830 | CZ | TYR | B | 79 | 28.014 | 32.806 | 18.514 | 1.00 | 55.57 | C |
| ATOM | 1831 | OH | TYR | B | 79 | 28.030 | 32.989 | 19.863 | 1.00 | 57.86 | O |
| ATOM | 1832 | CE2 | TYR | B | 79 | 29.121 | 32.269 | 17.875 | 1.00 | 56.26 | C |
| ATOM | 1833 | CD2 | TYR | B | 79 | 29.091 | 32.099 | 16.501 | 1.00 | 53.27 | C |
| ATOM | 1834 | C | TYR | B | 79 | 28.420 | 32.995 | 11.981 | 1.00 | 54.07 | C |
| ATOM | 1835 | O | TYR | B | 79 | 27.498 | 33.556 | 11.392 | 1.00 | 52.83 | O |
| ATOM | 1836 | N | LYS | B | 80 | 29.220 | 32.085 | 11.415 | 1.00 | 53.13 | N |
| ATOM | 1837 | CA | LYS | B | 80 | 28.866 | 31.360 | 10.191 | 1.00 | 52.07 | C |
| ATOM | 1838 | CB | LYS | B | 80 | 29.990 | 31.406 | 9.149 | 1.00 | 51.71 | C |
| ATOM | 1839 | CG | LYS | B | 80 | 30.016 | 32.654 | 8.280 | 1.00 | 56.46 | C |
| ATOM | 1840 | CD | LYS | B | 80 | 31.021 | 32.499 | 7.152 | 1.00 | 55.08 | C |
| ATOM | 1841 | CE | LYS | B | 80 | 30.814 | 33.552 | 6.082 | 1.00 | 59.80 | C |
| ATOM | 1842 | NZ | LYS | B | 80 | 30.966 | 34.942 | 6.603 | 1.00 | 62.51 | N |
| ATOM | 1843 | C | LYS | B | 80 | 28.605 | 29.907 | 10.582 | 1.00 | 51.02 | C |
| ATOM | 1844 | O | LYS | B | 80 | 29.467 | 29.247 | 11.169 | 1.00 | 49.01 | O |
| ATOM | 1845 | N | VAL | B | 81 | 27.425 | 29.402 | 10.255 | 1.00 | 48.00 | N |
| ATOM | 1846 | CA | VAL | B | 81 | 27.048 | 28.066 | 10.703 | 1.00 | 48.41 | C |
| ATOM | 1847 | CB | VAL | B | 81 | 25.668 | 28.073 | 11.413 | 1.00 | 45.24 | C |
| ATOM | 1848 | CG1 | VAL | B | 81 | 25.360 | 26.732 | 12.037 | 1.00 | 49.15 | C |
| ATOM | 1849 | CG2 | VAL | B | 81 | 25.649 | 29.118 | 12.482 | 1.00 | 46.44 | C |
| ATOM | 1850 | C | VAL | B | 81 | 27.101 | 27.130 | 9.509 | 1.00 | 50.06 | C |
| ATOM | 1851 | O | VAL | B | 81 | 26.679 | 27.501 | 8.411 | 1.00 | 50.73 | O |
| ATOM | 1852 | N | TYR | B | 82 | 27.620 | 25.923 | 9.722 | 1.00 | 50.39 | N |
| ATOM | 1853 | CA | TYR | B | 82 | 27.909 | 24.998 | 8.622 | 1.00 | 51.80 | C |
| ATOM | 1854 | CB | TYR | B | 82 | 29.422 | 24.860 | 8.414 | 1.00 | 50.05 | C |
| ATOM | 1855 | CG | TYR | B | 82 | 30.088 | 26.033 | 7.741 | 1.00 | 55.29 | C |
| ATOM | 1856 | CD1 | TYR | B | 82 | 30.609 | 27.094 | 8.492 | 1.00 | 60.37 | C |
| ATOM | 1857 | CE1 | TYR | B | 82 | 31.237 | 28.183 | 7.871 | 1.00 | 59.32 | C |
| ATOM | 1858 | CZ | TYR | B | 82 | 31.357 | 28.209 | 6.486 | 1.00 | 60.82 | C |
| ATOM | 1859 | OH | TYR | B | 82 | 31.973 | 29.283 | 5.860 | 1.00 | 56.78 | O |
| ATOM | 1860 | CE2 | TYR | B | 82 | 30.862 | 27.153 | 5.726 | 1.00 | 60.90 | C |
| ATOM | 1861 | CD2 | TYR | B | 82 | 30.221 | 26.079 | 6.361 | 1.00 | 56.29 | C |
| ATOM | 1862 | C | TYR | B | 82 | 27.352 | 23.617 | 8.889 | 1.00 | 51.81 | C |
| ATOM | 1863 | O | TYR | B | 82 | 27.264 | 23.196 | 10.039 | 1.00 | 52.65 | O |
| ATOM | 1864 | N | MET | B | 83 | 26.999 | 22.916 | 7.815 | 1.00 | 52.67 | N |
| ATOM | 1865 | CA | MET | B | 83 | 26.743 | 21.484 | 7.884 | 1.00 | 54.74 | C |
| ATOM | 1866 | CB | MET | B | 83 | 25.601 | 21.099 | 6.941 | 1.00 | 55.19 | C |
| ATOM | 1867 | CG | MET | B | 83 | 25.406 | 19.614 | 6.751 | 1.00 | 55.78 | C |
| ATOM | 1868 | SD | MET | B | 83 | 24.629 | 18.846 | 8.168 | 1.00 | 62.13 | S |
| ATOM | 1869 | CE | MET | B | 83 | 22.912 | 18.982 | 7.717 | 1.00 | 53.46 | C |
| ATOM | 1870 | C | MET | B | 83 | 28.047 | 20.750 | 7.522 | 1.00 | 55.00 | C |
| ATOM | 1871 | O | MET | B | 83 | 28.531 | 20.846 | 6.387 | 1.00 | 53.90 | O |
| ATOM | 1872 | N | VAL | B | 84 | 28.621 | 20.054 | 8.505 | 1.00 | 55.21 | N |
| ATOM | 1873 | CA | VAL | B | 84 | 29.917 | 19.375 | 8.347 | 1.00 | 52.74 | C |
| ATOM | 1874 | CB | VAL | B | 84 | 30.977 | 19.860 | 9.389 | 1.00 | 50.47 | C |
| ATOM | 1875 | CG1 | VAL | B | 84 | 31.411 | 21.268 | 9.096 | 1.00 | 46.44 | C |
| ATOM | 1876 | CG2 | VAL | B | 84 | 30.466 | 19.738 | 10.816 | 1.00 | 49.60 | C |
| ATOM | 1877 | C | VAL | B | 84 | 29.801 | 17.856 | 8.434 | 1.00 | 55.22 | C |
| ATOM | 1878 | O | VAL | B | 84 | 28.712 | 17.308 | 8.613 | 1.00 | 55.27 | O |
| ATOM | 1879 | N | ASP | B | 85 | 30.946 | 17.194 | 8.289 | 1.00 | 57.58 | N |
| ATOM | 1880 | CA | ASP | B | 85 | 31.090 | 15.759 | 8.523 | 1.00 | 59.75 | C |
| ATOM | 1881 | CB | ASP | B | 85 | 32.035 | 15.146 | 7.471 | 1.00 | 61.05 | C |
| ATOM | 1882 | CG | ASP | B | 85 | 33.409 | 15.825 | 7.436 | 1.00 | 65.18 | C |
| ATOM | 1883 | OD1 | ASP | B | 85 | 33.598 | 16.760 | 6.623 | 1.00 | 67.19 | O |
| ATOM | 1884 | OD2 | ASP | B | 85 | 34.298 | 15.421 | 8.224 | 1.00 | 68.81 | O |
| ATOM | 1885 | C | ASP | B | 85 | 31.603 | 15.485 | 9.949 | 1.00 | 57.67 | C |
| ATOM | 1886 | O | ASP | B | 85 | 32.138 | 16.374 | 10.606 | 1.00 | 59.66 | O |
| ATOM | 1887 | N | LYS | B | 86 | 31.433 | 14.256 | 10.423 | 1.00 | 57.11 | N |
| ATOM | 1888 | CA | LYS | B | 86 | 31.936 | 13.859 | 11.738 | 1.00 | 54.53 | C |
| ATOM | 1889 | CB | LYS | B | 86 | 31.798 | 12.349 | 11.916 | 1.00 | 52.85 | C |
| ATOM | 1890 | CG | LYS | B | 86 | 32.434 | 11.817 | 13.179 | 1.00 | 44.47 | C |
| ATOM | 1891 | CD | LYS | B | 86 | 32.959 | 10.389 | 12.950 | 1.00 | 41.89 | C |
| ATOM | 1892 | CE | LYS | B | 86 | 32.765 | 9.541 | 14.174 | 1.00 | 38.48 | C |
| ATOM | 1893 | NZ | LYS | B | 86 | 33.495 | 10.036 | 15.379 | 1.00 | 47.23 | N |
| ATOM | 1894 | C | LYS | B | 86 | 33.389 | 14.294 | 12.000 | 1.00 | 55.95 | C |
| ATOM | 1895 | O | LYS | B | 86 | 33.713 | 14.762 | 13.093 | 1.00 | 55.26 | O |
| ATOM | 1896 | N | ASP | B | 87 | 34.253 | 14.147 | 10.999 | 1.00 | 57.71 | N |
| ATOM | 1897 | CA | ASP | B | 87 | 35.673 | 14.480 | 11.149 | 1.00 | 60.04 | C |
| ATOM | 1898 | CB | ASP | B | 87 | 36.528 | 13.745 | 10.107 | 1.00 | 59.81 | C |
| ATOM | 1899 | CG | ASP | B | 87 | 37.035 | 12.406 | 10.606 | 1.00 | 64.01 | C |
| ATOM | 1900 | OD1 | ASP | B | 87 | 37.633 | 12.362 | 11.709 | 1.00 | 62.33 | O |
| ATOM | 1901 | OD2 | ASP | B | 87 | 36.847 | 11.399 | 9.882 | 1.00 | 62.56 | O |
| ATOM | 1902 | C | ASP | B | 87 | 35.941 | 15.980 | 11.078 | 1.00 | 60.97 | C |
| ATOM | 1903 | O | ASP | B | 87 | 36.953 | 16.449 | 11.581 | 1.00 | 61.10 | O |
| ATOM | 1904 | N | GLN | B | 88 | 35.057 | 16.738 | 10.446 | 1.00 | 61.32 | N |
| ATOM | 1905 | CA | GLN | B | 88 | 35.265 | 18.178 | 10.363 | 1.00 | 63.39 | C |
| ATOM | 1906 | CB | GLN | B | 88 | 34.473 | 18.784 | 9.208 | 1.00 | 63.94 | C |
| ATOM | 1907 | C | GLN | B | 88 | 34.837 | 18.810 | 11.667 | 1.00 | 64.84 | C |
| ATOM | 1908 | O | GLN | B | 88 | 35.222 | 19.929 | 11.983 | 1.00 | 65.42 | O |
| ATOM | 1909 | N | ALA | B | 89 | 34.040 | 18.061 | 12.417 | 1.00 | 65.49 | N |
| ATOM | 1910 | CA | ALA | B | 89 | 33.424 | 18.533 | 13.644 | 1.00 | 65.15 | C |
| ATOM | 1911 | CB | ALA | B | 89 | 31.989 | 18.034 | 13.731 | 1.00 | 65.66 | C |
| ATOM | 1912 | C | ALA | B | 89 | 34.212 | 18.022 | 14.824 | 1.00 | 64.18 | C |
| ATOM | 1913 | O | ALA | B | 89 | 34.544 | 18.765 | 15.735 | 1.00 | 63.09 | O |
| ATOM | 1914 | O | ASP | B | 90 | 36.855 | 17.048 | 17.406 | 1.00 | 20.00 | O |
| ATOM | 1915 | N | ASP | B | 90 | 34.503 | 16.731 | 14.795 | 1.00 | 20.00 | N |
| ATOM | 1916 | CA | ASP | B | 90 | 35.305 | 16.114 | 15.829 | 1.00 | 20.00 | C |
| ATOM | 1917 | C | ASP | B | 90 | 36.371 | 17.098 | 16.280 | 1.00 | 20.00 | C |
| ATOM | 1918 | CB | ASP | B | 90 | 35.928 | 14.827 | 15.303 | 1.00 | 20.00 | C |
| ATOM | 1919 | CG | ASP | B | 90 | 35.238 | 13.602 | 15.839 | 1.00 | 20.00 | C |
| ATOM | 1920 | OD1 | ASP | B | 90 | 34.100 | 13.741 | 16.329 | 1.00 | 20.00 | O |
| ATOM | 1921 | OD2 | ASP | B | 90 | 35.838 | 12.510 | 15.788 | 1.00 | 20.00 | O |
| ATOM | 1922 | O | ARG | B | 91 | 38.909 | 20.743 | 14.930 | 1.00 | 20.00 | O |
| ATOM | 1923 | N | ARG | B | 91 | 36.693 | 18.021 | 15.390 | 1.00 | 20.00 | N |
| ATOM | 1924 | CA | ARG | B | 91 | 38.029 | 18.543 | 15.278 | 1.00 | 20.00 | C |
| ATOM | 1925 | C | ARG | B | 91 | 37.934 | 19.999 | 14.865 | 1.00 | 20.00 | C |
| ATOM | 1926 | CB | ARG | B | 91 | 38.794 | 17.757 | 14.225 | 1.00 | 20.00 | C |
| ATOM | 1927 | N | CYS | B | 92 | 36.744 | 20.402 | 14.436 | 1.00 | 65.62 | N |
| ATOM | 1928 | CA | CYS | B | 92 | 36.377 | 21.803 | 14.449 | 1.00 | 63.54 | C |
| ATOM | 1929 | CB | CYS | B | 92 | 36.658 | 22.414 | 15.813 | 1.00 | 62.30 | C |
| ATOM | 1930 | SG | CYS | B | 92 | 35.415 | 22.029 | 17.030 | 1.00 | 61.91 | S |
| ATOM | 1931 | C | CYS | B | 92 | 37.103 | 22.560 | 13.364 | 1.00 | 63.64 | C |
| ATOM | 1932 | O | CYS | B | 92 | 37.751 | 23.557 | 13.622 | 1.00 | 62.94 | O |
| ATOM | 1933 | N | THR | B | 93 | 36.973 | 22.076 | 12.139 | 1.00 | 66.00 | N |
| ATOM | 1934 | CA | THR | B | 93 | 37.644 | 22.682 | 10.998 | 1.00 | 69.32 | C |
| ATOM | 1935 | CB | THR | B | 93 | 38.992 | 21.965 | 10.653 | 1.00 | 69.46 | C |
| ATOM | 1936 | OG1 | THR | B | 93 | 39.501 | 22.457 | 9.406 | 1.00 | 71.08 | O |
| ATOM | 1937 | CG2 | THR | B | 93 | 38.813 | 20.447 | 10.563 | 1.00 | 67.96 | C |
| ATOM | 1938 | C | THR | B | 93 | 36.711 | 22.682 | 9.798 | 1.00 | 70.96 | C |
| ATOM | 1939 | O | THR | B | 93 | 36.174 | 21.638 | 9.422 | 1.00 | 72.14 | O |
| ATOM | 1940 | N | ILE | B | 94 | 36.500 | 23.868 | 9.231 | 1.00 | 72.37 | N |
| ATOM | 1941 | CA | ILE | B | 94 | 35.749 | 24.019 | 7.985 | 1.00 | 73.47 | C |
| ATOM | 1942 | CB | ILE | B | 94 | 34.519 | 24.973 | 8.129 | 1.00 | 72.37 | C |
| ATOM | 1943 | CG1 | ILE | B | 94 | 34.943 | 26.416 | 8.425 | 1.00 | 71.50 | C |
| ATOM | 1944 | CD1 | ILE | B | 94 | 34.846 | 27.339 | 7.244 | 1.00 | 65.08 | C |
| ATOM | 1945 | CG2 | ILE | B | 94 | 33.571 | 24.464 | 9.211 | 1.00 | 72.82 | C |
| ATOM | 1946 | C | ILE | B | 94 | 36.699 | 24.430 | 6.851 | 1.00 | 74.87 | C |
| ATOM | 1947 | O | ILE | B | 94 | 36.281 | 24.598 | 5.701 | 1.00 | 74.36 | O |
| ATOM | 1948 | N | LYS | B | 95 | 37.979 | 24.587 | 7.201 | 1.00 | 76.69 | N |
| ATOM | 1949 | CA | LYS | B | 95 | 39.050 | 24.828 | 6.236 | 1.00 | 78.11 | C |
| ATOM | 1950 | CB | LYS | B | 95 | 40.345 | 25.209 | 6.955 | 1.00 | 77.98 | C |
| ATOM | 1951 | C | LYS | B | 95 | 39.231 | 23.555 | 5.414 | 1.00 | 79.85 | C |
| ATOM | 1952 | O | LYS | B | 95 | 40.318 | 22.964 | 5.359 | 1.00 | 80.52 | O |
| ATOM | 1953 | N | LYS | B | 96 | 38.132 | 23.164 | 4.770 | 1.00 | 80.67 | N |
| ATOM | 1954 | CA | LYS | B | 96 | 37.946 | 21.878 | 4.109 | 1.00 | 80.88 | C |
| ATOM | 1955 | CB | LYS | B | 96 | 38.263 | 20.732 | 5.106 | 1.00 | 80.35 | C |
| ATOM | 1956 | CG | LYS | B | 96 | 37.430 | 19.457 | 5.056 | 1.00 | 79.50 | C |
| ATOM | 1957 | CD | LYS | B | 96 | 36.505 | 19.334 | 6.265 | 1.00 | 77.17 | C |
| ATOM | 1958 | CE | LYS | B | 96 | 35.924 | 20.675 | 6.665 | 1.00 | 77.09 | C |
| ATOM | 1959 | NZ | LYS | B | 96 | 34.524 | 20.596 | 7.133 | 1.00 | 76.80 | N |
| ATOM | 1960 | C | LYS | B | 96 | 36.494 | 21.937 | 3.589 | 1.00 | 81.93 | C |
| ATOM | 1961 | O | LYS | B | 96 | 35.881 | 23.011 | 3.640 | 1.00 | 82.40 | O |
| ATOM | 1962 | N | GLU | B | 97 | 35.953 | 20.832 | 3.071 | 1.00 | 82.44 | N |
| ATOM | 1963 | CA | GLU | B | 97 | 34.574 | 20.804 | 2.541 | 1.00 | 82.95 | C |
| ATOM | 1964 | CB | GLU | B | 97 | 34.245 | 19.415 | 1.978 | 1.00 | 82.71 | C |
| ATOM | 1965 | C | GLU | B | 97 | 33.513 | 21.250 | 3.575 | 1.00 | 82.95 | C |
| ATOM | 1966 | O | GLU | B | 97 | 33.406 | 20.664 | 4.663 | 1.00 | 83.36 | O |
| ATOM | 1967 | N | ASN | B | 98 | 32.746 | 22.291 | 3.233 | 1.00 | 81.59 | N |
| ATOM | 1968 | CA | ASN | B | 98 | 31.794 | 22.911 | 4.171 | 1.00 | 79.66 | C |
| ATOM | 1969 | CB | ASN | B | 98 | 32.549 | 23.709 | 5.239 | 1.00 | 80.02 | C |
| ATOM | 1970 | C | ASN | B | 98 | 30.731 | 23.801 | 3.511 | 1.00 | 78.31 | C |
| ATOM | 1971 | O | ASN | B | 98 | 31.048 | 24.644 | 2.660 | 1.00 | 77.79 | O |
| ATOM | 1972 | N | THR | B | 99 | 29.478 | 23.620 | 3.932 | 1.00 | 76.30 | N |
| ATOM | 1973 | CA | THR | B | 99 | 28.321 | 24.312 | 3.327 | 1.00 | 74.52 | C |
| ATOM | 1974 | CB | THR | B | 99 | 27.334 | 23.301 | 2.663 | 1.00 | 74.67 | C |
| ATOM | 1975 | OG1 | THR | B | 99 | 27.823 | 21.957 | 2.811 | 1.00 | 73.98 | O |
| ATOM | 1976 | CG2 | THR | B | 99 | 27.139 | 23.624 | 1.182 | 1.00 | 73.78 | C |
| ATOM | 1977 | C | THR | B | 99 | 27.570 | 25.185 | 4.363 | 1.00 | 72.17 | C |
| ATOM | 1978 | O | THR | B | 99 | 26.920 | 24.645 | 5.274 | 1.00 | 72.60 | O |
| ATOM | 1979 | N | PRO | B | 100 | 27.641 | 26.530 | 4.214 | 1.00 | 68.26 | N |
| ATOM | 1980 | CA | PRO | B | 100 | 27.251 | 27.464 | 5.286 | 1.00 | 65.84 | C |
| ATOM | 1981 | CB | PRO | B | 100 | 28.052 | 28.743 | 4.960 | 1.00 | 65.48 | C |
| ATOM | 1982 | CG | PRO | B | 100 | 28.612 | 28.545 | 3.556 | 1.00 | 66.33 | C |
| ATOM | 1983 | CD | PRO | B | 100 | 28.070 | 27.252 | 3.005 | 1.00 | 68.07 | C |
| ATOM | 1984 | C | PRO | B | 100 | 25.752 | 27.753 | 5.352 | 1.00 | 62.59 | C |
| ATOM | 1985 | O | PRO | B | 100 | 25.240 | 28.596 | 4.605 | 1.00 | 63.79 | O |
| ATOM | 1986 | N | LEU | B | 101 | 25.071 | 27.056 | 6.259 | 1.00 | 59.41 | N |
| ATOM | 1987 | CA | LEU | B | 101 | 23.617 | 27.127 | 6.403 | 1.00 | 56.35 | C |
| ATOM | 1988 | CB | LEU | B | 101 | 23.105 | 25.985 | 7.290 | 1.00 | 56.25 | C |
| ATOM | 1989 | CG | LEU | B | 101 | 23.449 | 24.556 | 6.871 | 1.00 | 53.36 | C |
| ATOM | 1990 | CD1 | LEU | B | 101 | 22.629 | 23.566 | 7.659 | 1.00 | 57.33 | C |
| ATOM | 1991 | CD2 | LEU | B | 101 | 23.255 | 24.344 | 5.374 | 1.00 | 48.21 | C |
| ATOM | 1992 | C | LEU | B | 101 | 23.126 | 28.455 | 6.951 | 1.00 | 56.12 | C |
| ATOM | 1993 | O | LEU | B | 101 | 22.025 | 28.905 | 6.620 | 1.00 | 55.96 | O |
| ATOM | 1994 | N | LEU | B | 102 | 23.923 | 29.074 | 7.812 | 1.00 | 54.26 | N |
| ATOM | 1995 | CA | LEU | B | 102 | 23.641 | 30.435 | 8.253 | 1.00 | 52.03 | C |
| ATOM | 1996 | CB | LEU | B | 102 | 23.173 | 30.459 | 9.708 | 1.00 | 52.13 | C |
| ATOM | 1997 | CG | LEU | B | 102 | 21.954 | 29.702 | 10.235 | 1.00 | 50.45 | C |
| ATOM | 1998 | CD1 | LEU | B | 102 | 22.041 | 29.695 | 11.719 | 1.00 | 55.04 | C |
| ATOM | 1999 | CD2 | LEU | B | 102 | 20.692 | 30.392 | 9.827 | 1.00 | 60.55 | C |
| ATOM | 2000 | C | LEU | B | 102 | 24.870 | 31.323 | 8.140 | 1.00 | 52.59 | C |
| ATOM | 2001 | O | LEU | B | 102 | 25.984 | 30.933 | 8.523 | 1.00 | 51.55 | O |
| ATOM | 2002 | N | ASN | B | 103 | 24.655 | 32.521 | 7.621 | 1.00 | 53.64 | N |
| ATOM | 2003 | CA | ASN | B | 103 | 25.600 | 33.615 | 7.793 | 1.00 | 54.00 | C |
| ATOM | 2004 | CB | ASN | B | 103 | 25.998 | 34.252 | 6.467 | 1.00 | 54.78 | C |
| ATOM | 2005 | CG | ASN | B | 103 | 26.931 | 35.436 | 6.665 | 1.00 | 58.33 | C |
| ATOM | 2006 | OD1 | ASN | B | 103 | 28.084 | 35.271 | 7.084 | 1.00 | 58.48 | O |
| ATOM | 2007 | ND2 | ASN | B | 103 | 26.424 | 36.643 | 6.399 | 1.00 | 45.80 | N |
| ATOM | 2008 | C | ASN | B | 103 | 25.015 | 34.670 | 8.720 | 1.00 | 53.18 | C |
| ATOM | 2009 | O | ASN | B | 103 | 24.442 | 35.657 | 8.267 | 1.00 | 54.82 | O |
| ATOM | 2010 | N | CYS | B | 104 | 25.172 | 34.463 | 10.023 | 1.00 | 52.46 | N |
| ATOM | 2011 | CA | CYS | B | 104 | 24.649 | 35.394 | 11.007 | 1.00 | 51.44 | C |
| ATOM | 2012 | CB | CYS | B | 104 | 24.466 | 34.700 | 12.341 | 1.00 | 53.41 | C |
| ATOM | 2013 | SG | CYS | B | 104 | 23.832 | 33.036 | 12.178 | 1.00 | 49.61 | S |
| ATOM | 2014 | C | CYS | B | 104 | 25.604 | 36.556 | 11.139 | 1.00 | 53.20 | C |
| ATOM | 2015 | O | CYS | B | 104 | 26.565 | 36.513 | 11.915 | 1.00 | 51.74 | O |
| ATOM | 2016 | N | ALA | B | 105 | 25.335 | 37.578 | 10.338 | 1.00 | 52.80 | N |
| ATOM | 2017 | CA | ALA | B | 105 | 26.121 | 38.783 | 10.298 | 1.00 | 52.86 | C |
| ATOM | 2018 | CB | ALA | B | 105 | 27.202 | 38.669 | 9.219 | 1.00 | 53.20 | C |
| ATOM | 2019 | C | ALA | B | 105 | 25.188 | 39.945 | 10.006 | 1.00 | 53.19 | C |
| ATOM | 2020 | O | ALA | B | 105 | 25.501 | 40.812 | 9.184 | 1.00 | 54.31 | O |
| ATOM | 2021 | N | ARG | B | 106 | 24.030 | 39.929 | 10.668 | 1.00 | 54.23 | N |
| ATOM | 2022 | CA | ARG | B | 106 | 23.018 | 40.988 | 10.559 | 1.00 | 53.54 | C |
| ATOM | 2023 | CB | ARG | B | 106 | 21.984 | 40.637 | 9.480 | 1.00 | 53.56 | C |
| ATOM | 2024 | C | ARG | B | 106 | 22.319 | 41.211 | 11.900 | 1.00 | 53.50 | C |
| ATOM | 2025 | O | ARG | B | 106 | 21.322 | 40.545 | 12.206 | 1.00 | 53.50 | O |
| ATOM | 2026 | N | PRO | B | 107 | 22.847 | 42.136 | 12.715 | 1.00 | 53.15 | N |
| ATOM | 2027 | CA | PRO | B | 107 | 22.255 | 42.466 | 14.024 | 1.00 | 54.11 | C |
| ATOM | 2028 | CB | PRO | B | 107 | 23.089 | 43.666 | 14.488 | 1.00 | 55.59 | C |
| ATOM | 2029 | CG | PRO | B | 107 | 24.428 | 43.483 | 13.797 | 1.00 | 51.19 | C |
| ATOM | 2030 | CD | PRO | B | 107 | 24.091 | 42.894 | 12.459 | 1.00 | 52.06 | C |
| ATOM | 2031 | C | PRO | B | 107 | 20.745 | 42.804 | 14.041 | 1.00 | 56.24 | C |
| ATOM | 2032 | O | PRO | B | 107 | 20.048 | 42.480 | 15.012 | 1.00 | 57.46 | O |
| ATOM | 2033 | N | ASP | B | 108 | 20.245 | 43.423 | 12.978 | 1.00 | 56.84 | N |
| ATOM | 2034 | CA | ASP | B | 108 | 18.905 | 44.009 | 13.004 | 1.00 | 56.83 | C |
| ATOM | 2035 | CB | ASP | B | 108 | 18.933 | 45.389 | 12.354 | 1.00 | 58.06 | C |
| ATOM | 2036 | CG | ASP | B | 108 | 17.754 | 46.242 | 12.761 | 1.00 | 60.68 | C |
| ATOM | 2037 | OD1 | ASP | B | 108 | 17.196 | 46.004 | 13.857 | 1.00 | 64.10 | O |
| ATOM | 2038 | OD2 | ASP | B | 108 | 17.386 | 47.154 | 11.988 | 1.00 | 66.86 | O |
| ATOM | 2039 | C | ASP | B | 108 | 17.823 | 43.138 | 12.362 | 1.00 | 56.19 | C |
| ATOM | 2040 | O | ASP | B | 108 | 16.689 | 43.573 | 12.151 | 1.00 | 53.32 | O |
| ATOM | 2041 | N | GLN | B | 109 | 18.170 | 41.887 | 12.085 | 1.00 | 56.39 | N |
| ATOM | 2042 | CA | GLN | B | 109 | 17.298 | 41.022 | 11.324 | 1.00 | 55.55 | C |
| ATOM | 2043 | CB | GLN | B | 109 | 17.632 | 41.124 | 9.827 | 1.00 | 55.41 | C |
| ATOM | 2044 | CG | GLN | B | 109 | 17.483 | 39.837 | 9.023 | 1.00 | 58.46 | C |
| ATOM | 2045 | CD | GLN | B | 109 | 17.142 | 40.063 | 7.561 | 1.00 | 61.57 | C |
| ATOM | 2046 | OE1 | GLN | B | 109 | 17.045 | 41.204 | 7.089 | 1.00 | 66.19 | O |
| ATOM | 2047 | NE2 | GLN | B | 109 | 16.949 | 38.971 | 6.831 | 1.00 | 64.69 | N |
| ATOM | 2048 | C | GLN | B | 109 | 17.368 | 39.593 | 11.852 | 1.00 | 57.37 | C |
| ATOM | 2049 | O | GLN | B | 109 | 18.452 | 39.029 | 12.060 | 1.00 | 57.65 | O |
| ATOM | 2050 | N | ASP | B | 110 | 16.192 | 39.038 | 12.100 | 1.00 | 56.55 | N |
| ATOM | 2051 | CA | ASP | B | 110 | 16.039 | 37.669 | 12.534 | 1.00 | 57.00 | C |
| ATOM | 2052 | CB | ASP | B | 110 | 14.552 | 37.452 | 12.871 | 1.00 | 58.93 | C |
| ATOM | 2053 | CG | ASP | B | 110 | 14.329 | 36.698 | 14.179 | 1.00 | 61.20 | C |
| ATOM | 2054 | OD1 | ASP | B | 110 | 15.110 | 36.866 | 15.149 | 1.00 | 64.56 | O |
| ATOM | 2055 | OD2 | ASP | B | 110 | 13.335 | 35.946 | 14.236 | 1.00 | 62.44 | O |
| ATOM | 2056 | C | ASP | B | 110 | 16.481 | 36.773 | 11.360 | 1.00 | 55.17 | C |
| ATOM | 2057 | O | ASP | B | 110 | 15.835 | 36.771 | 10.316 | 1.00 | 56.87 | O |
| ATOM | 2058 | N | VAL | B | 111 | 17.581 | 36.039 | 11.523 | 1.00 | 51.13 | N |
| ATOM | 2059 | CA | VAL | B | 111 | 18.119 | 35.158 | 10.471 | 1.00 | 47.12 | C |
| ATOM | 2060 | CB | VAL | B | 111 | 19.647 | 35.389 | 10.217 | 1.00 | 48.87 | C |
| ATOM | 2061 | CG1 | VAL | B | 111 | 20.219 | 34.352 | 9.227 | 1.00 | 53.14 | C |
| ATOM | 2062 | CG2 | VAL | B | 111 | 19.938 | 36.818 | 9.728 | 1.00 | 42.07 | C |
| ATOM | 2063 | C | VAL | B | 111 | 17.921 | 33.693 | 10.852 | 1.00 | 46.36 | C |
| ATOM | 2064 | O | VAL | B | 111 | 18.266 | 33.289 | 11.975 | 1.00 | 42.74 | O |
| ATOM | 2065 | N | LYS | B | 112 | 17.400 | 32.898 | 9.915 | 1.00 | 43.12 | N |
| ATOM | 2066 | CA | LYS | B | 112 | 17.032 | 31.485 | 10.162 | 1.00 | 43.58 | C |
| ATOM | 2067 | CB | LYS | B | 112 | 15.546 | 31.455 | 10.558 | 1.00 | 42.82 | C |
| ATOM | 2068 | CG | LYS | B | 112 | 14.766 | 30.199 | 10.270 | 1.00 | 44.74 | C |
| ATOM | 2069 | CD | LYS | B | 112 | 13.271 | 30.542 | 10.259 | 1.00 | 48.96 | C |
| ATOM | 2070 | CE | LYS | B | 112 | 12.691 | 30.498 | 11.670 | 1.00 | 45.37 | C |
| ATOM | 2071 | NZ | LYS | B | 112 | 11.754 | 31.661 | 11.947 | 1.00 | 45.29 | N |
| ATOM | 2072 | C | LYS | B | 112 | 17.346 | 30.504 | 9.003 | 1.00 | 42.43 | C |
| ATOM | 2073 | O | LYS | B | 112 | 17.477 | 30.921 | 7.847 | 1.00 | 45.59 | O |
| ATOM | 2074 | N | PHE | B | 113 | 17.513 | 29.216 | 9.327 | 1.00 | 40.79 | N |
| ATOM | 2075 | CA | PHE | B | 113 | 17.572 | 28.148 | 8.331 | 1.00 | 38.79 | C |
| ATOM | 2076 | CB | PHE | B | 113 | 18.998 | 27.550 | 8.153 | 1.00 | 39.14 | C |
| ATOM | 2077 | CG | PHE | B | 113 | 19.104 | 26.543 | 6.997 | 1.00 | 34.85 | C |
| ATOM | 2078 | CD1 | PHE | B | 113 | 19.549 | 26.945 | 5.741 | 1.00 | 35.55 | C |
| ATOM | 2079 | CE1 | PHE | B | 113 | 19.640 | 26.015 | 4.697 | 1.00 | 38.24 | C |
| ATOM | 2080 | CZ | PHE | B | 113 | 19.250 | 24.709 | 4.892 | 1.00 | 34.91 | C |
| ATOM | 2081 | CE2 | PHE | B | 113 | 18.807 | 24.299 | 6.148 | 1.00 | 40.75 | C |
| ATOM | 2082 | CD2 | PHE | B | 113 | 18.737 | 25.208 | 7.177 | 1.00 | 44.17 | C |
| ATOM | 2083 | C | PHE | B | 113 | 16.634 | 27.073 | 8.800 | 1.00 | 39.23 | C |
| ATOM | 2084 | O | PHE | B | 113 | 16.731 | 26.593 | 9.956 | 1.00 | 43.15 | O |
| ATOM | 2085 | N | THR | B | 114 | 15.728 | 26.686 | 7.916 | 1.00 | 36.48 | N |
| ATOM | 2086 | CA | THR | B | 114 | 14.768 | 25.619 | 8.184 | 1.00 | 36.83 | C |
| ATOM | 2087 | CB | THR | B | 114 | 13.384 | 25.985 | 7.687 | 1.00 | 40.83 | C |
| ATOM | 2088 | OG1 | THR | B | 114 | 12.968 | 27.231 | 8.286 | 1.00 | 37.54 | O |
| ATOM | 2089 | CG2 | THR | B | 114 | 12.394 | 24.877 | 7.993 | 1.00 | 36.72 | C |
| ATOM | 2090 | C | THR | B | 114 | 15.214 | 24.313 | 7.502 | 1.00 | 38.84 | C |
| ATOM | 2091 | O | THR | B | 114 | 15.307 | 24.201 | 6.286 | 1.00 | 40.09 | O |
| ATOM | 2092 | N | ILE | B | 115 | 15.501 | 23.317 | 8.311 | 1.00 | 38.37 | N |
| ATOM | 2093 | CA | ILE | B | 115 | 15.934 | 22.042 | 7.802 | 1.00 | 38.31 | C |
| ATOM | 2094 | CB | ILE | B | 115 | 16.914 | 21.365 | 8.790 | 1.00 | 38.74 | C |
| ATOM | 2095 | OG1 | ILE | B | 115 | 18.233 | 22.145 | 8.874 | 1.00 | 40.45 | C |
| ATOM | 2096 | CD1 | ILE | B | 115 | 19.179 | 21.628 | 9.988 | 1.00 | 41.07 | C |
| ATOM | 2097 | CG2 | ILE | B | 115 | 17.189 | 19.957 | 8.376 | 1.00 | 37.17 | C |
| ATOM | 2098 | C | ILE | B | 115 | 14.696 | 21.197 | 7.693 | 1.00 | 39.26 | C |
| ATOM | 2099 | O | ILE | B | 115 | 13.905 | 21.146 | 8.626 | 1.00 | 40.19 | O |
| ATOM | 2100 | N | LYS | B | 116 | 14.508 | 20.566 | 6.539 | 1.00 | 38.88 | N |
| ATOM | 2101 | CA | LYS | B | 116 | 13.586 | 19.455 | 6.452 | 1.00 | 39.98 | C |
| ATOM | 2102 | CB | LYS | B | 116 | 12.647 | 19.601 | 5.253 | 1.00 | 39.37 | C |
| ATOM | 2103 | CG | LYS | B | 116 | 11.972 | 18.305 | 4.805 | 1.00 | 40.21 | C |
| ATOM | 2104 | CD | LYS | B | 116 | 10.761 | 18.025 | 5.630 | 1.00 | 40.79 | C |
| ATOM | 2105 | CE | LYS | B | 116 | 9.630 | 17.361 | 4.844 | 1.00 | 47.35 | C |
| ATOM | 2106 | NZ | LYS | B | 116 | 10.037 | 16.252 | 3.926 | 1.00 | 43.81 | N |
| ATOM | 2107 | C | LYS | B | 116 | 14.408 | 18.162 | 6.350 | 1.00 | 41.05 | C |
| ATOM | 2108 | O | LYS | B | 116 | 15.163 | 17.961 | 5.393 | 1.00 | 40.58 | O |
| ATOM | 2109 | N | PHE | B | 117 | 14.250 | 17.299 | 7.347 | 1.00 | 40.29 | N |
| ATOM | 2110 | CA | PHE | B | 117 | 14.894 | 15.997 | 7.368 | 1.00 | 40.81 | C |
| ATOM | 2111 | CB | PHE | B | 117 | 14.958 | 15.425 | 8.801 | 1.00 | 41.73 | C |
| ATOM | 2112 | CG | PHE | B | 117 | 15.737 | 16.291 | 9.761 | 1.00 | 39.87 | C |
| ATOM | 2113 | CD1 | PHE | B | 117 | 15.091 | 17.297 | 10.487 | 1.00 | 40.27 | C |
| ATOM | 2114 | CE1 | PHE | B | 117 | 15.791 | 18.118 | 11.354 | 1.00 | 36.48 | C |
| ATOM | 2115 | CZ | PHE | B | 117 | 17.174 | 17.971 | 11.468 | 1.00 | 40.12 | C |
| ATOM | 2116 | CE2 | PHE | B | 117 | 17.845 | 16.976 | 10.706 | 1.00 | 36.80 | C |
| ATOM | 2117 | CD2 | PHE | B | 117 | 17.121 | 16.151 | 9.874 | 1.00 | 34.89 | C |
| ATOM | 2118 | C | PHE | B | 117 | 14.109 | 15.078 | 6.451 | 1.00 | 42.46 | C |
| ATOM | 2119 | O | PHE | B | 117 | 13.074 | 14.534 | 6.839 | 1.00 | 42.34 | O |
| ATOM | 2120 | N | GLN | B | 118 | 14.599 | 14.956 | 5.220 | 1.00 | 43.34 | N |
| ATOM | 2121 | CA | GLN | B | 118 | 14.001 | 14.143 | 4.168 | 1.00 | 42.93 | C |
| ATOM | 2122 | CB | GLN | B | 118 | 13.223 | 15.038 | 3.177 | 1.00 | 47.34 | C |
| ATOM | 2123 | CG | GLN | B | 118 | 14.085 | 16.077 | 2.491 | 1.00 | 34.74 | C |
| ATOM | 2124 | CD | GLN | B | 118 | 13.336 | 16.995 | 1.531 | 1.00 | 41.59 | C |
| ATOM | 2125 | OE1 | GLN | B | 118 | 13.710 | 18.137 | 1.385 | 1.00 | 41.26 | O |
| ATOM | 2126 | NE2 | GLN | B | 118 | 12.277 | 16.508 | 0.896 | 1.00 | 37.39 | N |
| ATOM | 2127 | C | GLN | B | 118 | 15.137 | 13.401 | 3.453 | 1.00 | 43.86 | C |
| ATOM | 2128 | O | GLN | B | 118 | 16.281 | 13.882 | 3.422 | 1.00 | 40.85 | O |
| ATOM | 2129 | N | GLU | B | 119 | 14.817 | 12.239 | 2.873 | 1.00 | 45.99 | N |
| ATOM | 2130 | CA | GLU | B | 119 | 15.833 | 11.342 | 2.277 | 1.00 | 46.54 | C |
| ATOM | 2131 | CB | GLU | B | 119 | 15.266 | 9.924 | 2.128 | 1.00 | 49.19 | C |
| ATOM | 2132 | CG | GLU | B | 119 | 16.253 | 8.817 | 2.513 | 1.00 | 54.11 | C |
| ATOM | 2133 | CD | GLU | B | 119 | 15.932 | 8.188 | 3.859 | 1.00 | 62.40 | C |
| ATOM | 2134 | OE1 | GLU | B | 119 | 14.732 | 8.144 | 4.211 | 1.00 | 69.97 | O |
| ATOM | 2135 | OE2 | GLU | B | 119 | 16.864 | 7.725 | 4.560 | 1.00 | 62.46 | O |
| ATOM | 2136 | C | GLU | B | 119 | 16.375 | 11.802 | 0.913 | 1.00 | 45.75 | C |
| ATOM | 2137 | O | GLU | B | 119 | 17.486 | 11.447 | 0.523 | 1.00 | 46.08 | O |
| ATOM | 2138 | N | PHE | B | 120 | 15.579 | 12.606 | 0.212 | 1.00 | 41.97 | N |
| ATOM | 2139 | CA | PHE | B | 120 | 15.842 | 13.014 | −1.161 | 1.00 | 41.19 | C |
| ATOM | 2140 | CB | PHE | B | 120 | 15.190 | 11.988 | −2.102 | 1.00 | 41.38 | C |
| ATOM | 2141 | CG | PHE | B | 120 | 15.342 | 12.284 | −3.568 | 1.00 | 44.79 | C |
| ATOM | 2142 | CD1 | PHE | B | 120 | 16.433 | 11.791 | −4.276 | 1.00 | 46.25 | C |
| ATOM | 2143 | CE1 | PHE | B | 120 | 16.573 | 12.041 | −5.639 | 1.00 | 48.01 | C |
| ATOM | 2144 | CZ | PHE | B | 120 | 15.597 | 12.780 | −6.311 | 1.00 | 48.10 | C |
| ATOM | 2145 | CE2 | PHE | B | 120 | 14.491 | 13.269 | −5.614 | 1.00 | 48.86 | C |
| ATOM | 2146 | CD2 | PHE | B | 120 | 14.363 | 13.008 | −4.251 | 1.00 | 47.10 | C |
| ATOM | 2147 | C | PHE | B | 120 | 15.235 | 14.411 | −1.339 | 1.00 | 38.45 | C |
| ATOM | 2148 | O | PHE | B | 120 | 14.098 | 14.642 | −0.939 | 1.00 | 39.71 | O |
| ATOM | 2149 | N | SER | B | 121 | 16.001 | 15.338 | −1.908 | 1.00 | 38.75 | N |
| ATOM | 2150 | CA | SER | B | 121 | 15.503 | 16.687 | −2.222 | 1.00 | 35.73 | C |
| ATOM | 2151 | CB | SER | B | 121 | 16.336 | 17.720 | −1.482 | 1.00 | 41.04 | C |
| ATOM | 2152 | OG | SER | B | 121 | 16.075 | 19.038 | −1.979 | 1.00 | 32.57 | O |
| ATOM | 2153 | C | SER | B | 121 | 15.630 | 16.928 | −3.713 | 1.00 | 36.73 | C |
| ATOM | 2154 | O | SER | B | 121 | 16.697 | 16.644 | −4.245 | 1.00 | 37.53 | O |
| ATOM | 2155 | N | PRO | B | 122 | 14.549 | 17.421 | −4.414 | 1.00 | 35.00 | N |
| ATOM | 2156 | CA | PRO | B | 122 | 14.686 | 17.884 | −5.794 | 1.00 | 31.41 | C |
| ATOM | 2157 | CB | PRO | B | 122 | 13.251 | 18.193 | −6.211 | 1.00 | 33.87 | C |
| ATOM | 2158 | CG | PRO | B | 122 | 12.549 | 18.466 | −4.956 | 1.00 | 36.13 | C |
| ATOM | 2159 | CD | PRO | B | 122 | 13.128 | 17.460 | −4.018 | 1.00 | 31.71 | C |
| ATOM | 2160 | C | PRO | B | 122 | 15.605 | 19.080 | −6.037 | 1.00 | 34.20 | C |
| ATOM | 2161 | O | PRO | B | 122 | 15.924 | 19.376 | −7.206 | 1.00 | 28.62 | O |
| ATOM | 2162 | N | ASN | B | 123 | 16.063 | 19.713 | −4.952 | 1.00 | 35.06 | N |
| ATOM | 2163 | CA | ASN | B | 123 | 17.077 | 20.740 | −5.053 | 1.00 | 35.87 | C |
| ATOM | 2164 | CB | ASN | B | 123 | 17.094 | 21.655 | −3.821 | 1.00 | 36.31 | C |
| ATOM | 2165 | CG | ASN | B | 123 | 18.043 | 22.880 | −3.984 | 1.00 | 33.87 | C |
| ATOM | 2166 | OD1 | ASN | B | 123 | 18.656 | 23.093 | −5.032 | 1.00 | 32.22 | O |
| ATOM | 2167 | ND2 | ASN | B | 123 | 18.121 | 23.693 | −2.943 | 1.00 | 35.68 | N |
| ATOM | 2168 | C | ASN | B | 123 | 18.448 | 20.112 | −5.270 | 1.00 | 34.15 | C |
| ATOM | 2169 | O | ASN | B | 123 | 18.928 | 19.291 | −4.469 | 1.00 | 33.65 | O |
| ATOM | 2170 | N | LEU | B | 124 | 19.039 | 20.510 | −6.383 | 1.00 | 33.29 | N |
| ATOM | 2171 | CA | LEU | B | 124 | 20.394 | 20.196 | −6.723 | 1.00 | 37.26 | C |
| ATOM | 2172 | CB | LEU | B | 124 | 20.804 | 21.083 | −7.895 | 1.00 | 35.44 | C |
| ATOM | 2173 | CG | LEU | B | 124 | 22.203 | 20.963 | −8.484 | 1.00 | 40.34 | C |
| ATOM | 2174 | CD1 | LEU | B | 124 | 22.338 | 19.636 | −9.207 | 1.00 | 35.24 | C |
| ATOM | 2175 | CD2 | LEU | B | 124 | 22.360 | 22.114 | −9.433 | 1.00 | 38.11 | C |
| ATOM | 2176 | C | LEU | B | 124 | 21.341 | 20.364 | −5.524 | 1.00 | 36.68 | C |
| ATOM | 2177 | O | LEU | B | 124 | 22.267 | 19.587 | −5.378 | 1.00 | 38.26 | O |
| ATOM | 2178 | N | TRP | B | 125 | 21.088 | 21.354 | −4.662 | 1.00 | 37.81 | N |
| ATOM | 2179 | CA | TRP | B | 125 | 21.878 | 21.556 | −3.442 | 1.00 | 41.99 | C |
| ATOM | 2180 | CB | TRP | B | 125 | 22.371 | 22.993 | −3.331 | 1.00 | 44.52 | C |
| ATOM | 2181 | CG | TRP | B | 125 | 23.266 | 23.394 | −4.426 | 1.00 | 48.30 | C |
| ATOM | 2182 | CD1 | TRP | B | 125 | 24.609 | 23.183 | −4.507 | 1.00 | 48.48 | C |
| ATOM | 2183 | NE1 | TRP | B | 125 | 25.100 | 23.701 | −5.680 | 1.00 | 46.89 | N |
| ATOM | 2184 | CE2 | TRP | B | 125 | 24.065 | 24.261 | −6.381 | 1.00 | 48.31 | C |
| ATOM | 2185 | CD2 | TRP | B | 125 | 22.892 | 24.093 | −5.611 | 1.00 | 48.46 | C |
| ATOM | 2186 | CE3 | TRP | B | 125 | 21.675 | 24.579 | −6.110 | 1.00 | 51.01 | C |
| ATOM | 2187 | CZ3 | TRP | B | 125 | 21.674 | 25.222 | −7.336 | 1.00 | 47.38 | C |
| ATOM | 2188 | CH2 | TRP | B | 125 | 22.857 | 25.380 | −8.074 | 1.00 | 47.99 | C |
| ATOM | 2189 | CZ2 | TRP | B | 125 | 24.062 | 24.917 | −7.611 | 1.00 | 47.47 | C |
| ATOM | 2190 | C | TRP | B | 125 | 21.117 | 21.240 | −2.181 | 1.00 | 41.23 | C |
| ATOM | 2191 | O | TRP | B | 125 | 21.540 | 21.611 | −1.091 | 1.00 | 44.51 | O |
| ATOM | 2192 | N | GLY | B | 126 | 20.007 | 20.538 | −2.321 | 1.00 | 40.47 | N |
| ATOM | 2193 | CA | GLY | B | 126 | 19.175 | 20.183 | −1.183 | 1.00 | 40.35 | C |
| ATOM | 2194 | C | GLY | B | 126 | 19.900 | 19.357 | −0.144 | 1.00 | 41.68 | C |
| ATOM | 2195 | O | GLY | B | 126 | 20.904 | 18.705 | −0.452 | 1.00 | 41.73 | O |
| ATOM | 2196 | N | LEU | B | 127 | 19.384 | 19.378 | 1.086 | 1.00 | 41.78 | N |
| ATOM | 2197 | CA | LEU | B | 127 | 19.928 | 18.578 | 2.187 | 1.00 | 43.54 | C |
| ATOM | 2198 | CB | LEU | B | 127 | 19.885 | 19.371 | 3.502 | 1.00 | 45.56 | C |
| ATOM | 2199 | CG | LEU | B | 127 | 20.829 | 20.562 | 3.715 | 1.00 | 45.47 | C |
| ATOM | 2200 | CD1 | LEU | B | 127 | 20.761 | 21.056 | 5.145 | 1.00 | 47.06 | C |
| ATOM | 2201 | CD2 | LEU | B | 127 | 22.269 | 20.227 | 3.361 | 1.00 | 52.41 | C |
| ATOM | 2202 | C | LEU | B | 127 | 19.169 | 17.251 | 2.317 | 1.00 | 46.47 | C |
| ATOM | 2203 | O | LEU | B | 127 | 17.932 | 17.223 | 2.246 | 1.00 | 45.51 | O |
| ATOM | 2204 | N | GLU | B | 128 | 19.914 | 16.151 | 2.486 | 1.00 | 49.01 | N |
| ATOM | 2205 | CA | GLU | B | 128 | 19.358 | 14.785 | 2.392 | 1.00 | 50.26 | C |
| ATOM | 2206 | CB | GLU | B | 128 | 19.662 | 14.201 | 1.002 | 1.00 | 50.78 | C |
| ATOM | 2207 | CG | GLU | B | 128 | 19.339 | 15.167 | −0.175 | 1.00 | 52.96 | C |
| ATOM | 2208 | CD | GLU | B | 128 | 19.395 | 14.518 | −1.561 | 1.00 | 52.08 | C |
| ATOM | 2209 | OE1 | GLU | B | 128 | 20.089 | 13.484 | −1.727 | 1.00 | 54.11 | O |
| ATOM | 2210 | OE2 | GLU | B | 128 | 18.733 | 15.050 | −2.500 | 1.00 | 44.98 | O |
| ATOM | 2211 | C | GLU | B | 128 | 19.870 | 13.866 | 3.541 | 1.00 | 51.60 | C |
| ATOM | 2212 | O | GLU | B | 128 | 21.053 | 13.901 | 3.882 | 1.00 | 51.94 | O |
| ATOM | 2213 | N | PHE | B | 129 | 18.984 | 13.064 | 4.143 | 1.00 | 50.16 | N |
| ATOM | 2214 | CA | PHE | B | 129 | 19.301 | 12.410 | 5.428 | 1.00 | 50.35 | C |
| ATOM | 2215 | CB | PHE | B | 129 | 18.616 | 13.148 | 6.579 | 1.00 | 49.69 | C |
| ATOM | 2216 | CG | PHE | B | 129 | 19.000 | 14.602 | 6.689 | 1.00 | 53.84 | C |
| ATOM | 2217 | CD1 | PHE | B | 129 | 18.143 | 15.602 | 6.218 | 1.00 | 53.48 | C |
| ATOM | 2218 | CE1 | PHE | B | 129 | 18.497 | 16.963 | 6.321 | 1.00 | 50.73 | C |
| ATOM | 2219 | CZ | PHE | B | 129 | 19.718 | 17.324 | 6.879 | 1.00 | 51.56 | C |
| ATOM | 2220 | CE2 | PHE | B | 129 | 20.589 | 16.329 | 7.349 | 1.00 | 56.53 | C |
| ATOM | 2221 | CD2 | PHE | B | 129 | 20.223 | 14.982 | 7.253 | 1.00 | 53.44 | C |
| ATOM | 2222 | C | PHE | B | 129 | 18.985 | 10.907 | 5.487 | 1.00 | 52.61 | C |
| ATOM | 2223 | O | PHE | B | 129 | 17.967 | 10.449 | 4.950 | 1.00 | 51.12 | O |
| ATOM | 2224 | N | GLN | B | 130 | 19.849 | 10.151 | 6.172 | 1.00 | 56.51 | N |
| ATOM | 2225 | CA | GLN | B | 130 | 19.828 | 8.674 | 6.124 | 1.00 | 59.82 | C |
| ATOM | 2226 | CB | GLN | B | 130 | 21.247 | 8.116 | 5.889 | 1.00 | 60.33 | C |
| ATOM | 2227 | CG | GLN | B | 130 | 22.020 | 8.753 | 4.713 | 1.00 | 61.86 | C |
| ATOM | 2228 | CD | GLN | B | 130 | 23.338 | 8.053 | 4.385 | 1.00 | 60.72 | C |
| ATOM | 2229 | OE1 | GLN | B | 130 | 23.752 | 7.113 | 5.069 | 1.00 | 69.64 | O |
| ATOM | 2230 | NE2 | GLN | B | 130 | 23.997 | 8.508 | 3.322 | 1.00 | 59.67 | N |
| ATOM | 2231 | C | GLN | B | 130 | 19.170 | 7.969 | 7.328 | 1.00 | 60.84 | C |
| ATOM | 2232 | O | GLN | B | 130 | 19.206 | 8.461 | 8.465 | 1.00 | 59.63 | O |
| ATOM | 2233 | N | ALA | B | 131 | 18.610 | 6.790 | 7.038 | 1.00 | 63.58 | N |
| ATOM | 2234 | CA | ALA | B | 131 | 17.841 | 5.932 | 7.965 | 1.00 | 64.07 | C |
| ATOM | 2235 | CB | ALA | B | 131 | 17.808 | 4.489 | 7.434 | 1.00 | 64.33 | C |
| ATOM | 2236 | C | ALA | B | 131 | 18.239 | 5.947 | 9.453 | 1.00 | 63.60 | C |
| ATOM | 2237 | O | ALA | B | 131 | 17.463 | 6.390 | 10.298 | 1.00 | 64.85 | O |
| ATOM | 2238 | N | ASN | B | 132 | 19.426 | 5.437 | 9.765 | 1.00 | 61.85 | N |
| ATOM | 2239 | CA | ASN | B | 132 | 19.939 | 5.450 | 11.134 | 1.00 | 58.11 | C |
| ATOM | 2240 | CB | ASN | B | 132 | 19.985 | 4.033 | 11.716 | 1.00 | 57.70 | C |
| ATOM | 2241 | CG | ASN | B | 132 | 19.258 | 3.917 | 13.056 | 1.00 | 57.57 | C |
| ATOM | 2242 | OD1 | ASN | B | 132 | 19.863 | 4.027 | 14.125 | 1.00 | 52.75 | O |
| ATOM | 2243 | ND2 | ASN | B | 132 | 17.953 | 3.688 | 12.998 | 1.00 | 56.67 | N |
| ATOM | 2244 | C | ASN | B | 132 | 21.325 | 6.091 | 11.097 | 1.00 | 56.55 | C |
| ATOM | 2245 | O | ASN | B | 132 | 22.347 | 5.409 | 11.243 | 1.00 | 54.32 | O |
| ATOM | 2246 | N | LYS | B | 133 | 21.343 | 7.408 | 10.876 | 1.00 | 52.59 | N |
| ATOM | 2247 | CA | LYS | B | 133 | 22.584 | 8.143 | 10.583 | 1.00 | 51.69 | C |
| ATOM | 2248 | CB | LYS | B | 133 | 22.726 | 8.277 | 9.055 | 1.00 | 52.35 | C |
| ATOM | 2249 | CG | LYS | B | 133 | 23.788 | 9.235 | 8.509 | 1.00 | 54.30 | C |
| ATOM | 2250 | CD | LYS | B | 133 | 25.209 | 8.685 | 8.590 | 1.00 | 55.71 | C |
| ATOM | 2251 | CE | LYS | B | 133 | 26.123 | 9.361 | 7.560 | 1.00 | 56.13 | C |
| ATOM | 2252 | NZ | LYS | B | 133 | 26.218 | 10.847 | 7.729 | 1.00 | 55.26 | N |
| ATOM | 2253 | C | LYS | B | 133 | 22.690 | 9.507 | 11.316 | 1.00 | 50.35 | C |
| ATOM | 2254 | O | LYS | B | 133 | 21.680 | 10.157 | 11.581 | 1.00 | 45.66 | O |
| ATOM | 2255 | N | ASP | B | 134 | 23.925 | 9.906 | 11.646 | 1.00 | 50.68 | N |
| ATOM | 2256 | CA | ASP | B | 134 | 24.219 | 11.149 | 12.375 | 1.00 | 51.33 | C |
| ATOM | 2257 | CB | ASP | B | 134 | 25.246 | 10.914 | 13.497 | 1.00 | 53.85 | C |
| ATOM | 2258 | CG | ASP | B | 134 | 24.881 | 9.760 | 14.403 | 1.00 | 54.97 | C |
| ATOM | 2259 | OD1 | ASP | B | 134 | 23.787 | 9.792 | 15.001 | 1.00 | 61.14 | O |
| ATOM | 2260 | OD2 | ASP | B | 134 | 25.692 | 8.818 | 14.521 | 1.00 | 63.22 | O |
| ATOM | 2261 | C | ASP | B | 134 | 24.781 | 12.214 | 11.446 | 1.00 | 51.63 | C |
| ATOM | 2262 | O | ASP | B | 134 | 25.579 | 11.910 | 10.546 | 1.00 | 50.16 | O |
| ATOM | 2263 | N | TYR | B | 135 | 24.368 | 13.464 | 11.678 | 1.00 | 51.47 | N |
| ATOM | 2264 | CA | TYR | B | 135 | 24.844 | 14.617 | 10.903 | 1.00 | 50.95 | C |
| ATOM | 2265 | CB | TYR | B | 135 | 23.765 | 15.107 | 9.945 | 1.00 | 51.81 | C |
| ATOM | 2266 | CG | TYR | B | 135 | 23.375 | 14.080 | 8.901 | 1.00 | 53.87 | C |
| ATOM | 2267 | CD1 | TYR | B | 135 | 22.302 | 13.228 | 9.120 | 1.00 | 55.22 | C |
| ATOM | 2268 | CE1 | TYR | B | 135 | 21.930 | 12.280 | 8.175 | 1.00 | 55.50 | C |
| ATOM | 2269 | CZ | TYR | B | 135 | 22.633 | 12.174 | 6.991 | 1.00 | 55.59 | C |
| ATOM | 2270 | OH | TYR | B | 135 | 22.241 | 11.219 | 6.075 | 1.00 | 55.74 | O |
| ATOM | 2271 | CE2 | TYR | B | 135 | 23.716 | 13.008 | 6.738 | 1.00 | 55.39 | C |
| ATOM | 2272 | CD2 | TYR | B | 135 | 24.082 | 13.961 | 7.697 | 1.00 | 50.04 | C |
| ATOM | 2273 | C | TYR | B | 135 | 25.222 | 15.724 | 11.857 | 1.00 | 50.07 | C |
| ATOM | 2274 | O | TYR | B | 135 | 24.666 | 15.811 | 12.959 | 1.00 | 47.79 | O |
| ATOM | 2275 | N | TYR | B | 136 | 26.169 | 16.559 | 11.444 | 1.00 | 52.35 | N |
| ATOM | 2276 | CA | TYR | B | 136 | 26.726 | 17.566 | 12.352 | 1.00 | 53.64 | C |
| ATOM | 2277 | CB | TYR | B | 136 | 28.187 | 17.242 | 12.691 | 1.00 | 56.73 | C |
| ATOM | 2278 | CG | TYR | B | 136 | 28.423 | 15.870 | 13.310 | 1.00 | 60.71 | C |
| ATOM | 2279 | CD1 | TYR | B | 136 | 28.328 | 14.701 | 12.541 | 1.00 | 63.40 | C |
| ATOM | 2280 | CE1 | TYR | B | 136 | 28.547 | 13.453 | 13.099 | 1.00 | 63.12 | C |
| ATOM | 2281 | CZ | TYR | B | 136 | 28.892 | 13.353 | 14.431 | 1.00 | 61.25 | C |
| ATOM | 2282 | OH | TYR | B | 136 | 29.120 | 12.116 | 14.984 | 1.00 | 61.58 | O |
| ATOM | 2283 | CE2 | TYR | B | 136 | 29.008 | 14.494 | 15.216 | 1.00 | 64.20 | C |
| ATOM | 2284 | CD2 | TYR | B | 136 | 28.772 | 15.743 | 14.654 | 1.00 | 59.76 | C |
| ATOM | 2285 | C | TYR | B | 136 | 26.636 | 18.985 | 11.820 | 1.00 | 52.48 | C |
| ATOM | 2286 | O | TYR | B | 136 | 26.701 | 19.225 | 10.613 | 1.00 | 52.71 | O |
| ATOM | 2287 | N | ILE | B | 137 | 26.509 | 19.926 | 12.750 | 1.00 | 51.25 | N |
| ATOM | 2288 | CA | ILE | B | 137 | 26.430 | 21.344 | 12.443 | 1.00 | 48.75 | C |
| ATOM | 2289 | CB | ILE | B | 137 | 24.975 | 21.849 | 12.481 | 1.00 | 51.09 | C |
| ATOM | 2290 | CG1 | ILE | B | 137 | 24.223 | 21.481 | 11.180 | 1.00 | 50.13 | C |
| ATOM | 2291 | CD1 | ILE | B | 137 | 22.704 | 21.633 | 11.274 | 1.00 | 47.53 | C |
| ATOM | 2292 | CG2 | ILE | B | 137 | 24.941 | 23.358 | 12.811 | 1.00 | 40.73 | C |
| ATOM | 2293 | C | ILE | B | 137 | 27.240 | 22.060 | 13.510 | 1.00 | 48.65 | C |
| ATOM | 2294 | O | ILE | B | 137 | 26.978 | 21.897 | 14.709 | 1.00 | 46.02 | O |
| ATOM | 2295 | N | ILE | B | 138 | 28.208 | 22.864 | 13.067 | 1.00 | 46.40 | N |
| ATOM | 2296 | CA | ILE | B | 138 | 29.125 | 23.546 | 13.969 | 1.00 | 44.96 | C |
| ATOM | 2297 | CB | ILE | B | 138 | 30.534 | 22.873 | 13.957 | 1.00 | 46.61 | C |
| ATOM | 2298 | CG1 | ILE | B | 138 | 31.170 | 22.955 | 12.558 | 1.00 | 44.58 | C |
| ATOM | 2299 | CD1 | ILE | B | 138 | 32.693 | 22.657 | 12.515 | 1.00 | 36.53 | C |
| ATOM | 2300 | CG2 | ILE | B | 138 | 30.468 | 21.423 | 14.466 | 1.00 | 48.73 | C |
| ATOM | 2301 | C | ILE | B | 138 | 29.285 | 24.999 | 13.548 | 1.00 | 46.45 | C |
| ATOM | 2302 | O | ILE | B | 138 | 28.681 | 25.433 | 12.583 | 1.00 | 45.73 | O |
| ATOM | 2303 | N | SER | B | 139 | 30.093 | 25.756 | 14.283 | 1.00 | 46.68 | N |
| ATOM | 2304 | CA | SER | B | 139 | 30.615 | 27.033 | 13.767 | 1.00 | 50.66 | C |
| ATOM | 2305 | CB | SER | B | 139 | 29.706 | 28.215 | 14.146 | 1.00 | 51.05 | C |
| ATOM | 2306 | OG | SER | B | 139 | 30.290 | 29.441 | 13.717 | 1.00 | 52.58 | O |
| ATOM | 2307 | C | SER | B | 139 | 32.023 | 27.264 | 14.314 | 1.00 | 50.78 | C |
| ATOM | 2308 | O | SER | B | 139 | 32.239 | 27.119 | 15.520 | 1.00 | 51.39 | O |
| ATOM | 2309 | N | THR | B | 140 | 32.964 | 27.625 | 13.438 | 1.00 | 50.11 | N |
| ATOM | 2310 | CA | THR | B | 140 | 34.352 | 27.920 | 13.864 | 1.00 | 54.13 | C |
| ATOM | 2311 | CB | THR | B | 140 | 35.409 | 27.282 | 12.924 | 1.00 | 52.12 | C |
| ATOM | 2312 | OG1 | THR | B | 140 | 35.051 | 27.513 | 11.560 | 1.00 | 51.29 | O |
| ATOM | 2313 | CG2 | THR | B | 140 | 35.493 | 25.775 | 13.164 | 1.00 | 56.06 | C |
| ATOM | 2314 | C | THR | B | 140 | 34.629 | 29.421 | 14.072 | 1.00 | 53.66 | C |
| ATOM | 2315 | O | THR | B | 140 | 35.762 | 29.833 | 14.319 | 1.00 | 54.84 | O |
| ATOM | 2316 | N | SER | B | 141 | 33.568 | 30.220 | 13.978 | 1.00 | 53.68 | N |
| ATOM | 2317 | CA | SER | B | 141 | 33.640 | 31.661 | 14.128 | 1.00 | 52.39 | C |
| ATOM | 2318 | CB | SER | B | 141 | 32.400 | 32.311 | 13.503 | 1.00 | 54.33 | C |
| ATOM | 2319 | OG | SER | B | 141 | 32.157 | 31.779 | 12.205 | 1.00 | 47.17 | O |
| ATOM | 2320 | C | SER | B | 141 | 33.722 | 31.968 | 15.609 | 1.00 | 54.75 | C |
| ATOM | 2321 | O | SER | B | 141 | 33.090 | 31.288 | 16.428 | 1.00 | 57.16 | O |
| ATOM | 2322 | N | ASN | B | 142 | 34.509 | 32.969 | 15.975 | 1.00 | 52.29 | N |
| ATOM | 2323 | CA | ASN | B | 142 | 34.741 | 33.204 | 17.392 | 1.00 | 51.16 | C |
| ATOM | 2324 | CB | ASN | B | 142 | 36.220 | 33.539 | 17.661 | 1.00 | 50.07 | C |
| ATOM | 2325 | CG | ASN | B | 142 | 36.473 | 34.996 | 17.869 | 1.00 | 55.16 | C |
| ATOM | 2326 | OD1 | ASN | B | 142 | 36.678 | 35.743 | 16.915 | 1.00 | 61.85 | O |
| ATOM | 2327 | ND2 | ASN | B | 142 | 36.509 | 35.414 | 19.136 | 1.00 | 51.49 | N |
| ATOM | 2328 | C | ASN | B | 142 | 33.736 | 34.157 | 18.063 | 1.00 | 51.20 | C |
| ATOM | 2329 | O | ASN | B | 142 | 33.895 | 34.521 | 19.238 | 1.00 | 51.08 | O |
| ATOM | 2330 | N | GLY | B | 143 | 32.719 | 34.570 | 17.299 | 1.00 | 50.47 | N |
| ATOM | 2331 | CA | GLY | B | 143 | 31.643 | 35.424 | 17.795 | 1.00 | 50.52 | C |
| ATOM | 2332 | C | GLY | B | 143 | 31.764 | 36.925 | 17.590 | 1.00 | 51.28 | C |
| ATOM | 2333 | O | GLY | B | 143 | 30.825 | 37.669 | 17.903 | 1.00 | 50.26 | O |
| ATOM | 2334 | N | SER | B | 144 | 32.898 | 37.376 | 17.056 | 1.00 | 53.07 | N |
| ATOM | 2335 | CA | SER | B | 144 | 33.265 | 38.799 | 17.086 | 1.00 | 55.66 | C |
| ATOM | 2336 | CB | SER | B | 144 | 34.725 | 38.939 | 17.504 | 1.00 | 55.64 | C |
| ATOM | 2337 | OG | SER | B | 144 | 35.557 | 38.164 | 16.662 | 1.00 | 55.12 | O |
| ATOM | 2338 | C | SER | B | 144 | 33.037 | 39.605 | 15.805 | 1.00 | 57.74 | C |
| ATOM | 2339 | O | SER | B | 144 | 32.699 | 40.803 | 15.873 | 1.00 | 58.24 | O |
| ATOM | 2340 | N | LEU | B | 145 | 33.237 | 38.964 | 14.651 | 1.00 | 58.51 | N |
| ATOM | 2341 | CA | LEU | B | 145 | 33.204 | 39.638 | 13.329 | 1.00 | 59.93 | C |
| ATOM | 2342 | CB | LEU | B | 145 | 32.877 | 41.138 | 13.436 | 1.00 | 59.99 | C |
| ATOM | 2343 | CG | LEU | B | 145 | 32.790 | 42.022 | 12.187 | 1.00 | 58.59 | C |
| ATOM | 2344 | CD1 | LEU | B | 145 | 31.717 | 41.539 | 11.235 | 1.00 | 56.85 | C |
| ATOM | 2345 | CD2 | LEU | B | 145 | 32.557 | 43.492 | 12.576 | 1.00 | 60.03 | C |
| ATOM | 2346 | C | LEU | B | 145 | 34.515 | 39.443 | 12.583 | 1.00 | 60.20 | C |
| ATOM | 2347 | O | LEU | B | 145 | 34.543 | 38.819 | 11.524 | 1.00 | 60.65 | O |
| ATOM | 2348 | N | GLU | B | 146 | 35.604 | 39.974 | 13.134 | 1.00 | 61.34 | N |
| ATOM | 2349 | CA | GLU | B | 146 | 36.928 | 39.772 | 12.538 | 1.00 | 62.60 | C |
| ATOM | 2350 | CB | GLU | B | 146 | 37.998 | 40.590 | 13.266 | 1.00 | 61.85 | C |
| ATOM | 2351 | CG | GLU | B | 146 | 38.123 | 42.030 | 12.766 | 1.00 | 64.43 | C |
| ATOM | 2352 | CD | GLU | B | 146 | 37.060 | 42.965 | 13.338 | 1.00 | 67.41 | C |
| ATOM | 2353 | OE1 | GLU | B | 146 | 37.408 | 43.791 | 14.212 | 1.00 | 68.51 | O |
| ATOM | 2354 | OE2 | GLU | B | 146 | 35.884 | 42.880 | 12.916 | 1.00 | 67.92 | O |
| ATOM | 2355 | C | GLU | B | 146 | 37.292 | 38.287 | 12.520 | 1.00 | 62.26 | C |
| ATOM | 2356 | O | GLU | B | 146 | 38.134 | 37.850 | 11.723 | 1.00 | 62.28 | O |
| ATOM | 2357 | N | GLY | B | 147 | 36.635 | 37.522 | 13.393 | 1.00 | 60.53 | N |
| ATOM | 2358 | CA | GLY | B | 147 | 36.785 | 36.077 | 13.424 | 1.00 | 58.38 | C |
| ATOM | 2359 | C | GLY | B | 147 | 35.703 | 35.336 | 12.662 | 1.00 | 57.21 | C |
| ATOM | 2360 | O | GLY | B | 147 | 35.808 | 34.128 | 12.464 | 1.00 | 56.76 | O |
| ATOM | 2361 | N | LEU | B | 148 | 34.675 | 36.069 | 12.229 | 1.00 | 57.52 | N |
| ATOM | 2362 | CA | LEU | B | 148 | 33.487 | 35.517 | 11.552 | 1.00 | 55.98 | C |
| ATOM | 2363 | CB | LEU | B | 148 | 32.674 | 36.661 | 10.914 | 1.00 | 56.73 | C |
| ATOM | 2364 | OG | LEU | B | 148 | 31.380 | 36.448 | 10.111 | 1.00 | 54.78 | C |
| ATOM | 2365 | CD1 | LEU | B | 148 | 30.401 | 35.463 | 10.759 | 1.00 | 47.97 | C |
| ATOM | 2366 | CD2 | LEU | B | 148 | 30.710 | 37.791 | 9.842 | 1.00 | 53.84 | C |
| ATOM | 2367 | C | LEU | B | 148 | 33.817 | 34.432 | 10.524 | 1.00 | 56.07 | C |
| ATOM | 2368 | O | LEU | B | 148 | 33.181 | 33.388 | 10.489 | 1.00 | 55.33 | O |
| ATOM | 2369 | N | ASP | B | 149 | 34.832 | 34.674 | 9.704 | 1.00 | 57.37 | N |
| ATOM | 2370 | CA | ASP | B | 149 | 35.228 | 33.712 | 8.675 | 1.00 | 57.71 | C |
| ATOM | 2371 | CB | ASP | B | 149 | 35.433 | 34.427 | 7.333 | 1.00 | 58.37 | C |
| ATOM | 2372 | CG | ASP | B | 149 | 35.787 | 35.901 | 7.504 | 1.00 | 59.20 | C |
| ATOM | 2373 | OD1 | ASP | B | 149 | 36.457 | 36.258 | 8.508 | 1.00 | 58.54 | O |
| ATOM | 2374 | OD2 | ASP | B | 149 | 35.383 | 36.700 | 6.635 | 1.00 | 60.63 | O |
| ATOM | 2375 | C | ASP | B | 149 | 36.466 | 32.893 | 9.080 | 1.00 | 58.10 | C |
| ATOM | 2376 | O | ASP | B | 149 | 37.213 | 32.410 | 8.220 | 1.00 | 57.61 | O |
| ATOM | 2377 | N | ASN | B | 150 | 36.680 | 32.742 | 10.390 | 1.00 | 56.91 | N |
| ATOM | 2378 | CA | ASN | B | 150 | 37.712 | 31.834 | 10.874 | 1.00 | 56.84 | C |
| ATOM | 2379 | CB | ASN | B | 150 | 37.912 | 31.931 | 12.396 | 1.00 | 55.39 | C |
| ATOM | 2380 | CG | ASN | B | 150 | 38.659 | 33.192 | 12.799 | 1.00 | 54.03 | C |
| ATOM | 2381 | OD1 | ASN | B | 150 | 39.183 | 33.914 | 11.946 | 1.00 | 53.83 | O |
| ATOM | 2382 | ND2 | ASN | B | 150 | 38.698 | 33.473 | 14.099 | 1.00 | 56.00 | N |
| ATOM | 2383 | C | ASN | B | 150 | 37.367 | 30.430 | 10.447 | 1.00 | 57.87 | C |
| ATOM | 2384 | O | ASN | B | 150 | 36.336 | 29.880 | 10.840 | 1.00 | 58.19 | O |
| ATOM | 2385 | N | GLN | B | 151 | 38.222 | 29.876 | 9.599 | 1.00 | 57.57 | N |
| ATOM | 2386 | CA | GLN | B | 151 | 38.005 | 28.551 | 9.045 | 1.00 | 58.65 | C |
| ATOM | 2387 | CB | GLN | B | 151 | 38.760 | 28.417 | 7.716 | 1.00 | 59.29 | C |
| ATOM | 2388 | CG | GLN | B | 151 | 38.337 | 29.449 | 6.648 | 1.00 | 55.33 | C |
| ATOM | 2389 | CD | GLN | B | 151 | 37.597 | 28.832 | 5.475 | 1.00 | 61.37 | C |
| ATOM | 2390 | OE1 | GLN | B | 151 | 38.109 | 27.918 | 4.824 | 1.00 | 63.82 | O |
| ATOM | 2391 | NE2 | GLN | B | 151 | 36.394 | 29.340 | 5.184 | 1.00 | 50.29 | N |
| ATOM | 2392 | C | GLN | B | 151 | 38.365 | 27.428 | 10.036 | 1.00 | 59.36 | C |
| ATOM | 2393 | O | GLN | B | 151 | 38.176 | 26.237 | 9.730 | 1.00 | 58.04 | O |
| ATOM | 2394 | N | GLU | B | 152 | 38.862 | 27.810 | 11.223 | 1.00 | 59.43 | N |
| ATOM | 2395 | CA | GLU | B | 152 | 39.181 | 26.833 | 12.272 | 1.00 | 60.25 | C |
| ATOM | 2396 | CB | GLU | B | 152 | 40.602 | 26.256 | 12.078 | 1.00 | 61.48 | C |
| ATOM | 2397 | CG | GLU | B | 152 | 41.747 | 27.124 | 12.625 | 1.00 | 66.64 | C |
| ATOM | 2398 | CD | GLU | B | 152 | 42.467 | 27.885 | 11.504 | 1.00 | 68.50 | C |
| ATOM | 2399 | OE1 | GLU | B | 152 | 43.153 | 27.227 | 10.670 | 1.00 | 70.55 | O |
| ATOM | 2400 | OE2 | GLU | B | 152 | 42.360 | 29.142 | 11.471 | 1.00 | 70.22 | O |
| ATOM | 2401 | C | GLU | B | 152 | 38.979 | 27.296 | 13.735 | 1.00 | 59.91 | C |
| ATOM | 2402 | O | GLU | B | 152 | 39.092 | 28.489 | 14.055 | 1.00 | 57.60 | O |
| ATOM | 2403 | N | GLY | B | 153 | 38.670 | 26.328 | 14.603 | 1.00 | 59.88 | N |
| ATOM | 2404 | CA | GLY | B | 153 | 38.611 | 26.530 | 16.052 | 1.00 | 61.08 | C |
| ATOM | 2405 | C | GLY | B | 153 | 37.320 | 27.083 | 16.638 | 1.00 | 62.49 | C |
| ATOM | 2406 | O | GLY | B | 153 | 36.298 | 26.389 | 16.676 | 1.00 | 61.87 | O |
| ATOM | 2407 | N | GLY | B | 154 | 37.397 | 28.340 | 17.090 | 1.00 | 63.05 | N |
| ATOM | 2408 | CA | GLY | B | 154 | 36.402 | 29.036 | 17.933 | 1.00 | 62.98 | C |
| ATOM | 2409 | C | GLY | B | 154 | 34.981 | 28.521 | 18.070 | 1.00 | 62.97 | C |
| ATOM | 2410 | O | GLY | B | 154 | 34.373 | 28.096 | 17.087 | 1.00 | 64.66 | O |
| ATOM | 2411 | N | VAL | B | 155 | 34.452 | 28.571 | 19.295 | 1.00 | 61.10 | N |
| ATOM | 2412 | CA | VAL | B | 155 | 33.051 | 28.238 | 19.574 | 1.00 | 57.44 | C |
| ATOM | 2413 | CB | VAL | B | 155 | 32.086 | 29.191 | 18.770 | 1.00 | 59.75 | C |
| ATOM | 2414 | CG1 | VAL | B | 155 | 30.869 | 28.479 | 18.148 | 1.00 | 53.52 | C |
| ATOM | 2415 | CG2 | VAL | B | 155 | 31.694 | 30.398 | 19.640 | 1.00 | 59.92 | C |
| ATOM | 2416 | C | VAL | B | 155 | 32.774 | 26.725 | 19.473 | 1.00 | 56.32 | C |
| ATOM | 2417 | O | VAL | B | 155 | 32.358 | 26.102 | 20.454 | 1.00 | 55.79 | O |
| ATOM | 2418 | N | CYS | B | 156 | 33.067 | 26.126 | 18.319 | 1.00 | 54.93 | N |
| ATOM | 2419 | CA | CYS | B | 156 | 33.049 | 24.667 | 18.173 | 1.00 | 54.61 | C |
| ATOM | 2420 | CB | CYS | B | 156 | 33.494 | 24.270 | 16.754 | 1.00 | 52.18 | C |
| ATOM | 2421 | SG | CYS | B | 156 | 33.580 | 22.483 | 16.410 | 1.00 | 54.42 | S |
| ATOM | 2422 | C | CYS | B | 156 | 33.909 | 23.990 | 19.278 | 1.00 | 54.64 | C |
| ATOM | 2423 | O | CYS | B | 156 | 33.407 | 23.164 | 20.052 | 1.00 | 54.63 | O |
| ATOM | 2424 | N | GLN | B | 157 | 35.187 | 24.360 | 19.371 | 1.00 | 55.54 | N |
| ATOM | 2425 | CA | GLN | B | 157 | 36.065 | 23.825 | 20.426 | 1.00 | 56.56 | C |
| ATOM | 2426 | CB | GLN | B | 157 | 37.535 | 23.863 | 19.983 | 1.00 | 56.95 | C |
| ATOM | 2427 | C | GLN | B | 157 | 35.892 | 24.528 | 21.791 | 1.00 | 56.30 | C |
| ATOM | 2428 | O | GLN | B | 157 | 35.845 | 23.877 | 22.843 | 1.00 | 57.99 | O |
| ATOM | 2429 | N | THR | B | 158 | 35.780 | 25.856 | 21.755 | 1.00 | 54.56 | N |
| ATOM | 2430 | CA | THR | B | 158 | 35.832 | 26.703 | 22.947 | 1.00 | 51.87 | C |
| ATOM | 2431 | CB | THR | B | 158 | 36.082 | 28.197 | 22.528 | 1.00 | 53.75 | C |
| ATOM | 2432 | OG1 | THR | B | 158 | 37.496 | 28.431 | 22.411 | 1.00 | 49.36 | O |
| ATOM | 2433 | CG2 | THR | B | 158 | 35.470 | 29.201 | 23.512 | 1.00 | 52.72 | C |
| ATOM | 2434 | C | THR | B | 158 | 34.632 | 26.566 | 23.900 | 1.00 | 51.82 | C |
| ATOM | 2435 | O | THR | B | 158 | 34.800 | 26.579 | 25.128 | 1.00 | 52.22 | O |
| ATOM | 2436 | N | ARG | B | 159 | 33.441 | 26.403 | 23.335 | 1.00 | 49.63 | N |
| ATOM | 2437 | CA | ARG | B | 159 | 32.187 | 26.495 | 24.078 | 1.00 | 49.75 | C |
| ATOM | 2438 | CB | ARG | B | 159 | 31.544 | 27.873 | 23.850 | 1.00 | 47.71 | C |
| ATOM | 2439 | C | ARG | B | 159 | 31.223 | 25.388 | 23.657 | 1.00 | 50.64 | C |
| ATOM | 2440 | O | ARG | B | 159 | 30.016 | 25.478 | 23.906 | 1.00 | 52.26 | O |
| ATOM | 2441 | N | ALA | B | 160 | 31.780 | 24.338 | 23.045 | 1.00 | 49.93 | N |
| ATOM | 2442 | CA | ALA | B | 160 | 31.031 | 23.199 | 22.492 | 1.00 | 48.16 | C |
| ATOM | 2443 | CB | ALA | B | 160 | 30.681 | 22.213 | 23.589 | 1.00 | 47.09 | C |
| ATOM | 2444 | C | ALA | B | 160 | 29.781 | 23.593 | 21.693 | 1.00 | 47.13 | C |
| ATOM | 2445 | O | ALA | B | 160 | 28.729 | 22.967 | 21.837 | 1.00 | 46.20 | O |
| ATOM | 2446 | N | MET | B | 161 | 29.897 | 24.650 | 20.888 | 1.00 | 48.64 | N |
| ATOM | 2447 | CA | MET | B | 161 | 28.791 | 25.123 | 20.060 | 1.00 | 50.67 | C |
| ATOM | 2448 | CB | MET | B | 161 | 28.928 | 26.603 | 19.706 | 1.00 | 52.39 | C |
| ATOM | 2449 | CG | MET | B | 161 | 28.155 | 27.506 | 20.613 | 1.00 | 51.45 | C |
| ATOM | 2450 | SD | MET | B | 161 | 27.427 | 28.867 | 19.687 | 1.00 | 55.07 | S |
| ATOM | 2451 | CE | MET | B | 161 | 26.010 | 29.168 | 20.740 | 1.00 | 54.49 | C |
| ATOM | 2452 | C | MET | B | 161 | 28.658 | 24.300 | 18.803 | 1.00 | 49.34 | C |
| ATOM | 2453 | O | MET | B | 161 | 29.151 | 24.659 | 17.730 | 1.00 | 48.03 | O |
| ATOM | 2454 | N | LYS | B | 162 | 27.994 | 23.170 | 18.953 | 1.00 | 49.25 | N |
| ATOM | 2455 | CA | LYS | B | 162 | 27.692 | 22.303 | 17.820 | 1.00 | 48.55 | C |
| ATOM | 2456 | CB | LYS | B | 162 | 28.860 | 21.354 | 17.556 | 1.00 | 51.62 | C |
| ATOM | 2457 | CG | LYS | B | 162 | 29.575 | 20.893 | 18.800 | 1.00 | 49.28 | C |
| ATOM | 2458 | CD | LYS | B | 162 | 31.087 | 20.929 | 18.634 | 1.00 | 50.58 | C |
| ATOM | 2459 | CE | LYS | B | 162 | 31.753 | 20.166 | 19.772 | 1.00 | 50.05 | C |
| ATOM | 2460 | NZ | LYS | B | 162 | 33.254 | 20.284 | 19.818 | 1.00 | 49.37 | N |
| ATOM | 2461 | C | LYS | B | 162 | 26.373 | 21.547 | 18.060 | 1.00 | 49.72 | C |
| ATOM | 2462 | O | LYS | B | 162 | 25.916 | 21.410 | 19.222 | 1.00 | 43.36 | O |
| ATOM | 2463 | N | ILE | B | 163 | 25.736 | 21.121 | 16.963 | 1.00 | 47.99 | N |
| ATOM | 2464 | CA | ILE | B | 163 | 24.568 | 20.251 | 17.040 | 1.00 | 47.17 | C |
| ATOM | 2465 | CB | ILE | B | 163 | 23.306 | 20.835 | 16.329 | 1.00 | 46.99 | C |
| ATOM | 2466 | CG1 | ILE | B | 163 | 22.952 | 22.213 | 16.880 | 1.00 | 46.43 | C |
| ATOM | 2467 | CD1 | ILE | B | 163 | 21.965 | 22.944 | 16.019 | 1.00 | 56.53 | C |
| ATOM | 2468 | CG2 | ILE | B | 163 | 22.082 | 19.898 | 16.490 | 1.00 | 43.12 | C |
| ATOM | 2469 | C | ILE | B | 163 | 24.929 | 18.931 | 16.392 | 1.00 | 47.16 | C |
| ATOM | 2470 | O | ILE | B | 163 | 25.505 | 18.905 | 15.299 | 1.00 | 47.77 | O |
| ATOM | 2471 | N | LEU | B | 164 | 24.586 | 17.844 | 17.085 | 1.00 | 47.94 | N |
| ATOM | 2472 | CA | LEU | B | 164 | 24.639 | 16.492 | 16.538 | 1.00 | 47.40 | C |
| ATOM | 2473 | CB | LEU | B | 164 | 25.458 | 15.602 | 17.485 | 1.00 | 48.90 | C |
| ATOM | 2474 | CG | LEU | B | 164 | 25.515 | 14.084 | 17.312 | 1.00 | 48.11 | C |
| ATOM | 2475 | CD1 | LEU | B | 164 | 25.891 | 13.674 | 15.901 | 1.00 | 52.59 | C |
| ATOM | 2476 | CD2 | LEU | B | 164 | 26.497 | 13.532 | 18.324 | 1.00 | 46.10 | C |
| ATOM | 2477 | C | LEU | B | 164 | 23.210 | 15.947 | 16.335 | 1.00 | 48.56 | C |
| ATOM | 2478 | O | LEU | B | 164 | 22.455 | 15.805 | 17.301 | 1.00 | 47.48 | O |
| ATOM | 2479 | N | MET | B | 165 | 22.836 | 15.666 | 15.078 | 1.00 | 49.17 | N |
| ATOM | 2480 | CA | MET | B | 165 | 21.500 | 15.120 | 14.757 | 1.00 | 48.85 | C |
| ATOM | 2481 | CB | MET | B | 165 | 20.840 | 15.899 | 13.620 | 1.00 | 48.68 | C |
| ATOM | 2482 | CG | MET | B | 165 | 20.442 | 17.324 | 14.038 | 1.00 | 51.96 | C |
| ATOM | 2483 | SD | MET | B | 165 | 20.732 | 18.576 | 12.780 | 1.00 | 52.67 | S |
| ATOM | 2484 | CE | MET | B | 165 | 22.520 | 18.375 | 12.593 | 1.00 | 54.06 | C |
| ATOM | 2485 | C | MET | B | 165 | 21.509 | 13.624 | 14.460 | 1.00 | 47.92 | C |
| ATOM | 2486 | O | MET | B | 165 | 22.112 | 13.162 | 13.481 | 1.00 | 43.94 | O |
| ATOM | 2487 | N | LYS | B | 166 | 20.820 | 12.893 | 15.332 | 1.00 | 49.57 | N |
| ATOM | 2488 | CA | LYS | B | 166 | 20.791 | 11.437 | 15.337 | 1.00 | 50.86 | C |
| ATOM | 2489 | CB | LYS | B | 166 | 21.118 | 10.881 | 16.748 | 1.00 | 50.14 | C |
| ATOM | 2490 | CG | LYS | B | 166 | 22.499 | 11.269 | 17.311 | 1.00 | 44.29 | C |
| ATOM | 2491 | CD | LYS | B | 166 | 22.993 | 10.265 | 18.367 | 1.00 | 51.13 | C |
| ATOM | 2492 | CE | LYS | B | 166 | 24.398 | 9.728 | 18.020 | 1.00 | 44.96 | C |
| ATOM | 2493 | NZ | LYS | B | 166 | 25.262 | 9.450 | 19.224 | 1.00 | 51.45 | N |
| ATOM | 2494 | C | LYS | B | 166 | 19.437 | 10.891 | 14.869 | 1.00 | 52.27 | C |
| ATOM | 2495 | O | LYS | B | 166 | 18.388 | 11.185 | 15.452 | 1.00 | 49.95 | O |
| ATOM | 2496 | N | VAL | B | 167 | 19.466 | 10.116 | 13.793 | 1.00 | 53.68 | N |
| ATOM | 2497 | CA | VAL | B | 167 | 18.484 | 9.043 | 13.659 | 1.00 | 55.69 | C |
| ATOM | 2498 | CB | VAL | B | 167 | 17.754 | 8.987 | 12.300 | 1.00 | 53.78 | C |
| ATOM | 2499 | CG1 | VAL | B | 167 | 16.393 | 8.309 | 12.485 | 1.00 | 51.87 | C |
| ATOM | 2500 | CG2 | VAL | B | 167 | 17.564 | 10.378 | 11.706 | 1.00 | 60.81 | C |
| ATOM | 2501 | C | VAL | B | 167 | 19.221 | 7.728 | 13.947 | 1.00 | 56.53 | C |
| ATOM | 2502 | O | VAL | B | 167 | 20.288 | 7.731 | 14.582 | 1.00 | 55.95 | O |
| ATOM | 2503 | O | HOH | C | 1 | −1.068 | 19.700 | −2.247 | 1.00 | 20.00 | O |
| ATOM | 2504 | O | HOH | D | 2 | 15.644 | 38.458 | −11.327 | 1.00 | 20.00 | O |
| ATOM | 2505 | O | HOH | D | 3 | −8.095 | 49.449 | −9.306 | 1.00 | 20.00 | O |
| ATOM | 2506 | O | HOH | D | 4 | 14.879 | 14.522 | 19.283 | 1.00 | 20.00 | O |
| ATOM | 2507 | O | HOH | D | 5 | 19.086 | 43.782 | 7.312 | 1.00 | 20.00 | O |
| ATOM | 2508 | O | HOH | D | 6 | 3.762 | 36.483 | −0.689 | 1.00 | 20.00 | O |
| ATOM | 2509 | O | HOH | D | 7 | 5.977 | 28.240 | −23.712 | 1.00 | 20.00 | O |
| ATOM | 2510 | O | HOH | D | 9 | 38.280 | 30.933 | 15.129 | 1.00 | 20.00 | O |
| ATOM | 2511 | O | HOH | D | 10 | −2.614 | 8.973 | −12.882 | 1.00 | 20.00 | O |
| ATOM | 2512 | O | HOH | D | 11 | 33.557 | 12.755 | 6.451 | 1.00 | 20.00 | O |
| ATOM | 2513 | O | HOH | D | 12 | 13.880 | 42.897 | 11.851 | 1.00 | 20.00 | O |
| ATOM | 2514 | O | HOH | D | 13 | 19.942 | 29.901 | −7.927 | 1.00 | 20.00 | O |
| ATOM | 2515 | O | HOH | D | 14 | 20.613 | 48.185 | 26.172 | 1.00 | 20.00 | O |
| ATOM | 2516 | O | HOH | D | 15 | 23.384 | 36.996 | 17.915 | 1.00 | 20.00 | O |
| ATOM | 2517 | O | HOH | D | 16 | 12.029 | 8.173 | 11.014 | 1.00 | 20.00 | O |
| ATOM | 2518 | O | HOH | D | 18 | 17.554 | 21.123 | −23.737 | 1.00 | 20.00 | O |
| ATOM | 2519 | O | HOH | D | 19 | 34.755 | 13.969 | 18.956 | 1.00 | 20.00 | O |
| ATOM | 2520 | O | HOH | D | 20 | 19.454 | 17.871 | −19.578 | 1.00 | 20.00 | O |
| ATOM | 2521 | O | HOH | D | 21 | 16.241 | 30.564 | 5.560 | 1.00 | 20.00 | O |
| ATOM | 2522 | O | HOH | D | 22 | 4.906 | 11.442 | 0.110 | 1.00 | 20.00 | O |
| ATOM | 2523 | O | HOH | D | 23 | 0.407 | 21.483 | −3.061 | 1.00 | 20.00 | O |
| ATOM | 2524 | O | HOH | D | 24 | 11.613 | 15.180 | −2.107 | 1.00 | 20.00 | O |
| ATOM | 2525 | O | HOH | D | 25 | 3.322 | 26.535 | 2.926 | 1.00 | 20.00 | O |
| ATOM | 2526 | O | HOH | D | 26 | 0.515 | 33.846 | 2.133 | 1.00 | 20.00 | O |
| ATOM | 2527 | O | HOH | D | 27 | 20.755 | 10.411 | 1.428 | 1.00 | 20.00 | O |
| ATOM | 2528 | O | HOH | D | 28 | 17.637 | 11.370 | −10.183 | 1.00 | 20.00 | O |
| ATOM | 2529 | O | HOH | D | 29 | 25.321 | 5.436 | 7.341 | 1.00 | 20.00 | O |
| ATOM | 2530 | O | HOH | D | 30 | 12.205 | 9.020 | −7.002 | 1.00 | 20.00 | O |
| ATOM | 2531 | O | HOH | D | 31 | 10.319 | 15.291 | −12.878 | 1.00 | 20.00 | O |
| ATOM | 2532 | O | HOH | D | 32 | 13.474 | 26.530 | 21.387 | 1.00 | 20.00 | O |
| ATOM | 2533 | O | HOH | D | 33 | −5.385 | 36.132 | 3.825 | 1.00 | 20.00 | O |
| ATOM | 2534 | O | HOH | D | 34 | −12.238 | 40.692 | −6.393 | 1.00 | 20.00 | O |
| ATOM | 2535 | O | HOH | D | 35 | −7.910 | 35.441 | 3.507 | 1.00 | 20.00 | O |
| ATOM | 2536 | O | HOH | D | 36 | 16.595 | 21.733 | 4.395 | 1.00 | 20.00 | O |
| ATOM | 2537 | O | HOH | D | 37 | 6.286 | 16.055 | −2.458 | 1.00 | 20.00 | O |
| ATOM | 2538 | O | HOH | D | 38 | 5.079 | 15.735 | −7.772 | 1.00 | 20.00 | O |
| ATOM | 2539 | O | HOH | D | 39 | 0.584 | 7.759 | −10.414 | 1.00 | 20.00 | O |
| ATOM | 2540 | O | HOH | D | 40 | 11.416 | 24.606 | 13.912 | 1.00 | 20.00 | O |
| ATOM | 2541 | O | HOH | D | 43 | 9.303 | 12.604 | −3.017 | 1.00 | 20.00 | O |
| ATOM | 2542 | O | HOH | D | 45 | 28.991 | 19.384 | 1.268 | 1.00 | 20.00 | O |
| ATOM | 2543 | O | HOH | D | 46 | 21.356 | 43.725 | 10.404 | 1.00 | 20.00 | O |
| ATOM | 2544 | O | HOH | D | 49 | −12.300 | 34.162 | −0.864 | 1.00 | 20.00 | O |
| END | | | | | | | | | | | |

TABLE 2
Crystallographic Statistics for the EphB4-ephrinB2 Complex

| | |
|---|---|
| Resolution (Å)¹ | 20-2.0 (2.1-2.0) |
| Wavelength (Å) | 0.98 |
| Space Group | P4₁ |
| Unit Cell Dimensions (Å) | a = b = 81.09 81.09 c = 50.95 |
| Completeness (%) | 99.6 (99.9) |
| R_{sym} (%)² | 3.9 (20.8) |
| l/σ | 4.8 |
| Mean Redundancy | 4.7 |
| No. Reflections | 19,785 |
| R_{cryst} (%)³ | 22.6 (26.5) |
| R_{free} (%)⁴ | 29.5 (30.0) |
| R.m.s. deviations | |
| Bond length (Å) | 0.02 |
| Bond angle (°) | 1.7 |
| Average B factor (Å²) | 56.6 |
| Number of atoms | |
| Protein | 4,992 |

¹Number in parentheses is for the highest shell.
²R_{sym} = Σ|I − <I>|/ΣI, where I is the observed intensity and <I> is the average intensity of multiple symmetry-related observations of that reflection.
³R_{cryst} = Σ||F_{obs}| − |F_{calc}||/Σ|F_{obs}|, where F_{obs} and F_{calc} are the observed and calculated structure factors. R_{sym} = Σ|I − <I>|/ΣI, where I is the observed intensity and <I> is the average intensity of multiple symmetry-related observations of that reflection.
⁴R_{free} = Σ||F_{obs}| − |F_{calc}||/Σ|F_{obs}| for 10% of the data not used at any stage of structural refinement.

TABLE 3
Effects of EphB4 mutation on binding to Alexa-532-TNYL-RAW

| EphB4 mutant | Kd, nM |
|---|---|
| WT | 5 ± 0.9 |
| L95R | ND* |
| T147F | 25 ± 5.6 |
| R148S | 4.5 ± 0.7 |
| K149Q | 23 ± 8 |
| R150V | 6.1 ± 0.8 |
| RKR148-150SQV | 21 ± 6 |
| A186S | 16 ± 4 |

*FP window is not significant to accurately determine Kd

TABLE 4
Binding of peptide (TNYL-RAW) and human ephrinB2 to the human EphB4
ephrin-binding domain and EphB4 mutants. Experiments were performed at
25° C. in 50 mM Tris pH 7.8, 150 mM NaCl, 1 mM CaCl₂.
All values represent the average of at least two experiments.

| | | Kd | ΔG | ΔH | TΔS |
|---|---|---|---|---|---|
| Receptor | Ligand | (nM) | (kcal mol−1) | (kcal mol−1) | (kcal mol−1) |
| EphB4 (wt) | ephrinB2 | 40 ± 20 | −10.2 ± 0.3 | 3.3 ± 0.1 | 13.4 ± 0.4 |
| EphB4 | ephrinB2 | 20 ± 10 | −10.6 ± 0.4 | 3.7 ± 0.2 | 14.4 ± 0.3 |
| (K149Q) | | | | | |
| EphB4 | ephrinB2 | 1900 ± 1100 | −7.8 ± 0.3 | 5.2 ± 0.7 | 13.0 ± 0.8 |
| (L95R) | | | | | |
| EphB4 (wt) | TNYL-RAW | 71 ± 14 | −9.8 ± 0.1 | −14.7 ± 0.2 | −4.9 ± 0.2 |
| EphB4 | TNYL-RAW | 160 ± 120 | −9.4 ± 0.5 | −12.5 ± 1.2 | −3.2 ± 1.3 |
| (K149Q) | | | | | |
| EphB4 | TNYL-RAW | 35900 ± 5000 | −6.1 ± 0.1 | −12.0 ± 0.3 | −5.8 ± 0.4 |
| (L95R) | | | | | |

### EXAMPLES

Aspects of the present teachings may be further understood in light of the following examples, which should not be construed as limiting the scope of the present teachings in any way.

### Example 1

### Construct Design, Expression and Purification of EphB4

Twelve sequential 4 amino acid truncations in human EphB4 were designed based on EphB4-EphB2 sequence alignment in the region C-terminal to the last β-strand in the EphB2 structure. The resulting fragments were cloned into the insect cell expression vector pBAC6 (Novagen, Wis.) under control of the heterologous GP64 signal peptide and containing a N-terminal six histidine tag. Constructs were sequence verified, and baculovirus was generated using homologous recombination into Sapphire Baculovirus DNA (Orbigen, Calif.) using the manufacturers protocol. After 3 rounds of viral amplification, a small scale expression screen was conducted for all constructs in both Sf9 and Hi5 insect cells. Briefly, 5×10⁶ cells were infected with baculovirus at an MOI of 2 in 38 mm tissue culture dishes; cells were harvested at 48 hours post infection and supernatant containing secreted EphB4 was concentrated 10-fold and buffer exchanged into 50 mM Tris pH 7.8, 400 mM NaCl, and 5 mM imidazole using an Amicon Ultra 5K concentrator (Millipore, MS). The secreted protein was bound to Ni-NTA magnetic beads (Qiagen, CA), washed with 50 mM Tris pH 7.8, 400 mM NaCl, 20 mM Imidazole buffer and eluted with 50 mM Tris pH 7.8, 400 mM NaCl, 250 mM Imidazole. Based on analysis of immobilized metal affinity chromatography (IMAC) elutes, the EphB4 (17-196) construct was identified as the highest expressor at ˜6 mg/L in Hi5 insect cells. Large scale expression was conducted using Wave Bioreactors (Wave Biotech LLC, NJ) at a MOI of 2 for 48 hours in Hi5 insect cells. Media containing secreted EphB4 was concentrated and buffer exchanged using a Hydrosart Crossflow filter (Sartorius, NY). Following IMAC purification on ProBond resin (Invitrogen, CA) as described above, EphB4 was concentrated to 5 mg/ml and loaded on a Superdex 75 16/60 column (GE HealthCare, N.Y.). A small amount of aggregated material was removed by preparative size exclusion chromatography, while most of the sample eluted in a single peak corresponding to an EphB4 (17-196) monomer. The complete removal of the GP64 secretion sequence and protein identity were confirmed by MALDI analysis.

The wtEphB4 construct was used as a template for the generation of site specific mutants. The human ephrinB2 (extracellular domain; residues 25-187) was designed based on the previously published EphB2-ephrinB2 structure and cloned into a modified pFastBac1 vector containing a GP67 leader peptide. Recombinant baculovirus was generated using the Bac-to-Bac system (Invitrogen, CA). Briefly, large scale expression of ephrinB2 was carried out using Wave Bioreactors on a 5 L scale at an MOI of 5 for 48 hr. resulting in 10 mg of ephrinB2 per liter of Hi-5 insect cells (Invitrogen, CA). Media containing secreted ephrinB2 protein was concentrated and buffer exchanged using a Hydrostart Crossflow Filter (Sartorius Edgewood, N.Y.). The ligand was purified by immobilized metal affinity chromatography (IMAC), and cleaved overnight with TEV protease. Material was further re-purified by IMAC chromatography to remove the protease and an N-terminal fragment containing the histidine tag. The EphB4-ephrinB2 complex was formed with a 1.5-fold molar excess of ephrinB2 overnight at 4° C. in buffer containing 50 mM Tris, pH 7.8, 100 mM NaCl, and 10 mM Imidazole. The complex was purified by IMAC chromatography, followed by size exclusion chromatography to remove trace aggregates (Phenomenex S2000).

### Example 2

### Crystallization, Data Collection, and Structure Solution

The EphB4-ephrinB2 complex was concentrated to 20 mg/mL and crystallized by sitting drop vapor diffusion at 20° C. against a precipitant of 2.2 M ammonium sulfate and 100 mM tris, pH 7.8. Crystals formed in the P4₁ spacegroup and contained one monomer of receptor and one monomer of ligand in the asymmetric unit. Data were collected at the Advance Photon Source (Argonne, IL) on beamline GM/CA-CAT. Images were processed and reduced using HKL2000 (31). The structure was solved by molecular replacement with MolRep (CCP4i), using the EphB2-ephrinB2 structure (PDB: 1 KGY) as a search model (10,32). The structure was refined by a rigid body refinement, followed by model building in 0 and iterative refinements with refmac (32,33). The structure exhibits good geometry with no Ramachandran outliers.

### Example 3

### Isothermal Titration Calorimetry

All mutants and ligands were dialyzed into buffer containing 50 mM Tris-Cl (pH 7.8), 150 mM NaCl, and 1 mM CaCl₂ prior to use in isothermal titration calorimetry (ITC) experiments. All experiments were performed with a Microcal MCS ITC at 25° C. Titrations were completed as described in Example 4. EphB4 (wild-type or mutant) was present in the sample cell at a concentration of 12 to 15 μM and the injection syringe contained either 127 μM ephrinB2 or 200 μM TNYL-RAW. Titrations of TNYL-RAW with the L95R mutant of EphB4 were performed with 2 mM TNYL-RAW in the injection syringe and 15 μM EphB4 (L95R) in the sample cell. Data for these titrations were fit assuming a stoichiometry of 1 and at least 60% saturation at the final peptide concentration as described (19,34).

### Example 4

Isothermal titration calorimetry and ELISA experiments: EphB4 and ephrin-B2 were either dialyzed or buffer exchanged into 50 mM Tris-Cl (pH 7.8 at 25° C.), 150 mM NaCl, 1 mM CaCl₂, prior to use in calorimetry experiments. Peptides were dissolved into the same buffer used for the dialysis of EphB4. The concentration of EphB4, ephrin-B2 and the peptides was determined by measuring the A₂₈₀ and using the theoretical extinction coefficient (Gill and von Hippel, 1989). ITC experiments were performed with a Microcal MCS ITC at 25° C. Following an initial injection of 2 μl, titrations were performed by making 20 13 μl injections of peptide into EphB4 in the sample cell to produce an approximate final 2:1 ratio of injectant to sample in the cell. For most titrations the sample cell contained 15 μM EphB4 and the injection syringe contained a 200 μM solution of the peptide. Titrations with ephrin-B2 contained 13 μM EphB4 in the sample cell and 290 μM ephrin-B2 in the syringe. Prior to loading the sample cell, EphB4 was centrifuged at 18,000 g for 5 min at 4° C. to remove aggregates and degassed for 5 minutes at room temperature. Corrections for heats of dilution for the peptides and ephrin-B2 were determined by performing titrations of peptide or ephrin-B2 solutions into buffer. Dilution data were fit to a line and subtracted from the corresponding titration data. Titration data were analyzed using Origin ITC software (Version 5.0, Microcal Software Inc.) and curves were fit to a single binding site model (Wiseman et al., 1989). The low affinity of the TNYL peptide and the limited availability of EphB4 (17-196) precluded accurate determination of the K_{d} for this interaction by ITC. A lower limit for the binding constant was determined by performing a titration in which the sample cell contained 30 μM EphB4 and the injection syringe contained a 1.45 mM solution of the peptide, producing a final ratio of peptide to EphB4 of 10:1. The data was fit assuming a stoichiometry of 1 and at least 60% saturation of binding at the final peptide concentration (Turnbull and Daranas, 2003).

The ability of peptides to compete the binding of mouse ephrin-B2 alkaline phosphatase to immobilized mouse EphB4-Fc-His (R&D Systems) was measured by ELISA as previously described (Koolpe et al., 2005).

### Example 5

This Example illustrates fluorescence polarization (FP) assays using a fluorescently-labeled reporter peptide to measure binding of various ligands to the EphB4-LBD.

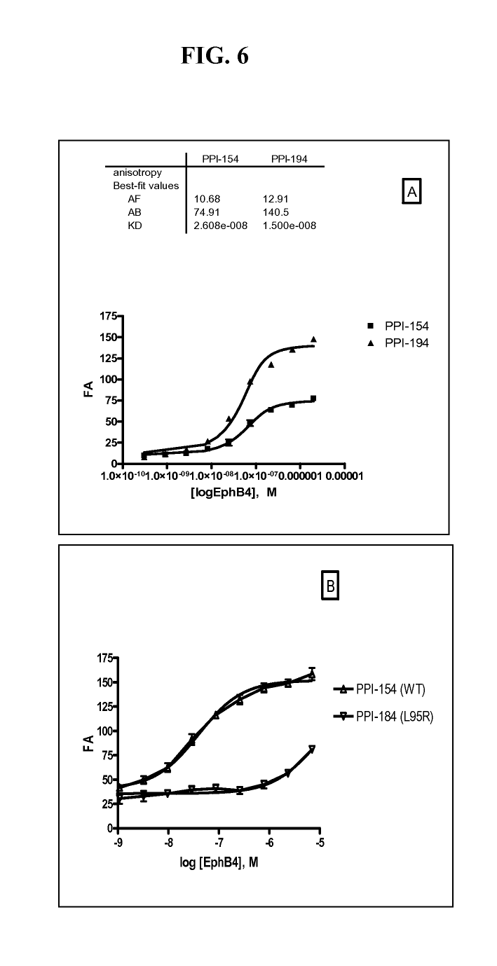

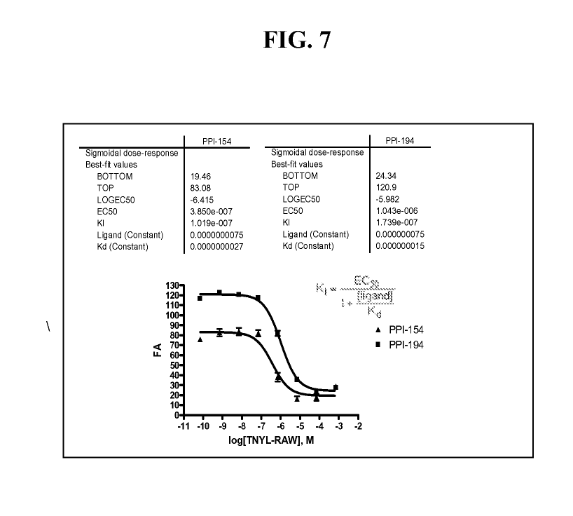

We have evaluated TMR and Alexa-532 labeled peptides, and experimentally confirmed the preference of Alexa-532-TNYL-RAW peptide for the assay because of the better dynamic range. We have also evaluated mutants predicted to have altered binding affinity to the TNYL-RAW peptide based on structural observations. The dose-response curve in FIG. 6A shows the wild-type EphB4 and EphB4 K149Q mutant signal upon binding to labeled TNYL-RAW peptide. AK149Q mutant has a greater dynamic range and slightly lower affinity for the labeled peptide than wtEphB4. In competition experiments, the affinity for the TNYL-RAW peptide is 170 nM, which is slightly lower than for wtEphB4 (100 nM) (FIG. 7). Without being bound by a particular theory, a better dynamic range is likely a result of the interaction of this specific mutant with the Alexa-532 fluorophore of the reporter peptide. These characteristics make it an attractive tool for high throughput screening. The assay windows are approximately 6-fold for wtEphB4 and 12-fold for the EphB4 (K149Q) mutant. In addition we have validated the assay by studying binding of an L95R mutant, which was shown to have Kdephrin-B2=2 uM by ITC analysis. We have not detected a significant signal in FP analysis to accurately calculate KdAlexa-TNYL-RAW (FIG. 6B). This analysis also further validates the use of labeled TNYL-RAW peptide as a surrogate ligand for studies of ephrin-B2-EphB4 binding.

In this and other examples involving Alexa-532-TNYL-RAW peptide, A serial dilution of EphB4 was prepared in Assay Buffer (50 mM Tris pH 7.8, 150 mM NaCl, 1 mM CalCl2, 0.1% Pluronic 124). TNYL-RAW-Alexa-532 labeled peptide was prepared as a 100 μM stock solution in the Assay Buffer and a 300 nM working solution was made fresh prior to the measurements by dilution in the assay buffer. 5 μL of serially diluted EphB4 (9 nM-2362 nM concentration range) was combined with 5 μL of labeled peptide (final concentration 75 nM) in the final volume of 20 μL (Assay plate, 384 well flat bottom, black polystyrene, non-binding surface, Corning, cat #3654) in the absence and presence of 200 μM TNYL-RAW as a control for non-specific binding. The mixture was allowed to equilibrate for 30 min at room temperature, and measurements were performed with a Tecan Genios Pro (Tecan Instruments) using 535 nm excitation and 580 nm emission wavelength. All experimental data were analyzed using Prism 4.0 software (GraphPad Software Inc., San Diego, Calif.) and Kd values were generated by fitting the experimental data using a one-site binding hyperbola nonlinear regression model or equation 8.10 (.www.invitrogen.com/downloads/FP8.pdf).

In these experiments, the human EphB4 (17-196) ligand binding domain was cloned into the insect cell expression vector pBAC6 (Novagen, San Diego, Calif.) under control of the heterologous GP64 signal peptide and containing an N-terminal six histidine tag. The construct was sequence verified, and baculovirus was generated with homologous recombination into Sapphire Baculovirus DNA (Orbigen, San Diego, Calif.) following the manufacturer's protocol (10). The wtEphB4 construct was used as a template for generation of site specific mutants.

Large-scale expression was conducted with Wave Bioreactors (Wave Biotech LLC, Somerset, N.J.) at an MOI of 2 for 48 hr in Hi5 insect cells. Media containing secreted EphB4 (17-196) was concentrated and buffer exchanged with a Hydrosart Crossflow filter (Sartorius, Edgewood, N.Y.). Following immobilized metal affinity chromatography (IMAC) purification on ProBond resin (Invitrogen, Carlsbad, Calif.), EphB4 was concentrated to 5 mg/ml and loaded on a Superdex 75 16/60 column (GE HealthCare, Chicago, Ill.). A small amount of aggregated material was removed by preparative size exclusion chromatography, while most of the sample eluted in a single peak corresponding to an EphB4 (17-196) monomer. The complete removal of the GP64 secretion sequence and protein identity were confirmed by MALDI analysis.

The TNYL-RAW peptide was labeled with Alexa-532 (Biopeptides Inc., San Diego, Calif.). All peptides are purified to >95% purity, and supplied with rigorous analytical specifications, including HPLC and MS analysis.

### Example 6

This Example illustrates that the fluorescence polarization assay (Example 5) is tolerant of organic solvents.

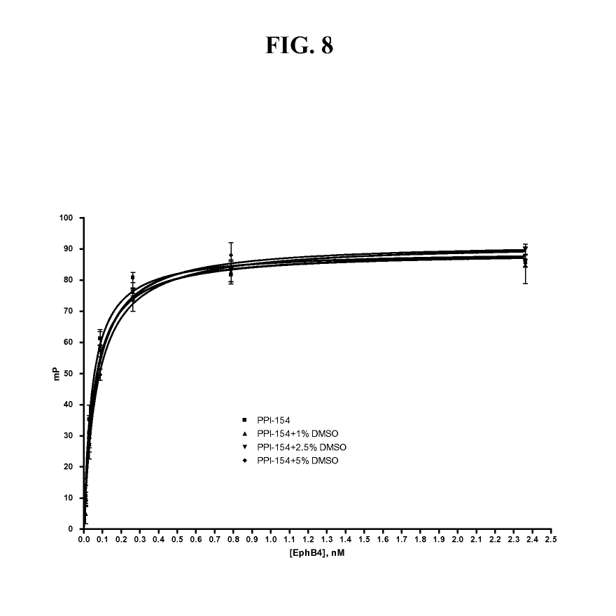

In these experiments, dimethylsulfoxide (DMSO) was added in various concentrations to a solution comprising Eph4 and Alexa-532-TNYL-RAW peptide, and FP was measured. As shown in FIG. 8, the FP assay is tolerant to 5% DMSO (filled diamonds in FIG. 8) as indicated by analysis of EphB4 binding in the presence of various concentrations of DMSO. We have also been successful in the crystallization of EphB4 with the TNYL-RAW peptide in the presence of 5% DMSO, which is indicative of excellent tolerance of this specific interaction to DMSO.

### Example 7

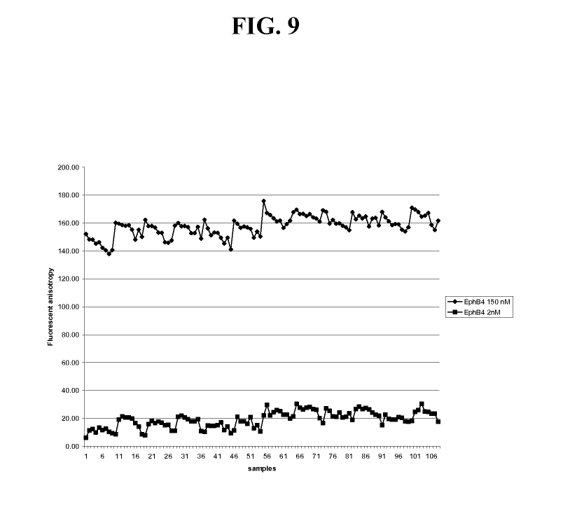

This Example illustrates determination of Z-factor at protein concentrations representing upper and lower plateaus of the dose response curve for the EphB4 K149Q mutant (FIG. 6A). The calculated Z-factor for 108 samples, each at 2 different protein concentrations, is 0.715 (FIG. 9). The range of Z-factor between 0.5 and 1 is considered to be representative of a high quality assay.

### Example 8

This Example illustrates thermodynamic characterization of TNYL-RAW peptide binding to EphB4-ligand binding domain (EphB4-LBD).

In these experiments, we monitored the binding of EphB4-LBD to ephrin-B2 and peptide ligands using isothermal titration calorimetry (ITC). The interaction between EphB4 (17-196) and ephrin-B2 yielded a Kd of 40 nM and a ΔHo of +3.3 kcal mol-1. This is slightly lower than the affinity reported for the interaction between the entire mouse EphB4 extracellular domain and mouse or human ephrin-B2. Without being limited by theory, we hypothesize that the difference may be explained by the existence of a third low affinity Eph-ephrin interface located outside the ephrin-binding domain (Smith, F. M., et al., J. Biol. Chem. 279: 9522-9531, 2004. In addition, N- and C-terminal truncations of the peptide, as well as targeted mutations in the center of the peptide, were synthesized in order to biophysically validate individual effects of the peptide upon EphB4 binding. Table 5 presents data of a thermodynamic analysis of wtEphB4 and mutant EphB4 binding to ephrin-B2 and TNYL-RAW and related peptides. The table shows the results of isothermal titration calorimetry (ITC) analysis. The Kd values reported from this method compare well with the Kd values determined from the FP assays (FIGS. 6 and 7). Three regions of interactions proved critical for receptor binding: The N-terminal Tyr, the Phe/IIe amino acids in the center of the peptide, and the high-affinity C-terminal RAW sequence. The N- and C-terminal truncations appear detrimental due to the loss of stability at the D-E (N-terminal) and J-K (C-terminal) loops, while the Phe/IIe mutations resulted in a loss of stability at an imperative disulfide bridge critical to EphB4 LBD stability.

TABLE 5

| | | | ΔG (kcal | | TΔS (kcal |
|---|---|---|---|---|---|
| Receptor | Ligand | Kd (nM) | mol−1) | ΔH (koal mol−1) | mol−1) |
| EphB4 (wt) | ephrin-B2 | 40 ± 20 | −10.2 ± 0.3 | 3.3 ± 0.1 | 13.4 ± 0.4 |
| EphB4 | ephrin-B2 | 20 ± 10 | −10.5 ± 0.4 | 3.6 ± 0.1 | 14.1 ± 0.4 |
| (K149Q) | | | | | |
| EphB4 | ephrin-B2 | 1900 ± 1100 | −7.8 ± 0.3 | 5.2 ± 0.7 | 13.0 ± 0.8 |
| (L95R) | | | | | |
| EphB4 (wt) | TNYL-RAW | 71 ± 14 | −9.8 ± 0.1 | −14.7 ± 0.2 | −4.9 ± 0.2 |
| EphB4 | TNYL-RAW | 250 ± 50 | −9.0 ± 0.1 | −11.7 ± 0.2 | −2.7 ± 0.2 |
| (K149Q) | | | | | |
| EphB4 (wt) | NYLF-RAW | 65 ± 7 | −9.8 ± 0.1 | −15.5 ± 0.1 | −5.7 ± 0.1 |
| EphB4 (wt) | YLFS-RAW | 80 ± 36 | −9.7 ± 0.2 | −13.8 ± 0.5 | −4.1 ± 0.4 |
| EphB4 (wt) | LFSP-RAW | 3,500 ± 680 | −7.4 ± 0.1 | −5.3 ± 0.5 | 2.1 ± 0.4 |
| EphB4 (wt) | TNYL | ≧140,000 | ND | −9.6 ± 0.3 | ND |
| EphB4 (wt) | LFSP-RAW(F to | ≧500,000 | ND | −7.9 ± 0.9 | ND |
| | A) | | | | |
| EphB4 (wt) | LFSP-RAW(I to | 60,000 ± 20,000 | −5.7 ± 0.1 | −2.7 ± 0.3 | 3.0 ± 0.4, |
| | A) | | | | |

Results

Anti-EphB4-ephrinB2 therapeutic development can be accomplished by providing the three dimensional crystal structure of the EphB4-ephrinB2 complex at a high resolution. EphB4 specificity can also be probed using the three dimensional crystal structure. In addition, site-directed mutagenesis and biophysical analyses were conducted to investigate the role of several residues within the ligand binding cavity of EphB4 in contributing to the binding of both ephrinB2 and the antagonistic TNYL-RAW peptide. These results allow the development of predictive models for structure-based drug design of small molecule compounds for use as therapeutics and to probe the biology of EphB4-ephrinB2 bi-directional signaling.

Overall Structure

EphB4 and ephrinB2 were co-concentrated to 20 mg/mL and crystallized by sitting drop vapor diffusion against a precipitant of 2.2 M ammonium sulfate and 100 mM Tris, pH 7.8 at 20° C. The co-crystal structure was refined to 2.0 Å resolution with an R-factor of 22.6% and a free R factor of 29.5% (Table 1). Unlike crystals of the of the EphB2-ephrinB2 complex, which consisted of a heterotetramer, crystals of the EphB4-ephrinB2 complex consist of a heterodimer. Previously, formation of ephrinB2-EphB2 tetramers was observed for a concentration range around 1 mM using size exclusion chromatography analysis (SEC), while analytical ultracentrifugation demonstrated that the EphB2-ephrinB2 complex was a heterodimer at concentrations in the low micromolar range (10). The SEC analysis of the of the present invention provides the EphB4-ephrinB2 complex in a concentration range up to 500 μM indicating that the EphB4-ephrinB2 complex exsists as a heterodimer (data not shown). The overall structure of the EphB4-ephrinB2 complex is similar to that of the EphB2-ephrinB2 complex, with an r.m.s. deviation of 5.0 Å over 316 equivalent Cα positions. Significant deviation is evident, however, throughout the structure of the loop regions compared with the EphB4-TNYL-RAW and EphB2-ephrinB2 structures, with r.m.s. deviations of 1.8 and 5.3 Å, respectively in the J-K loop. The ephrinB2 ligand deviates minimally between previously described apo and receptor-bound structures, shifting only 0.91 and 0.90 Å respectively (10, 17).

EphB4-EphrinB2 Interface

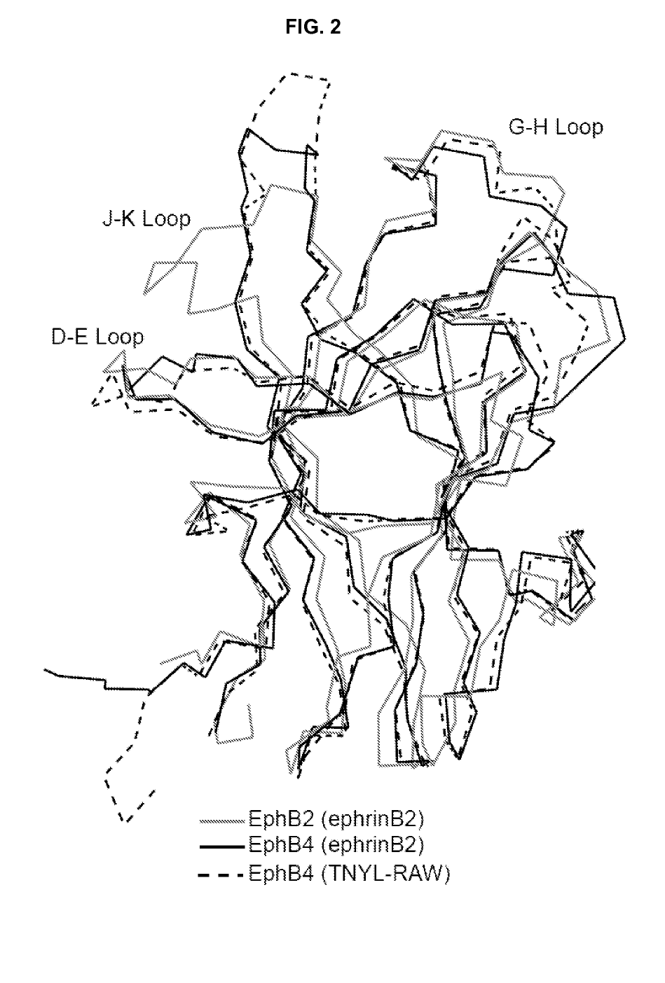

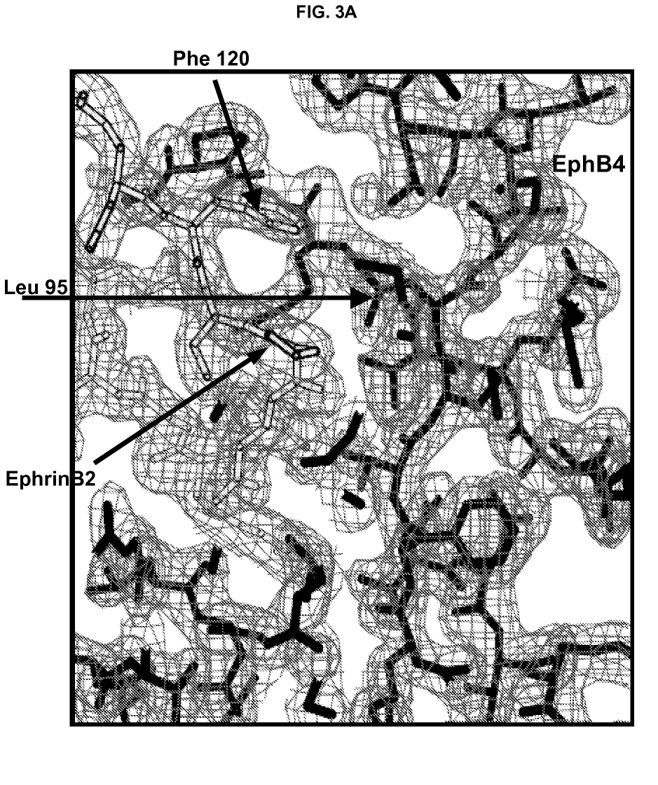

Although the overall shape of the EphB4-ephrinB2 interaction interface is in good agreement with that previously described in the EphB2-ephrinB2 structure, marked differences exist within the receptor loops that frame the ligand binding channel. The EphB4 J-K loop assumes a distinct position compared to previously described crystal structures, and is situated directly above the ligand G-H loop and 15 Å from the D-E loop (FIG. 2). The corresponding J-K loop from the EphB2-ephrinB2 structure, on the other hand, is shifted 6.4 Å from the D-E loop, and therefore maintains a more compact binding cavity. In fact, the J-K loops differ in position by up to 10 Å from furthest positions between the two ephrinB2-bound complex structures. Not surprisingly, the J-K loop shows remarkable flexibility in each structure described, also shifting in position by up to 20 Å from furthest positions between the EphB4-TNYL-RAW structure and the EphB4-ephrinB2 structure. Furthermore, crystallization trials with the apo form of EphB4 failed to produce diffracting crystals, likely because of the inherent flexibility of the J-K and D-E loops. A feature unique to EphB4 is a three residue insert in the J-K loop, which is absent in all other Eph receptors. It has been speculated that this insert contributes to the ligand binding specificity inherent to the EphB4 receptor (35). Indeed, two of the three residues (Pro-151, Gly-152, Ala-153) form the tip of the J-K loop, and make contacts with the ligand: Pro-151 R(R. receptor; L, ligand) forms a hydrophobic contact with Phe-120L, while Gly-152R makes a main-chain to side-chain polar contact with Glu-152L. In addition, the G-H and D-E loops, which form two walls of the ligand binding cleft, also shift in order to accommodate the ligand. The G-H loop is shifted by over 4.5 Å between the EphB4 and EphB2-bound ephrinB2 structures, while the D-E loop only deviates by 1.5 Å between the two structures.

The high affinity EphB4-ephrinB2 heterodimer is formed by insertion of the solvent exposed ligand G-H loop into the upper convex and hydrophobic surface of the EphB4 receptor, positioned above receptor strands E and M. Hydrophobic contacts drive receptor-ligand binding in this region. Ligand residues Phe-120, Pro-122, Trp-125 and Leu-127 participate in van der Waals interactions with receptor residues lining the receptor binding cavity in the D-E, G-H and J-K loops (FIG. 4). Phe-120L forms hydrophobic interactions with Leu-95R (see below), Leu-100R, and Pro-101R, while Leu-124L interacts with Thr-147R from the receptor J-K loop. Meanwhile, Trp-125L extends to the surface of the receptor, in-between the J-K and G-H loops, participating in hydrophobic interactions with residues Leu-48R, Glu-50R, Val-159R, Leu-188R, and Ala-186R. In addition, Pro-122L, similar to all previous crystal structures, maintains its position by participating in a direct interaction with the receptor Cys61-Cys-184 disulfide bridge. Few polar contacts are formed at the receptor-ligand dimer interface. Ser-121 L forms a side-chain side-chain hydrogen bond with Glu-59R as well as a main-chain side-chain hydrogen bond with Lys-149R, while Asn-123L forms a hydrogen bond with the main-chain oxygen of Leu-48R. Additionally, Lys-149R extends to the body of the ephrinB2 G-H loop, forming side-chain side-chain hydrogen bonds with Glu-128L, and side-chain main-chain hydrogen bonds with Ser-121L and Asn-123L, wich are both part of the high affinity ligand FSPN sequence (FIG. 3). The introduction of this new interaction at the EphB4-ephrinB2 interface is certain to contribute to the high affinity of this receptor-ligand complex.

Similar to the EphB2-ephrinB2 structure, a second portion of the high affinity heterodimerization interface exists immediately adjacent to the ligand binding cavity, formed by ligand strands C, G. and F. and receptor strands B-C, E, and D. This region of the complex deviates minimally from the corresponding structure of in the EphB2-ephrinB2 complex, with a maximum of 2.1 Å from furthest atoms, and is predominantly characterized by backbone-backbone, backbone-sidechain, and sidechain sidechain hydrogen bonds. In particular, sidechain-sidechain interactions between Glu-59R (Glu-68 in EphB2)-Gln-118L and Ser-121 L, Asp-29R (Glu-40 in EphB2)-Lys-112L, and Glu-43 (Glu-52, EphB2)-Lys 116L provide the binding potential characteristic of this low nanomolar interaction. Sidechain-mainchain interactions between Ser-55 and Lys-116L, and Glu-44R and Lys-60R complete the binding network in this region.

EphB4 Specificity

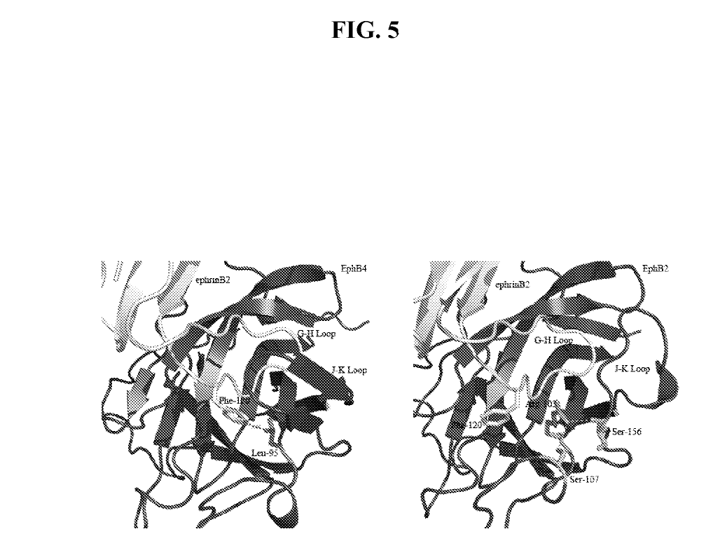

Sequence comparison and structural analysis of the EphB4 and EphB2 receptors suggested that one residue in EphB4 is particularly important in determining the specificity of the EphB4-ephrinB2 interaction: Leu-95. The corresponding residue in EphB2, Arg-103, is strictly conserved across both A and B subclasses, and deviates only in the EphB4 receptor. Arg-103R participates in hydrogen bonds with residues from the high affinity ephrin G-H loop, including Ser-121L and Glu-128L, and is situated in proximity to Phe-120L, a residue critical for receptor binding. However, superposition of Arg to Leu-95 in the EphB4-ephrinB2 structure suggests that a steric clash would result between an arginine at position 95R and Phe-120L. The corresponding Leu-95R, on the other hand, is able to form a 3.2 Å van der Waals interaction with Phe-120L due to its position within the ligand binding cavity. Thus, Arg-95R would also sterically clash with the phenylalanine from the TNYL-RAW peptide in the EphB4-TNYL-RAW structure (19), while the smaller Leu-95R forms favorable contacts with the peptide. Interestingly, the highly conserved Phe-120L is shifted in position by ˜90° as compared to previous complex structures (8, 10, 19) (FIG. 5) and is buried within the hydrophobic cleft of the receptor, unlike its position in the EphB2-ephrinB2 complex structure, where it is directed towards the surface. In addition, the position of Arg-103R requires the J-K loop of the EphB2 receptor to extend away from the ligand G-H loop and towards the receptor D-E loop to avoid steric interference with residues lining the ephrin-B2 G-H loop. The smaller Leu-95R, together with the Phe-120L, allows the J-K loop of EphB4 to adopt a novel position directly above the ligand G-H loop.

Biophysical Characterization of EphB4 Specificity: Enthalpic vs. Entropic Contributions

A series of site-specific mutations was generated by changing residues lining the EphB4 G-H and J-K loops to the corresponding residues found in EphB2 (Table 3). The EphB4 mutants were rank-ordered based on their binding to fluorescently labeled Alexa-532-TNYL-RAW peptide. Fluorescence Polarization (FP) analysis corroborated the prediction that Leu-95 is a critical determinant for binding of the TNYL-RAW peptide because the Leu95Arg mutant did not exhibit significant binding of the peptide in our assay. EphB4 mutants Thr147Phe, Ala186Ser and Lys149Gln showed approximately 4-5 fold reduction in binding affinity of the fluorescently labeled peptide. The reduction in affinity due to mutation of these residues is consistent with what would be expected based on the structural information. A Thr-147-Phe mutation would impose steric constraints between the receptor J-K loop and the ephrinB2 G-H loop, as well as with Leu-95R due to the position of the receptor J-K loop. Interestingly, EphB4 possesses an alanine at position 186, which is conserved across the A-subclass while other B-subclass receptors have a conserved Ser. Ala-186R forms a van der Waals interaction with the main chain carbon of Asn-123 of the high affinity ligand G-H loop. A Ser at position 186 of EphB4 would cause a polar redistribution at the heterodimerization interface with ephrinB2 and result in the displacement of the receptor G-H loop due to a steric clash with Thr-93L and potential displacement of the ephrinB2 G-H loop. Finally, Lys-149R forms interactions at the dimer interface with ephrinB2 residues Ser-121L, Asn-123L and Gln-128L. Mutation to Gln should not result in steric interference with the ligand G-H loop, but could result in a slight readjustment of the J-K loop in order to accommodate the bulkier Gln side chain.

Based on the structural information and preliminary binding characterization, two EphB4 mutants, Leu95Arg and Lys149Gln, were chosen for detailed thermodynamic analysis of their binding to both ephrinB2 and the TNYL-RAW peptide ligand using isothermal titration calorimetry (ITC). As reported previously, EphB4 binds to ephrinB2 with an affinity of 40 nM and a ΔH_{obs} of 3.3 kcal mol⁻¹ (19). Mutation of EphB4 Lys-149 to Gln has no effect on the binding affinity or enthalpy of ephrinB2 binding (Table 4). In contrast, mutation of EphB4 Leu-95 to Arg reduces the binding affinity of ephrinB2 by nearly two orders of magnitude. Binding of ephrinB2 to all forms of EphB4 is endothermic, and the binding of ephrinB2 is more endothermic with the L95R mutation in EphB4 (5.2 kcal mol⁻¹ versus 3.3 kcal mol⁻¹ for wild-type EphB4). Preliminary experiments carried out in a buffer with different enthalpy of ionization showed a similar enthalpy change to that reported here. For example, binding of ephrinB2 to EphB4 (K149Q) results in a ΔH_{obs} of 3.9 (±0.1) kcal mol⁻¹ in phosphate (ΔHᵢₒₙ=0.8 kcal mol⁻¹) compared to the ΔH_{obs} of 3.7 (+0.2) kcal mol⁻¹ value obtained in Tris (ΔHᵢₒₙ=11.34 kcal mol⁻¹) (Table 4). Thus, the protonation/deprotonation is not coupled to ephrinB2 binding under the conditions of the ITC experiments.

Binding of the TNYL-RAW peptide to the wild-type, Lys149Gln, and Leu95Arg forms of EphB4 was also monitored by ITC. TNYL-RAW binds to EphB4 with an affinity of 70 nM and a ΔH_{obs} of −14.7 kcal mol⁻¹ (19). In contrast to the different effects of mutations in EphB4 on the interaction of EphB4 with ephrinB2, mutation of EphB4 of either Lys-149 to Gln or Leu-95 to Arg reduces the affinity of the EphB4-TNYL-RAW interaction (three-fold and 500-fold, respectively; Table 4). Binding of the TNYL-RAW peptide to all three forms of EphB4 is characterized by an exothermic enthalpy.

Thus, thermodynamic analysis reveals that TNYL-RAW binding to the EphB4 ligand binding domain is an enthalpically driven process, while ephrinB2 binding to EphB4 is an entropically driven process. The differences in the binding thermodynamics are consistent with the available structural information. Burial of the hydrophobic ligand G-H loop within the hydrophobic receptor binding cleft could entropically drive the interaction through the release of water, increasing the solvent entropy. In addition, the ephrinB2 ligand G-H loop is quite rigid, both in apo and receptor-bound structures, minimizing massive conformational entropy losses. The small loss of conformational entropy counteracts the heterodimerization process by ordering the otherwise flexible receptor J-K, D-E, and G-H loops. Unlike ephrinB2, however, the free peptide ligand loses significant conformational degrees of freedom upon EphB4 binding, resulting in an entropy loss. This is compensated by an enthalpic gain due to the formation of favorable interactions, both polar and nonpolar, at the receptor-peptide interface.

It should be noted that we produced the ephrinB2 extracellular domain in insect cells in a glycosylated form, while the ephrins used for previous crystal structure determinations were produced in E. coli and therefore not glycosylated. A conserved glycosylation site exists in ephrinB2 at residue Asn-39, in proximity of the low affinity tetramerization interface. Consistent with its possible glycosylation, Asn-39 is located near the surface of the protein and its side chain extends toward the surface of the complex. Although the carbohydrate was not observed in our electron density map, most likely because it was disordered, there is a theoretical possibility that a sugar at this location could interfere with the formation of receptor-ligand tetramers in the crystal lattice. However, previous reports have suggested that carbohydrate moieties would play more a favorable than an unfavorable role in tetramerization (36).

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

Citation of a reference herein shall not be construed as an admission that such is prior art to the present invention. Specifically intended to be within the scope of the present invention, and incorporated herein by reference in its entirety, is the following publication: Chrencik, J. E., Brooun, A., Kraus, M. L., Recht, M. I., Kolatkar, A. R., Han, G. W., Seifert, J. M., Widmer, H., Auer, M., Kuhn, P. Structural and Biophysical Characterization of the EphB4-EphrinB2 Protein-Protein Interaction and Receptor Specificity. (2006) J. Biol. Chem. 281:28185-28192.

Publications referred to herein include:
1. Dodelet, V. C., and Pasquale, E. B. (2000) Oncogene 19, 5614-5619
2. Pasquale, E. B. (2005) Nat Rev Mol Cell Biol 6, 462-475
3. Wilkinson, D. G. (2001) Nat Rev Neurosci 2, 155-164
4. Wilkinson, D. G. (2000) Int Rev Cytol 196, 177-244
5. Blits-Huizing a, C. T., Nelersa, C. M., Malhotra, A., and Liebl, D. J. (2004) IUBMB Life 56, 257-265
6. Gale, N. W., Holland, S. J., Valenzuela, D. M., Flenniken, A., Pan, L., Ryan, T. E., Henkemeyer, M., Strebhardt, K., Hirai, H., Wilkinson, D. G., Pawson, T., Davis, S., and Yancopoulos, G. D. (1996) Neuron 17, 9-19
7. Committee, E. N. (1997) Cell 90, 403-404
8. Himanen, J. P., Chumley, M. J., Lackmann, M., Li, C., Barton, W. A., Jeffrey, P. D., Vearing, C., Geleick, D., Feldheim, D. A., Boyd, A. W., Henkemeyer, M., and Nikolov, D. B. (2004) Nat Neurosci 7, 501-509
9. Takemoto, M., Fukuda, T., Sonoda, R., Murakami, F., Tanaka, H., and Yamamoto, N. (2002) Eur J Neurosci 16, 1168-1172
10. Himanen, J. P., Rajashankar, K. R., Lackmann, M., Cowan, C. A., Henkemeyer, M., and Nikolov, D. B. (2001) Nature 414, 933-938
11. Hopkins, A. L., Mason, J. S., and Overington, J. P. (2006) Curr Opin Struct Biol 16, 127-136
12. Holland, S. J., Gale, N. W., Mbamalu, G., Yancopoulos, G. D., Henkemeyer, M., and Pawson, T. (1996) Nature 383, 722-725
13. Schmucker, D., and Zipursky, S. L. (2001) Cell 105, 701-704
14. Kalo, M. S., and Pasquale, E. B. (1999) Biochemistry 38, 14396-14408
15. Davy, A., Gale, N. W., Murray, E. W., Klinghoffer, R. A., Soriano, P., Feuerstein, C., and Robbins, S. M. (1999) Genes Dev 13, 3125-3135
16. Chin-Sang, I. D., George, S. E., Ding, M., Moseley, S. L., Lynch, A. S., and Chisholm, A. D. (1999) Cell 99, 781-790
17. Toth, J., Cutforth, T., Gelinas, A. D., Bethoney, K. A., Bard, J., and Harrison, C. J. (2001) Dev Cell 1, 83-92
18. Himanen, J. P., Henkemeyer, M., and Nikolov, D. B. (1998) Nature 396, 486-491
19. Chrencik, J. E., Brooun, A., Recht, M. I., Kraus, M. L., Koolpe, M., Kolatkar, A. R., Bruce, R. H., Martiny-Baron, G., Widmer, H., Pasquale, E. B., and Kuhn, P. (2006) Structure 14, 321-330
20. Nakamoto, M., and Bergemann, A. D. (2002) Microsc Res Tech 59, 58-67
21. Liu, W., Ahmad, S. A., Jung, Y. D., Reinmuth, N., Fan, F., Bucana, C. D., and Ellis, L. M. (2002) Cancer 94, 934-939
22. Berclaz, G., Karamitopoulou, E., Mazzucchelli, L., Rohrbach, V., Dreher, E., Ziemiecki, A., and Andres, A. C. (2003) Ann Oncol 14, 220-226
23. Andres, A. C., Reid, H. H., Zurcher, G., Blaschke, R. J., Albrecht, D., and Ziemiecki, A. (1994) Oncogene 9, 1461-1467
24. Nikolova, Z., Djonov, V., Zuercher, G., Andres, A. C., and Ziemiecki, A. (1998) J Cell Sci 111 (Pt 18), 2741-2751
25. Munarini, N., Jager, R., Abderhalden, S., Zuercher, G., Rohrbach, V., Loercher, S., Pfanner-Meyer, B., Andres, A. C., and Ziemiecki, A. (2002) J Cell Sci 115, 25-37
26. Berclaz, G., Andres, A. C., Albrecht, D., Dreher, E., Ziemiecki, A., Gusterson, B. A., and Crompton, M. R. (1996) Biochem Biophys Res Commun 226, 869-875
27. Noren, N. K., Lu, M., Freeman, A. L., Koolpe, M., and Pasquale, E. B. (2004) Proc Natl Acad Sci USA 101, 5583-5588
28. Kertesz, N., Krasnoperov, V., Reddy, R., Leshanski, L., Kumar, S. R., Zozulya, S., and Gill, P. S. (2006) Blood 107, 2330-2338
29. Martiny-Baron, G., Korff, T., Schaffner, F., Esser, N., Eggstein, S., Marme, D., and Augustin, H. G. (2004) Neoplasia 6, 248-257
30. Koolpe, M., Burgess, R., Dail, M., and Pasquale, E. B. (2005) J Biol Chem 280, 17301-17311
31. Otwinowski, Z., Minor, W. (1997) Processing of x-ray diffraction data collected in oscillation mode. Methods in Enzymology, Macromolecular Crystallography, part A (Sweet, C. W. C. R. M., Ed.), 276, Academic Press, New York
32. CCP4. (1994) Acta Crystallogr D Biol Crystallogr 50, 760-763
33. Jones, T. A., Zou, J. Y., Cowan, S. W., and Kjeldgaard. (1991) Acta Crystallogr A 47 (Pt 2), 110-119
34. Turnbull, W. B., and Daranas, A. H. (2003) J Am Chem Soc 125, 14859-14866
35. Nikolov, D. B., Li, C., Barton, W. A., and Himanen, J. P. (2005) Biochemistry 44, 10947-10953
36. Himanen, J. P., and Nikolov, D. B. (2002) Acta Crystallogr D Biol Crystallogr 58, 533-535
37. Day, B., To, C., Himanen, J. P., Smith, F. M., Nikolov, D. B., Boyd, A. W., and Lackmann, M. (2005) J Biol Chem 280, 26526-26532
38. Smith, F. M., Vearing, C., Lackmann, M., Treutlein, H., Himanen, J., Chen, K., Saul, A., Nikolov, D., and Boyd, A. W. (2004) J Biol Chem 279, 9522-9531

What is claimed is:

1. A method for selecting a candidate drug which interferes with an activity of an EphB4 receptor, the method comprising:
(a) providing a three-dimensional crystal structure of the EphB4 receptor in complex with an ephrinB2; and
(b) designing a compound predicted to bind the EphB4 receptor configured in the EphB4 receptor and ephrinB2 complex.

2. A method for designing a compound which interferes with an activity of an EphB4 receptor, the method comprising:
(a) providing on a digital computer a three-dimensional structure of a receptor-ligand complex comprising the EphB4 receptor and an ephrinB2; and
(b) using software comprised by the digital computer to design a compound which is predicted to bind to the EphB4 receptor.

3. A method according to claim 2, further comprising:
(c) synthesizing the compound; and
(d) evaluating the compound for an ability to interfere with an activity of the EphB4 receptor.

4. A method according to claim 2, wherein the compound is designed by computational interaction with reference to a three-dimensional site of the structure of the receptor-ligand complex, wherein the three-dimensional site is selected from the group consisting of EphB4 D-E and J-K loops.

5. A method according to claim 4, wherein the three-dimensional site comprises Leu-95 of SEQ ID NO: 4.

6. A method according to claim 2, wherein the EphB4 receptor is a human EphB4 receptor.

7. A method for generating a model of a three-dimensional structure of an EphB-ligand complex, the method comprising:
(a) providing an amino acid sequence of a reference EphB4 protein, atomic coordinates of the reference EphB4 protein, and an amino acid sequence of a target EphB, wherein the reference EphB4 is comprised by an EphB4-ephrinB2 complex;
(b) identifying one or more structurally conserved regions shared between the reference EphB4 amino acid sequence and the target EphB amino acid sequence; and
(c) assigning atomic coordinates from the one or more structurally conserved regions to a complex comprising the target EphB and an EphB ligand.

8. A method in accordance with claim 7, wherein the EphB4 protein comprises an amino acid sequence as set forth in SEQ ID NOs: 2 or 3.

9. A method in accordance with claim 7, wherein the EphB4 protein consists essentially of an amino acid sequence as set forth in SEQ ID NOs: 2 or 3.

10. A method in accordance with claim 7, wherein the reference EphB4-ephrinB2 complex comprises a three-dimensional structure described by atomic coordinates that substantially conform to atomic coordinates set forth in Table 1.

11. A method according to claim 7, wherein the EphB4 polypeptide is a human EphB4 polypeptide.

12. A method for generating a model of a three-dimensional structure of an EphB receptor-ligand complex, the method comprising:
(a) providing an amino acid sequence of a known EphB4 receptor in complex with ephrinB2;
(b) providing an amino acid sequence of a target EphB receptor;
(c) identifying structurally conserved regions shared between the known receptor-ligand complex amino acid sequence and the target receptor-ligand complex amino acid sequence; and
(d) assigning atomic coordinates of the conserved regions to the target receptor-ligand complex.

13. A method in accordance with claim 12, wherein the known EphB4 receptor comprises an amino acid sequence as set forth in SEQ ID NOs: 2 or 3.

14. A method in accordance with claim 12, wherein the known EphB4 receptor consists essentially of an amino acid sequence as set forth in SEQ ID NOs: 2 or 3.

15. A method according to claim 12, wherein the known EphB4 receptor is a human EphB4 receptor.

16. A method according to claim 12, wherein the known receptor-ligand complex comprises a three-dimensional structure described by atomic coordinates that substantially conform to atomic coordinates set forth in Table 1.

17. A crystal comprising an EphB4 ligand binding domain and ephrinB2.

18. A crystal according to claim 17, wherein the EphB4 ligand binding domain is a polypeptide having a sequence of SEQ ID NOs: 2 or 3.

19. A crystal according to claim 17, wherein the EphB4 ligand binding domain consists essentially of EphB4 D-E and J-K loops.

20. A crystal according to claim 17, wherein the EphB4 ligand binding domain consists essentially of Leu-48, Cys-61, Leu-95, Ser-99 Leu-100, Pro-101, Thr-147, Lys-149, Ala-155, and Cys-184 of SEQ ID NO: 4.

21. A crystal according to claim 17, wherein the EphB4 ligand binding domain is a human EphB4 ligand binding domain.

22. A crystal according to claim 17, wherein the ephrinB2 comprises Phe-120, Pro-122, Leu-124, Trp-125, and Leu-127 of SEQ ID NO: 6.

23. A crystal in accordance with claim 17, wherein the crystal comprises space group P4₁ so as to form a unit cell of dimensions a=81.09 Å, b=81.09 Å, and c=50.95 Å.

24. A crystal comprising a polypeptide having SEQ ID NOs: 2 or 3 complexed with an ephrinB2, wherein the crystal is sufficiently pure to determine atomic coordinates of the complex by X-ray diffraction to a resolution of about 1.65 Å.

25. A crystal according to claim 24, wherein the ephrinB2 comprises Phe-120, Pro-122, Leu-124, Trp-125, and Leu-127 of ephrinB2.

26. A polypeptide having SEQ ID NOs: 2 or 3 in complex with ephrinB2.

27. A complex according to claim 26, wherein the ephrin-B2 comprises Phe-120, Pro-122, Leu-124, Trp-125, and Leu-127.

28. A therapeutic compound that inhibits an activity of an EphB4 receptor, wherein the compound is selected by
a) performing a structure based drug design using a three-dimensional structure determined for a crystal comprising an EphB4 receptor and ephrinB2;
b) contacting a sample comprising the EphB4 receptor with the compound, and
c) detecting inhibition of at least one activity of the EphB4 receptor.

29. A compound according to claim 28, wherein the EphB4 is a polypeptide having SEQ ID NOs: 2 or 3.

30. A compound according to claim 28, wherein the EphB4 receptor is a human EphB4 receptor.

31. A three-dimensional computer image of the three-dimensional structure of an EphB4-ephrinB2 complex, wherein the structure substantially conforms to the three-dimensional coordinates listed in Table 1.

32. A computer-readable medium encoded with a set of three-dimensional coordinates set forth in Table 1, wherein, using a graphical display software program, the three-dimensional coordinates of Table 1 create an electronic file that can be visualized on a computer capable of representing said electronic file as a three-dimensional image.

33. A computer-readable medium encoded with a set of three-dimensional coordinates of a three-dimensional structure which substantially conforms to the three-dimensional coordinates represented in Table 1, wherein, using a graphical display software program, the set of three-dimensional coordinates create an electronic file that can be visualized on a computer capable of representing said electronic file as a three-dimensional image.

34. A method for crystallizing an EphB4 receptor, the method comprising:
a) providing an EphB4 receptor in contact with an ephrinB2; and
b) contacting the EphB4 receptor in contact with the polypeptide with a compound of claim 27,
wherein the EphB4 receptor in contact with the ephrinB2 and the compound forms an EphB4 receptor crystal.

35. A composition comprising EphB4 receptor, an ephrinB2, and a compound of claim 54.

36. A composition according to claim 28, wherein the EphB4 receptor is a polypeptide having SEQ ID NOs: 2 or 3.

37. A composition according to claim 36, wherein the EphB4 receptor consists essentially of EphB4 D-E and J-K loops.

38. A composition according to claim 36, wherein the EphB4 receptor consists essentially of Leu-48, Cys-61, Leu-95, Ser-99 Leu-100, Pro-101, Thr-147, Lys-149, Ala-155, and Cys-184 of SEQ ID NO: 4.

39. A composition according to claim 36, wherein the EphB4 receptor is a human EphB4 receptor.

40. A composition according to claim 36, wherein the ephrinB2 is a polypeptide having SEQ ID NO: 6.

41. A mutant EphB4 which binds ephrinB2, wherein the affinity for ephrinB2 is less than the affinity of wild type EphB4 for ephrinB2.

42. A mutant EphB4 in accordance with claim 41, comprising at least one mutation selected from the group consisting of T147F, K149Q and A186S.

43. A mutant EphB4 in accordance with claim 41, wherein the mutant EphB4 binds an EphB4 ligand, and wherein the dynamic range of binding of the ligand is greater than that of the ligand binding to wild type EphB4.

44. A mutant EphB4 in accordance with claim 43, wherein the dynamic range of ligand binding is about 2-fold.

45. A method of assaying a candidate compound for mutant EphB4 binding activity, the method comprising:
contacting a complex comprising a mutant EphB4 and a labeled EphB4 ligand; and
detecting release of the labeled EphB4 ligand from the complex.

46. A method of assaying a candidate compound for EphB4 binding activity, the method comprising:
contacting a complex comprising an EphB4 and an EphB4 ligand which comprises a fluorescent label with the candidate compound; and
determining fluorescence polarization, wherein a shift in fluorescence polarization indicates EphB4 binding activity.

47. A high throughput assay method for screening candidate compounds for EphB4 binding activity, comprising testing a plurality of candidate compounds for EphB4 binding activity, wherein each compound is tested by the method of claim 45.
